SEQUENCE LISTING (624 sequences)

SEQ ID NO: 1 - DNA, 461 bp, Homo sapiens
ttagagaggcacagaaggaagaagagttaaaagcagcaaagccgggtttttttgttttgttttgttttgttttgttttgagatggagtctcactctgttgcccaagctggagtacaacggcatgatctcagctcgctgcaacctccgcctcccacgttcaagtgattctcctgcctcagcctcccaagtagctgggattacaggcgcccgccaccacgctcagctaattttttttgtatttttagtagagacagggtttcaccaggttggccaggctgctcttgaactcctgacctcaggtgatccacccgcctcggcctcccaaagtgctgggattacaggcgtgagccaccacgcccggcccccaaagctgtttcttttgtctttagcgtaaagctctcctgccatgcagtatctacataactgacgtgactgccagcaagctcagtcactccgtggtc

SEQ ID NO: 2 - DNA, 540 bp, Homo sapiens
taggatgtgttggaccctctgtgtcaaaaaaaacctcacaaagaatcccctgctcattacagaagaagatgcatttaaaatatgggttattttcaactttttatctgaggacaagtatccattaattattgtgtcagaagagattgaatacctgcttaagaagcttacagaagctatgggaggaggttggcagcaagaacaatttgaacattataaaatcaactttgatgacagtaaaaatggcctttctgcatgggaacttattgagcttattggaaatggacagtttagcaaaggcatggaccggcagactgtgtctatggcaattaatgaagtctttaatgaacttatattagatgtgttaaagcagggttacatgatgaaaaagggccacagacggaaaaactggactgaaagatggtttgtactaaaacccaacataatttcttactatgtgagtgaggatctgaaggataagaaaggagacattctcttggatgaaaattgctgtgtagagtccttgcctgacaaagatggaaa

SEQ ID NO: 3 - DNA, 461 bp, Homo sapiens; identical to SEQ ID NO: 1

SEQ ID NO: 4 - DNA, 531 bp, Homo sapiens
  misc_feature 454, 492, 526: n = A,T,C or G
tctttttctttcgatttccttcaatttgtcacgtttgattttatgaagttgttcaagggctaactgctgtgtattatagctttctctgagttccttcagctgattgttaaatgaatccatttctgagagcttagatgcagtttctttttcaagagcatctaattgttctttaagtctttggcataattcttccttttctgatgactttttatgaagtaaactgatccctgaatcaggtgtgttactgagctgcatgtttttaattctttcgtttaatagctgcttctcagggaccagatagataagcttattttgatattccttaagctcttgttgaagttgtttgatttccataatttccaggtcacactgtttatccaaaacttctagctcagtcttttgtgtttgctttctgatttggacatcttgtagtctgcctgagatctgctgatgntttccattcactgcttccagttccaggtggagactttnctttctggagctcagcctgacaatgccttcttgntccct

SEQ ID NO: 5 - DNA, 531 bp, Homo sapiens
agccagatggctgagagctgcaagaagaagtcaggatcatgatggctcagtttcccacagcgatgaatggagggccaaatatgtgggctattacatctgaagaacgtactaagcatgataaacagtttgataacctcaaaccttcaggaggttacataacaggtgatcaagcccgtacttttttcctacagtcaggtctgccggccccggttttagctgaaatatgggccttatcagatctgaacaaggatgggaagatggaccagcaagagttctctatagctatgaaactcatcaagttaaagttgcagggccaacagctgcctgtagtcctccctcctatcatgaaacaaccccctatgttctctccactaatctctgctcgttttgggatgggaagcatgcccaatctgtccattcatcagccattgcctccagttgcacctatagcaacacccttgtcttctgctacttcagggaccagtattcctcccctaatgatgcctgctcccctagtgccttctgttagta

SEQ ID NO: 6 - DNA, 531 bp, Homo sapiens
aatagatttaatgcagagtgtcaacttcaattgattgatagtggctgcctagagtgctgtgttgagtaggtttctgaggatgcaccctggcttgaagagaaagactggcaggattaacaatatctaaaatctcacttgtaggagaaaccacaggcaccagagctgccactggtgctggcaccagctccaccaaggccagcgaagagcccaaatgtgagagtggcggtcaggctggcaccagcactgaagccaccactggtgctggcactggcactggcactgttattggtactggtactggcaccagtgctggcactgccactctcttgggctttggctttagcttctgctcccgcctggatccgggctttggcccagggtccgatatcagcttcgtcccagttgcagggcccggcagcattctccgagccgagcccaatgcccattcgagctctaatctcggccctagccttggcttcagctgcagcctcagctgcagccttcaaatccgcttccatcgcctctcggtac

SEQ ID NO: 7 - DNA, 531 bp, Homo sapiens
gccaagaaagcccgaaaggtgaagcatctggatggggaagaggatggcagcagtgatcagagtcaggcttctggaaccacaggtggccgaagggtctcaaaggccctaatggcctcaatggcccgcagggcttcaaggggtcccatagccttttgggcccgcagggcatcaaggactcggttggctgcttgggcccggagagccttgctctccctgagatcacctaaagcccgtaggggcaaggctcgccgtagagctgccaagctccagtcatcccaagagcctgaagcaccaccacctcgggatgtggcccttttgcaagggagggcaaatgatttggtgaagtaccttttggctaaagaccagacgaagattcccatcaagcgctcggacatgctgaaggacatcatcaaagaatacactgatgtgtaccccgaaatcattgaacgagcaggctattccttggagaaggtatttgggattcaattgaaggaaattgataagaatgaccacttgtacattcttctcagc

SEQ ID NO: 8 - DNA, 531 bp, Homo sapiens
  misc_feature 481: n = A,T,C or G
gaggtctcactatgttgcccaggctgttcttgaactcctgggatcaagcaatccacccatgttggtctccaaaagtgctgggatcataggcgtgagccacctcacccagccaccaattttcaatcaggaagactttttccttcttcaagaagtgaagggtttccagagtatagctacactattgcttgcctgagggtgactacaaaattgcttgctaaaaggttaggatgggtaaagaattagattttctgaatgcaaaaataaaatgtgaactaatgaactttaggtaatacatattcataaaataattattcacatatttcctgatttatcacagaaataatgtatgaaatgctttgagtttcttggagtaaactccattactcatcccaagaaaccatattataagtatcactgataataagaacaacaggaccttgtcataaattctggataagagaaatagtctctgggtgtttgntcttaattgataaaatttacttgtccatcttttagttcagaatcacaaaa

SEQ ID NO: 9 - DNA, 531 bp, Homo sapiens
  misc_feature 528: n = A,T,C or G
aagcggaaatgagaaaggagggaaaatcatgtggtattgagcggaaaactgctggatgacagggctcagtcctgttggagaactctgggtggtgctgtagaacagggccactcacagtggggtgcacagaccagcacggctctgtgacctgtttgttacaggtccatgatgaggtaaacaatacactgagtataagggttggtttagaaactcttacagcaatttgacaaagtaatcttctgtgcagtgaatctaagaaaaaaattggggctgtatttgtatgttcctttttttcatttcatgttctgagttacctatttttattgcattttacaaaagcatccttccatgaaggaccggaagttaaaaacaaagcaggtcctttatcacagcactgtcgtagaacacagttcagagttatccacccaaggagccagggagctgggctaaaccaaagaattttgcttttggttaatcatcaggtacttgagttggaattgttttaatcccatcattaccaggctggangtg

SEQ ID NO: 10 - DNA, 861 bp, Homo sapiens
ccgcggctcctgtccagaccctgaccctccctcccaaggctcaaccgtcccccaacaaccgccagccttgtactgatgtcggctgcgagagcctgtgcttaagtaagaatcaggccttattggagacattcaagcaaaggttggacaactacttttccagaacagaaaggaaactcatgcatcagaaaaggtgactaataaaggtaccagaagaatatggctgcacaaataccagaatctgatcagataaaacagtttaaggaatttctggggacctacaataaacttacagagacctgctttttggactgtgttagagacttcacaacaagagaagtaaaacctgaagagaccacctgttcagaacattgcttacagaaatatttaaaaatgacacaaagaatatccatgagatttcaggaatatcatattcagcagaatgaagccctggcagccaaagcaggactccttggccaaccacgatagagaagtcctgatggatgaacttttgatgaaagattgccaacagctgctttattggaaatgaggactcatctgatagaatcccctgaaagcagtagccaccatgttcaaccatctgtcatgactgtttggcaaatggaaaccgctggagaaacaaaattgctatttaccaggaataatcacaatagaaggtcttattgttcagtgaaataataagatgcaacatttgttgaggccttatgattcagcagcttggtcacttgattagaaaaataaaccattgtttcttcaattgtgactgttaattttaaagcaacttatgtgttcgatcatgtatgagatagaaaaatttttattactcaaagtaaaataaatgga

SEQ ID NO: 11 - DNA, 541 bp, Homo sapiens
gaaaaaaaatataaaacacacttttgcgaaaacggtggccctaaaagaggaaaagaatttcaccaatataaatccaattttatgaaaactgacaatttaatccaagaatcacttttgtaaatgaagctagcaagtgatgatatgataaaataaacgtggaggaaataaaaacacaagacttggcataagatatatccacttttgatattaaacttgtgaagcatattcttcgacaaattgtgaaagcgttcctgatcttgcttgttctccatttcaaataaggaggcatatcacatcccaagagtaacagaaaaagaaaaaagacatttttgcattttgagatgaaccaaagacacaaaacaaaacgaacaaagtgtcatgtctaattctagcctctgaaataaaccttgaacatctcctacaaggcaccgtgatttttgtaattctaacctgaagaaatgtgatgacttttgtggacatgaaaatcagatgagaaaactgtggtctttccaaagcctgaactcccctgaaaacctttgca

SEQ ID NO: 12 - DNA, 541 bp, Homo sapiens
ctgggatcatttctcttgatgtcataaaagactcttcttcttcctcttcatcctcttcttcatcctcttctgtacagtgctgccgggtacaacggctatctttgtctttatcctgagatgaagatgatgcttctgtttctcctaccataactgaagaaatttcgctggaagtcgtttgactggctgtttctctgacttcaccttctttgtcaaacctgagtctttttacctcatgcccctcagcttccacagcatcttcatctggatgtttatttttcaaagggctcactgaggaaacttctgattcagaggtcgaagagtcactgtgatttttctcctcattttgctgcaaatttgcctctttgctgtctgtgctctcaggcaacccatttgttgtcatgggggctgacaaagaaacctttggtcgattaagtggcctgggtgtcccaggcccatttatattagacctctcagtatagcttggtgaatttccaggaaacataacaccattcattcgatttaaactattggaattggtttt

SEQ ID NO: 13 - DNA, 441 bp, Homo sapiens
gagggttggtggtagcggcttggggaggtgctcgctctgtcggtcttgctctctcgcacgcttcccccggctcccttcgtttcccccccccggtcgcctgcgtgccggagtgtgtgcgagggagggggagggcgtcgggggggtggggggaggcgttccggtccccaagagacccgcggagggaggcggaggctgtgagggactccgggaagccatggacgtcgagaggctccaggaggcgctgaaagattttgagaagagggggaaaaaggaagtttgtcctgtcctggatcagtttctttgtcatgtagccaagactggagaaacaatgattcagtggtcccaatttaaaggctattttattttcaaactggagaaagtgatggatgatttcagaacttcagctcctgagccaagaggtcctcccaaccctaatgtcga

SEQ ID NO: 14 - DNA, 131 bp, Homo sapiens
  misc_feature 126: n = A,T,C or G
aagcaggcggctcccgcgctcgcagggccgtgccacctgcccgcccgcccgctcgctcgctcgcccgccgcgccgcgctgccgaccgccagcatgctgccgagagtgggctgccccgcgctgccgntgccg

SEQ ID NO: 15 - DNA, 692 bp, Homo sapiens
atctcttgtatgccaaatatttaatataaatctttgaaacaagttcagatgaaataaaaatcaaagtttgcaaaaacgtgaagattaacttaattgtcaaatattcctcattgccccaaatcagtattttttttatttctatgcaaaagtatgccttcaaactgcttaaatgatatatgatatgatacacaaaccagttttcaaatagtaaagccagtcatcttgcaattgtaagaaataggtaaaagattataagacaccttacacacacacacacacacacacacgtgtgcacgccaatgacaaaaaacaatttggcctctcctaaaataagaacatgaagacccttaattgctgccaggagggaacactgtgtcacccctccctacaatccaggtagtttcctttaatccaatagcaaatctgggcatatttgagaggagtgattctgacagccacgttgaaatcctgtggggaaccattcatgtccacccactggtgccctgaaaaaatgccaataatttttcgctcccacttctgctgctgtctcttccacatcctcacatagaccccagacccgctggcccctggctgggcatcgcattgctggtagagcaagtcataggtctcgtctttgacgtcacagaagcgatacaccaaattgcctggtcggtcattgtcataaccagaga

SEQ ID NO: 16 - DNA, 728 bp, Homo sapiens
cagacggggtttcactatgttggctaggctggtcttgaactcctgacttcaggtgatctgcctgccttggcctcccaaagtgctgggattacaggcataagccactgcgcccggctgatctgatggtttcataaggcttttcccccttttgctcagcacttctccttcctgccgccatgtgaagaaggacatgtttgcttccccttccaccacgattgtaagttgtttcctgaggcctccccggccatgctgaactgtgagtcaattaaacctctttcctttataaattatccagttttgggtatgtctttattagtagaatgagaacagactaatacaacccttaaaggagactgacggagaggattcttcctggatcccagcacttcctctgaatgctactgacattcttcttgaggactttaaactgggagatagaaaacagattccatggctcagcagcctgagagcagggagggagccaagctatagatgacatgggcagcctcccctgaggccaggtgtggccgaacctgggcagtgctgccacccaccccaccagggccaagtcctgtccttggagagccaagcctcaatcactgctagcctcaagtgtccccaagccacagtggctagggggactcagggaacagttcccagtctgccctacttctcttacctttacccctcatacctccaaagtagaccatgttcatgaggtccaaagg

SEQ ID NO: 17 - DNA, 531 bp, Homo sapiens
  misc_feature 518, 528: n = A,T,C or G
aagcgaggaagccactgcggctcctggctgaaaagcggcgccaggctcgggaacagagggaacgcgaagaacaggagcggaagctgcaggctgaaagggacaagcgaatgcgagaggagcagctggcccgggaggctgaagcccgggctgaacgtgaggccgaggcgcggagacgggaggagcaggaggctcgagagaaggcgcaggctgagcaggaggagcaggagcgactgcagaagcagaaagaggaagccgaagcccggtcccgggaagaagctgagcgccagcgccaggagcgggaaaagcactttcagaaggaggaacaggagagacaagagcgaagaaagcggctggaggagataatgaagaggactcggaaatcagaagccgccgaaaccaagaagcaggatgcaaaggagaccgcagctaacaattccggcccagacccttgtgaaagctgtagagactcggccctctgggcttccagaaaggattctattgcagaaaggaaggagctnggccccccangga

SEQ ID NO: 18 - DNA, 1041 bp, Homo sapiens
  misc_feature 544: n = A,T,C or G
ctctgtggaaaactgatgaggaatgaatttaccattacccatgttctcatccccaagcaaagtgctgggtctgattactgcaacacagagaacgaagaagaacttttcctcatacaggatcagcagggcctcatcacactgggctggattcatactcaccccacacagaccgcgtttctctccagtgtcgacctacacactcactgctcttaccagatgatgttgccagagtcagtagccattgtttgctcccccaagttccaggaaactggattctttaaactaactgaccatggactagaggagatttcttcctgtcgccagaaaggatttcatccacacagcaaggatccacctctgttctgtagctgcagccacgtgactgttgtggacagagcagtgaccatcacagaccttcgatgagcgtttgagtccaacaccttccaagaacaacaaaaccatatcagtgtactgtagccccttaatttaagctttctagaaagctttggaagtttttgtagatagtagaaaggggggcatcacntgagaaagagctgattttgtatttcaggtttgaaaagaaataactgaacatattttttaggcaagtcagaaagagaacatggtcacccaaaagcaactgtaactcagaaattaagttactcagaaattaagtagctcagaaattaagaaagaatggtataatgaacccccatatacccttccttctggattcaccaattgttaacatttttttcctctcagctatccttctaatttctctctaatttcaatttgtttatatttacctctgggctcaataagggcatctgtgcagaaatttggaagccatttagaaaatcttttggattttcctgtggtttatggcaatatgaatggagcttattactggggtgagggacagcttactccatttgaccagattgtttggctaacacatcccgaagaatgattttgtcaggaattattgttatttaataaatatttcaggatatttttcctctacaataaagtaacaat

SEQ ID NO: 19 - DNA, 1043 bp, Homo sapiens
ctctgtggaaaactgatgaggaatgaatttaccattacccatgttctcatccccaagcaaagtgctgggtctgattactgcaacacagagaacgaagaagaacttttcctcatacaggatcagcagggcctcatcacactgggctggattcatactcaccccacacagaccgcgtttctctccagtgtcgacctacacactcactgctcttaccagatgatgttgccagagtcagtagccattgtttgctcccccaagttccaggaaactggattctttaaactaactgaccatggactagaggagatttcttcctgtcgccagaaaggatttcatccacacagcaaggatccacctctgttctgtagctgcagccacgtgactgttgtggacagagcagtgaccatcacagaccttcgatgagcgtttgagtccaacaccttccaagaacaacaaaaccatatcagtgtactgtagccccttaatttaagctttctagaaagctttggaagtttttgtagatagtagaaaggggggcatcacctgagaaagagctgattttgtatttcaggtttgaaaagaaataactgaacatattttttaggcaagtcagaaagagaacatggtcacccaaaagcaactgtaactcagaaattaagttactcagaaattaagtagctcagaaattaagaaagaatggtataatgaacccccatatacccttccttctggattcaccaattgttaacatttttttcctctcagctatccttctaatttctctctaatttcaatttgtttatatttacctctgggctcaataagggcatctgtgcagaaatttggaagccatttagaaaatcttttggattttcctgtggtttatggcaatatgaatggagcttattactggggtgagggacagcttactccatttgaccagattgtttggctaacacatcccgaagaatgattttgtcaggaattattgttatttaataaatatttcaggatatttttcctctacaataaagtaacaatta

SEQ ID NO: 20 - DNA, 448 bp, Homo sapiens
ggacgacaaggccatggcgatatcggatccgaattcaagcctttggaattaaataaacctggaacagggaaggtgaaagttggagtgagatgtcttccatatctatacctttgtgcacagttgaatgggaactgtttgggtttagggcatcttagagttgattgatggaaaaagcagacaggaactggtgggaggtcaagtggggaagttggtgaatgtggaataacttacctttgtgctccacttaaaccagatgtgttgcagctttcctgacatgcaaggatctactttaattccacactctcattaataaattgaataaaagggaatgttttggcacctgatataatctgccaggctatgtgacagtaggaaggaatggtttcccctaacaagcccaatgcactggtctgactttataaattatttaataaaatgaactattatc

SEQ ID NO: 21 - DNA, 411 bp, Homo sapiens
ggcagtgacattcaccatcatgggaaccaccttcccttttcttcaggattctctgtagtggaagagagcacccagtgttgggctgaaaacatctgaaagtagggagaagaacctaaaataatcagtatctcagagggctctaaggtgccaagaagtctcactggacatttaagtgccaacaaaggcatactttcggaatcgccaagtcaaaactttctaacttctgtctctctcagagacaagtgagactcaagagtctactgctttagtggcaactacagaaaactggtgttacccagaaaaacaggagcaattagaaatggttccaatatttcaaagctccgcaaacaggatgtgctttcctttgcccatttagggtttcttctctttcctttctctttattaaccact

SEQ ID NO: 22 - DNA, 896 bp, Homo sapiens
  misc_feature 230, 320: n = A,T,C or G
tgcgctgaaaacaacggcctcctttactgttaaaatgcagccacaggtgcttagccgtgggcatctcaaccaccagcctctgtggggggcaggtgggcgtccctgtgggcctctgggcccacgtccagcctctgtcctctgccttccgttcttcgacagtgttcccggcatccctggtcacttggtacttggcgtgggcctcctgtgctgctccagcagctcctccaggnggtcggcccgcttcaccgcagcctcatgttgtgtccggaggctgctcacggcctcctccttcctcgcgagggctgtcttcaccctccggngcacctcctccagctccagctgctggcgggcctgcagcgtggccagctcggccttggcctgccgcgtctcctcctcaraggctgccagccggtcctcgaactcctggcggatcacctgggccaggttgctgcgctcgctagaaagctgctcgttcaccgcctgcgcatcctccagcgcccgctccttctgccgcacaaggccctgcagacgcagattctcgccctcggcctccccaagctggcccttcagctccgagcaccgctcctgaagcttccgctccgactgctccagctcggagagctcggcctcgtacttgtcccgtaagcgcttgatgcggctctcggcagccttctcactctcctccttggccagcgccatgtcggcctccagccggtgaatgaccagctcaatctccttgtcccggcctttccggatttcttccctcagctcctgttcccggttcagcagccacgcctcctccttcctggtgcggccggcctcccacgcctgcctctccagctccagctgctgcttcagggtattcagctccatctggcgggcctgcagcgtggcca

SEQ ID NO: 23 - DNA, 111 bp, Homo sapiens
caacttattacttgaaattataatatagcctgtccgtttgctgtttccaggctgtgatatattttcctagtggtttgactttaaaaataaataaggtttaattttctcccc

SEQ ID NO: 24 - DNA, 531 bp, Homo sapiens
  misc_feature 472, 494: n = A,T,C or G
tgcaagtcacgggagtttatttatttaatttttttccccagatggagactctgtcgcccaggctggagtgcaatggtgtgatcttggctcactgcaacctccacctcctgggttcaagcgattctcctgccacagcctcccgagtagctgggattacaggtgcccgccaccacacccagctaatttttatatttttagtaaagacagggtttccccatgttggccaggctggtcttgaacttctgacctcaggtgatccacctgcctcggcctcccaaagtgttgggattacaggcgtgagctacccgtgcctggccagccactggagtttaaaggacagtcatgttggctccagcctaaggcggcattttcccccatcagaaagcccgcggctcctgtacctcaaaatagggcacctgtaaagtcagtcagtgaagtctctgctctaactggccacccggggccattggcntctgacacagccttgccaggangcctgcatctgcaaaagaaaagttcacttcctttccg

SEQ ID NO: 25 - DNA, 471 bp, Homo sapiens
  misc_feature 377: n = A,T,C or G
cagagaatctkagaaagatgtcgcgttttcttttaatgaatgagagaagcccatttgtatccctgaatcattgagaaaaggcggcggtggcgacagcggcgacctagggatcgatctggagggacttggggagcgtgcagagacctctagctcgagcgcgagggacctcccgccgggatgcctggggagcagatggaccctactggaagtcagttggattcagatttctctcagcaagatactccttgcctgataattgaagattctcagcctgaaagccaggttctagaggatgattctggttctcacttcagtatgctatctcgacaccttcctaatctccagacgcacaaagaaaatcctgtgttggatgttgngtccaatccttgaacaaacagctggagaagaacgaggagaccggtaatagtgggttcaatgaacatttgaaagaaaaccaggttgcagaccctg

SEQ ID NO: 26 - DNA, 541 bp, Homo sapiens
gactgtcctgaacaagggacctctgaccagagagctgcaggagatgcagagtggtggcaggagtggaagccaaagaacacccaccttcctcccttgaaggagtagagcaaccatcagaagatactgttttattgctctggtcaaacaagtcttcctgagttgacaaaacctcaggctctggtgacttctgaatctgcagtccactttccataagttcttgtgcagacaactgttcttttgcttccatagcagcaacagatgctttggggctaaaaggcatgtcctctgaccttgcaggtggtggattttgctcttttacaacatgtacatccttactgggctgtgctgtcacagggatgtccttgctggactgttctgctatggggatatcttcgttggactgttcttcatgcttaattgcagtattagcatccacatcagacagcctggtataaccagagttggtggttactgattgtagctgctctttgtccacttcatatggcacaagtattttcctcaacatcctggctctgggaag

SEQ ID NO: 27 - DNA, 461 bp, Homo sapiens
  misc_feature 367: n = A,T,C or G
gaaatgtatatttaatcattctcttgaacgatcagaactctraaatcagttttctataacarcatgtaatacagtcaccgtggctccaaggtccaggaaggcagtggttaacacatgaagagtgtgggaagggggctggaaacaaagtattcttttccttcaaagcttcattcctcaaggcctcaattcaagcagtcattgtccttgctttcaaaagtctgtgtgtgcttcatggaaggtatatgtttgttgccttaatttgaattgtggccaggaagggtctggagatctaaattcagagtaagaaaacctgagctagaactcaggcatttctcttacagaacttggcttgcagggtagaatgaanggaaagaaacttagaagctcaacaagctgaagataatcccatcaggcatttcccataggccttgcaactctgttcactgagagatgttatcctg

SEQ ID NO: 28 - DNA, 541 bp, Homo sapiens
agtctggagtgagcaaacaagagcaagaaacaarragaagccaaaagcagaaggctccaatatgaacaagataaatctatcttcaaagacatattagaagttgggaaaataattcatgtgaactagacaagtgtgttaagagtgataagtaaaatgcacgtggagacaagtgcatccccagatctcagggacctccccctgcctgtcacctggggagtgagaggacaggatagtgcatgttctttgtctctgaatttttagttatatgtgctgtaatgttgctctgaggaagcccctggaaagtctatcccaacatatccacatcttatattccacaaattaagctgtagtatgtaccctaagacgctgctaattgactgccacttcgcaactcaggggcggctgcattttagtaatgggtcaaatgattcactttttatgatgcttcccaaggtgccttggcttctcttcccaactgacaaatgcccaagttgagaaaaatgatcataattttagcataaaccgagcaatcggcgacccc

SEQ ID NO: 29 - DNA, 411 bp, Homo sapiens
tagctgtcttcctcactcttatggcaatgaccccatatcttaatggattaagataatgaaagtgtatttcttacactctgtatctatcaccagaagctgaggtgatagcccgcttgtcattgtcatccatattctgggactcaggcgggaactttctggaatattgccagggagcatggcagaggggcacagtgcattctgggggaatgcacattggctcagcctgggtaatgagtgatatacattacctctgttcacaactcattgcccagcaccagtcacaaggccccaccaaataccagagcccaagaaatgtagtcctgttgatatggttttgctgtgtcccaacccaaatctcatcttgaattgtaagctcccataattcccatgtgttgtgggagggacctggtg

SEQ ID NO: 30 - DNA, 511 bp, Homo sapiens
atcatgaggatgttaccaaagggatggtactaaaccatttgtattcgtctgttttcacactgctttgaagatactacctgagactgggtaatttataaacaaaagagatttaattgactcacagttctgcatggctgaagaggcctcaggaaacttacagtcatggtggaaggcaaaggaggagcaaggcatgtcttacatgtcagtaggagagagagcgagagcaggagaacctgccacttataaaccattcagatctcataactccctatcatgagaaaaacatggaggaaaccaccctcatgatccaatcacctcccgccaggtccctccctcgacacgtggggattataattcaggattagagggacacagagacaaaccatatcatcattcatgagaaatccaccctcatagtccaatcagctcctaccaggccccacctccaacactggggattgcaattcaacatgagatttggatggggacacagattcaaaccatatcatac

SEQ ID NO: 31 - DNA, 827 bp, Homo sapiens
catggcctttctccttagaggccagaggtgctgccctggctgggagtgaagctccaggcactaccagctttcctgattttcccgtttggtccatgtgaagagctaccacgagccccagcctcacagtgtccactcaagggcagcttggtcctcttgtcctgcagaggcaggctggtgtgaccctgggaacttgacccgggaacaacaggtggcccagagtgagtgtggcctggcccctcaacctagtgtccgtcctcctctctcctggagccagtcttgagtttaaaggcattaagtgttagatacaagctccttgtggctggaaaaacacccctctgctgataaagctcagggggcactgaggaagcagaggccccttgggggtgccctcctgaagagagcgtcaggccatcagctctgtccctctggtgctcccacgtctgttcctcaccctccatctctgggagcagctgcacctgactggccacgcgggggcagtggaggcacaggctcagggtggccgggctacctggcaccctatggcttacaaagtagagttggcccagtttccttccacctgaggggagcactctgactcctaacagtcttccttgccctgccatcatctggggtggctggctgtcaagaaaggccgggcatgctttctaaacacagccacaggaggcttgtagggcatcttccaggtggggaaacagtcttagataagtaaggtgacttgcctaaggcctcccagcacccttgatcttggagtctcacagcagactgcatgtsaacaactggaaccgaaaacatgcctcagtataaaa

SEQ ID NO: 32 - DNA, 291 bp, Homo sapiens
ccagaacctccttctctttggagaatggggaggcctcttggagacacagagggtttcaccttggatgacctctagagaaattgcccaagaagcccaccttctggtcccaacctgcagaccccacagcagtcagttggtcaggccctgctgtagaaggtcacttggctccattgcctgcttccaaccaatgggcaggagagaaggcctttatttctcgcccacccattctcctgtaccagcacctccgttttcagtcagygttgtccagcaacggtaccgtttacacagtca

SEQ ID NO: 33 - DNA, 491 bp, Homo sapiens
tgcatgtagttttatttatgtgttttsgtctggaaaaccaagtgtcccagcagcatgactgaacatcactcacttcccctacttgatctacaaggccaacgccgagagcccagaccaggattccaaacacactgcacgagaatattgtggatccgctgtcaggtaagtgtccgtcactgacccaracgctgttacgtggcacatgactgtacagtgccacgtaacagcactgtacttttctcccatgaacagttacctgccatgtatctacatgattcagaacattttgaacagttaattctgacacttgaataatcccatcaaaaaccgtaaaatcactttgatgtttgtaacgacaacatagcatcactttacgacagaatcatctggaaaaacagaacaacgaatacatacatcttaaaaaatgctggggtgggccaggcacagcttcacgcctgtaatcccagcactttgggaggcttaagcgggtg

SEQ ID NO: 34 - DNA, 521 bp, Homo sapiens
  misc_feature 453, 476, 487: n = A,T,C or G
tggggcggaaagaagccaaggccaaggagctggtgcggcagctgcagctggaggccgaggagcagaggaagcagaagaagcggcagagtgtgtcgggcctgcacagataccttcacttgctggatggaaatgaaaattacccgtgtcttgtggatgcagacggtgatgtgatttccttcccaccaataaccaacagtgagaagacaaaggttaagaaaacgacttctgatttgtttttggaagtaacaagtgccaccagtctgcagatttgcaaggatgtcatggatgccctcattctgaaaatggcaagaaatgaaaaagtacactttagaaaataaagaggaaggatcactctcagatactgaagccgatgcagtctctggacaacttccagatcccacaacgaatcccagtgctggaaaggacgggcccttccttctggtggtggaacangtcccggtggtggatcttggaanggaacctgaangtggtgtaccccgtccaaggccgaccttggccac

SEQ ID NO: 35 - DNA, 161 bp, Homo sapiens
  misc_feature 18: n = A,T,C or G
tcccgcgctcgcagggcncgtgccacctgccygtccgcccgctcgctcgctcgcccgccgcgccgcgctgccgaccgycagcatgctgccgagagtgggctgccccgcgctgccgctgccgccgccgccgctgctgccgctgctgccgctgctgctgctgc

SEQ ID NO: 36 - DNA, 341 bp, Homo sapiens
ggcgggtaggcatggaactgagaagaacgaagaagctttcagactacgtggggaagaatgaaaaaaccaaaattatcgccaagattcagcaaaggggacagggagctccagcccgagagcctattattagcagtgaggagcagaagcagctgatgctgtactatcacagaagacaagaggagctcaagagattggaagaaaatgatgatgatgcctatttaaactcaccatgggcggataacactgctttgaaaagacattttcatggagtgaaagacataaagtggagaccaagatgaagttcaccagctgatgacacttccaaagagattagctcacct

SEQ ID NO: 37 - DNA, 521 bp, Homo sapiens
  misc_feature 516: n = A,T,C or G
tctgaaggttaaatgtttcatctaaatagggataatgrtaaacacctatagcatagagttgtttgagattaaatgagataatacatgtaaaattatgtgcctggcatacagcaagattgttgttgttgttgatgatgatgatgatgatgataatatttttctatccccagtgcacaactgcttgaacctattagataatcaatacatgtttcttgaactgagatcaatttccccatgttgtctgactgatgaagccctacattttcttctagaggagatgacatttgagcaagatcttaaagaaaatcagatgccttcacctgaccactgcttggtgatcccatggcactttgtacatctctccattagctctcatctcaccagcccatcattattgtatgtgctgccttctgaagcttgcagctggctaccatcmggtagaataaaaatcatcctttcataaaatagtgaccctccttttttatttgcatttcccaaagccaagcaccgtggganggtag

SEQ ID NO: 38 - DNA, 461 bp, Homo sapiens
tatgaagaagggaaaagaagataatttgtgaaagaaatgggtccagttactagtctttgaaaagggtcagtctgtagctcttcttaatgagaataggcagctttcagttgctcagggtcagatttccttagtggtgtatctaatcacaggaaacatctgtggttccctccagtctctttctgggggacttgggcccacttctcatttcatttaattagaggaaatagaactcaaagtacaatttactgttgtttaacaatgccacaaagacatggttgggagctatttcttgatttgtgtaaaatgctgtttttgtgtgctcataatggttccaaaaattgggtgctggccaaagagagatactgttacagaagccagcaagaagacctctgttcattcacacccccggggatatcaggaattgactccagtgtgtgcaaatccagtttggcctatcttct

SEQ ID NO: 39 - DNA, 769 bp, Homo sapiens
tgagggactgattggtttgctctctgctattcaattccccaagcccacttgttcctgcagcgtcctccttctcattccctttagttgtaccctctctttcatctgagacctttccttcttgatgtcgccttttcttcttcttgctttttctgatgttctgctcagcatgttctgggtgcttctcatctgcatcattcctttcagatgctgtagcttcttcctcctctttctgcctccttttctttttcttttttttggggggcttgctctctgactgcagttgaggggccccagggtcctggcctttgagacgagccaggaaggcctgctcctgggcctctaggcgagcaagcttggccttcattgtgatcccaagacgggcagccttgtgtgctgttcgcccctcacaggcttggagcagcatctcatcagtcagaatctttggggacttggacccctggttgtcgtcatcactgcagctctccaagtctttgtttggcttctctccacctgaagtcaatgtagccatcttcacaaacttctgatacagcaagttgggcttgggatgattataacgggtggtctccttagaaaggctccttatctgtactccatcctgcccagtttccactaccaagttggccgcagtcttgttgaagagctcattccaccagtggtttgtgaactccttggcagggtcatgtcctaccccatgagtgtcttgcttcagygtcaccctgagagcctgagtgataccattctccttccg

SEQ ID NO: 40 - DNA, 292 bp, Homo sapiens
gacaacatgaaataaatcctagaggacaaaattaaactcaatagagtgtagtctagttaaaaactcgaaaaatgagcaagtctggtgggagtggaggaagggctatactataaatccaagtgggcctcctgatcttaacaagccatgctcattatacacatctctgaactggacataccacctttacgcaggaaacagggcttggaacttctaagggaaattaacatgcaccacccacatctaacctacctgccgggtaggtaccatccctgcttcgctgaaatcagtgctc

SEQ ID NO: 41 - DNA, 406 bp, Homo sapiens
ttggaattaaataaacctggaacagggaaggtgaaagttggagtgagatgtcttccatatctatacctttgtgcacagttgaatgggaactgtttgggtttagggcatcttagagttgattgatggaaaaagcagacaggaactggtgggaggtcaagtggggaagttggtgaatgtggaataacttacctttgtgctccacttaaaccagatgtgttgcagctttcctgacatgcaaggatctactttaattccacactctcattaataaattgaataaaagggaatgttttggcacctgatataatctgccaggctatgtgacagtaggaaggaatggtttcccctaacaagcccaatgcactggtctgactttataaattatttaataaaatgaactattatc

SEQ ID NO: 42 - DNA, 381 bp, Homo sapiens
aaactggacctgcaacagggacatgaatttactgcarggtctgagcaagctcagcccctctacctcagggccccacagccatgactacctcccccaggagcgggagggtgaagggggcctgtctctgcaagtggagccagagtggaggaatgagctctgaagacacagcacccagccttctcgcaccagccaagccttaactgcctgcctgaccctgaaccagaacccagctgaactgcccctccaagggacaggaaggctgggggagggagtttacaacccaagccattccaccccctcccctgctggggagaatgacacatcaagctgctaacaattgggggaaggggaaggaagaaaactctgaaaacaaaatcttgt

SEQ ID NO: 43 - DNA, 451 bp, Homo sapiens
catgcgtttcaccactgttggccaggctggtctcgaactcctggcctcaagcaatccacccgcctcagcctccaaaagtgctgggattacagatgtgagccatggcaccatgccaaaaggctatattcctggctctgtgtttccgagactgcttttaatcccaacttctctacatttagattaaaaaatattttattcatggtcaatctggaacataattactgcatcttaagtttccactgatgtatatagaaggctaaaggcacaatttttatcaaatctagtagagtaaccaaacataaaatcattaattactttcaacttaataactaattgacattcctcaaaagagctgttttcaatcctgataggttctttattttttcaaaatatatttgccatgggatgctaatttgcaataaggcgcataatgagaataccccaaactgga

SEQ ID NO: 44 - DNA, 521 bp, Homo sapiens
gttggacccccagggactggaaagacacttcttgcccgagctgtggcgggagaagctgatgttcctttttattatgcttctggatccgaatttgatgagatgtttgtgggtgtgggagccagccgtatcagaaatctttttagggaagcaaaggcgaatgctccttgtgttatatttattgatgaattagattctgttggtgggaagagaattgaatctccaatgcatccatattcaaggcagaccataaatcaacttcttgctgaaatggatggttttaaacccaatgaaggagttatcataataggagccacaaacttcccagaggcattagataatgccttaataccgtcctggtcgttttgacatgcaagttacagttccaaggccagatgtaaaaggtcgaacagaaattttgaaatggtatctcaataaaataaagtttgatcaatcccgttgatccagaaattatagcctcgaggtactggtggcttttccggaagcagagttgggagaatctt

SEQ ID NO: 45 - DNA, 585 bp, Homo sapiens
gcctacaacatccagaaagagtctaccctgcacctggtgctscgtctcagaggtgggatgcagatcttcgtgaagaccctgactggtaagaccatcactctcgaagtggagccgagtgacaccatygagaacgtcaaagcaaagatccargacaaggaaggcrtycctcctgaccagcagaggttgatctttgccggaaagcagctggaagatggdcgcaccctgtctgactacaacatccagaaagagtcyaccctgcacctggtgctccgtctcagaggtgggatgcaratcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcmacaaatttcattgcactttcctttcaataaagttgttgcattccc

SEQ ID NO: 46 - DNA, 481 bp, Homo sapiens
gaactgggccctgagcccaagtcatgccttgtgtccgcatctgccgtgtcacctctgtkcctgcccctcacccctccctcctggtcttctgagccagcaccatctccaaatagcctattccttcctgcaaatcacacacacatgcgggccacacatacctgctgccctggagatggggaagtaggagagatgaatagaggcccatacattgtacagaaggaggggcaggtgcagataaaagcagcagacccagcggcagctgaggtgcatggagcacggttggggccggcattgggctgagcacctgatgggcctcatctcgtgaatcctcgaggcagcgccacagcagaggagttaagtggcacctgggccgagcagagcaggagactgagggtcagagtggaggctaagctgccctggaactcctcaatcttgcctgccccctagtatgaagcccccttcctgcccctacaattcctga

SEQ ID NO: 47 - DNA, 461 bp, Homo sapiens
  misc_feature 128: n = A,T,C or G
atggatcttactttgccacccaggttggagtgcagtgctgcaatcttggctcactgcagccttaacctcccaggctcaagctatcctcctgccaaagccttccacatagctgggactacaggtacacngccaccacacccagctaaaatttttgtattttttgtagagacgggatctcgccacgttgcccaggctggtcccatcctgacctcaagcagatctgcccacctcagccccccaacgtgctaggattacaggcgtgagccaccgcacccagcctttgttttgcttttaatggaatcaccagttcccctccgtgtctcagcagcagctgtgagaaatgctttgcatctgtgacctttatgaaggggaacttccatgctgaatgagggtaggattacatgctcctgtttcccgggggtcaagaaagcctcagactccagcatgataagcagggtgag

SEQ ID NO: 48 - DNA, 571 bp, Homo sapiens
ataggggctttaaggagggaattcaggttcaatgaggtcgtaaggccagggctcttatccagtaagactggggtccttagatgagaaagagacacccgaggtccttctctctgccgtgtgaggatgcatcaagaaggcggccgtctgcaagcgaaggagaggccgcaccagaaaccgacaccttcatcttggacttgcagcctctagaactgagaaaataactgtctgttggttaagccacccagtttgtagtattctcttatggcttcctaagcagactaacaaacaaacacccaaaattaactgatggcttcgctgtcttctgtaaaaattgctatgagagaacttttcactcactgttttgcagtttctccctcagtccctggttctttcttctcacataatcccaatttcaatttatagttcatggcccaggcagagtcattcatcacggcatctcctgagctaaaccagcacctgctctgctcacttcttgactggctgctcatcatcagccctcttgcagagatttcatttcctcccgtgccaggtacttcacgcaccaagctca

SEQ ID NO: 49 - DNA, 511 bp, Homo sapiens
ggataatgaagttgttttatttagcttggacaaaaaggcatattcctctattttcttatacaacaaatatccccaaaataaagcaagcatatatatcttgaatgtgtaataatccagtgataaacaagagcagtactttaaaagaaaaaaaaatatgtatttctgtcaggttaaaatgagaatcaaaaccatttactctgctaactcattattttttgctttctttttggttaagagaggcaatgcaatacactgaaaaaggtttttatcttatctggcattggaattagacatattcaaaccccagcccccatttccaaactttaagaccacaaacaagtaatttacttttctgaacattggttttttctggaaaatgggaattataaaatagactttgcagactcttatgagattaaataagataatgtatgaaattctttcttcttttttacttctttttcctttttgagatggagtctcaccccgtcacccaggctggagtacagtg

SEQ ID NO: 50 - DNA, 561 bp, Homo sapiens
ccactgcactccagcctgggtgacggagtgagactctgtctcaaaaaaacaaacaaacaaacaaacaaaaaactgaaaaggaaatagagttcctctttcctcatatatgaatatattatttcaacagattgttgatcacctaccatatgcttggtattgttctaattgctggggatacagcaagaggttctgcagaacttcatggagcatgaaagtaaataaacaaagttaatttcaaggccaggcatggttgctcacacctttagtcccagcactttgggaggctgaggcaggtggatcacttgggcccaggagttcaaggctgcagtgagccaagattgtgccactactctccaggctgggcaacagagcaagaccctgtctcagggggaacaaaaagttaatttcagattttgttaagtgctgtaaaggaagtaaataggttgatattcaagagagcacctgaaggccaggcgtggtggctcacgcctgtggtctaacgctttgggaagcccgagcgggcggatcacaaggtcaggagaattttggccaggcatggtg

SEQ ID NO: 51 - DNA, 451 bp, Homo sapiens
agaatccatttattgggttttaaactagttacacaactgaaatcagtttggcactactttatacagggattacgcctgtgtatgccgacacttaaatactgtaccaggaccactgctgtgcttaggtctgtattcagtcattcagcatgtagatactaaaaatatactgtagtgttcctttaaggaagactgtacagggtgtgttgcaagatgacattcaccaatttgtgaattatttcaacccagaagatacctttcactctataaacttgtcataggcaaacatgtggtgttagcattgagagatgcacacaaaaatgttacataaaagttcagacattctaatgataagtgaactgaaaaaaaaaaaaaccccacatctcaatttttgtaacaagataaagaaaataatttaaaaacacaaaaaatggcattcagtgggtacaaagcc

SEQ ID NO: 52 - DNA, 682 bp, Homo sapiens
caaatatttaatataaatctttgaaacaagttcagakgaaataaaaatcaaagtttgcaaaaacgtgaagattaacttaattgtcaaatattcctcattgccccaaatcagtattttttttatttctatgcaaaagtatgccttcaaactgcttaaatgatatatgatatgatacacaaaccagttttcaaatagtaaagccagtcatcttgcaattgtaagaaataggtaaaagattataagacaccttacacacacacacacacacacacacacacgtgtgcaccgccaatgacaaaaaacaatttggcctctcctaaaataagaacatgaagacccttaattgctgccaggagggaacactgtgtcacccctccctacaatccaggtagtttcctttaatccaatagcaaatctgggcatatttgagaggagtgattctgacagccacsgttgaaatcctgtggggaaccattcatgtccacccactggtgccctgaaaaaatgccaataatttttcgctcccacttctgctgctgtctcttccacatcctcacatagaccccagacccgctggcccctggctgggcatcgcattgctggtagagcaagtcataggtctcgtctttgacgtcacagaagcgatacaccaaattgcctggtcggtcattgtcataaccag

SEQ ID NO: 53 - DNA, 311 bp, Homo sapiens
  misc_feature 208: n = A,T,C or G
tttgactttagtaggggtctgaactatttattttactttgccmgtaatatttaraccytatatatctttcattatgccatcttatcttctaatgbcaagggaacagwtgctaamctggcttctgcattwatcacattaaaaatggctttcttggaaaatcttcttgatatgaataaaggatcttttavagccatcatttaaagcmggnttctctccaacacgagtctgctsasggggggkgagctgtgaactctggctgaaggctttcccatacacactgcaatgacmtggtttctgaccagbgtgagtta

SEQ ID NO: 54 - DNA, 561 bp, Homo sapiens
agagaagccccataaatgcaatcagtgtgggaaggccttcagtcagagctcaagccttttcctccatcatcgggttcatactggagagaaaccctatgtatgtaatgaatgcggcagagcctttggttttaactctcatcttactgaacacgtaaggattcacacaggagaaaaaccctatgtttgtaatgagtgcggcaaagcctttcgtcggagttccactcttgttcagcatcgaagagttcacactggggagaagccctaccagtgcgttgaatgtgggaaagctttcagccagagctcccagctcaccctacatcagccgagttcacactggagagaagccctatgactgtggtgactgtgggaaggccttcagccggaggtcaaccctcattcagcatcagaaagttcacagcggagagactcgtaagtgcagaaaacatggtccagcctttgttcatggctccagcctcacagcagatggacagattcccactggagagaagcacggcagaacctttaaccatggtgcaaatctcattctgcgctggacagttc

SEQ ID NO: 55 - DNA, 811 bp, Homo sapiens
gagacagggtctcactttgtcacccaggctggaatgcagtggtgcgatcttacgtagctcactgcagccctgacctcctggactcaaacaattctcctgcctcagccctgcaagtagctgggactgtgggtgcatgccaccatgcctggctaacttttgtagtttttgtaaagatggggttttgccatgttgcacatgctggtcttgaactcctgagctcaaacgatctgcccacctcggcctcccagaatgttgggattacaggggtaaaccaccacgcctggccccattagggtattcttagcatccacttgctcactgagattaatcataagagatgataagcactggaagaaaaaaatttttactaggctttggatatttttttcctttttcagctttatacagaggattggatctttagttttcctttaactgataataaaacattgaaaggaaataagtttacctgagattcacagagataaccggcatcactcccttgctcaattccagtctttaccacatcaattattttcagaggtgcaggataaaggcctttagtctgctttcgcactttttcttccacttttttgtaaacctgttgcctgacaaatggaattgacagcgtatgccatgactattccatttgtcaggcatacgctgtcaatttttccaccaatcccttgtctctctttggagagatcttcttatcagctagtcctttggcaaaagtaattgcaacttcttctaggtattctattgtccgttccactggtggaacccctgggaccaggactaaaacctccag

SEQ ID NO: 56 - DNA, 591 bp, Homo sapiens
  misc_feature 45, 477, 490, 561: n = A,T,C or G
atctcatatatatatttcttcctgactttatttgcttgcttctgncacgcatttaaaatatcacagagaccaaaatagagcggctttctggtggaacgcatggcagtcacaggacaaaatacaaaactagggggctctgtcttctcatacatcatacaattttcaagtattttttttatgtacaaagagctactctatctgaaaaaaaattaaaaaataaatgagacaagatagtttatgcatcctaggaagaaagaatgggaagaaagaacggggcagttgggtacagattcctgtcccctgttcccagggaccactaccttcctgccactgagttcccccacagcctcacccatcatgtcacagggcaagtgccagggtaggtggggaccagtggagacaggaaccagcaacatactttggcctggaagataaggagaaagtctcagaaacacactggtgggaagcaatcccacnggccgtgccccangagcttcccacctgctgctggctccctgggtggctttgggaacagcttgggcaggcccttttgggtggggnccaactgggcctttgggcccgtgtggaaag

SEQ ID NO: 57 - DNA, 481 bp, Homo sapiens
aaacattgagatggaatgatagggtttcccagaatcaggtccatattttaactaaatgaaaattatgatttatagccttctcaaatacctgccatacttgatatctcaaccagagctaattttacctctttacaaattaaataagcaagtaactggatccacaatttataatacctgtcaattttttctgtattaaacctctatcatagtttaagcctattagggtacttaatccttacaaataaacaggtttaaaatcacctcaataggcaactgcccttctggttttcttctttgactaaacaatctgaatgcttaagattttccactttgggtgctagcagtacacagtgttacactctgtattccagacttcttaaattatagaaaaaggaatgtacactttttgtattctttctgagcagggccgggaggcaacatcatctaccatggtagggacttgtatgcatggactacttta

SEQ ID NO: 58 - DNA, 141 bp, Homo sapiens
actctgtcgcccaggctggagcccabtggmgcgatctcgactccctgcaagctmcgcctcacaggwtcatgccattctcctgcctcagcatctggagtagctgggactacaggcgccagccaccatgcccagctaattttt

SEQ ID NO: 59 - DNA, 191 bp, Homo sapiens
accttaaagacataggagaatttatactgggagagaaagcttacaaatgtaaggtttctgacaagacttgggagtgattcacacctggaacaacatactggacttcacactggabagaaaccttacaagtgtaatgagtgtggcaaagcctttggcaagcagtcaacacttattcaccatcaggcaattca

SEQ ID NO: 60 - DNA, 480 bp, Homo sapiens
agtcaggatcatgatggctcagtttcccacagcgatgaatggagggccaaatatgtgggctattacatctgaagaacgtactaagcatgataaacagtttgataacctcaaaccttcaggaggttacataacaggtgatcaagcccgtacttttttcctacagtcaggtctgccggccccggttttagctgaaatatgggccttatcagatctgaacaaggatgggaagatggaccagcaagagttctctatagctatgaaactcatcaagttaaagttgcagggccaacagctgcctgtagtcctccctcctatcatgaaacaaccccctatgttctctccactaatctctgctcgttttgggatgggaagcatgcccaatctgtccattcatcagccattgcctccagttgcacctatagcaacacccttgtcttctgctacttcagggaccagtattcctccctaatgatgcctgct

SEQ ID NO: 61 - DNA, 381 bp, Homo sapiens
ctttcgatttccttcaatttgtcacgtttgattttatgaagttgttcaagggctaactgctgtgtattatagctttctctgagttccttcagctgattgttaaatgaatccatttctgagagcttagatgcagtttctttttcaagagcatctaattgttctttaagtctttggcataattcttccttttctgatgactttctatgaagtaaactgatccctgaatcaggtgtgttactgagctgcatgtttttaattctttcgtttaatagctgcttctcagggaccagatagataagcttattttgatattccttaagctcttggtgaagttgttcgatttccataatttccaggtcacactggttatcccaaacttct

SEQ ID NO: 62 - DNA, 906 bp, Homo sapiens
gtggaggtgaaacggaggcaagaaagggggctacctcaggagcgagggacaaagggggcgtgaggcacctaggccgcggcaccccggcgacaggaagccgtcctgaaccgggctaccgggtaggggaagggcccgcgtagtcctcgcagggccccagagctggagtcggctccacagccccgggccgtcggcttctcacttcctggacctccccggcgcccgggcctgaggactggctcggcggagggagaagaggaaacagacttgagcagctccccgttgtctcgcaactccactgccgaggaactctcatttcttccctcgctccttcaccccccacctcatgtagaaaggtgctgaagcgtccggagggaagaagaacctgggctaccgtcctggccttcccmcccccttcccggggcgctttggtgggcgtggagttggggttgggggggtgggtgggggttcttttttggagtgctggggaacttttttcccttcttcaggtcaggggaaagggaatgcccaattcagagagacatgggggcaagaaggacgggagtggaggagcttctggaactttgcagccgtcatcgggaggcggcagctctaacagcagagagcgtcaccgcttggtatcgaagcacaagcggcataagtccaaacactccaaagacatggggttggtgacccccgaagcagcatccctgggcacagttatcaaacctttggtggagtatgatgatatcagctctgattccgacaccttctccgatgacatggccttcaaactagaccgaagggagaacgacgaacgtcgtggatcagatcggagcgaccgcctgcacaaacatcgtcaccaccagcacaggcgttcccgggacttactaaaagctaaacagaccg

SEQ ID NO: 63 - DNA, 491 bp, Homo sapiens
gacatgtttgcctgcaggggaccagagacaatgggattagccagtgctcactgttctttatgcttccagagaggatggggacagctctcaggtcagaatccaggctgagaaggccatgctggttgggggcccccggaagcacggtccggatcctccctggcatcagcgtagacccgctgctcaggcttggggtaccaaactcatgctctgtactgttttggccccatgcggtgagaggaaaacctagaaaaagattggtcgtgctaaggaatcagctgccccctcatcctccgcatccaatgctggtgacaacatattccctctcccaggacacagactcggtgactccacactgggctgagtggcctctggaggctcgtggcctaaggcagggctccgtaaggctgatcggctgaactgggtggggtgagggtttctgacccttcgcttcccatcccataaccgctgtcaatgagctcacactgtggtca

SEQ ID NO: 64 - DNA, 511 bp, Homo sapiens
gatggcatggtcgttgctaatgtgcctgctgggatggagcacttcctcctgtgagcccaggggacccgcctgtccctggagcttggggcaaggagggaagagtgataccaggaaggtggggctgcagccaggggccagagtcagttcagggagtggtcctcggccctcaaagctcctccggggactgctcaggagtgatggtgccctggagtttgccccaacttccctggccaccctggaaggtgcctggctgctccaggcctctaggctgggctgatgggtttctccaggacacaagtatcattaaagccaccctctcctcagcttgtcaggccgcacatgtgggacaggctgtgctcacaaccccctcgcctgccctgccctccatcaggaggagccagtggaaccttcggaaagctcccagcatctcagcagccctcaaaagtcgtcctggggcaagctctggttctcctgactggaggtcatctgggcttggcctgctctctctcgc

SEQ ID NO: 65 - DNA, 394 bp, Homo sapiens
taaaaaagtgtaacaaaggtttatttagactttcttcatgcccccagatccaggatgtctatgtaaaccgttatcttacaaagaaagcacaatatttggtataaactaagtcagtgacttgcttaactgaaatagcgtccatccaaaagtgggtttaaggtaaaactacctgacgatattggcggggatcctgcagtttggactgcttgccgggtttgtccagggttccgggtctgttcttggcactcatggggacaggcatcctgctcgtctgtggggccccgctggagcccttacgtgaagctgaaggtatcgaccstagggggctctagggcagtgggaccttcatccggaactaacaagggtcggggagaggcctcttgggctatgtggg

SEQ ID NO: 66 - DNA, 359 bp, Homo sapiens
caagcgttcctttatggatgtaaattcaaacagtcatgctgagccatcccgggctgacagtcacgttwaagacactaggtcgggcgccacagtgccacccaaggagaagaagaatttggaatttttccatgaagatgtacggaaatctgatgttgaatatgaaaatggcccccaaatggaattccaaaaggttaccacaggggctgtaagacctagtgaccctcctaagtgggaaagaggaatggagaatagtatttctgatgcatcaagaacatcagaatataaaactgagatcataatgaaggaaaattccatatccaatatgagtttactcagagacagtagaaactattcccagg

SEQ ID NO: 67 - DNA, 450 bp, Homo sapiens
  misc_feature 425: n = A,T,C or G
taggaataacaaatgtttattcagaaatggataagtaatacataatcacccttcatctcttaatgccccttcctctccttctgcacaggagacacagatgggtaacatagaggcatgggaagtggaggaggacacaggactagcccaccaccttctcttcccggtctcccaagatgactgcttatagagtggaggaggcaaacaggtcccctcaatgtaccagatggtcacctatagcaccagctccagatggccacgtggttgcagctggactcaatgaaactctgtgacaaccagaagatacctgctttgggatgagagggaggataaagccatgcagggaggatatttaccatccctaccctaagcacagtgcaagcagtgagcccccggctcccagtacctgaaaaaccaaggcctactgncttttggatgctctcttgggccacg

SEQ ID NO: 68 - DNA, 511 bp, Homo sapiens
aagcctcctgccctggaaatctggagccccttggagctgagctggacggggcagggaggggctgagaggcaagaccgtctccctcctgctgcagctgcttccccagcagccactgctgggcacagcagaaacgccagcagagaaaatgggagccgagagtccttagccctggagctgaggctgcctctgggctgacccgctggctgtacgtggccagaactggggttggcatctggcatccatttgaggccagggtggaggaaagggaggccaacagaggaaaacctattcctgctgtgacaacacagcccttgtcccacgcagcctaagtgcagggagcgtgatgaagtcaggcagccagtcggggaggacgaggtaactcagcagcaatgtcaccttgtagcctatgcgctcaatggcccggaggggcagcaaccccccgcacacgtcagccaacagcagtgcctctgcaggcaccaagagagcgatgatggacttgagcgccgtgttc

SEQ ID NO: 69 - DNA, 511 bp, Homo sapiens
gtttggcagaagacatgtttaataacattttcatatttaaaaaatacagcaacaattctctatctgtccaccatcttgccttgcccttcctggggctgaggcagacaaaggaaaggtaatgaggttagggcccccaggcgggctaagtgctattggcctgctcctgctcaaagagagccatagccagctgggcacggccccctagcccctccaggttgctgaggcggcagcggtggtagagttcttcactgagccgtgggctgcagtctcgcagggagaacttctgcaccagccctggctctacggcccgaaagaggtggagccctgagaaccggaggaaaacatccatcacctccagcccctccagggcttcctcctcttcctggcctgccagttcacctgccagccgggctcgggccgccaggtagtcagcgttgtagaagcagccctccgcagaagcctgccggtcaaatctccccgctataggagccccccgggaggggtcagcacc

SEQ ID NO: 70 - DNA, 511 bp, Homo sapiens
caagttgaacgtcaggcttggcagaggtggagtgtagatgaaaacaaaggtgtgattatgaagaggatgtgagtcctttgggtgtaggagagaaaggctgttgagcttctatttcaagatacttttacctgtgcaaaaagcacattttccacctccttctcatggcatttgtgtaaggtgagtatgattcctattccatctgcattttagaggtgaagaataacgtacaagggattcagtgattagcaagggacccctcactaagtgttgatggagttaggacagagctcagctgtttgaatctcagagcccaggcagctggagctgggtaggatcctggagctggcactaatgtgaggtgcattccctccaacccaggctcagatccggaacctgaccgtgctgacccccgaaggggaggcagggctgagctggcccgttgggctccctgctcctttcacaccacactctcgctttgaggtgctgggctgggactacttcacagagcagc

SEQ ID NO: 71 - DNA, 511 bp, Homo sapiens
tggcctgggcaggattgggagagaggtagctacccggatgcagtcctttgggatgaagactatagggtatgaccccatcatttccccagaggtctcggcctcctttggtgttcagcagctgcccctggaggagatctggcctctctgtgatttcatcactgtgcacactcctctcctgccctccacgacaggcttgctgaatgacaacacctttgcccagtgcaagaagggggtgcgtgtggtgaactgtgcccgtggagggatcgtggacgaaggcgccctgctccgggccctgcagtctggccagtgtgccggggctgcactggacgtgtttacggaagagccgccacgggaccgggccttggtggaccatgagaatgtcatcagctgtccccacctgggtgccagcaccaaggaggctcagagccgctgtggggaggaaattgctgttcagttcgtggacatggtgaaggggaaatctctcacgggggttgtgaatgcccaggccctt

SEQ ID NO: 72 - DNA, 2017 bp, Homo sapiens
agccagatggctgagagctgcaagaagaagtcaggatcatgatggctcagtttcccacagcgatgaatggagggccaaatatgtgggctattacatctgaagaacgtactaagcatgataaacagtttgataacctcaaaccttcaggaggttacataacaggtgatcaagcccgtacttttttcctacagtcaggtctgccggccccggttttagctgaaatatgggccttatcagatctgaacaaggatgggaagatggaccagcaagagttctctatagctatgaaactcatcaagttaaagttgcagggccaacagctgcctgtagtcctccctcctatcatgaaacaaccccctatgttctctccactaatctctgctcgttttgggatgggaagcatgcccaatctgtccattcatcagccattgcctccagttgcacctatagcaacacccttgtcttctgctacttcagggaccagtattcctcccctaatgatgcctgctcccctagtgccttctgttagtacatcctcattaccaaatggaactgccagtctcattcagcctttatccattccttattcttcttcaacattgcctcatgcatcatcttacagcctgatgatgggaggatttggtggtgctagtatccagaaggcccagtctctgattgatttaggatctagtagctcaacttcctcaactgcttccctctcagggaactcacctaagacagggacctcagagtgggcagttcctcagccttcaagattaaagtatcggcaaaaatttaatagtctagacaaaggcatgagcggatacctctcaggttttcaagctagaaatgcccttcttcagtcaaatctctctcaaactcagctagctactatttggactctggctgacatcgatggtgacggacagttgaaagctgaagaatttattctggcgatgcacctcactgacatggccaaagctggacagccactaccactgacgttgcctcccgagcttgtccctccatctttcagagggggaaagcaagttgattctgttaatggaactctgccttcatatcagaaaacacaagaagaagagcctcagaagaaactgccagttacttttgaggacaaacggaaagccaactatgaacgaggaaacatggagctggagaagcgacgccaagtgttgatggagcagcagcagagggaggctgaacgcaaagcccagaaagagaaggaagagtgggagcggaaacagagagaactgcaagagcaagaatggaagaagcagctggagttggagaaacgcttggagaaacagagagagctggagagacagcgggaggaagagaggagaaaggagatagaaagacgagaggcagcaaaacaggagcttgagagacaacgccgtttagaatgggaaagactccgtcggcaggagctgctcagtcagaagaccagggaacaagaagacattgtcaggctgagctccagaaagaaaagtctccacctggaactggaagcagtgaatggaaaacatcagcagatctcaggcagactacaagatgtccaaatcagaaagcaaacacaaaagactgagctagaagttttggataaacagtgtgacctggaaattatggaaatcaaacaacttcaacaagagcttaaggaatatcaaaataagcttatctatctggtccctgagaagcagctattaaacgaaagaattaaaaacatgcagctcagtaacacacctgattcagggatcagtttacttcataaaaagtcatcagaaaaggaagaattatgccaaagacttaaagaacaattagatgctcttgaaaaagaaactgcatctaagctctcagaaatggattcatttaacaatcagctgaaggaactcagagaaagctataatacacagcagttagcccttgaacaacttcataaaatcaaacgtgacaaattgaaggaaatcgaaagaaaaagattagagcaaaaaaaaaaaaa

SEQ ID NO: 73 - DNA, 414 bp, Homo sapiens
atggcagtgacattcaccatcatgggaaccaccttcccttttcttcaggattctctgtagtggaagagagcacccagtgttgggctgaaaacatctgaaagtagggagaagaacctaaaataatcagtatctcagagggctctaaggtgccaagaagtctcactggacatttaagtgccaacaaaggcatactttcggaatcgccaagtcaaaactttctaacttctgtctctctcagagacaagtgagactcaagagtctactgctttagtggcaactacagaaaactggtgttacccagaaaaacaggagcaattagaaatggttccaatatttcaaagctccgcaaacaggatgtgctttcctttgcccatttagggtttcttctctttcctttctctttattaaccacta

SEQ ID NO: 74 - DNA, 1567 bp, Homo sapiens
atatctagaagtctggagtgagcaaacaagagcaagaaacaaaaagaagccaaaagcagaaggctccaatatgaacaagataaatctatcttcaaagacatattagaagttgggaaaataattcatgtgaactagacaagtgtgttaagagtgataagtaaaatgcacgtggagacaagtgcatccccagatctcagggacctccccctgcctgtcacctggggagtgagaggacaggatagtgcatgttctttgtctctgaatttttagttatatgtgctgtaatgttgctctgaggaagcccctggaaagtctatcccaacatatccacatcttatattccacaaattaagctgtagtatgtaccctaagacgctgctaattgactgccacttcgcaactcaggggcggctgcattttagtaatgggtcaaatgattcactttttatgatgcttccaaaggtgccttggcttctcttcccaactgacaaatgccaaagttgagaaaaatgatcataattttagcataaacagagcagtcggcgacaccgattttataaataaactgagcaccttctttttaaacaaacaaatgcgggtttatttctcagatgatgttcatccgtgaatggtccagggaaggacctttcaccttgactatatggcattatgtcatcacaagctctgaggcttctcctttccatcctgcgtggacagctaagacctcagttttcaatagcatctagagcagtgggactcagctggggtgatttcgccccccatctccgggggaatgtctgaagacaattttgttacctcaatgagggagtggaggaggatacagtgctactaccaactagtggataaaggccagggatgctgctcaacctcctaccatgtacaggacgtctccccattacaactacccaatccgaagtgtcaactgtgtcaggactaagaaaccctggttttgagtagaaaagggcctggaaagaggggagccaacaaatctgtctgcttcctcacattagtcattggcaaataagcattctgtctctttggctgctgcctcagcacagagagccagaactctatcgggcaccaggataacatctctcagtgaacagagttgacaaggcctatgggaaatgcctgatgggattatcttcagcttgttgagcttctaagtttctttcccttcattctaccctgcaagccaagttctgtaagagaaatgcctgagttctagctcaggttttcttactctgaatttagatctccagacccttcctggccacaattcaaattaaggcaacaaacatataccttccatgaagcacacacagacttttgaaagcaaggacaatgactgcttgaattgaggccttgaggaatgaagctttgaaggaaaagaatactttgtttccagcccccttcccacactcttcatgtgttaaccactgccttcctggaccttggagccacggtgactgtattacatgttgttatagaaaactgattttagagttctgatcgttcaagagaatgattaaatatacatttccta

SEQ ID NO: 75 - DNA, 240 bp, Homo sapiens
tcgagcggccgcccgggcaggtccttcagacttggactgtgtcacactgccaggcttccagggctccaacttgcagacggcctgttgtgggacagtctctgtaatcgcgaaagcaaccatggaagacctgggggaaaacaccatggttttatccaccctgagatctttgaacaacttcatctctcagcgtgcggagggaggctctggactggatatttctacctcggccgcgaccacgct

SEQ ID NO: 76 - DNA, 330 bp, Homo sapiens
  misc_feature 288: n = A,T,C or G
tagcgyggtcgcggccgaggyctgcttytctgtccagcccagggcctgtggggtcagggcggtgggtgcagatggcatccactccggtggcttccccatctttctctggcctgagcaaggtcagcctgcagccagagtacagagggccaacactggtgttcttgaacaagggccttagcaggccctgaaggrccctctctgtagtgttgaacttcctggagccaggccacatgttctcctcataccgcaggytagygatggtgaagttgagggtgaaatagtattmangragatggctggcaracctgcccgggcggccgctcsaaatcc

SEQ ID NO: 77 - DNA, 361 bp, Homo sapiens
agcgtggtcgcggccgaggtgtccttcagggtctgcttatgcccttgttcaagaacaccagtgtcagctctctgtactctggttgcagactgaccttgctcaggcctgagaaggatggggcagccaccagagtggatgctgtctgcacccatcgtcctgaccccaaaagccctggactggacagagagcggctgtactggaagctgagccagctgacccacggcatcactgagctgggcccctacaccctggacagggacagtctctatgtcaatggtttcacccatcggagctctgtacccaccaccagcaccggggtggtcagcgaggagccattcaacctgcccgggcggccgctcga

SEQ ID NO: 78 - DNA, 356 bp, Homo sapiens
  misc_feature 7, 346, 350, 353: n = A,T,C or G
ttggggntttmgagcggccgcccgggcaggtaccggggtggtcagcgaggagccattcacactgaacttcaccatcaacaacctgcggtatgaggagaacatgcagcaccctggctccaggaagttcaacaccacggagagggtccttcagggcctgctcaggtccctgttcaagagcaccagtgttggccctctgtactctggctgcagactgactttgctcagacttgagaaacatggggcagccactggagtggacgccatctgcaccctccgccttgatcccactggtcctggactggacagagagcggctatactgggagctgagccagtcctctggcggngacnccnctt

SEQ ID NO: 79 - DNA, 226 bp, Homo sapiens
agcgtggtcgcggccgaggtccagtcgcagcatgctctttctcctgcccactggcacagtgaggaagatctctgctgtcagtgagaaggctgtcatccactgagatggcagtcaaaagtgcatttaatacacctaacgtatcgaacatcatagcttggcccaggttatctcatatgtgctcagaacacttacaatagcctgcagacctgcccgggcggccgctcga

SEQ ID NO: 80 - DNA, 444 bp, Homo sapiens
  misc_feature 23: n = A,T,C or G
tgtggtgttgaacttcctggagncagggtgacccatgtcctccccatactgcaggttggtgatggtgaagttgagggtgaatggtaccaggagagggccagcagccataattgtsgrgckgsmgmssgaggmwggwgtyycwgaggttcyrarrtccactgtggaggtcccaggagtgctggtggtgggcacagagstcygatgggtgaaaccattgacatagagactgttcctgtccagggtgtaggggcccagctcttyratgycattggycagttkgctyagctcccagtacagccrctctckgyygmgwccagsgcttttggggtcaagatgatggatgcagatggcatccactccagtggctgctccatccttctcggacctgagagaggtcagtctgcagccagagtacagagggccaacactggtgttctttgaata

SEQ ID NO: 81 - DNA, 310 bp, Homo sapiens
tcgagcggccgcccgggcaggtcaggaagcacattggtcttagagccactgcctcctggattccacctgtgctgcggacatctccagggagtgcagaagggaagcaggtcaaactgctcagatcagtcagactggctgttctcagttctcacctgagcaaggtcagtctgcagccagagtacagagggccaacactggtgttcttgaacaagggcttgagcagaccctgcagaaccctcttccgtggtgttgaacttcctggaaaccagggtgttgcatgtttttcctcataatgcaaggttggtgatgg

SEQ ID NO: 82 - DNA, 571 bp, Homo sapiens
  misc_feature 202: n = A,T,C or G
acggtttcaatggacacttttattgtttacttaatggatcatcaattttgtctcactacctacaaatggaatttcatcttgtttccatgctgagtagtgaaacagtgacaaagctaatcataataacctacatcaaaagagaactaagctaacactgctcactttctttttaacaggcaaaatataaatatatgcactctanaatgcacaatggtttagtcactaaaaaattcaaatgggatcttgaagaatgtatgcaaatccagggtgcagtgaagatgagctgagatgctgtgcaactgtttaagggttcctggcactgcatctcttggccactagctgaatcttgacatggaaggttttagctaatgccaagtggagatgcagaaaatgctaagttgacttaggggctgtgcacaggaactaaaaggcaggaaagtactaaatattgctgagagcatccaccccaggaaggactttaccttccaggagctccaaactggcaccacccccagtgctcacatggctgactttatcctccgtgttccatttggcacagcaagtggcagtg

SEQ ID NO: 83 - DNA, 551 bp, Homo sapiens
aaggctggtgggtttttgatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatga

SEQ ID NO: 84 - DNA, 571 bp, Homo sapiens
tttgttccttacatttttctaaagagttacttaaatcagtcaactggtctttgagactcttaagttctgattccaacttagctaattcattctgagaactgtggtataggtggcgtgtctcttctagctgggacaaaagttctttgttttccccctgtagagtatcacagaccttctgctgaagctggacctctgtctgggccttggactcccaaatctgcttgtcatgttcaagcctggaaatgttaatctttaattcttccatatggatggacatctgtctaagttgatcctttagaacactgcaattatcttctttgagtctaatttcttcttctttgctttgaatcgcatcactaaacttcctctcccatttcttagcttcatctatcaccctgtcacgatcatcctggagggaagacatgctcttagtaaaggctgcaagctgggtcacagtactgtccaagttttcctgaagttgctgaacttccttgtctttcttgttcaaagtaacctgaatctctccaattgtctcttccaagtggactttttctctgcgcaaagcatccag

SEQ ID NO: 85 - DNA, 561 bp, Homo sapiens
tcattgcctgtgatggcatctggaatgtgatgagcagccaggaagttgtagatttcattcaatcaaaggattcagcatgtggtggaagctgtgaggcaagagaaacaagaactgtatggcaagttaagaagcacagaggcaaacaagaaggagacagaaaagcagttgcaggaagctgagcaagaaatggaggaaatgaaagaaaagatgagaaagtttgctaaatctaaacagcagaaaatcctagagctggaagaagagaatgaccggcttagggcagaggtgcaccctgcaggagatacagctaaagagtgtatggaaacacttctttcttccaatgccagcatgaaggaagaacttgaaagggtcaaaatggagtatgaaaccctttctaagaagtttcagtctttaatgtctgagaaagactctctaagtgaagaggttcaagatttaaagcatcagatagaaggtaatgtatctaaacaagctaacctagaggccaccgagaaacatgataaccaaacgaatgtcactgaagagggaacacagtctataccaggt

SEQ ID NO: 86 - DNA, 795 bp, Homo sapiens
aagccaataatcaccatttattacttaatatatgccaaccactgtacttggcagttcacaaattctcaccgttacaacaaccccatgaggtatttattcccattctatagatagggaaaccacagctcaagtaagttaggaaactgagccaagtatacacagaatacgaagtggcaaaactagaaggaaagactgacactgctatctgctggcctccagtgtcctggctcttttcacacgggttcaatgtctccagcgctgctgctgctgctgcattaccatgccctcattgtttttcttcctctggtgttcaactgcatccttcaaagaatctaactcattccagagaccacttatttctttctctctttctgaaattacttttaataattcttcatgagggggaaaagaagatgcctgttggtagttttgttgtttaagctgctcaatttgggacttaaacaatttgttttcatcttgtacatcctgtaacagctgtgttttgctagaaagatcactctccctctcttttagcatggcttctaacctcttcaattcattttccttttctttcaacacaatctcaagttcttcaaactgtgatgcagaagaggcctctttcaagttatgttgtgctacttcctgaacatgtgcttttaaagattcattttcttcttgaagatcctgtaaccacttccctgtattggctaggtctttctctttctcttccaaaacagccttcatggtattcatctgttcctcttttccttttaataagttcaggagcttcagaac

SEQ ID NO: 87 - DNA, 594 bp, Homo sapiens
caagctttttttttttttttaaaaagtgttagcattaatgttttattgtcacgcagatggcaactgggtttatgtcttcatattttatatttttgtaaattaaaaaaattacaagttttaaatagccaatggctggttatattttcagaaaacatgattagactaattcattaatggtggcttcaagcttttccttattggctccagaaaattcacccaccttttgtcccttcttaaaaaactggaatgttggcatgcatttgacttcacactctgaagcaacatcctgacagtcatccacatctacttcaaggaatatcacgttggaatacttttcagagagggaatgaaagaaaggcttgatcattttgcaaggcccacaccacgtggctgagaagtcaactactacaagtttatcacctgcagcgtccaaggcttcctgaaaagcagtcttgctctcgatctgcttcaccatcttggctgctggagtctgacgagcggctgtaaggaccgatggaaatggatccaaagcaccaaacagagcttcaagactcgctgcttggcttgaattcggatccgatatcgccatggcct

SEQ ID NO: 88 - DNA, 557 bp, Homo sapiens
aagtgttagcattaatgttttattgtcacgcagatggcaactgggtttatgtcttcatattttatatttttgtaaattaaaaaaattmcaagttttaaatagccaatggctggttatattttcagaaaacatgattagactaattcattaatggtggcttcaagcttttccttattggctccagaaaattcacccaccttttgtcccttcttaaaaaactggaatgttggcatgcatttgacttcacactctgaagcaacatcctgacagtcatccacatctacttcaaggaatatcacgttggaatacttttcagagagggaatgaaagaaaggcttgatcattttgcaaggcccacaccacgtggctgagaagtcaactactacaagtttatcacctgcagcgtccaaggcttcctgaaaagcagtcttgctctcgatctgcttcaccatcttggctgctggagtctgacgagcggctgtaaggaccgatggaaatggatccaaagcaccaaacagagcttcaagactcgctgcttggcatgaattcggatccga

SEQ ID NO: 89 - DNA, 561 bp, Homo sapiens
  misc_feature 544, 551: n = A,T,C or G
tacaaactttattgaaacgcacacgcgcacacacacaaacacccctgtggatagggaaaagcacctggccacagggtccactgaaacggggaggggatggcagcttgtaatgtggcttttgccacaacccccttctgacagggaaggccttagattgaggccccacctcccatggtgatggggagctcagaatggggtccagggagaatttggttagggggaggtgctagggaggcatgagcagagggcaccctccgagtggggtcccgagggctgcagagtcttcagtactgtccctcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttcccacacttcacgtccttcacacccacgtganggctacnggccaggaag

SEQ ID NO: 90 - DNA, 561 bp, Homo sapiens
cccgtgggtgccatccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtctgcctgcgtccgactccgcggtccttgggccctgatggttctacctgctgtgagctgcccagtgggaagtatggctgctgccaatgcccaacgccacctgctgctccgatcacctgcactgctgccccaagacactgtgtgtgacctgatccagagtaagtgcctctccaaggagaacg

SEQ ID NO: 91 - DNA, 541 bp, Homo sapiens
  misc_feature 480, 491: n = A,T,C or G
gaatcacctttctggtttagctagtactttgtacagaacaatgaggtttcccacagcggagtctccctgggctctgtttggctctcggtaaggcaggcctacaccttttcctctcctctatggagaggggaatatgcattaaggtgaaaagtcaccttccaaaagtgagaaagggattcgattgctgcttcaggactgtggaattatttggaatgttttacaaatggttgctacaaaacaacaaaaaaggtaattacaaaatgtgtacatcacaacatgctttttaaagacattatgcattgtgctcacattcccttaaatgttgtttccaaaggtgctcagcctctagcccagctggattctccgggaagaggcagagacagtttggcgaaaaagacacagggaaggagggggtggtgaaaggagaaagcagccttccagttaaagatcagccctcagttaaaggtcagcttcccgcangctggcctcangcggagtctgggtcagagggaggagcagcagcagggtgggactggggcgt

SEQ ID NO: 92 - DNA, 551 bp, Homo sapiens
aaccggagcgcgagcagtagctgggtgggcaccatggctgggatcaccaccatcgaggcggtgaagcgcaagatccaggttctgcagcagcaggcagatgatgcagaggagcgagctgagcgcctccagcgagaagttgagggagaaaggcgggcccgggaacaggctgaggctgaggtggcctccttgaaccgtaggatccagctggttgaagaagagctggaccgtgctcaggagcgcctggccactgccctgcaaaagctggaagaagctgaaaaagctgctgatgagagtgagagaggtatgaaggttattgaaaaccgggccttaaaagatgaagaaaagatggaactccaggaaatccaactcaaagaagctaagcacattgcagaagaggcagataggaagtatgaagaggtggctcgtaagttggtgatcattgaaggagacttggaacgcacagaggaacgagctgagctggcagagtcccgttgccgagagatggatgagcagattagactgatggaccagaacctgaagtgtctgagtgc

SEQ ID NO: 93 - DNA, 531 bp, Homo sapiens
gagaacttggcctttattgtgggcccaggagggcacaaaggtcaggaggcccaagggagggatctggttttctggatagccaggtcatagcatgggtatcagtaggaatccgctgtagctgcacaggcctcacttgctgcagttccggggagaacacctgcactgcatggcgttgatgacctcgtggtacacgacagagccattggtgcagtgcaagggcacgcgcatgggctccgtcctcgagggcaggcagcaggagcattgctcctgcacatcctcgatgtcaatggagtacacagctttgctggcacactttccctggcagtaatgaatgtccacttcctcttgggacttacaatctcccactttgatgtactgcaccttggctgtgatgtctttgcaatcaggctcctcacatgtgtcacagcaggtgcctggaattttcacgattttgcctccttcagccagacacttgtgttcatcaaatggtgggcagcccgtgaccctcttctcccagatgtactctcctct

SEQ ID NO: 94 - DNA, 531 bp, Homo sapiens
  misc_feature 517: n = A,T,C or G
gcctggaccttgccggatcagtgccacacagtgacttgcttggcaaatggccagaccttgctgcagagtcatcgtgtcaattgtgaccatggaccccggccttcatgtgccaacagccagtctcctgttcgggtggaggagacgtgtggctgccgctggacctgcccttgtgtgtgcacgggcagttccactcggcacatcgtcaccttcgatgggcagaatttcaagcttactggtagctgctcctatgtcatctttcaaaacaaggagcaggacctggaagtgctcctccacaatggggcctgcagccccggggcaaaacaagcctgcatgaagtccattgagattaagcatgctggcgtctctgctgagctgcacagtaacatggagatggcagtggatgggagactggtccttgccccgtacgttggtgaaaacatggaagtcagcatctacggcgctatcatgtatgaagtcaggtttacccatcttggccacatcctcacatacaccgccncaaaacaacgagtt

SEQ ID NO: 95 - DNA, 605 bp, Homo sapiens
agatcaacctctgctggtcaggaggaatgccttccttgtcttggatctttgctttgacgttctcgatagtrwcaactkkrytsramskmaagkgyratgrwmttksywgwrasyktmwwmrsgrarayttagacaycccmcctcwgagacgsagkaccargtgcagaggtggactctttctggatgttgtagtcagacagggtgcgtccatcttccagctgtttcccagcaaagatcaacctctgctgatcaggagggatgccttccttatcttggatctttgccttgacattctcgatggtgtcactgggctccacctcgagggtgatggtcttaccagtcagggtcttcacgaagatytgcatcccacctctgagacggagcaccaggtgcagggtrgactctttctggatgttgtagtcagacagggtgcgyccatcttccagctgctttccsagcaaagatcaacctctgctggtcaggaggratgccttccttgtcytggatctttgcyttgacrttctcratggtgtcactcggctccacttcgagagtgatggtcttaccagtcagggtcttcacgaagatctgcatcccacctctaa

SEQ ID NO: 96 - DNA, 531 bp, Homo sapiens
aagtcacaaacagacaaagattattaccagctgcaagctatattagaagctgaacgaagagacagaggtcatgattctgagatgattggagaccttcaagctcgaattacatctttacaagaggaggtgaagcatctcaaacataatctcgaaaaagtggaaggagaaagaaaagaggctcaagacatgcttaatcactcagaaaaggaaaagaataatttagagatagatttaaactacaaacttaaatcattacaacaacggttagaacaagaggtaaatgaacacaaagtaaccaaagctcgtttaactgacaaacatcaatctattgaagaggcaaagtctgtggcaatgtgtgagatggaaaaaaagctgaaagaagaaagagaagctcgagagaaggctgaaaatcgggttgttcagattgagaaacagtgttccatgctagacgttgatctgaagcaatctcagcagaaactagaacatttgactggaaataaagaaaggatggaggatgaagttaagaatcta

SEQ ID NO: 97 - DNA, 1017 bp, Homo sapiens
  misc_feature 963, 995, 1001, 1008, 1010: n = A,T,C or G
cgcctccaccatgtccatcagggtgacccagaagtcctacaaggtgtccacctctggcccccgggccttcagcagccgctcctacacgagtgggcccggttcccgcatcagctcctcgagcttctcccgagtgggcagcagcaactttcgcggtggcctgggcggcggctatggtggggccagcggcatgggaggcatcaccgcagttacggtcaaccagagcctgctgagcccccttgtcctggaggtggaccccaacatccaggccgtgcgcacccaggagaaggagcagatcaagaccctcaacaacaagtttgcctccttcatagacaaggtacggttcctggagcagcagaacaagatgctggagaccaagtggagcctcctgcagcagcagaagacggctcgaagcaacatggacaacatgttcgagagctacatcaacarccttaggcggcagctggagactctgggccaggagaagctgaagctggaggcggagcttggcaacatgcaggggctggtggaggacttcaagaacaagtatgaggatgagatcaataagcgtacagagatggagaacgaatttgtcctcatcaagaaggatgtggatgaagcttacatgaacaaggtagagctggagtctcgcctggaagggctgaccgacgagatcaacttcctcaggcagctgtatgaagaggagatccgggagctgcagtcccagatctcggacacatctgtggtgctgtccatggacaacagccgctccctggacatggacagcatcattgctgaggtcaaggcacagtacgaggatattgccaaccgcagccgggctgaggctgagagcatgtaccaggtcaagtatgaggagctgcagagcctggctgggaagcacggggatgacctgcggcgcacaaagactgagatctctgagatgaacccggaacatcagcccggctncaggctgagattgagggcctcaaaggccaganggcttncctggangnccgccat

SEQ ID NO: 98 - DNA, 561 bp, Homo sapiens
cccggagccagccaacgagcggaaaatggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccctggagcacctggagcttatcccggagcacctgcacctggagtctacccagggccacccagcggccctggggcctacccatcttctggacagccaagtgccaccggagcctaccctgccactggcccctatggcgcccctgctgggccactgattgtgccttataacctgcctttgcctgggggagtggtgcctcgcatgctgataacaattctgggcacggtgaagcccaatgcaaacagaattgctttagatttccaaagagggaatgatgttgccttccactttaacccacgcttcaatgagaacaacaggagagtcattggttgcaatacaaagctggataa

SEQ ID NO: 99 - DNA, 636 bp, Homo sapiens
gggaatgcaacaactttattgaaaggaaagtgcaatgaaatttgttgaaaccttaaaaggggaaacttagacaccccccctcragcgmagkaccargtgcaraggtggactctttctggatgttgtagtcagacagggtrcgwccatcttccagctgtttyccrgcaaagatcaacctctgctgatcaggaggratgccttccttatcttggatctttgccttgacattctcgatggtgtcactgggctccacctcgagggtgatggtcttaccagtcagggtcttcacgaagatytgcatcccacctctgagacggagcaccaggtgcagggtrgactctttctggatgttgtagtcagacagggtgcgyccatcttccagctgctttccsagcaaagatcaacctctgctggtcaggaggratgccttccttgtcytggatctttgcyttgacrttctcaatggtgtcactcggctccacttcgagagtgatggtcttaccagtcagggtcttcacgaagatctgcatcccacctctaagacggagcaccaggtgcagggtggactctttctggatggttgtagtcagacagggtgcgtccatcttccagctgtttcccagcaaagatcaacct

SEQ ID NO: 100 - DNA, 697 bp, Homo sapiens
aggttgatctttgctgggaaacagctggaagatggacgcaccctgtctgactacaaccatccagaaagagtccaccctgcacctggtgctccgtcttagaggtgggatgcagatcttcgtgaagaccctgactggtaagaccatcactctcgaagtggagccgagtgacaccattgagaaygtcaargcaaagatccargacaaggaaggcatycctcctgaccagcagaggttgatctttgctsggaaagcagctggaagatggrcgcaccctgtctgactacaacatccagaaagagtcyaccctgcacctggtgctccgtctcagaggtgggatgcaratcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccacctytgcacytggtmctbcgtctyagaggkgggrtgcaaatctwmgtkwagacactcactkkyaagryyatcamcmwtgakktcgakyscastkwcactwtcrakaamgtyrwwgcawagatccmagacaaggaaggcattcctcctgaccagcagaggttgatct

SEQ ID NO: 101 - DNA, 451 bp, Homo sapiens
atggagtctcactctgtcgaccaggctggagcgctgtggtgcgatatcggctcactgcagtctccacttcctgggttcaagcgatcctcctgcctcagcctcccgagtagctgggactacaggcaggcgtcaccataatttttgtatttttagtagagacatggtttcgccatgttggctgggctggtctcgaactcctgacctcaagtgatctgtcctggcctcccaaagtgttgggattacaggcgaaagccaacgctcccggccagggaacaactttagaatgaaggaaatatgcaaaagaacatcacatcaaggatcaattaattaccatctattaattactatatgtgggtaattatgactatttcccaagcattctacgttgactgcttgagaagatgtttgtcctgcatggtggagagtggagaagggccaggattcttaggtt

SEQ ID NO: 102 - DNA, 571 bp, Homo sapiens
agcgcggtcttccggcgcgagaaagctgaaggtgatgtggccgccctcaaccgacgcatccagctcgttgaggaggagttggacagggctcaggaacgactggccacggccctgcagaagctggaggaggcagaaaaagctgcagatgagagtgagagaggaatgaaggtgatagaaaaccgggccatgaaggatgaggagaagatggagattcaggagatgcagctcaaagaggccaagcacattgcggaagaggctgaccgcaaatacgaggaggtagctcgtaagctggtcatcctggagggtgagctggagagggcagaggagcgtgcggaggtgtctgaactaaaatgtggtgacctggaagaagaactcaagaatgttactaacaatctgaaatctctggaggctgcatctgaaaagtattctgaaaaggaggacaaatatgaagaagaaattaaacttctgtctgacaaactgaaagaggctgagacccgtgctgaatttgcagagagaacggttgcaaaactggaaaagacaattgatgacctggaagagaaacttgcccagc

SEQ ID NO: 103 - DNA, 451 bp, Homo sapiens
gtgcacaggtcccatttattgtagaaaataataataattacagtgatgaatagctcttcttaaattacaaaacagaaaccacaaagaaggaagaggaaaaaccccaggacttccaagggtgaagctgtcccctcctccctgccaccctcccaggctcattagtgtccttggaaggggcagaggactcagaggggatcagtctccaggggccctgggctgaagcgggtgaggcagagagtcctgaggccacagagctgggcaacctgagccgcctctctggccccctcccccaccactgcccaaacctgtttacagcaccttcgcccctcccctctaaacccgtccatccactctgcacttcccaggcaggtgggtgggccaggcctcagccatactcctgggcgcgggtttcggtgagcaaggcacagtcccagaggtgatatcaaggcct

SEQ ID NO: 104 - DNA, 441 bp, Homo sapiens
gcaaggaactggtctgctcacacttgctggcttgcgcatcaggactggctttatctcctgactcacggtgcaaaggtgcactctgcgaacgttaagtccgtccccagcgcttggaatcctacggcccccacagccggatcccctcagccttccaggtcctcaactcccgtggacgctgaacaatggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgactcgctgctggcactgccgcaggacctgcaggcggcccgcgccctcgtcatcatca

SEQ ID NO: 105 - DNA, 509 bp, Homo sapiens
  misc_feature 195: n = A,T,C or G
tgcaaaagggacacaggggttcaaaaataaaaatttctcttccccctccccaaacctgtaccccagctccccgaccacaacccccttcctcccccggggaaagcaagaaggagcaggtgtggcatctgcagctgggaagagagaggccggggaggtgccgagctcggtgctggtctctttccaaatataaatacntgtgtcagaactggaaaatcctccagcacccaccacccaagcactctccgttttctgccggtgtttggagaggggcggggggcaggggcgccaggcaccggctggctgcggtctactgcatccgctgggtgtgcaccccgcgagcctcctgctgctcattgtagaagagatgacactcggggtccccccggatggtgggggctccctggatcagcttcccggtgttggggttcacacaccagcactccccacgctgcccgttcagagacatcttgcactgtttgaggttgtacaggccatgcttgtcacagttg

SEQ ID NO: 106 - DNA, 571 bp, Homo sapiens
gggttggagggactggttctttatttcaaaaagacacttgtcaatattcagtatcaaaacagttgcactattgatttctctttctcccaatcggccccaaagagaccacataaaaggagagtacattttaagccaataagctgcaggatgtacacctaacagacctcctagaaaccttaccagaaaatggggactgggtagggaaggaaacttaaaagatcaacaaactgccagcccacggactgcagaggctgtcacagccagatggggtggccagggtgccacaaacccaaagcaaagtttcaaaataatataaaatttaaaaagttttgtacataagctattcaagatttctccagcactgactgatacaaagcacaattgagatggcacttctagagacagcagcttcaaacccagaaaagggtgatgagatgagtttcacatggctaaatcagtggcaaaaacacagtcttctttctttctttctttcaaggaggcaggaaagcaattaagtggtcacctcaacataagggggacatgatccattctgtaagcagttgtgaagggg

SEQ ID NO: 107 - DNA, 555 bp, Homo sapiens
caggaaccggagcgcgagcagtagctgggtgggcaccatggctgggatcaccaccatcgaggcggtgaagcgcaagatccaggttctgcagcagcaggcagatgatgcagaggagcgagctgagcgcctccagcgagaagttgagggagaaaggcgggcccgggaacaggctgaggctgaggtggcctccttgaaccgtaggatccagctggttgaagaagagctggaccgtgctcaggagcgcctggccactgccctgcaaaagctggaagaagctgaaaaagctgctgatgagagtgagagaggtatgaaggttattgaaaaccgggccttaaaagatgaagaaaagatggaactccaggaaatccaactcaaagaagctaagcacattgcagaagaggcagataggaagtatgaagaggtggctcgtaagttggtgatcattgaaggagacttggaacgcacagaggaacgagctgagctggcagagtcccgttgccgagagatggatgagcagattagactgatggaccagaacctgaagtgtctgagtgc

SEQ ID NO: 108 - DNA, 541 bp, Homo sapiens
atctacgtcatcaatcaggctggagacaccatgttcaatcgagctaagctgctcaatattggctttcaagaggccttgaaggactatgattacaactgctttgtgttcagtgatgtggacctcattccgatggacgaccgtaatgcctacaggtgtttttcgcagccacggcacatttctgttgcaatggacaagttcgggtttagcctgccatatgttcagtattttggaggtgtctctgctctcagtaaacaacagtttcttgccatcaatggattccctaataattattggggttggggaggagaagatgacgacatttttaacagattagttcataaaggcatgtctatatcacgtccaaatgctgtagtagggaggtgtcgaatgatccggcattcaagagacaagaaaaatgagcccaatcctcagaggtttgaccggatcgcacatacaaaggaaacgatgcgcttcgatggtttgaactcacttacctacaaggtgttggatgtcagagatacccgttatatacccaaatcac

SEQ ID NO: 109 - DNA, 411 bp, Homo sapiens
ctagacctctaattaaaaggcacaatcatgctggagaatgaacagtctgaccccgagggccacagcgaattttagggaaggaggcaaagaggtgagaagggaaaggaaagaaggaaggaaggagaacaataagaactggagacgttgggtgggtcagggagtgtggtggaggctcggagagatggtaaacaaacctgactgctatgagttttcaaccccatagtctagggccatgagggcgtcagttcttggtggctgagggtccttccacccagcccacctgggggagtggagtggggagttctgccaggtaagcagatgttgtctcccaagttcctgacccagatgtctggcaggataacgctgacctgttccctcaacaagggacctgaaagtaattttgctctttac

SEQ ID NO: 110 - DNA, 451 bp, Homo sapiens
ccgaattcaagcgtcaacgatccytcccttaccatcaaatcaattggccaccaatggtactgaacctacgagtacaccgactacgggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaagccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctag

SEQ ID NO: 111 - DNA, 541 bp, Homo sapiens
gctcttcacacttttattgttaattctcttcacatggcagatacagagctgtcgtcttgaagaccaccactgaccaggaaatgccacttttacaaaatcatccccccttttcatgattggaacagttttcctgaccgtctgggagcgttgaagggtgaccagcacatttgcacatgcaaaaaaggagtgaccccaaggcctcaaccacacttcccagagctcaccatgggctgcaggtgacttgccaggtttggggttcgtgagctttccttgctgctgcggtggggaggccctcaagaactgagaggccggggtatgcttcatgagtgttaacatttacgggacaaaagcgcatcattaggataaggaacagccacagcacttcatgcttgtgagggttagctgtaggagcgggtgaaaggattccagtttatgaaaatttaaagcaaacaacggtttttagctgggtgggaaacaggaaaactgtgatgtcggccaatgaccaccatttttctgcccatgtgaaggtccccatgaaacc

SEQ ID NO: 112 - DNA, 521 bp, Homo sapiens
caagcgcttggcgtttggacccagttcagtgaggttcttgggttttgtgcctttggggattttggtttgacccaggggtcagccttaggaaggtcttcaggaggaggccgagttccccttcagtaccacccctctctccccactttccctctcccggcaacatctctgggaatcaacagcatattgacacgttggagccgagcctgaacatgcccctcggccccagcacatggaaaacccccttccttgcctaaggtgtctgagtttctggctcttgaggcatttccagacttgaaattctcatcagtccattgctcttgagtctttgcagagaacctcagatcaggtgcacctgggagaaagactttgtccccacttacagatctatctcctcccttgggaagggcagggaatggggacggtgtatggaggggaagggatctcctgcgcccttcattgccacacttggtgggaccatgaacatctttagtgtctgagcttctcaaattactgcaatagga

SEQ ID NO: 113 - DNA, 568 bp, Homo sapiens
agcgtcaaatcagaatggaaaagactcaaaaccatcatcaacaccaagatcaaaaggacaagratccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtgggsmgacaaaaatatacatgtgaaataa

SEQ ID NO: 114 - DNA, 483 bp, Homo sapiens
tccgaattccaagcgaattatggacaaacgattccttttagaggattacttttttcaatttcggttttagtaatctaggctttgcctgtaaagaatacaacgatggattttaaatactgtttgtggaatgtgtttaaaggattgattctagaacctttgtatatttgatagtatttctaactttcatttctttactgtttgcagttaatgttcatgttctgctatgcaatcgtttatatgcacgtttctttaatttttttagattttcctggatgtatagtttaaacaacaaaaagtctatttaaaactgtagcagtagtttacagttctagcaaagaggaaagttgtggggttaaactttgtattttctttcttatagaggcttctaaaaaggtatttttatatgttctttttaacaaatattgtgtacaacctttaaaacatcaatgtttggatcaaaacaagacccagcttattttctgc

SEQ ID NO: 115 - DNA, 521 bp, Homo sapiens
tgtggtggcgcgggctgaggtggaggcccaggactctgaccctgcccctgccttcagcaaggcccccggcagcgccggccactacgaactgccgtgggttgaaaaatataggccagtaaagctgaatgaaattgtcgggaatgaagacaccgtgagcaggctagaggtctttgcaagggaaggaaatgtgcccaacatcatcattgcgggccctccaggaaccggcaagaccacaagcattctgtgcttggcccgggccctgctgggcccagcactcaaagatgccatgttggaactcaatgcttcaaatgacaggggcattgacgttgtgaggaataaaattaaaatgtttgctcaacaaaaagtcactcttcccaaaggccgacataagatcatcattctggatgaagcagacagcatgaccgacggagcccagcaagccttgaggagaaccatggaaatctactctaaaaccactcgttcgcccttgcttgtaatgcttcggataagatcatcgagcc

SEQ ID NO: 116 - DNA, 501 bp, Homo sapiens
ctttgcaaagcttttatttcatgtctgcggcatggaatccacctgcacatggcatcttagctgtgaaggagaaagcagtgcacgagaaggaatgagtgggcggaaccaacggcctccacaagctgccttccagcagcctgccaaggccatggcagagagagactgcaaacaaacacaagcaaacagagtctcttcacagctggagtctgaaagctcatagtggcatgtgtgaatctgacaaaattaaaagtgtgcatagtccattacatgcataaaacactaataataatcctgtttacacgtgactgcagcaggcaggtccagctccaccactgccctcctgccacatcacatcaagtgccatggtttagagggtttttcatatgtaattcttttattctgtaaaaggtaacaaaatatacagaacaaaactttccctttttaaaactaatgttacaaatctgtattatcacttggatataaatagtatataagctgatc

SEQ ID NO: 117 - DNA, 451 bp, Homo sapiens
  misc_feature 320: n = A,T,C or G
caagggatatatgttgagggtacrgrgtgacactgaacagatcacaaagcacgagaaacattagttctctccctccccagcgtctccttcgtctccctggttttccgatgtccacagagtgagattgtccctaagtaactgcatgatcagagtgctgkctttataagactcttcattcagcgtatccaattcagcaattgcttcatcaaatgccgtttttgccaggctacaggccttttcaggagagtttagaatctcatagtaaaagactgagaaatttagtgccagaccaagacgaattgggtgtgtaggctgcattnctttcttactaatttcaaatgcttcctggtaagcctgctgggagttcgacacaagtggtttgtttgttgctccagatgccacttcagaaagatacctaaaataatctcctttcattttcaaagtagaacac

SEQ ID NO: 118 - DNA, 501 bp, Homo sapiens
tccggagccggggtagtcgccgccgccgccgccggtgcagccactgcaggcaccgctgccgccgcctgagtagtgggcttaggaaggaagaggtcatctcgctcggagcttcgctcggaagggtctttgttccctgcagccctcccacgggaatgacaatggataaaagtgagctggtacagaaagccaaactcgctgagcaggctgagcgatatgatgatatggctgcagccatgaaggcagtcacagaacaggggcatgaactctccaacgaagagagaaatctgctctctgttgcctacaagaatgtggtaaggccgcccgccgctcttcctggcgtgtcatctccagcattgagcagaaaacagagaggaatgagaagaagcagcagatgggcaaagagtaccgtgagaagatagaggcagaactgcaggacatctgcaatgatgttctggagcttgttggacaaatatcttattccaatgctacacaacccagaaa

SEQ ID NO: 119 - DNA, 391 bp, Homo sapiens
aaaaagcagcargttcaacacaaaatagaaatctcaaatgtaggatagaacaaaaccaagtgtgtgaggggggaagcaacagcaaaaggaagaaatgagatgttgcaaaaaagatggaggagggttcccctctcctctggggactgactcaaacactgatgtggcagtatacaccattccagagtcaggggtgttcattcttttttgggagtaagaaaaggtggggattaagaagacgtttctggaggcttagggaccaaggctggtctctttcccccctcccaacccccttgatccctttctctgatcaggggaaaggagctcgaatgagggaggtagagttggaaagggaaaggattccacttgacagaatgggacagactccttccca

SEQ ID NO: 120 - DNA, 421 bp, Homo sapiens
  misc_feature 409: n = A,T,C or G
tggcaatagcacagccatccaggagctcttcargcgcatctcggagcagttcactgccatgttccgccggaaggccttcctccactggtacacaggcgagggcatggacgagatggagttcaccgaggctgagagcaacatgaacgacctcgtctctgagtatcaagcagtaccaggatgccaccgcagaagaggaggaggatttcggtgaggaggccgaagaggaggcctaaggcagagcccccatcacctcaggcttctcagttcccttagccgtcttactcaactgcccctttcctctccctcagaatttgtgtttgctgcctctatcttgttttttgttttttcttctgggggggtctagaacagtgcctggcacatagtaggcgctcaataaatacttggttgntgaatgtctcct

SEQ ID NO: 121 - DNA, 206 bp, Homo sapiens
agctggcgctagggctcggttgtgaaatacagcgtrgtcagcccttgcgctcagtgtagaaacccacgcctgtaaggtcggtcttcgtccatctgcttttttctgaaatacactaagagcagccacaaaactgtaacctcaaggaaaccataaagcttggagtgccttaatttttaaccagtttccaataaaacggtttactacct

SEQ ID NO: 122 - DNA, 131 bp, Homo sapiens
ggagatgaagatgaggaagctgagtcagctacgggcargcgggcagctgaagatgatgaggatgacgatgtcgataccaagaagcagaagaccgacgaggatgactagacagcaaaaaaggaaaagttaaa

SEQ ID NO: 123 - DNA, 231 bp, Homo sapiens
  misc_feature 166, 202, 222, 225: n = A,T,C or G
gatgaaaattaaatacttaaattaatcaaaaggcactacgataccacctaaaacctactgcctcagtggcagtakgctaakgaagatcaagctacagsacatyatctaatatgaatgttagcaattacatakcargaagcatgtttgctttccagaagactatggnacaatggtcattwgggcccaagaggatatttggccnggaaaggatcaagatagatnaangtaaag

SEQ ID NO: 124 - DNA, 521 bp, Homo sapiens
  misc_feature 284, 412, 513: n = A,T,C or G
gagtagcaacgcaaagcgcttggtattgagtctgtgggsgacttcggttccggtctctgcagcagccgtgatcgcttagtggagtgcttagggtagttggccaggatgccgaatatcaaaatcttcagcaggcagctcccaccaggacttatctcasaaaattgctgaccgcctgggcctggagctaggcaaggtggtgactaagaaattcagcaaccaggagacctgtgtggaaattggtgaaagtgtaccgtggagaggatgtctacattgttcagagtggntgtggcgaaatcaatgacaatttaatggagcttttgatcatgattaatgcctgcaagattgcttcagccagccgggttactgcagtcatcccatgcttcccttatgccccggcaggataagaaagatnagagccgggccgccaatctcagccaagcttggtgcaaatatgctatctgtagcagtgcagatcatattatcaccatggacctacatgcttctcaaattcanggcttttt

SEQ ID NO: 125 - DNA, 341 bp, Homo sapiens
  misc_feature 277: n = A,T,C or G
atgcaaaaggggacacagggggttcaaaaataaaaatttctcttccccctccccaaacctgtaccccagctccccgaccacaacccccttcctcccccggggaaagcaagaaggagcaggtgtggcatctgcagctgggaagagagaggccggggaggtgccgagctcggtgctggtctctttccaaatataaatacgtgtgtcagaactggaaaatcctccagcacccaccacccaagcactctccgttttctgccggtgtttggagaggggcggngggcaggggcgccaggcaccggctggctgcggtctactgcatccgctgggtgtgcaccccgcga

SEQ ID NO: 126 - DNA, 521 bp, Homo sapiens
  misc_feature 353, 399, 455: n = A,T,C or G
aggttggagaaggtcatgcaggtgcagattgtccaggskcagccacagggtcaagcccaacaggcccagagtggcactggacagaccatgcaggtgatgcagcagatcatcactaacacaggagagatccagcagatcccggtgcagctgaatgccggccagctgcagtatatccgcttagcccagcctgtatcaggcactcaagttgtgcagggacagatccagacacttgccaccaatgctcaacagattacacagacagaggtccagcaaggacagcagcagttcaagccagttcacaagatggacagcagctctaccagatccagcaagtcaccatgcctgcgggccangacctcgccagcccatgttcatccagtcaagccaaccagcccttcnacgggcaggccccccaggtgaccggcgactgaagggcctgagctggcaaggccaangacacccaacacaatttttgccatacagcccccaggcaatgggcacagcctttcttcccagaggac

SEQ ID NO: 127 - DNA, 351 bp, Homo sapiens
tgagatttattgcatttcatgcagcttgaagtccatgcaaaggrgactagcacagtttttaatgcatttaaaaaataaaagggaggtgggcagcaaacacacaaagtcctagtttcctgggtccctgggagaaaagagtgtggcaatgaatccacccactctccacagggaataaatctgtctcttaaatgcaaagaatgtttccatggcctctggatgcaaatacacagagctctggggtcagagcaagggatggggagaggaccacgagtgaaaaagcagctacacacattcacctaattccatctgagggcaagaacaacgtggcaagtcttgggggtagcagctgtt

SEQ ID NO: 128 - DNA, 521 bp, Homo sapiens
tccagacatgctcctgtcctaggcggggagcaggaaccagacctgctatgggaagcagaaagagttaagggaaggtttcctttcattcctgttccttctcttttgcttttgaacagtttttaaatatactaatagctaagtcatttgccagccaggtcccggtgaacagtagagaacaaggagcttgctaagaattaattttgctgtttttcaccccattcaaacagagctgccctgttccctgatggagttccattcctgccagggcacggctgagtaacacgaagccattcaagaaaggcgggtgtgaaatcactgccaccccatggacagacccctcactcttccttcttagccgcagcgctacttaataaatatatttatactttgaaattatgataaccgatttttcccatgcggcatcctaagggcacttgccagctcttatccggacagtcaagcactgttgttggacaacagataaaggaaaagaaaaagaagaaaacaaccgcaacttctgt

SEQ ID NO: 129 - DNA, 521 bp, Homo sapiens
tgagacggaccactggcctggtcccccctcatktgctgtcgtaggacctgacatgaaacgcagatctagtggcagagaggaagatgatgaggaacttctgagacgtcggcagcttcaagaagagcaattaatgaagcttaactcaggcctgggacagttgatcttgaaagaagagatggagaaagagagccgggaaaggtcatctctgttagccagtcgctacgattctcccatcaactcagcttcacatattccatcatctaaaactgcatctctccctggctatggaagaaatgggcttcaccggcctgtttctaccgacttcgctcagtataacagctatggggatgtcagcgggggagtgcgagattaccagacacttccagatggccacatgcctgcaatgagaatggaccgaggagtgtctatgcccaacatgttggaaccaaagatatttccatatgaaatgctcatggtgaccaacagagggccgaaaccaaatctcagagaggtggacagaa

SEQ ID NO: 130 - DNA, 270 bp, Homo sapiens
tcactttatttttcttgtataaaaaccctatgttgtagccacagctggagcctgagtccgctgcacggagactctggtgtgggtcttgacgaggtggtcagtgaactcctgatagggagacttggtgaatacagtctccttccagaggtcgggggtcaggtagctgtaggtcttagaaatggcatcaaaggtggccttggcgaagttgcccagggtggcagtgcagccccgggctgaggtgtagcagtcatcgataccagccatcatgag

SEQ ID NO: 131 - DNA, 341 bp, Homo sapiens
ctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaaca

SEQ ID NO: 132 - DNA, 844 bp, Homo sapiens
  misc_feature 37: n = A,T,C or G
tgaatggggaggagctgacccaggaaatggagcttgnggagaccaggcctgcaggggatggaaccttccagaagtgggcatctgtggtggtgcctcttgggaaggagcagaagtacacatgccatgtggaacatgaggggctgcctgagcccctcaccctgagatggggcaaggaggagcctccttcatccaccaagactaacacagtaatcattgctgttccggttgtccttggagctgtggtcatccttggagctgtgatggcttttgtgatgaagaggaggagaaacacaggtggaaaaggaggggactatgctctggctccaggctcccagagctctgatatgtctctcccagattgtaaagtgtgaagacagctgcctggtgtggacttggtgacagacaatgtcttcacacatctcctgtgacatccagagacctcagttctctttagtcaagtgtctgatgttccctgtgagtctgcgggctcaaagtgaagaactgtggagcccagtccacccctgcacaccaggaccctatccctgcactgccctgtgttcccttccacagccaaccttgctgctccagccaaacattggtggacatctgcagcctgtcagctccatgctaccctgaccttcaactcctcacttccacactgagaataataatttgaatgtgggtggctggagagatggctcagcgctgactgctcttccaaaggtcctgagttcaaatcccagcaaccacatggtggctcacaaccatctgtaatgggatctaataccctcttctgcagtgtctgaagacasctacagtgtacttacatataataataaataag

SEQ ID NO: 133 - DNA, 601 bp, Homo sapiens
ggccgggcgcgcgcgcccccgccacacgcacgccgggcgtgccagtttataaagggagagagcaagcagcgagtcttgaagctctgtttggtgctttggatccatttccatcggtccttacagccgctcgtcagactccagcagccaagatggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaatcatgttttctgaaaatataaccagccattggctatttaaaacttgtaatttttttaatttacaaaaatataaaatatgaagacataaacccmgttgccatctgcgtgacaataaaacattaatgctaacactt

SEQ ID NO: 134 - DNA, 421 bp, Homo sapiens
tcacataagaaatttaagcaagttacrctatcttaaaaaacacaacgaatgcattttaatagagaaacccttccctccctccacctccctcccccaccctcctcatgaattaagaatctaagagaagaagtaaccataaaaccaagttttgtggaatccatcatccagagtgcttacatggtgattaggttaatattgccttcttacaaaatttctattttaaaaaaaattataaccttgattgcttattacaaaaaaattcagtacaaaagttcaatatattgaaaaatgcttttcccctccctcacagcaccgttttatatatagcagagaataatgaagagattgctagtctagatggggcaatcttcaaattacaccaagacgcacagtggtttatttaccctccccttctcataag

SEQ ID NO: 135 - DNA, 511 bp, Homo sapiens
ggaaaggattcaagaattagaggacttgcttgctrragaaaaagacaactctcgtcgcatgctgacagacaaagagagagagatggcggaaataagggatcaaatgcagcaacagctgaatgactatgaacagcttcttgatgtaaagttagccctggacatggaaatcagtgcttacaggaaactcttagaaggcgaagaagagaggttgaagctgtctccaagcccttcttcccgtgtgacagtatcccgagcatcctcaagtcgtagtgtaccgtacaactagaggaaagcggaagagggttgatgtggaagaatcagaggcgaagtagtagtgttagcatctctcattccgcctcaaccactggaaatgtttgcatcgaagaaattgatgttgatgggaaatttatcccgcttgaagaacacttctgaacaggatcaaccaatgggaaggcttgggagatgatcagaaaaattggagacacatcagtcagttataaatatacctcaa

SEQ ID NO: 136 - DNA, 341 bp, Homo sapiens
catgggtttcaccaggttggccaggctgctcttgaactsctgacctcaggtgatccacccgcctcggcctcccaaagtgctgggattacaggcgtgagccaccacgcccggcccccaaagctgtttcttttgtctttagcgtaaagctctcctgccatgcagtatctacataactgacgtgactgccagcaagctcagtcactccgtggtctttttctctttccagttcttctctctctcttcaagttctgcctcagtgaaagctgcaggtccccagttaagtgatcaggtgagggttctttgaacctggttctatcagtcgaattaatccttcatgatgg

SEQ ID NO: 137 - DNA, 551 bp, Homo sapiens
gatgtgttggaccctctgtgtcaaaaaaaacctcacaaagaatcccctgctcattacagaagaagatgcatttaaaatatgggttattttcaactttttatctgaggacaagtatccattaattattgtgtcagaagagattgaatacctgcttaagaagcttacagaagctatgggaggaggttggcagcaagaacaatttgaacattataaaatcaactttgatgacagtaaaaatggcctttctgcatgggaacttattgagcttattggaaatggacagtttagcaaaggcatggaccggcagactgtgtctatggcaattaatgaagtctttaatgaacttatattagatgtgttaaagcagggttacatgatgaaaaagggccacagacggaaaaactggactgaaagatggtttgtactaaaacccaacataatttcttactatgtgagtgaggatctgaaggataagaaaggagacattctcttggatgaaaattgctgtgtagaagtccttgcctgacaaaagatggaaagaaatgcctttt

SEQ ID NO: 138 - DNA, 531 bp, Homo sapiens
  misc_feature 490: n = A,T,C or G
gactggttctttatttcaaaaagacacttgtcaatattcagtrtcaaaacagttgcactattgatttctctttctcccaatcggccccaaagagaccacataaaaggagagtacattttaagccaataagctgcaggatgtacacctaacagacctcctagaaaccttaccagaaaatggggactgggtagggaaggaaacttaaaagatcaacaaactgccagcccacggactgcagaggctgtcacagccagatggggtggccagggtgccacaaacccaaagcaaagtttcaaaataatataaaatttaaaaagttttgtacataagctattcaagatttctccagcactgactgatacaaagcacaattgagatggcacttctagagacagcagcttcaaacccagaaaagggtgatgagatgaagtttcacatggctaaatcagtggcaaaaacacagtcttctttctttctttctttcaaggangcaggaaagcaattaagtggtcaccttaacataagggggac

SEQ ID NO: 139 - DNA, 521 bp, Homo sapiens
  misc_feature 517: n = A,T,C or G
tgggtgggcaccatggctgggatcaccaccatcgaggcggtgaagcgcaagatccaggttctgcagcagcaggcagatgatgcagaggagcgagctgagcgcctccagcgagaagttgagggagaaaggcgggcccgggaacaggctgaggctgaggtggcctccttgaaccgtaggatccagctggttgaagaagagctggaccgtgctcaggagcgcctggccactgccctgcaaaagctggaagaagctgaaaaagctgctgatgagagtgagagaggtatgaaggttattgaaaaccgggccttaaaagatgaagaaaagatggaactccaggaaatccaactcaaagaagctaagcacattgcagaagaggcagataggaagtatgaagaggtggctcgtaagttggtgatcattgaaggagacttggaaccgcacagaaggaacgagcttgagcttggcaaaagtcccgttgcccagagatgggatgaaccagattagactgatggaccanaacc

SEQ ID NO: 140 - DNA, 571 bp, Homo sapiens
  misc_feature 7: n = A,T,C or G
aggggcngcgggtgcgtgggccactgggtgaccgacttagcctggccagactctcagcacctggaagcgccccgagagtgacagcgtgaggctgggagggaggacttggcttgagcttgttaaactctgctctgagcctccttgtcgcctgcatttagatggctcccgcaaagaagggtggcgagaagaaaaagggccgttctgccatcaacgaagtggtaacccgagaatacaccatcaacattcacaagcgcatccatggagtgggcttcaagaagcgtgcacctcgggcactcaaagagattcggaaatttgccatgaaggagatgggaactccagatgtgcgcattgacaccaggctcaacaaagctgtctgggccaaaggaataaggaatgtgccataccgaatccggtgtgcggctgtccagaaaacgtaatgaggatgaagattcaccaaataagctatatactttggttacctatgtacctgttaccactttcaaaaatctacagacagtcaatgtggatgagaactaatcgctgatcgtcagatcaaataaagttataaaat

SEQ ID NO: 141 - DNA, 531 bp, Homo sapiens
tcgggagccacacttggccctcttcctctccaaagsgccagaacctccttctctttggagaatggggaggcctcttggagacacagagggtttcaccttggatgacctctagagaaattgcccaagaagcccaccttctggtcccaacctgcagaccccacagcagtcagttggtcaggccctgctgtagaaggtcacttggctccattgcctgcttccaaccaatgggcaggagagaaggcctttatttctcgcccacccattcctcctgtaccagcacctccgttttcagtcagtgttgtccagcaacggtaccgtttacacagtcacctcagacacaccatttcacctcccttgccaagctgttagccttagagtgattgcagtgaacactgtttacacaccgtgaatccattcccatcagtccattccagttggcaccagcctgaaccatttggtacctggtgttaactggagtcctgtttacaaggtggagtcggggcttgctgacttctcttcatttgagggcac

SEQ ID NO: 142 - DNA, 491 bp, Homo sapiens
  misc_feature 410: n = A,T,C or G
acctagacagaaggtgggtgagggaggactggtaggaggctgaggcaattccttggtagtttgtcctgaaaccctactggagaagtcagcatgaggcacctactgagagaagtgcccagaaactgctgactgcatctgttaagagttaacagtaaagaggtagaagtgtgtttctgaatcagagtggaagcgtctcaagggtcccacagtggaggtccctgagctacctcccttccgtgagtgggaagagtgaagcccatgaagaactgagatgaagcaaggatggggttcctgggctccaggcaagggctgtgctctctgcagcagggagccccacgagtcagaagaaaagaactaatcatttgttgcaagaaaccttgcccggatactagcggaaaactggaggcggnggtgggggcacaggaaagtggaagtgatttgatggagagcagagaagcctatgcacagtggccgagtccacttgtaaagtg

SEQ ID NO: 143 - DNA, 515 bp, Homo sapiens
ttcaagcaattgtaacaagtatatgtagattagagtgagcaaaatcatatacaattttcatttccagttgctattttccaaattgttctgtaatgtcgttaaaattacttaaaaattaacaaagccaaaaattatatttatgacaagaaagccatccctacattaatcttacttttccactcaccggcccatctccttcctctttttcctaactatgccattaaaactgttctactgggccgggcgtgtggctcatgcctgtaatcccagcattttgggaggccaaggcaggcggatcatgaggtcaagagattgagaccatcctggccaacatggtgaaaccccgcctcgactaagaatacaaaaattagctgggcatggtggcgcatgcctgtagtctcagctactcgggaggctgaggcagaagaatcgcttgaacccgggaggcagaggatgcagtgagccccgatcgcgccactgcactctagcctgggcgacagactgagactctgctc

SEQ ID NO: 144 - DNA, 340 bp, Homo sapiens
tgtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagttgcccaggccaaccccatggaacaagggcattttgccagcc

SEQ ID NO: 145 - DNA, 630 bp, Homo sapiens
tgtaaaaacttgtttttaattttgtataaaataaaggtggtccatgcccacgggggctgtaggaaatccaagcagaccagctggggtggggggatgtagcctacctcgggggactgtctgtcctcaaaacgggctgagaaggcccgtcaggggcccaggtcccacagagaggcctgggatactcccccaacccgaggggcagactgggcagtggggagcccccatcgtgccccagaggtggccacaggctgaaggaggggcctgaggcaccgcagcctgcaacccccagggctgcagtccactaactttttacagaataaaaggaacatggggatggggaaaaaagcaccaggtcaggcagggcccgagggccccagatcccaggagggccaggactcaggatgccagcaccaccctagcagctcccacagctcctggcacaggaggccgccacggattggcacaggccgctgctggccatcacgccacatttggagaacttgtcccgacagaggtcagctcggaggagctcctcgtgggcacacactgtacgaacacagatctccttgttaatgacgtacacacggcggaggctgcggggacagggcacgggaggtctcagccccactt

SEQ ID NO: 146 - DNA, 521 bp, Homo sapiens
atggctgctggatttaggtggtaataggggctgtgggccataaatctgaagccttgagaaccttgggtctggagagccatgaagagggaaggaaaagagggcaagtcctgaacctaaccaatgacctgatggattgctcgaccaagacacagaagtgaagtctgtgtctgtgcacttcccacagactggagtttttggtgctgaatagagccagttgctaaaaaattgggggtttggtgaagaaatctgattgttgtgtgtattcaatgtgtgattttaaaaataaacagcaacaacaataaaaaccctgactggctgttttttccctgtattctttacaactattttttgaccctctgaaaattattatacttcacctaaatggaagactgctgtgtttgtggaaattttgtaattttttaatttattttattctctctcctttttattttgcctgcagaatccgttgagagactaataaggcttaatatttaattgatttgtttaatatgtatataaat

SEQ ID NO: 147 - DNA, 562 bp, Homo sapiens
ggcatgcgagcgcactcggcggacgcaagggcggcggggagcacacggagcactgcaggcgccgggttgggacagcgtcttcgctgctgctggatagtcgtgttttcggggatcgaggatactcaccagaaaccgaaaatgccgaaaccaatcaatgtccgagttaccaccatggatgcagagctggagtttgcaatccagccaaatacaactggaaaacagctttttgatcaggtggtaaagactatcggcctccgggaagtgtggtactttggcctccactatgtggataataaaggatttcctacctggctgaagctggataagaaggtgtctgcccaggaggtcaggaaggagaatcccctccagttcaagttccgggccaaagttctaccctgaagatgtggctgaggagctcatccaggacatcacccagaaacttttcttccttcaagtgaaggaaggaatccttagcgatgagatctactgccccccttgaractgccgtgctcttggggtcctacgcttgtgcatgccaagtttggggactaccaccaagaag

SEQ ID NO: 148 - DNA, 820 bp, Homo sapiens
gaaggagtcgggatactcagcattgatgcaccccaatttcaaagcggcattcttcggcaggtctctgggacaatctctagggtcactacctggaaactcgttagggtacaactgaatgctgaaaggaaagaacacctgcagaaccggacagaaattcaccccggcgatcagctgattgatctcggtcgaccagaagtcatggctaaagatgacgaggacgttgtcaattccctgggcttttcgaagtgagtccagcagcagtctgaggtattcgggccggttatgcacctggaccaccagcaccagctcccggggggcccaggtgccagccttatctacattcctcagggtctgatcaaagttcagctggtacaccagggaccggtaccgcagcgtcaggttgtccgctcgggctgggggaccgccgggaccagggaagccgccgacacgttggagaccctgcggatgcccacagccacagaggggtggtccccaccgcggccgccggcaccccgcgcgggttcggcgtccagcaacggtggggcgagggcctcgttcttcctttgtcgcccattgctgctccagaggacgaagccgcaggcggccaccacgagcgtcaggattagcaccttccgtttgtagatgcggaacctcatggtctccagggccgggagcgcagctacagctcgagcgtcggcgccgccgctaggagccgcggctcggcttcgtctccgtcctctccattcagcaccacgggtcccggaaaaagctcagccscggtcccaaccgcaccctagcttcgttacctgcgcctcgcttg

SEQ ID NO: 149 - DNA, 501 bp, Homo sapiens
cagatttttatttgcagtcgtcactggggccgtttcttgctgcttatttgtctgctagcctgctcttccagctgcatggccaggcgcaaggccttgatgacatctcgcagggctgagaaatgcttggcttgctgggccagagcagattccgctttgttcacaaaggtctccaggtcatagtctggctgctcggtcatctcagagagctcaagccagtctggtccttgctgtatgatctccttgagctcttccatagccttctcctccagctccctgatctgagtcatggcttcgttaaagctggacatctgggaagacagttcctcctcttccttggataaattgcctggaatcagcgccccgttagagcaggcttccatctcttctgtttccatttgaatcaactgctctccactgggcccactgtgggggctcagctccttgaccctgctgcatatcttaagggtgtttaaaggatattcacaggagcttatgcctggt

SEQ ID NO: 150 - DNA, 511 bp, Homo sapiens
  misc_feature 457, 479: n = A,T,C or G
ctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggccttccgggacgtcttcttctgaagaatcaaccctgctaccggaagttgggcctggaagtctatgtgacattcttcgagatctacaatgggaagctgtttgacctgctcaacaagaaggccaagcttgcgcgtgctggaagacggcaagcaacaggtgcaagtggtgggggcttgcaggaacatctggntaactctgcttgatgatggcantcaagatgatcgacatgggcagcgcctgcaga

SEQ ID NO: 151 - DNA, 566 bp, Homo sapiens
tcccgaattcaagcgacaaattggawagtgaaatggaagatgcctatcatgaacatcaggcaaatcttttgcgccaagatctgatgagacgacaggaagaattaagacgcatggaagaacttcacaatcaagaaatgcagaaacgtaaagaaatgcaattgaggcaagaggaggaacgacgtagaagagaggaagagatgatgattcgtcaacgtgagatggaagaacaaatgaggcgccaaagagaggaaagttacagccgaatgggctacatggatccacgggaaagagacatgcgaatgggtggcggaggagcaatgaacatgggagatccctatggttcaggaggccagaaatttccacctctaggaggtggtggtggcataggttatgaagctaatcctggcgttccaccagcaaccatgagtggttccatgatgggaagtgacatgcgtactgagcgctttgggcagggaggtgcggggcctgtgggtggacagggtcctagaggaatggggcctggaactccagcaggatatggtagagggagagaagagtacgaaggc

SEQ ID NO: 152 - DNA, 518 bp, Homo sapiens
ttcgtgaagaccctgactggtaagaccatcactctcgaagtggagcccgagtgacaccattgagaatgtcaaggcaaagatccaagacaaggaaggcatccctcctgaccagcakaggttgatctttgctgggaaacagctggaagatggacgcaccctgtctgactacaacatccagaaagagtccaccctgcacctggtgctccgtctcagaggtgggatgcaaatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattc

SEQ ID NO: 153 - DNA, 542 bp, Homo sapiens
gcgcgggtgcgtgggccactgggtgaccgacttagcctggccagactctcagcacctggaagcgccccgagagtgacagcgtgaggctgggagggaggacttggcttgagcttgttaaactctgctctgagcctccttgtcgcctgcatttagatggctcccgcaaagaagggtggcgagaagaaaaagggccgttctgccatcaacgaagtggtaacccgagaatacaccatcaacattcacaagcgcatccatggagtgggcttcaagaagcgtgcacctcgggcactcaaagagattcggaaatttgccatgaaggagatgggaactccagatgtgcgcattgacaccaggctcaacaaagctgtctgggccaaaggaataaggaatgtgccataccgaatccgtgtgcggctgtccagaaaacgtaatgaggatgaagattcaccaaataagctatatactttggttacctatgtacctgttaccactttcaaaaatctacagacagtcaatgtggatgagaactaatcgctgatcgt

SEQ ID NO: 154 - DNA, 411 bp, Homo sapiens
aattctttatttaaatcaacaaactcatcttcctcaagccccagaccatggtaggcagccctccctctccatcccctcaccccaccccttagccacagtgaagggaatggaaaatgagaagccacgagggcccctgccagggaaggctgccccagatgtgtggtgagcacagtcagtgcagctgtggctggggcagcagctgccacaggctcctccctataaattaagttcctgcagccacagctgtgggagaagcatacttgtagaagcaaggccagtccagcatcagaaggcagaggcagcatcagtgactcccagccatggaatgaacggaggacacagagctcagagacagaacaggccagggggaagaaggagagacagaataggccagggcatggcggtgaggga

SEQ ID NO: 155 - DNA, 421 bp, Homo sapiens
  misc_feature 173: n = A,T,C or G
tgatgaatctgggtgggctggcagtagcccgagatgatgggctcttctctggggatcccaactggttccctaagaaatccaaggagaatcctcggaacttctcggataaccagctgcaagagggcaagaacgtgatcgggttacagatgggcaccaaccgcggggcgtctcangcaggcatgactggctacgggatgccacgccagatcctctgatcccaccccaggccttgcccctgccctcccacgaatggttaatatatatgtagatatatattttagcagtgacattcccagagagccccagagctctcaagctcctttctgtcagggtggggggttcaagcctgtcctgtcacctctgaagtgcctgctggcatcctctcccccatgcttactaatacattcccttccccatagcc

SEQ ID NO: 156 - DNA, 670 bp, Homo sapiens
agcggagctccctcccctggtggctacaacccacacacgccaggctcaggcatcgagcagaactccagcgactgggtaaccactgacattcaggtgaaggtgcgggacacctacctggatacacaggtggtgggacagacaggtgtcatccgcagtgtcacggggggcatgtgctctgtgtacctgaaggacagtgagaaggttgtcagcatttccagtgagcacctggagcctatcacccccaccaagaacaacaaggtgaaagtgatcctgggcgaggatcgggaagccacgggcgtcctactgagcattgatggtgaggatggcattgtccgtatggaccttgatgagcagctcaagatcctcaacctccgcttcctggggaagctcctggaagcctgaagcaggcagggccggtggacttcgtcggatgaagagtgatcctccttccttccctggcccttggctgtgacacaagatcctcctgcagggctaggcggattgttctggatttccttttgtttttccttttaggtttccatcttttccctccctggtgctcattggaatctgagtagagtctgggggagggtccccaccttcctgtacctcctccccacagcttgcttttgttgtaccgtctttcaataaaaagaagctgtttggtcta

SEQ ID NO: 157 - DNA, 421 bp, Homo sapiens
ggttcacagcactgctgcttgtgtgttgccggccaggaattccaggctcacaaggctatcttagcagctcgttctccggtttttagtgccatgtttgaacatgaaatggaggagagcaaaaagaatcgagttgaaatcaatgatgtggagcctgaagtttttaaggaaatgatgtgcttcatttacacggggaaggctccaaacctcgacaaaatggctgatgatttgctggcagctgctgacaagtatgccctggagcgcttaaaggtcatgtgtgaggatgccctctgcagtaacctgtccgtggagaacgctgcagaaattctcatcctggccgacctccacagtgcagatcagttgaaaactcaggcagtggatttcatcaactatcatgcttcggatgtcttggagacctcttggg

SEQ ID NO: 158 - DNA, 321 bp, Homo sapiens
tcgtagccatttttctgcttctttggagaatgacgccacactgactgctcattgtcgttggttccatgccaattggtgaaatagaacctcatccggtagtggagccggagggacatcttgtcatcaacggtgatggtgcgatttggagcataccagagcttggtgttctcgccatacagggcaaagaggttgtgacaaagaggagagatacggcatgcctgtgcagccctgatgcacagttcctctgctgtgtactctccactgcccagccggaggggctccctgtccgacagatagaagatcacttccacccctggcttg

SEQ ID NO: 159 - DNA, 596 bp, Homo sapiens
tggcacactgctcttaagaaactatgawgatctgagatttttttgtgtatgtttttgactcttttgagtggtaatcatatgtgtctttatagatgtacatacctccttgcacaaatggaggggaattcattttcatcactgggagtgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaattcattgtttaaagatggtcgtgtgtgtgtgtgtgtgtgtgtgtgttgtgttgtgttttgttttttaagggagggaatttattatttaccgttgcttgaaattactgkgtaaatatatgtytgataatgatttgctytttgvcmactaaaattaggvctgtataagtwctaratgcmtccctgggkgttgatyttccmagatattgatgatamcccttaaaattgtaaccygcctttttccctttgctytcmattaaagtctattcmaaag

SEQ ID NO: 160 - DNA, 515 bp, Homo sapiens
gggggtaggctctttattagacggttattgctgtactacagggtcagagtgcagtgtaagcagtgtcagaggcccgcgttcagcccaagaatgtggattttctctccctattgatcacagtgggtgggtttcttcagaaaagccccagaggcagggaccagtgagctccaaggttagaagtggaactggaaggcttcagtcacatgctgcttccacgcttccaggctgggcagcaaggaggagatgcccatgacgtgccaggtctccccatctgacaccagtgaagtctggtaggacagcagccgcacgcctgcctctgccaggaggccaatcatggtaggcagcattgcagggtcagaggtctgagtccggaataggagcaggggcaggtccctgcggagaggcacttctggcctgaagacagctccattgagcccctgcagtacaggygtagtgccttggaccaagcccacagcctggtaaggggcgcctgccagggccacggccaggaggca

SEQ ID NO: 161 - DNA, 936 bp, Homo sapiens
taatttcttagtcgtttggaatccttaagcatgcaaaagctttgaacagaagggttcacaaaggaaccagggttgtcttatggcatccagttaagccagagctgggaatgcctctgggtcatccacatcaggagcagaagcacttgacttgtcggtcctgctgccacggtttgggcgcccaccacgcccacgtccacctcgtcctcccctgccgccacgtcctgggcggccaaggtctccaaaattgatctccagctgagacgttatatcatttgctggcttccggaaatgatggtccataaccgaatcttcagcatgagcctcttcactctttgatttatgaagaacaaatcccttcttccactgcccatcagcaccttcatttggttttcggatattaaattctacttttgcccggtccttattttgaatagccttccactcatccaaagtcatctcttttggaccctcctcttttacctcttcaacttcattctccttattttcagtgtctgccactggatgatgttcttcaccttcaggtgtttcctcagtcacatttgattgatccaagtcagttaattcgtctttgacagttccccagttgtgagatccgctacctccacgtttgtcctcgtgcttcaggccagatctatcacttccactatgcctatcaaattcacgtttgccacgagaatcaaatccatctcctcggcccattccacgtccacggccccctcgacctcttccaagaccaccacgacctcgaataggtcggtcaataatcggtctatcaactgaaaattcgcctccttcacccttttcttcaagtggcttttcgaatcttcgttcacgaggtggtcgcctttctggtcttctatcaattattttcccttcaccctgaagttgttgatcaggtcttcttccaactcgtgc

SEQ ID NO: 162 - DNA, 950 bp, Homo sapiens
aagcggatggacctgagtcagccgaatcctagccccttcccttgggcctgctgtggtgctcgacatcagtgacagacggaagcagcagaccatcaaggctacgggaggcccggggcgcttgcgaagatgaagtttggctgcctctccttccggcagccttatgctggctttgtcttaaatggaatcaagactgtggagacgcgctggcgtcctctgctgagcagccagcggaactgtaccatcgccgtccacattgctcacagggactgggaaggcgatgcctgtcgggagctgctggtggagagactcgggatgactcctgctcagattcaggccttgctcaggaaaggggaaaagtttggtcgaggagtgatagcgggactcgttgacattggggaaactttgcaatgccccgaagacttaactcccgatgaggttgtggaactagaaaatcaagctgcactgaccaacctgaagcagaagtacctgactgtgatttcaaaccccaggtggttactggagcccatacctaggaaaggaggcaaggatgtattccaggtagacatcccagagcacctgatccctttggggcatgaagtgtgacaagtgtgggctcctgaaaggaatgttccrgagaaaccagctaaatcatggcaccttcaatttgccatcgtgacgcagacctgtataaattaggttaaagatgaatttccactgctttggagagtcccacccactaagcactgtgcatgtaaacaggttcctttgctcagatgaaggaagtagggggtggggctttccttgtgtgatgcctccttaggcacacaggcaatgtctcaagtactttgaccttagggtagaaggcaaagctgccagtaaatgtctcagcattgctgctaattttggtcctgctagtttctggattgtacaaataaatgtgttgtagatga

SEQ ID NO: 163 - DNA, 475 bp, Homo sapiens
  misc_feature 301, 317, 331, 458, 464, 470: n = A,T,C or G
tcgagcggccgcccgggcaggtgtcggagtccagcacgggaggcgtggtcttgtagttgttctccggctgcccattgctctcccactccacggcgatgtcgctgggatagaagcctttgaccaggcaggtcaggctgacctggttcttggtcatctcctcccgggatgggggcagggtgtacacctgtggttctcggggctgccctttggctttggagatggttttctcgatgggggctgggagggctttgttggagaccttgcacttgtactccttgccattcaaccagtcctggtgcangacggtgaggacgctnaccacacggtacgngctggtgtactgctcctcccgcggctttgtcttggcattatgcacctccacgccgtccacgtaccaattgaacttgacctcagggtcttcgtggctcacgtccaccaccacgcatgtaacctcaaanctcggncgcgancacgc

SEQ ID NO: 164 - DNA, 476 bp, Homo sapiens
agcgtggtcgcggccgaggtctgaggttacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgcccgtggagtgggagagcaatgggcagccggagaacaactacaagaccacgcctcccgtgctggactccgacacctgccgggcggccgctcga

SEQ ID NO: 165 - DNA, 256 bp, Homo sapiens
  misc_feature 10, 37, 249: n = A,T,C or G
agcgtggttncggccgaggtcccaaccaaggctgcancctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggacctgcccgggcggncgctcga

SEQ ID NO: 166 - DNA, 332 bp, Homo sapiens
agcgtggtcgcggccgaggtcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggacctgcccgggcggccgctcga

SEQ ID NO: 167 - DNA, 332 bp, Homo sapiens
  misc_feature 77, 109, 136, 184, 198: n = A,T,C or G
tcgagcggtcgcccgggcaggtccacatcggcagggtcggagccctggccgccatactcgaactggaatccatcggncatgctctcgccgaaccagacatgcctcttgnccttggggttcttgctgatgtaccagntcttctgggccacactgggctgagtggggtacacgcaggtctcaccantctccatgttgcanaagactttgatggcatccaggttgcagccttggttggggtcaatccagtactctccactcttccagacagagtggcacatcttgaggtcacggcaggtgcgggcggggttcttgacctcggtcgcgaccacgct

SEQ ID NO: 168 - DNA, 276 bp, Homo sapiens
  misc_feature 72, 84: n = A,T,C or G
tcgagcggccgcccgggcaggtcctcctcagagcggtagctgttcttattgccccggcagcctccatagatnaagttattgcangagttcctctccacgtcaaagtaccagcgtgggaaggatgcacggcaaggcccagtgactgcgttggcggtgcagtattcttcatagttgaacatatcgctggagtggacttcagaatcctgccttctgggagcacttgggacagaggaatccgctgcattcctgctggtggacctcggccgcgaccacgct

SEQ ID NO: 169 - DNA, 276 bp, Homo sapiens
agcgtggtcgcggccgaggtccaccagcaggaatgcagcggattcctctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggacctgcccgggcggccgctcga

SEQ ID NO: 170 - DNA, 332 bp, Homo sapiens
  misc_feature 294: n = A,T,C or G
tcgagcggccgcccgggcaggtccacatcggcagggtcggagccctggccgccatactcgaactggaatccatcggtcatgctctcgccgaaccagacatgcctcttgtccttggggttcttgctgatgtaccagttcttctgggccacactgggctgagtggggtacacgcaggtctcaccagtctccatgttgcagaagactttgatggcatccaggttgcagccttggttggggtcaatccagtactctccactcttccagccagaatggcacatcttgaggtcacggcangtgcgggcggggttcttgacctcggccgcgaccacgct

SEQ ID NO: 171 - DNA, 333 bp, Homo sapiens
agcgtggtcgcggccgaggtcaagaaaccccgcccgcacctgccgtgacctcaagatgtgccactctggctggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggctcggcgagagcatgaccgatggattccagttcgagtatggcggccagggctccgaccctgccgatgtggacctgcccgggcggccgctcga

SEQ ID NO: 172 - DNA, 527 bp, Homo sapiens
  misc_feature 46, 125, 140, 148, 220, 229, 291, 388, 456: n = A,T,C or G
agcgtggtcgcggccgaggtcctgtcagagtggcactggtagaagntccaggaaccctgaactgtaagggttcttcatcagtgccaacaggatgacatgaaatgatgtactcagaagtgtcctgnaatggggcccatganatggttgnctgagagagagcttcttgtcctacattcggcgggtatggtcttggcctatgccttatgggggtggccgttgngggcggtgnggtccgcctaaaaccatgttcctcaaagatcatttgttgcccaacactgggttgctgaccanaagtgccaggaagctgaataccatttccagtgtcatacccagggtgggtgacgaaaggggtcttttgaactgtggaaggaacatccaagatctctgntccatgaagattggggtgtggaagggttaccagttggggaagctcgctgtctttttccttccaatcangggctcgctcttctgaatattcttcagggcaatgacataaattgtatattcggttcccggttccaggccag

SEQ ID NO: 173 - DNA, 635 bp, Homo sapiens
  misc_feature 444, 453, 517, 540, 546, 551, 573, 593: n = A,T,C or G
tcgagcggccgcccgggcaggtccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgangaacatggntttaggcggaccacaccggccacaacgggcacccccataaggcataggccaagaacatacccgncgaatgtaggacaagaagctctntctcanacaancatctcatgggccccattccangacacttctgagtacatcanttcatggcatcctggtggcactgataaaaacccttacagtta

SEQ ID NO: 174 - DNA, 572 bp, Homo sapiens
  misc_feature 457, 511, 520, 552, 568: n = A,T,C or G
agcgtggtcgcgggcgaggtcctgtcagagtggcactggtagaagttccaggaaccctgaactgtaagggttcttcatcagtgccaacaggatgacatgaaatgatgtactcagaagtgtcctggaatggggcccatgagatggttgtctgagagagagcttcttgtcctacattcggcgggtatggtcttggcctatgccttatgggggtggccgttgtgggcggtgtggtccgcctaaaaccatgttcctcaaagatcatttgttgcccaacactgggttgctgaccagaagtgccaggaagctgaataccatttccagtgtcatacccagggtgggtgacgaaaggggtcttttgaactgtggaaggaacatccaagatctctggtccatgaagattggggtgtggaagggttaccagttggggaagctcgtctgtctttttccttccaatcangggctcgctcttctgattattcttcagggcaatgacataaattgtatattcggntcccgggtncagccaataataataaccctctgtgacaccanggcggggccgaaggancact

SEQ ID NO: 175 - DNA, 372 bp, Homo sapiens
  misc_feature 247: n = A,T,C or G
agcgtggtcgcggccgaggtcctcaccagaggtaccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttangctttggaagtggtcatttcagatgtgattcatctagatggtgccatgacaatggtgtgaactacaagattggagagaagtgggaccgtcagggagaaaatggacctgcccgggcggccgctcga

SEQ ID NO: 176 - DNA, 372 bp, Homo sapiens
  misc_feature 251: n = A,T,C or G
tcgagcggccgcccgggcaggtccattttctccctgacggtcccacttctctccaatcttgtagttcacaccattgtcatggcaccatctagatgaatcacatctgaaatgaccacttccaaagcctaagcactggcacaacagtttaaagcctgattcagacattcgttcccactcatctccaacggcataatgggaaactgtgtaggggtcaaagcacgagtcatccgtaggttggttcaagccttcgntgacagagttgcccacggtaacaacctcttcccgaaccttatgcctctgctggtctttcagtgcctccactatgatgttgtaggtggtacctctggtgaggacctcggccgcgaccacgct

SEQ ID NO: 177 - DNA, 269 bp, Homo sapiens
  misc_feature 94, 225: n = A,T,C or G
agcgtggccgcggccgaggtccattggctggaacggcatcaacttggaagccagtgatcgtctcagccttggttctccagctaatggtgatggnggtctcagtagcatctgtcacacgagcccttcttggtgggctgacattctccagagtggtgacaacaccctgagctggtctgcttgtcaaagtgtccttaagagcatagacactcacttcatatttggcgnccaccataagtcctgatacaaccacggaatgacctgtcaggaac

SEQ ID NO: 178 - DNA, 529 bp, Homo sapiens
tcgagcggccgcccgggcaggtcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggcggccaccaaatatgaagtgagtgtctatgctcttaaggacactttgacaagcagaccagctcagggtgttgtcaccactctggagaatgtcagcccaccaagaagggctcgtgtgacagatgctactgagaccaccatcaccattagctggagaaccaagactgagacgatcactggcttccaagttgatgccgttccagccaatggacctcggccgcgaccacgctt

SEQ ID NO: 179 - DNA, 454 bp, Homo sapiens
  misc_feature 64: n = A,T,C or G
agcgtggtcgcggccgaggtctggccgaactgccagtgtacagggaagatgtacatgttatagntcttctcgaagtcccgggccagcagctccacggggtggtctcctgcctccaggcgcttctcattctcatggatcttcttcacccgcagcttctgcttctcagtcagaaggttgttgtcctcatccctctcatacagggtgaccaggacgttcttgagccagtcccgcatgcgcagggggaattcggtcagctcagagtccaggcaaggggggatgtatttgcaaggcccgatgtagtccaagtggagcttgtggcccttcttggtgccctccaaggtgcactttgtggcaaagaagtggcaggaagagtcgaaggtcttgttgtcattgctgcacaccttctcaaactcgccaatgggggctgggcagacctgcccgggcggccgctcga

SEQ ID NO: 180 - DNA, 454 bp, Homo sapiens
  misc_feature 55, 299, 317, 332, 342, 348: n = A,T,C or G
tcgagcggccgcccgggcaggtctgcccagcccccattggcgagtttgagaaggngtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcanaagctgcgggtgaagaanatccatgagaatganaagcgcctgnaggcangagaccaccccgtggagctgctggcccgggacttcgagaagaactataacatgtacatcttccctgtacactggcagttcggccagacctcggccgcgaccacgct

SEQ ID NO: 181 - DNA, 102 bp, Homo sapiens
  misc_feature 8, 47, 60, 67: n = A,T,C or G
agcgtggntgcggacgacgcccacaaagccattgtatgtagttttanttcagctgcaaanaataccnccagcatccaccttactaaccagcatatgcagaca

SEQ ID NO: 182 - DNA, 337 bp, Homo sapiens
  misc_feature 169, 195, 253, 314: n = A,T,C or G
tcgagcggtcgcccgggcaggtctgggcggatagcaccgggcatattttggaatggatgaggtctggcaccctgagcagcccagcgaggacttggtcttagttgagcaatttggctaggaggatagtatgcagcacggttctgagtctgtgggatagctgccatgaagnaacctgaaggaggcgctggctggtangggttgattacagggctgggaacagctcgtacacttgccattctctgcatatactggntagtgaggcgagcctggcgctcttctttgcgctgagctaaagctacatacaatggctttgnggacctcggccgcgaccacgctt

SEQ ID NO: 183 - DNA, 374 bp, Homo sapiens
tcgagcggccgcccgggcaggtccattttctccctgacggtcccacttctctccaatcttgtagttcacaccattgtcatgacaccatctagatgaatcacatctgaaatgaccacttccaaagcctaagcactggcacaacagtttaaagcctgattcagacattcgttcccactcatctccaacggcataatgggaaactgtgtaggggtcaaagcacgagtcatccgtaggttggttcaagccttcgttgacagaagttgcccacggtaacaacctcttcccgaaccttatgcctctgctggtctttcaagtgcctccactatgatgttgtaggtggcacctctggtgaggacctcggccgcgaccacgct

SEQ ID NO: 184 - DNA, 375 bp, Homo sapiens
  misc_feature 30, 174, 248, 285, 306, 332, 345, 368: n = A,T,C or G
agcgtggtttgcggccgaggtcctcaccanaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagnttcccattatgccgttggagatgagtgggaacgaatgtctgaatcaggctttaaactgttgtgccagtgcttangctttggaagtggtcatttcagatgtgattcatctanatggtgtcatgacaatggtgngaactacaagattggagagaagtggnaccgtcagggganaaaatggacctgcccgggcggcncgctcga

SEQ ID NO: 185 - DNA, 148 bp, Homo sapiens
  misc_feature 28, 36, 86: n = A,T,C or G
agcgtggtcgcggccgaggtctggcttnctgctcangtgattatcctgaaccatccaggccaaataagcgccggctatgcccctgnattggattgccacacggctcacattgcatgcaagtttgctgagctgaaggaaaagattgatc

SEQ ID NO: 186 - DNA, 397 bp, Homo sapiens
  misc_feature 78: n = A,T,C or G
tcgagcggccgcccgggcaggtccaattgaaacaaacagttctgagaccgttcttccaccactgattaagagtggggnggcgggtattagggataatattcatttagccttctgagctttctgggcagacttggtgaccttgccagctccagcagccttctggtccactgctttgatgacacccaccgcaactgtctgtctcatatcacgaacagcaaagcgacccaaaggtggatagtctgagaagctctcaacacacatgggcttgccaggaaccatatcaacaatgggcagcatcaccagacttcaagaatttaagggccatcttccagctttttaccagaacggcgatcaatcttttccttcagctcagcaaacttgcatgcaatgtgagccg

SEQ ID NO: 187 - DNA, 584 bp, Homo sapiens
  misc_feature 145, 286, 363, 365, 425, 433, 452, 462, 471, 512, 514,534, 536, 540, 565, 583: n = A,T,C or G
tcgagcggccgcccgggcaggtccagagggctgtgctgaagtttgctgctgccactggagccactccaattgctggccgcttcactcctggaaccttcactaaccagatccaggcagccttccgggagccacggcttcttgtggntactgaccccagggctgaccaccagcctctcacggaggcatcttatgttaacctacctaccattgcgctgtgtaacacagattctcctctgcgctatgtggacattgccatcccatgcaacaacaagggagctcactcagnggggtttgatgtggtggatgctggctcgggaagttctgcgcatgcgtggcaccatttcccgtgaacacccatgggangncatgcctgatctggacttctacagagatcctgaagagattgaaaaagaagaacaggctgnttgctganaaagcaagtgaccaaggangaaatttcangggtgaaanggactgctcccgctcctgaattcactgctactcaacctgangntgcagactggtcttgaaggngnacangggccctctgggcctatttaagcancttcggtcgcgaacacgnt

SEQ ID NO: 188 - DNA, 579 bp, Homo sapiens
  misc_feature 7, 136, 486: n = A,T,C or G
agcgtgngtcgcggccgaggtgctgaataggcacagagggcacctgtacaccttcagaccagtctgcaacctcaggctgagtagcagtgaactcaggagcgggagcagtccattcaccctgaaattcctccttggncactgccttctcagcagcagcctgctcttctttttcaatctcttcaggatctctgtagaagtacagatcaggcatgacctcccatgggtgttcacgggaaatggtgccacgcatgcgcagaacttcccgagccagcatccaccacatcaaacccactgagtgagctcccttgttgttgcatgggatgggcaatgtccacatagcgcagaggagaatctgtgttacacagcgcaatggtaggtaggttaacataagatgcctccgcgagaagctggtggtcagccctggggtcaagtaaccacaagaagccgtggctcccggaaggctgcctggatctggttagtgaaggntccaggagtgaagcggccaacaattggagtggcttcagtggcaagcagcaaacttcagcacaagccctctggacctgcccggcggccgctcga

SEQ ID NO: 189 - DNA, 374 bp, Homo sapiens
  misc_feature 41, 280, 314, 330, 350, 353: n = A,T,C or G
tcgagcggccgcccgggcaggtccattttctccctgacggncccacttctctccaatcttgtagttcacaccattgtcatggcaccatctagatgaatcacatctgaaatgaccacttccaaagcctaagcactggcacaacagtttaaagcctgattcagacattcgttcccactcatctccaacggcataatgggaaactgtgtaggggtcaaagcacgagtcatccgtaggttggttcaagccttcgttgacagagttgcccacggtaacaacctcntccccgaaccttatgcctctgctgggctttcagngcctccactatgatgntgtaggggggcacctctggngangacctcggccgcgaccacgct

SEQ ID NO: 190 - DNA, 373 bp, Homo sapiens
  misc_feature 247, 304, 306, 332, 337: n = A,T,C or G
agcgtggtcgcggccgaggtcctcaccagaggtgccacctacaacatcatagtggaggcactgaaagaccagcagaggcataaggctcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttangctttggaagtgggtcatttcagatgtgattcatctagatggtgccatgacaatggngngaactacaagattggagagaagtggnaccgncagggagaaaatggacctgcccgggcggccgctcga

SEQ ID NO: 191 - DNA, 354 bp, Homo sapiens
  misc_feature 218, 299, 306, 326, 333, 337, 341: n = A,T,C or G
agcgtggtcgcggccgaggtccacatcggcagggtcggagccctggccgccatactcgaactggaatccatcggtcatgctctcgccgaaccagacatgcctcttgtccttggggttcttgctgatgtaccagttcttctgggccacactgggctgagtggggtacacgcaggtctcaccagtctccatgttgcagaagactttgatggcatccaggntgcaaccttggttggggtcaatccagtactctccactcttccagccagagtggcacatcttgaggtcacggcaggtgcggncgggggnttttgcggctgccctctggncttcggntgtnctcnatctgctggctca

SEQ ID NO: 192 - DNA, 587 bp, Homo sapiens
  misc_feature 276: n = A,T,C or G
tcgagcggccgcccgggcaggtctcgcggtcgcactggtgatgctggtcctgttggtccccccggccctcctggacctcctggcccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagncgcaagaaccccgcccgcacctgccgtgacctcaagatgtgccactctgactggaagagtggagagtactggattgaccccaaccaagctgcaacctggatgccatcaaagtcttctgcaacatggagactggtgagacctgcgtgtaccccactcagcccagtgtggcccaaaagaactggtacatcagcaagaaccccaaggacaagaagcatgtctggttcggcgagaacatgaccgatggattccagttcgagtatggcgggcagggctccgaccctgccgatggggaccttggccgcgaacacgct

SEQ ID NO: 193 - DNA, 98 bp, Homo sapiens
  misc_feature 8, 9, 33, 58, 71, 90: n = A,T,C or G
agcgtggnngcggccgaggtataaatatccagnccatatcctccctccacacgctganagatgaagctgtncaaagatctcagggtgganaaaaccat

SEQ ID NO: 194 - DNA, 240 bp, Homo sapiens; identical to SEQ ID NO: 75

SEQ ID NO: 195 - DNA, 400 bp, Homo sapiens
  misc_feature 22, 37, 39, 105, 268, 276, 302, 323, 331, 335, 347, 351,371, 378: n = A,T,C or G
cgagcgggcgaccgggcaggtncagactccaatccananaaccatcaagccagatgtcagaagctacaccatcacaggtttacaaccaggcactgactacaaganctacctgcacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggtacatcatcnagtatganaagcctgggcctcctcccagagaagnggtccctcggccccgccctgntgtcccanaggntactattactgngccngcaaccggcaaccgatatcnattttgncattggccttcaacaataatta

SEQ ID NO: 196 - DNA, 494 bp, Homo sapiens
  misc_feature 19, 83, 168, 252, 271, 292, 430: n = A,T,C or G
agcgtggttcgcggccgangtcctgtcagagtggcactggtagaagttccaggaaccctgaactgtaagggttcttcatcagngccaacaggatgacatgaaatgatgtactcagaagtgtcctggaatggggcccatgagatggttgtctgagagagagcttcttgncctgtctttttccttccaatcaggggctcgctcttctgattattcttcagggcaatgacataaattgtatattcgggtcccggntccaggccagtaatagtancctctgtgacaccagggcggngccgagggaccacttctctgggaggagacccaggcttctcatacttgatgatgtaaccggtaatcctggcacgtggcggctgccatgataccagcaaggaattggggtgtggtggccaggaaacgcaggttggatggngcatcaatggcagtggaggccgtcgatgaccacagggggagctccgacattgtcattcaaggtg

SEQ ID NO: 197 - DNA, 118 bp, Homo sapiens
  misc_feature 8, 71, 96: n = A,T,C or G
agcgtggncgcggccgaggtgcagcgcgggctgtgccaccttctgctctctgcccaacgataaggagggtncctgcccccaggagaacattaactntccccagctcggcctctgccgg

SEQ ID NO: 198 - DNA, 403 bp, Homo sapiens
  misc_feature 41, 53, 98, 195, 350: n = A,T,C or G
tcgagcggccgcccgggcaggttttttttgctgaaagtggntactttattggntgggaaagggagaagctgtggtcagcccaagagggaatacagagncccgaaaaaggggagggcaggtgggctggaaccagacgcagggccaggcagaaactttctctcctcactgctcagcctggtggtggctggagctcanaaattgggagtgacacaggacaccttcccacagccattgcggcggcatttcatctggccaggacactggctgtccacctggcactggtcccgacagaagcccgagctggggaaagttaatgttcacctgggggcaggaaccctccttatcattgngcagagagcagaaggtggcacagcccgcgctgcacctcggccgcgaccacgct

SEQ ID NO: 199 - DNA, 167 bp, Homo sapiens
  misc_feature 92, 107: n = A,T,C or G
tcgagcggccgcccgggcaggtccaccataagtcctgatacaaccacggatgagctgtcaggagcaaggttgatttctttcattggtccggncttctccttgggggncacccgcactcgatatccagtgagctgaacattgggtggcgtccactgggcgctcaggct

SEQ ID NO: 200 - DNA, 252 bp, Homo sapiens
  misc_feature 210, 226, 227, 230, 236: n = A,T,C or G
tcgagcggttcgcccgggcaggtccaccacacccaattccttgctggtatcatggcagccgccacgtgccaggattaccggctacatcatcaagtatgagaagcctgggtctcctcccagagaagcggtccctcggccccgccctggtgtcacagaggctactattactggcctggaaccgggaaccgaatatacaatttatgtcattgncctgaagaataatcannaanagcgancccctgattggaagga

SEQ ID NO: 201 - DNA, 91 bp, Homo sapiens
agcgtggtcgcggccgaggttgtacaagctttttttttttttttttttttttttttttttttttttttttttttttttttttttttttttt

SEQ ID NO: 202 - DNA, 368 bp, Homo sapiens
  misc_feature 9, 354: n = A,T,C or G
tcgagcggncgcccgggcaggtctgccaacaccaagattggcccccgccgcatccacacagtccgtgtgcggggaggtaacaagaaataccgtgccctgaggttggacgtggggaatttctcctggggctcagagtgttgtactcgtaaaacaaggatcatcgatgttgtctacaatgcatctaataacgagctggttcgtaccaagaccctggtgaagaattgcatcgtgctcatcgacagcacaccgtaccgacagtggtacgagtcccactatgcgctgcccctgggccgcaagaagggagccaagctgactcctgaggaagaagagattttaaacaaaaaacgatctaanaaaaaaaaaacaat

SEQ ID NO: 203 - DNA, 340 bp, Homo sapiens
agcgtggtcgcggccgaggtgaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggacaagaagctctctctcagacaaccatctcatgggccccattccaggacacttctgagtacatcatttcatgtcatcctgttggcactgatgaagaacccttacagttcagggttcctggaacttctaccagtgccactctgacaggacctgcccgggcggccgctcga

SEQ ID NO: 204 - DNA, 341 bp, Homo sapiens
tcgagcggccgcccgggcaggtcctgtcagagtggcactggtagaagttccaggaaccctgaactgtaagggttcttcatcagtgccaacaggatgacatgaaatgatgtactcagaagtgtcctggaatggggcccatgagatggttgtctgagagagagcttcttgtcctacattcggcgggtatggtcttggcctatgccttatgggggtggccgttgtgggcggtgtggtccgcctaaaaccatgttcctcaaagatcatttgttgcccaacactgggttgctgaccagaagtgccaggaagctgaataccatttcacctcggccgcgaccacgcta

SEQ ID NO: 205 - DNA, 770 bp, Homo sapiens
  misc_feature 529, 591, 623, 626, 629, 630, 656, 702, 709, 712, 717,743, 746, 749, 759, 762, 766: n = A,T,C or G
tcgagcggccgcccgggcaggtctcccttcttgcggcccaggggcagcgcatagtgggactcgtaccactgtcggtacggtgtgctgtcgatgagcacgatgcaattcttcaccagggtcttggtacgaaccagctcgttattagatgcattgtagacaacatcgatgatccttgttttacgagtacaacactctgagccccaggagaaattccccacgtccaacctcagggcacggtatttcttgttacctccccgcacacggactgtgtggatgcggcgggggccaagctgactcctgaggaagaagagattttaaacaaaaaacgatctaaaaaaattcagaagaaatatgatgaaaggaaaaagaatgccaaaatcagcagtctcctggaggagcagttccagcagggcaagcttcttgcgtgcatcgcttcaaggccgggacagtgtgaccgagcagatggctatgtgctagagggcaaagaagtggagttctatcttaagaaaatcagggcccagaatggtgngtcttcaactaatccaaaggggagtttcagaccagtgcaatcagcaaaaacattgatactgntggccaaatttattggtgcagggcttgcacantangannggctgggtcttggggcttggattggnacaagctttggcagccttttctttggttttgccaaaaaccttttgntgaaganganacctngggcggaccccttaaccgattccacnccnggnggcgttctanggncccncttg

SEQ ID NO: 206 - DNA, 810 bp, Homo sapiens
  misc_feature 574, 621, 625, 636, 668, 673, 704, 728, 743, 767, 772,786, 789, 807, 809, 810: n = A,T,C or G
agcgtggtcgcggccgaggtctgctgcttcagcgaagggtttctggcataaccaatgataaggctgccaaagactgttccaataccagcaccagaaccagccactcctactgttgcagcacctgcaccaataaatttggcagcagtatcaatgtctctgctgattgcactggtctgaaactccctttggattagctgagacacaccattctgggccctgattttcctaagatagaactccaactctttgccctctagcacatagccatctgctcggtcacactgtcccggccttgaagcgatgcacgcaagaagcttgccctgctggaactgctcctccaggagactgctgattttggcattctttttcctttcatcatatttcttctgaatttttttagatcgttttttgtttaaaatctcttcttcctcaggagtcagcttggcccccgccgcatccacacagtccgtgtgcggggaggtaacaagaaataccgtgccctgaggttggacgtggggaatttctcctggggctcagagtggtgtactcgtaaaacaaggatcatcgatggtgnctacaatgcatctaataacgagctgggtcggacccaaagaacctggngaanaaatggatcgnctcatcgacaggacaccgtacccgacaggggnacgantcccactatgcgcttgcccctgggccgcaanaaaggaaaactgcccgggcggccntcgaaagcccaattntggaaaaaatccatcacactgggnggccngtcgagcatgcatntanaggggcccattccccctnann

SEQ ID NO: 207 - DNA, 257 bp, Homo sapiens
tcgagcggccgcccgggcaggtccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggacctcggccgcgaccacgct

SEQ ID NO: 208 - DNA, 257 bp, Homo sapiens
agcgtggtcgcggccgaggtccacatcggcagggtcggagccctggccgccatactcgaactggaatccatcggtcatgctctcgccgaaccagacatgcctcttgtccttggggttcttgctgatgtaccagttcttctgggccacactgggctgagtggggtacacgcaggtctcaccagtctccatgttgcagaagactttgatggcatccaggttgcagccttggttggggacctgcccgggcggccgctcga

SEQ ID NO: 209 - DNA, 747 bp, Homo sapiens
  misc_feature 453, 538, 540, 542, 546, 554, 556, 598, 659, 670, 679,689, 693, 711, 723, 724, 731, 747: n = A,T,C or G
tcgagcggccgcccgggcaggtccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggntttaggcggaccacaccgcccacaacggccacccccataaggcataggccaagaccatacccgccgaatgtaggacaagaagctntntntcanacaccatntnatgggccccattccaggacacttctgagtacatcatttatgncatctgtggcacttgatgaaaacccttacagttcagggttctggaacttttaccaggcctnttacaggactnggccggacnccttaagccnattncaccctggggcgttctanggtcccactcgnncactggngaaaatggctactgtn

SEQ ID NO: 210 - DNA, 872 bp, Homo sapiens
  misc_feature 165, 174, 181, 256, 260, 269, 271, 277, 286, 289, 294,298, 300, 301, 303, 308, 311, 321, 325, 328, 329, 333, 338, 342,346, 349, 351, 357, 359, 364, 366, 379, 385, 395, 396, 397,407, 408, 410, 414, 415, 429, 431, 434, 435, 440, 443: n = A,T,C or G
  misc_feature 444, 446, 447, 448, 449, 450, 451, 464, 470, 472, 475,479, 483, 484, 485, 488, 494, 496, 497, 504, 508, 509, 511, 513,517, 522, 524, 526, 532, 533, 542, 543, 553, 559, 566, 567,571, 572, 578, 582, 588, 591, 594, 595, 596, 600, 606: n = A,T,C or G
  misc_feature 612, 614, 617, 618, 629, 630, 631, 652, 654, 655, 661,663, 664, 666, 671, 673, 678, 679, 681, 688, 690, 691, 698, 706,707, 708, 714, 719, 721, 723, 726, 741, 751, 761, 762, 769,770, 778, 779, 781, 782, 785, 791, 802, 807, 808, 812: n = A,T,C or G
  misc_feature 815, 820, 827, 828, 838, 841, 844, 851, 857, 864, 866,869, 872: n = A,T,C or G
agcgtggtcgcggccgaggtccactagaggtctgtgtgccattgcccaggcagagtctctgcgttacaaactcctaggagggcttgctgtgcggagggcctgctatggtgtgctgcggttcatcatggagagtggggccaaaggctgcgaggttgtggtgtctgngaaactccnaggacangagggctaaattccatgaagtttgtggatggcctgatgatccacaatcggagaccctgttaactactaccgtctnaccncctgctgtncncccccntttctgctnaanacatngggntnntncttgnccntccttgggtngaanatnnaatngcctncccnttcntancnctactngntccananttggcctttaaanaatccnccttgccttnnncactgttcanntntttnntcgtaaaccctatnanttnnattanatnntnnnnnnctcacccccctcntcattnanccnatangctnnnaantccttnanncctcccncccnntncnctcntactnantncttctnncccattacnnagctctttcntttaanataatgnngccnngctctncatntctacnatntgnnnaatncccccncccccnancgnntttttgacctnnnaacctcctttcctcttccctncnnaaattncnnanttccncnttccnncntttcggntnntcccatnctttccannncttcantctancncnctncaacttattttcctntcatcccttnttctttacannccccctnntctactcnncnnttncattanatttgaaactnccacnnctanttncctcnctctacnnttttattttncgntcnctctacntaatantttaatnanttntcn

SEQ ID NO: 211 - DNA, 517 bp, Homo sapiens
  misc_feature 462, 464, 506: n = A,T,C or G
tcgagcggccgcccgggcaggtctgccaaggagaccctgttatgctgtggggactggctggggcatggcaggcggctctggcttcccacccttctgttctgagatgggggtggtgggcagtatctcatctttgggttccacaatgctcacgtggtcaggcaggggcttcttagggccaatcttaccagttgggtcccagggcagcatgatcttcaccttgatgcccagcacaccctgtctgagcaacacgtggcgcacaagcagtgtcaacgtagtaagttaacagggtctccgctgtggatcatcaggccatccacaaacttcatggatttagccctctgtcctcggagtttcccagacaccacaacctcgcagcctttggccccactctccatgatgaaccgcagcacaccatagcaggccctccgcacaagcaagccctcctaagaatttgtaacgcananactctgctggcaatggcacacaaacctctagtggacctcggncgcgaccacgc

SEQ ID NO: 212 - DNA, 695 bp, Homo sapiens
  misc_feature 432, 476, 522, 547, 621, 624, 647, 679: n = A,T,C or G
tcgagcggccgcccgggcaggtctggtccaggatagcctgcgagtcctcctactgctactccagacttgacatcatatgaatcatactggggagaatagttctgaggaccagtagggcatgattcacagattccaggggggccaggagaaccaggggaccctggttgtcctggaataccagggtcaccatttctcccaggaataccaggagggcctggatctcccttggggccttgaggtccttgaccattaggagggcgagtaggagcagttggaggctgtgggcaaactgcacaacattctccaaatggaatttctgggttggggcagtctaattcttgatccgtcacatattatgtcatcgcagagaacggatcctgagtcacagacacatatttggcatggttctggcttccagacatctctatccgncataggactgaccaagatgggaacatcctccttcaacaagcttnctgttgtgccaaaaataatagtgggatgaagcagaccgagaagtanccagctcccctttttgcacaaagcntcatcatgtctaaatatcagacatgagacttctttgggcaaaaaaggagaaaaagaaaaagcagttcaaagtanccnccatcaagttggttccttgcccnttcagcacccgggccccgttataaaacacctngggccggacccccctt

SEQ ID NO: 213 - DNA, 804 bp, Homo sapiens
  misc_feature 552, 555, 592, 624, 629, 633, 658, 695, 697, 698, 700,702, 745, 753, 755, 762, 773, 786, 788, 793, 795: n = A,T,C or G
agcgtggtcgcggccgaggtgttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatcnggngaatcatgccctactggtcctcaaactattctcccanatgattcatatgatgtcaagtctgggatagcnagtangganggactcgcaggctattctggaccanacctgccgggggggcgttcgaaagcccgaatctgcananntncnttcacactggcggccgtcgagctgctttaaaagggccattccncctttagngngggggantacaattactnggcggcgttttanancgcgngnctgggaaat

SEQ ID NO: 214 - DNA, 594 bp, Homo sapiens
  misc_feature 452, 509, 585: n = A,T,C or G
agcgtggtcgcggccgaggtccacatcggcagggtcggagccctggccgccatactcgaactggaatccatcggtcatgctctcgccgaaccagacatgcctcttgtccttggggttcttgctgatgtaccagttcttctgggccacactgggctgagtggggtacacgcaggtctcaccagtctccatgttgcagaagactttgatggcatccaggttgcagccttggttggggtcaatccagtactctccactcttccagtcagagtggcacatcttgaggtcacggcaggtgcgggcggggttcttgcggctgccctctgggctccggatgttctcgatctgctggctcaggctcttgagggtggtgtccacctcgaggtcacggtcacgaaccacattggcatcatcagcccggtagtagcggccaccatcgtgagccttctcttgangtggctggggcaggaactgaagtcgaaaccagcgctgggaggaccagggggaccaanaggtccaggaagggcccgggggggaccaacaggaccagcatcaccaagtgcgacccgcgagaacctgcccggccgnccgctcgaa

SEQ ID NO: 215 - DNA, 590 bp, Homo sapiens
  misc_feature 8, 9: n = A,T,C or G
tcgagcgnncgcccgggcaggtctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctcccaccctgccgatgtggacctccggccgcgaccaccctt

SEQ ID NO: 216 - DNA, 801 bp, Homo sapiens
  misc_feature 2, 22, 25, 26, 328, 373, 385, 440, 473, 534, 571, 572,573, 582, 587, 589, 593, 600, 605, 617, 633, 642, 653, 672, 681,685, 696, 699, 709, 715, 717, 726, 731, 739, 742, 745, 758,769, 772, 778, 780, 788, 789, 791, 793, 796: n = A,T,C or G
tngagcggccgcccgggcaggntgnnaacgctggtcctgctggtcctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggaccgtgttggtgcccctggcccanacctcggccgcgaccacgctaagcccgaatttccagcacactggnggccgttactantggatccgagctcggtaccaagcttggcgtaatcatggtcatagctgtttcctgngtgaaattgttatccgctcacaatttcacacancatacgaagccggaaagcataaagtgtaaagccttggggtgctaatgagtgagctaactcncattaaattgcgttgcgctcactgcccgcttttccannngggaaaccntggcntngccngcttgcnttaantgaaatccgccnacccccggggaaaagncggtttgcngtattggggcnctttttccctttcctcggnttacttganttantgggctttggncgnttcgggttgnggcgancnggttcaacntcacnccaaaggnggnaanacggttttcccanaatccgggggntancccaangnaaaacatnngncnaangggct

SEQ ID NO: 217 - DNA, 349 bp, Homo sapiens
  misc_feature 10, 157, 170: n = A,T,C or G
agcgtggttngcggccgaggtctgggccaggggcaccaacacgtcctctctcaccaggaagcccacgggctcctgtttgacctggagttccattttcaccaggggcaccaggttcacccttcacaccaggagcaccgggctgtcccttcaatccatncagaccattgtgncccctaatgcctttgaagccaggaagtccaggagttccagggaaaccaccgagcaccctgtggtccaacaactcctctctcaccaggtcgtccgggttttccagggtgaccatcttcaccagccttgccaggaggaccagcaggaccagcgttaccaacctgcccgggcggccgctcga

SEQ ID NO: 218 - DNA, 372 bp, Homo sapiens
tcgagcggccgcccgggcaggtccattttctccctgacggtcccacttctctccaatcttgtagttcacaccattgtcatggcaccatctagatgaatcacatctgaaatgaccacttccaaagcctaagcactggcacaacagtttaaagcctgattcagacattcgttcccactcatctccaacggcataatgggaaactgtgtaggggtcaaagcacgagtcatccgtaggttggttcaagccttcgttgacagagttgcccacggtaacaacctcttcccgaaccttatgcctctgctggtctttcagtgcctccactatgatgttgtaggtggcacctctggtgaggacctcggccgcgaccacgct

SEQ ID NO: 219 - DNA, 374 bp, Homo sapiens
agcgtggtcgcggccgaggtcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcaagatgtgattcatctagatggtgccatgacaatggtgtgaactacaagattggagagaagtgggaccgtcagggagaaaatggacctgcccgggccggccgctcga

SEQ ID NO: 220 - DNA, 828 bp, Homo sapiens
  misc_feature 8, 9, 557, 571, 587, 588, 601, 642, 643, 647, 654, 664,681, 688, 698, 719, 720, 725, 734, 738, 743, 744, 757, 765, 773,778, 780, 782, 783, 793, 798, 805, 809, 822, 827: n = A,T,C or G
tcgagcgnncgcccgggcaggtccagtagtgccttcgggactgggttcacccccaggtctgcggcagttgtcacagcgccagccccgctggcctccaaagcatgtgcaggagcaaatggcaccgagatattccttctgccactgttctcctacgtggtatgtcttcccatcatcgtaacacgttgcctcatgagggtcacacttgaattctccttttccgttcccaagacatgtgcagctcatttggctggctctatagtttggggaaagtttgttgaaactgtgccactgacctttacttcctccttctctactggagctttcgtaccttccacttctgctgttggtaaaatggtggatcttctatcaatttcattgacagtacccacttctcccaaacatccagggaaatagtgatttcagagcgattaggagaaccaaattatggggcagaaataaggggcttttccacaggttttcctttggaggaagatttcagtggtgactttaaaagaatactcaacagtgtcttcatccccatagcaaaagaagaaacngtaaatgatggaangcttctggagatgccnncatttaagggacncccagaacttcaccatctacaggacctacttcagtttacannaagncacatantctgactcanaaaggacccaagtagcnccatggncagcactttnagcctttcccctggggaaaannttacnttcttaaancctnggccnngacccccttaagnccaaattntggaaaanttccntncnnctggggggcngttcnacatgcntttnaagggcccaattnccccnt

SEQ ID NO: 221 - DNA, 476 bp, Homo sapiens
tcgagcggccgcccgggcaggtgtcggagtccagcacgggaggcgtggtcttgtagttgttctccggctgcccattgctctcccactccacggcgatgtcgctgggatagaagcctttgaccaggcaggtcaggctgacctggttcttggtcatctcctcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtaacctcagacctcggccgcgaccacgct

SEQ ID NO: 222 - DNA, 477 bp, Homo sapiens
agcgtggtcgcggccgaggtctgaggttacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcaagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacacctgcccgggcggccgctcga

SEQ ID NO: 223 - DNA, 361 bp, Homo sapiens
tcgagcggccgcccgggcaggttgaatggctcctcgctgaccaccccggtgctggtggtgggtacagagctccgatgggtgaaaccattgacatagagactgtccctgtccagggtgtaggggcccagctcagtgatgccgtgggtcagctggctcagcttccagtacagccgctctctgtccagtccagggcttttggggtcaggacgatgggtgcagacagcatccactctggtggctgccccatccttctcaggcctgagcaaggtcagtctgcaaccagagtacagagagctgacactggtgttcttgaacaagggcataagcagaccctgaaggacacctcggccgcgaccacgct

SEQ ID NO: 224 - DNA, 361 bp, Homo sapiens; identical to SEQ ID NO: 77

SEQ ID NO: 225 - DNA, 766 bp, Homo sapiens
  misc_feature 574, 610, 631, 643, 657, 660, 666, 688, 712, 735, 747: n = A,T,C or G
agcgtggtcgcggccgaggtcctgtcagagtggcactggtagaagttccaggaaccctgaactgtaagggttcttcatcagtgccaacaggatgacatgaaatgatgtactcagaagtgtcctggaatggggcccatgagatggttgtctgagagagagcttcttgtcctacattcggcgggtatggtcttggcctatgccttatgggggtggccgttgtgggcggtgtggtccgcctaaaaccatgttcctcaaagatcatttgttgcccaacactgggttgctgaccagaagtgccaggaagctgaataccatttccagtgtcatacccagggtgggtgacgaaaggggtcttttgaactgtggaaggaacatccaagatctctggtccatgaagattggggtgtggaagggttaccagttggggaagctcgtctgtctttttccttccaatcaggggctcgctcttctgattattcttcagggcaatgacataaattgtatattcggtcccggttccaggccagtaatagtagcctctgtgacaccagggcggggccgagggacccttctnttggaagagaccagcttctcatacttgatgatgagnccggtaatcctggcacgtggnggttgcatgatnccaccaaggaaatnggngggggnggacctgcccggcggccgttcnaaagcccaattccacacacttggnggccgtactatggatcccactcngtccaacttggnggaatatggcataactttt

SEQ ID NO: 226 - DNA, 364 bp, Homo sapiens
tcgagcggccgcccgggcaggtccttgaccttttcagcaagtgggaaggtgtaatccgtctccacagacaaggccaggactcgtttgtacccgttgatgatagaatggggtactgatgcaacagttgggtagccaatctgcagacagacactggcaacattgcggacaccctccaggaagcgagaatgcagagtttcctctgtgatatcaagcacttcagggttgtagatgctgccattgtcgaacacctgctggatgaccagcccaaaggagaagggggagatgttgagcatgttcagcagcgtggcttcgctggctcccactttgtctccagtcttgatcagacctcggccgcgaccacgct

SEQ ID NO: 227 - DNA, 275 bp, Homo sapiens
agcgtggtcgcggccgaggtctgtcctacagtcctcaggactctactccctcagcagcgtggtgaccgtgccctccagcaacttcggcacccagacctacacctgcaacgtagatcacaagcccagcaacaccaaggtggacaagagagttgagcccaaatcttgtgacaaaactcacacatgcccaccgtgcccagcacctgaactcctggggggaccgtcagtcttcctcttcccccgcatcccccttccaaacctgcccgggcggccgctcg

SEQ ID NO: 228 - DNA, 275 bp, Homo sapiens
cgagcggccgcccgggcaggtttggaagggggatgcgggggaagaggaagactgacggtccccccaggagttcaggtgctgggcacggtgggcatgtgtgagttttgtcacaagatttgggctcaactctcttgtccaccttggtgttgctgggcttgtgatctacgttgcaggtgtaggtctgggtgccgaagttgctggagggcacggtcaccacgctgctgagggagtagagtcctgaggactgtaggacagacctcggccgcgaccacgct

SEQ ID NO: 229 - DNA, 40 bp, Homo sapiens
  misc_feature 1, 4, 5, 13, 15, 17, 29: n = A,T,C or G
nggnnggtccggncngncaggaccactcntcttcgaaata

SEQ ID NO: 230 - DNA, 208 bp, Homo sapiens
agcgtggtcgcggccgaggtcctcacttgcctcctgcaaagcaccgatagctgcgctctggaagcgcagatctgttttaaagtcctgagcaatttctcgcaccagacgctggaagggaagtttgcgaatcagaagttcagtggacttctgataacgtctaatttcacggagcgccacagtaccaggacctgcccgggcggccgctcga

SEQ ID NO: 231 - DNA, 208 bp, Homo sapiens
  misc_feature 33: n = A,T,C or G
tcgagcggccgcccgggcaggtcctggtactgnggcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcaggaggcaagtgaggacctcggccgcgaccacgct

SEQ ID NO: 232 - DNA, 332 bp, Homo sapiens
tcgagcggccgcccgggcaggtccacatcggcagggtcggagccctggccgccatactcgaactggaatccatcggtcatgctctcgccgaaccagacatgcctcttgtccttggggttcttgctgatgtaccagttcttctgggccacactgggctgagtggggtacacgcaggtctcaccagtctccatgttgcagaagactttgatggcatccaggttgcagccttggttggggtcaatccagtactctccactcttccagtcagagtggcacatcttgaggtcacggcaggtgcgggcggggttcttgacctcggccgcgaccacgct

SEQ ID NO: 233 - DNA, 415 bp, Homo sapiens
  misc_feature 6, 15, 19, 21: n = A,T,C or G
gtgggnttgaacccntttnanctccgcttggtaccgagctcggatccactagtaacggccgccagtgtgctggaattcggcttagcgtggtcgcggccgaggtcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggacctgcccgggcggccgctcga

SEQ ID NO: 234 - DNA, 776 bp, Homo sapiens
  misc_feature 505, 550, 574, 601, 604, 608, 612, 649, 656, 657, 680,711, 750, 776: n = A,T,C or G
agcgtggtcgcggccgaggtctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttaagtgtctatgctcagaatccaagcggagagaagtcagcctctggttcagactgnaagtaaccaacattgatcgcctaaaggactggcattcactgatgnggatgccgattccatcaaaattgnttgggaaaacccacaggggcaagtttncangtcnaggnggacctactcgagccctgaggatggaatccttgactnttccttnncctgatggggaaaaaaaaccttnaaaacttgaaggacctgcccgggcggccgtncaaaacccaattccacccccttgggggcgttctatgggncccactcggaccaaacttggggtaan

SEQ ID NO: 235 - DNA, 805 bp, Homo sapiens
  misc_feature 637, 684, 705, 724, 733, 756, 778, 793, 796, 804: n = A,T,C or G
tcgagcggccgcccgggcaggtccttgcagctctgcagtgtcttcttcaccatcaggtgcagggaatagctcatggattccatcctcagggctcgagtaggtcaccctgtacctggaaacttgcccctgtgggctttcccaagcaattttgatggaatcggcatccacatcagtgaatgccagtcctttagggcgatcaatgttggttactgcagtctgaaccagaggctgactctctccgcttggattctgagcatagacactaaccacatactccactgtgggctgcaagccttcaatagtcatttctgtttgatctggacctgcagttttagtttttgttggtcctggtccatttttgggagtggtggttactctgtaaccagtaacaggggaacttgaaggcagccacttgacactaatgctgttgtcctgaacatcggtcacttgcatctgggatggtttgtcaatttctgttcggtaattaatggaaattggcttgctgcttgcggggcttgtctccacggccagtgacagcatacacagtgatggtataatcaactccaggtttaagccgctgatggtagctgaaactttgctccaggcacaagtgaactcctgacagggctatttcctnctgttctccgtaagtgatcctgtaatatctcactgggacagcaggangcattccaaaacttcgggcgngaccccctaagccgaattntgcaatatncatcacactggcgggcgctcgancattcattaaaaggcccaatcncccctatagggagtntantacaattng

SEQ ID NO: 236 - DNA, 262 bp, Homo sapiens
tcgagcggccgcccgggcaggtcacttttggtttttggtcatgttcggttggtcaaagataaaaactaagtttgagagatgaatgcaaaggaaaaaaatattttccaaagtccatgtgaaattgtctcccatttttttggcttttgagggggttcagtttgggttgcttgtctgtttccgggttggggggaaagttggttgggtgggagggagccaggttgggatggagggagtttacaggaagcagacagggccaacgtcg

SEQ ID NO: 237 - DNA, 372 bp, Homo sapiens
agcgtggtcgcggccgaggtcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggacctgcccgggcggccgctcga

SEQ ID NO: 238 - DNA, 372 bp, Homo sapiens; identical to SEQ ID NO: 218

SEQ ID NO: 239 - DNA, 720 bp, Homo sapiens
  misc_feature 478, 557, 563, 566, 620, 660, 663, 672, 673, 684, 693,695: n = A,T,C or G
tcgagcggccgcccgggcaggtccaccataagtcctgatacaaccacggatgagctgtcaggagcaaggttgatttctttcattggtccggtcttctccttgggggtcacccgcactcgatatccagtgagctgaacattgggtggtgtccactgggcgctcaggcttgtgggtgtgacctgagtgaacttcaggtcagttggtgcaggaatagtggttactgcagtctgaaccagaggctgactctctccgcttggattctgagcatagacactaaccacatactccactgtgggctgcaagccttcaatagtcatttctgtttgatctggacctgcagttttagtttttgttggtcctggtccatttttgggagtggtggttactctgtaaccagtaacaggggaacttgaaggcagccacttgacactaatgctgttgtcctgaacatcggtcacttgcatctgggatggtttgncaatttctgttcggtaattaatggaaattggcttgctgcttgcggggctgtctccacggccagtgacagcatacacagngatggnatnatcaactccaagtttaaggccctgatggtaactttaaacttgctcccagccagngaacttccggacagggtatttcttctggttttccgaaagngancctggaatnntctccttggancagaaggancntccaaaacttgggccggaacccctt

SEQ ID NO: 240 - DNA, 691 bp, Homo sapiens
  misc_feature 564, 582, 640, 651, 666, 669, 690: n = A,T,C or G
agcgtggtcgcggccgaggtcctgtcagagtggcactggtagaagttccaggaaccctgaactgtaagggttcttcatcagtgccaacaggatgacatgaaatgatgtactcagaagtgtcctggaatggggcccatgagatggttgtctgagagagagcttcttgtcctacattcggcgggtatggtcttggcctatgccttatgggggtggccgttgtgggcggtgtggtccgcctaaaaccatgttcctcaaagatcatttgttgcccaacactgggttgctgaccagaagtgccaggaagctgaataccatttccagtgtcatacccagggtgggtgacgaaaggggtcttttgaactgtggaaggaacatccaagatctctggtccatgaagattggggtgtggaagggttaccagttggggaagctcgtctgtctttttccttccaatcaggggctcgctcttctgattattcttcagggcaatgacataaattgtatattcggttcccggttccaggccagtaatagtagcctcttgtgacaccaggcggggcccanggaccacttctctgggangagacccagcttctcatacttgatgatgtaacccggtaatcctgcacgtggcggctgncatgataccancaaggaattgggtgnggnggacctgcccggcggccctcna

SEQ ID NO: 241 - DNA, 808 bp, Homo sapiens
  misc_feature 680, 715, 721, 728, 735, 749, 757, 762, 772, 776, 779,781, 792, 796, 800, 808: n = A,T,C or G
agcgtggtcgcggccgaggtctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccactattcctgcaccaactgacctgaagttcactcaggtcacacccacaagcctgagccgccagtggacaccacccaatgttcactcactggatatcgagtgcgggtgacccccaaggagaagacccggacccatgaaagaaatcaaccttgctcctgacagctcatccgngggtgtatcaggacttatgggggactgccccggcnggccgntcgaaancgaattntgaaatttccttcncactgggnggcgnttcgagcttncttntananggcccaattcncctntagngggtcgtn

SEQ ID NO: 242 - DNA, 26 bp, Homo sapiens
  misc_feature 22: n = A,T,C or G
agcgtggtcgcggccgaggtcnagga

SEQ ID NO: 243 - DNA, 697 bp, Homo sapiens
  misc_feature 496, 541, 624, 662, 679, 688: n = A,T,C or G
tcgagcggccgcccgggcaggtccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacgggcacccccataaggnataggccaagaccataccccgccgaatgtaggacaagaagctctntctcaacaaccatctcatgggccccattccaggacacttctgagtacatcatttcatgtcatcctggtgggcacttgatgaanaacccttacagttcagggttcctggaacttctaccagngccacttctgacaggancttgggcgngaccaccct

SEQ ID NO: 244 - DNA, 373 bp, Homo sapiens
agcgtggtcgcggccgaggtccattttctccctgacggtcccacttctctccaatcttgtagttcacaccattgtcatggcaccatctagatgaatcacatctgaaatgaccacttccaaagcctaagcactggcacaacagtttaaagcctgattcagacattcgttcccactcatctccaacggcataatgggaaactgtgtaggggtcaaagcacgagtcatccgtaggttggttcaagccttcgttgacagagttgcccacggtaacaacctcttcccgaaccttatgcctctgctggtctttcagtgcctccactatgatgttgtaggtggcacctctggtgaggacctgcccgggcggcccgctcga

SEQ ID NO: 245 - DNA, 307 bp, Homo sapiens
agcgtggtcgcggccgaggtgtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaaatgggagacaatttcacatggactttggaaaatatttttttcctttgcattcatctctcaaacttagtttttatctttgaccaaccgaacatgaccaaaaaccaaaagtgacctgcccgggcggccgctcga

SEQ ID NO: 246 - DNA, 372 bp, Homo sapiens
tcgagcggccgcccgggcaggtcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggacctcggccgcgaccacgct

SEQ ID NO: 247 - DNA, 348 bp, Homo sapiens
  misc_feature 284, 297, 299, 322, 325, 338, 342, 345: n = A,T,C or G
tcgagcggccgcccgggcaggtaccggggtggtcagcgaggagccattcacactgaacttcaccatcaacaacctgcggtatgaggagaacatgcagcaccctggctccaggaagttcaacaccacggagagggtccttcagggcctgctcaggtccctgttcaagagcaccagtgttggccctctgtactctggctgcagactgactttgctcagacctgagaaacatggggcagccactggagtggacgccatctgcaccctccgccttgatcccactggtnctggactggacananagcggctatacttgggagctganccnaacctttggcggngacnccnctt

SEQ ID NO: 248 - DNA, 304 bp, Homo sapiens
  misc_feature 125: n = A,T,C or G
gaggactggctcagctcccagtatagccgctctctgtccagtccaggaccagtgggatcaaggcggagggtgcagatggcgtccactccagtggctgccccatgtttctcaagtctgagcaaagncagtctgcagccagagtacagagggccaacactggtgctcttgaacagggacctgagcaggccctgaaggaccctctccgtggtgttgaacttcctggagccagggtgctgcatgttctcctcataccgcaggttgttgatggtgaagttcagtgtgaatggctcctcgctgaccaccc

SEQ ID NO: 249 - DNA, 400 bp, Homo sapiens
  misc_feature 308, 310, 312, 320, 331, 336, 383, 392, 396: n = A,T,C or G
agcgtggtcgcggccgaggtccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccananancttggatngtcctttcacnggttnaaaaaacccttttcgcccccccaccttggggattaaccttgggaaanggggatttnaccnttcc

SEQ ID NO: 250 - DNA, 400 bp, Homo sapiens
  misc_feature 338, 357, 361, 369, 388, 394: n = A,T,C or G
tcgagcggccgcccgggcaggtcctgtcagagtggcactggtagaagttccaggaaccctgaactgtaagggttcttcatcagtgccaacaggatgacatgaaatgatgtactcagaagtgtcctggaatggggcccatgagatggttgtctgagagagagcttcttgtcctacattcggcgggtatggtcttggcctatgccttatgggggtggccgttgtgggcggtgtggtccgcctaaaaccatgttcctcaaagatcatttgttgcccaacactgggttgctgaccagaagtgccaggaagctgaataccatttccagtgtcatacccagggngggtgaccaaagggggtcntttngacctggngaaaggaaccatccaaaanctctgncccatg

SEQ ID NO: 251 - DNA, 514 bp, Homo sapiens
  misc_feature 8, 107, 312, 338, 351, 352, 357, 363, 366, 373, 380,405, 421, 444, 508: n = A,T,C or G
agcgtggncgcggccgaggtctgaggatgtaaactcttcccaggggaaggctgaagtgctgaccatggtgctactgggtccttctgagtcagatatgtgactgatgngaactgaagtaggtactgtagatggtgaagtctgggtgtccctaaatgctgcatctccagagccttccatcattaccgtttcttcttttgctatgggatgagacactgttgagtattctctaaagtcaccactgaaatcttcctccaaaggaaaacctgtggaaaagccccttatttctgccccataatttggttctcctaatcnctctgaaatcactatttccctggaangtttgggaaaaanngggcnacctgncantggaaantggatanaaagatcccaccattttacccaacnagcagaaagtgggaanggtaccgaaaagctccaagtaanaaaaaggagggaagtaaaggtcaagtgggcaccagtttcaaacaaaactttccccaaactatanaaccca

SEQ ID NO: 252 - DNA, 501 bp, Homo sapiens
  misc_feature 20, 21, 25, 44, 343, 347, 356, 362, 387, 391, 398, 409,428, 430, 453, 494: n = A,T,C or G
aagcggccgcccgggcaggnncagnagtgccttcgggactgggntcacccccaggtctgcggcagttgtcacagcgccagccccgctggcctccaaagcatgtgcaggagcaaatggcaccgagatattccttctgccactgttctcctacgtggtatgtcttcccatcatcgtaacacgttgcctcatgagggtcacacttgaattctccttttccgttcccaagacatgtgcagctcatttggctggctctatagtttggggaaagtttgttgaaactgtgccactgacctttacttcctccttctctactggagctttccgtaccttccacttctgctgntggnaaaaagggnggaacntcttatcaatttcattggacagtancccnctttctncccaaaacatncaagggaaaatattgattncnagagcggattaaggaacaacccnaattatgggggccagaaataaagggggcttttccacaggtnttttcct

SEQ ID NO: 253 - DNA, 226 bp, Homo sapiens
tcgagcggccgcccgggcaggtctgcaggctattgtaagtgttctgagcacatatgagataacctgggccaagctatgatgttcgatacgttaggtgtattaaatgcacttttgactgccatctcagtggatgacagccttctcactgacagcagagatcttcctcactgtgccagtgggcaggagaaagagcatgctgcgactggacctcggccgcgaccacgct

SEQ ID NO: 254 - DNA, 226 bp, Homo sapiens; identical to SEQ ID NO: 79

SEQ ID NO: 255 - DNA, 427 bp, Homo sapiens
  misc_feature 327, 403: n = A,T,C or G
cgagcggccgcccgggcaggtccagactccaatccagagaaccaccaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgncacagaagctactattactggcctggaaccgggaaccgaatatacaatttatgtcattgccctgaagaataatcanaagagcgagcccctgattggaagg

SEQ ID NO: 256 - DNA, 535 bp, Homo sapiens
  misc_feature 347, 456, 475: n = A,T,C or G
agcgtggtcgcggccgaggtcctgtcagagtggcactggtagaagttccaggaaccctgaactgtaagggttcttcatcagtgccaacaggatgacatgaaatgatgtactcagaagtgtcctggaatggggcccatgagatggttgtctgagagagagcttcttgtcctgtctttttccttccaatcaggggctcgctcttctgattattcttcagggcaatgacataaattgtatattcggttcccggttccaggccagtaatagtagcctctgtgacaccagggcggggccgagggaccacttctctgggaggagacccaggcttctcatacttgatgatgtanccggtaatcctggcaccgtggcggctgccatgataccagcaaggaattgggtgtggtggccaagaaacgcaggttggatggtgcatcaatggcagtggaggcgtcgatnaccacaggggagctccgancattgtcattcaaggtggacaggtagaatcttgtaatcaggtgcctggtttgtaaacctg

SEQ ID NO: 257 - DNA, 544 bp, Homo sapiens
  misc_feature 495, 511: n = A,T,C or G
tcgagcggccgcccgggcaggtttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaagcatgtctggttcggcgaaagcatgaccgatggattccagttcgagtatggcggccagggctccgaccctgccgatgtggacctcggccgcgaccacgctaagcccgaattccagcacactggcggccgttactagtgggatccgagcttcggtaccaagcttggcgtaatcatgggncatagctgtttcctgngtgaaaatggtattccgcttcacaatttcccac

SEQ ID NO: 258 - DNA, 418 bp, Homo sapiens
agcgtggtcgcggccgaggtccacatcggcagggtcggagccctggccgccatactcgaactggaatccatcggtcatgctctcgccgaaccagacatgcctcttgtccttggggttcttgctgatgtaccagttcttctgggccacactgggctgagtggggtacacgcaggtctcaccagtctccatgttgcagaagactttgatggcatccaggttgcagccttggttggggtcaatccagtactctccactcttccagtcagagtggcacatcttgaggtcacggcaggtgcgggcggggttcttgcggctgccctctgggctccggatgttctcgatctgctggctcaagctcttgaagggtggtgtccacctcgaggtcacggtcacgaaacctgcccgggcggccgctcga

SEQ ID NO: 259 - DNA, 377 bp, Homo sapiens
  misc_feature 320, 326, 342, 352: n = A,T,C or G
agcgtggtcgcggccgaggtcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggacctgcccgngccggnccgctcgaaaagcccnaatttccagncacacttggccggccgttactactg

SEQ ID NO: 260 - DNA, 332 bp, Homo sapiens; identical to SEQ ID NO: 232

SEQ ID NO: 261 - DNA, 94 bp, Homo sapiens
cgagcggccgcccgggcaggtccccccccttttttttttttttttttttttttttttttttttttttttttttttttttttttttttttttttt

SEQ ID NO: 262 - DNA, 650 bp, Homo sapiens
  misc_feature 412, 582, 612, 641, 646: n = A,T,C or G
agcgtggtcgcggccgaggtctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgntcctcttgttctaatcttggcaaccagtgcaagtgaccgacaaaattccagttatttatttccaaaatgtttggaaacagtataatttgacaaagaaaaaaggatacttctctttttttggctggtccaccaaatacaattcaaaaggctttttggttttatttttttanccaattccaatttcaaaatgtctcaatggngcttataataaaataaactttcacccttnttttntgat

SEQ ID NO: 263 - DNA, 573 bp, Homo sapiens
  misc_feature 453, 458, 544: n = A,T,C or G
agcgtggtcgcggccgaggtctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagaagtaaccaccactcccaaaaatggaccaggaccaacaaaaactaaaactgcaggtccagatcaaacagaaaatggactattgaaggcttgcagcccacagtggaagtatgtggntaggngtctatgctcagaatcccaagccggagaaagtcagccttctggtttagactgcagtaaccaacattgatcgccctaaaggactggncattcacttggatggtggatgtccaattc

SEQ ID NO: 264 - DNA, 550 bp, Homo sapiens
  misc_feature 39, 174, 352, 526: n = A,T,C or G
tcgagcggccgcccgggcaggtccttgcagctctgcagngtcttcttcaccatcaggtgcagggaatagctcatggattccatcctcagggctcgagtaggtcaccctgtacctggaaacttgcccctgtgggctttcccaagcaattttgatggaatcgacatccacatcagngaatgccagtcctttagggcgatcaatgttggttactgcagtctgaaccagaggctgactctctccgcttggattctgagcatagacactaaccacatactccactgtgggctgcaagccttcaatagtcatttctgtttgatctggacctgcagttttaagtttttggtggtcctgncccatttttgggaagtggggggttactctgtaaccagtaacaggggaacttgaaggcagccacttgacactaatgctgttgtcctgaacatcggtcacttgcatctggggatggttttgacaatttctggttcggcaaattaatggaaattggcttgctgcttggcggggctgnctccacgggccagtgacagcatac

SEQ ID NO: 265 - DNA, 596 bp, Homo sapiens
  misc_feature 347, 352, 353, 534, 555, 587: n = A,T,C or G
tcgagcggccgcccgggcaggtccttgcagctctgcagtgtcttcttcaccatcaggtgcagggaatagctcatggattccatcctcagggctcgagtaggtcaccctgtacctggaaacttgcccctgtgggctttcccaagcaattttgatggaatcgacatccacatcagtgaatgccagtcctttagggcgatcaatgttggttactgcagtctgaaccagaggctgactctctccgcttggattctgagcatagacactaaccacatactccactgtgggctgcaagccttcaatagtcatttctgtttgatctggacctgcagttttaagtttttgttggncctgnnccatttttggggaaggggtggttactcttgtaaccagtaacaggggaacttgaagcagccacttgacactaatgctggtggcctgaacatcggtcacttgcatctgggatggtttggtcaatttctgttcggtaattaatgggaaattggcttactggcttgcgggggctgtctccacggncagtgacaagcatacacaggngatgggtataatcaactccaggtttaaggccnctgatggta

SEQ ID NO: 266 - DNA, 506 bp, Homo sapiens
  misc_feature 393, 473: n = A,T,C or G
agcgtggtcgcggccgaggtctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagtaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatgggaccaggaccaacaaaaaactaaaactgcanggtccagatcaaacagaaatgactattgaaggcttgcagcccacagtggagtatgtgggttagtgtctatgctcagaatnccaagcggagagagtcagcctctggttcagact

SEQ ID NO: 267 - DNA, 548 bp, Homo sapiens
  misc_feature 346, 358, 432, 510, 512: n = A,T,C or G
tcgagcggccgcccgggcaggtcagcgctctcaggacgtcaccaccatggcctgggctctgctcctcctcaccctcctcactcagggcacagggtcctgggcccagtctgccctgactcagcctccctccgcgtccgggtctcctggacagtcagtcaccatctcctgcactggaaccagcagtgacgttggtgcttatgaatttgtctcctggtaccaacaacacccaggcaaggcccccaaactcatgatttctgaggtcactaagcggccctcaggggtccctgatcgcttctctggctccaagtctggcaacacggcctccctgaccgtctctgggctccangctgaggatgangctgattattactggaagctcatatgcaggcaacaacaattgggtgttcggcggaagggaccaagctgaccgtnctaaggtcaagcccaaggcttgcccccctcggtcactctgttcccaccctcctctgaagaagctttcaagccaacaangncacactgggtgtgtctcataagtggactttctaccc

SEQ ID NO: 268 - DNA, 584 bp, Homo sapiens
  misc_feature 98, 380, 421, 454, 495, 506, 512, 561, 565, 579: n = A,T,C or G
agcgtggtcgcggccgaggtctgtagcttctgtgggacttccactgctcaggcgtcaggctcaggtagctgctggccgcgtacttgttgttgctttgnttggagggtgtggtggtctccactcccgccttgacggggctgctatctgccttccaggccactgtcacggctcccgggtagaagtcacttatgagacacaccagtgtggccttgttggcttgaagctcctcagaggagggtgggaacagagtgaccgagggggcagccttgggctgacctaggacggtcagcttggtccctccgccgaacacccaattgttgttgcctgcatatgagctgcagtaataatcagcctcatcctcagcctggagcccagagacngtcaagggaggcccgtgtttgccaagacttggaagccaganaagcgatcagggacccctgagggccgctttacngacctcaaaaaatcatgaatttggggggcctttgcctgggngttggttggtnaccagnaaaacaaaatttcataaagcaccaacgtcactgctggtttccagtgcangaanatggtgaactgaantgtcc

SEQ ID NO: 269 - DNA, 368 bp, Homo sapiens
  misc_feature 265, 329: n = A,T,C or G
agcgtggtcgcggccgaggtccagcatcaggagccccgccttgccggctctggtcatcgcctttctttttgtggcctgaaacgatgtcatcaattcgcagtagcagaactgccgtctccactgctgtcttataagtctgcagcttcacagccaatggctcccatatgcccagttccttcatgtccaccaaagtacccgtctcaccatttacaccccaggtctcacagttctcctgggtgtgcttggcccgaagggaggtaagtanacggatggtgctggtcccacagttctggatcagggtacgaggaatgacctctagggcctgggcnacaagccctgtatggacctgcccgggcgggcccgctcga

SEQ ID NO: 270 - DNA, 368 bp, Homo sapiens
  misc_feature 54, 163, 219, 229, 316: n = A,T,C or G
tcgagcggccgcccgggcaggtccatacagggctgttgcccaggccctagaggncattccttgtaccctgatccagaactgtgggaccagcaccatccgtctacttacctcccttcgggccaagcacacccaggagaactgtgagacctggggtgtaaatggngagacgggtactttggtggacatgaaggaactgggcatatgggagccattggctgngaagctgcanacttataagacagcagtggagacggcagttctgctactgcgaattgatgacatcgtttcaggccacaaaaagaaaggcgatgaccanagccggcaaggcggggcttcctgatgctggacctcggccgccgaccacgctt

SEQ ID NO: 271 - DNA, 424 bp, Homo sapiens
  misc_feature 279, 329, 362, 384, 400: n = A,T,C or G
agcgtggtcgcggccgaggtccactagaggtctgtgtgccattgcccaggcagagtctctgcgttacaaactcctaggagggcttgctgtgcggagggcctgctatggtgtgctgcggttcatcatggagagtggggccaaaggctgcgaggttgtggtgtctgggaaactccgaggacagagggctaaatccatgaagtttgtggatggcctgatgatccacagcggagaccctgttaactactacgttgacactgctgtgcgccacgtgttgctcanacagggtgtgctgggcatcaaggtgaagatcatgctgccctgggacccanctggcaaaaatggcccttaaaaaccccttgccntgaccacgtgaaccatttgtgngaaccccaagatgaanatacttgcccaccaccccccattc

SEQ ID NO: 272 - DNA, 541 bp, Homo sapiens
  misc_feature 422, 442, 510, 513, 515, 525: n = A,T,C or G
tcgagcggccgcccgggcaggtctgccaaggagaccctgttatgctgtggggactggctggggcatggcaggcggctctggcttcccacccttctgttctgagatgggggtggtgggcagtatctcatctttgggttccacaatgctcacgtggtcaggcaggggcttcttagggccaatcttaccagttgggtcccagggcagcatgatcttcaccttgatgcccagcacaccctgtctgagcaacacgtggcgcacagcagtgtcaacgtagtagttaacagggtctccgctgtggatcatcaggccatccacaaacttcatggatttagccctctgtcctcggagtttcccaaaacaccacaacctcgccagcctttgggccccacttcttcatgaatgaaaccgcagcacaccattancaaggcccttccgcacaggnaagcccttcctaaggagttttgtaaacgcaaaaaactcttgcctggggcaaatgggcacacagacctntantnggaccttggnccgcgaaccaccgctt

SEQ ID NO: 273 - DNA, 579 bp, Homo sapiens
  misc_feature 223, 265, 277, 308, 329, 346, 360, 366, 429, 448, 517,524, 531, 578: n = A,T,C or G
agcgtggtcgcggccgaggtctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggngcccctggtgaaaatggaactccaggtcaaacaggagcccgngggcttcctggngagagaggacgtgttggtgcccctggcccanacctgcccgggcggccgctcnaaaagccgaaatccagnacactggcggccgntactantggaatccgaacttcggtaccaaagcttggccgtaatcatggccatagcttgttccctggggnggaaattggtattccgctnccaattccacacaacataccgaacccggaaagcattaaagtgtaaaagccctgggggggcctaaatgangtgagcntaactcncatttaattggcgttgcgcttcactgccccgcttttccagtccgggna

SEQ ID NO: 274 - DNA, 330 bp, Homo sapiens
  misc_feature 171: n = A,T,C or G
tcgagcggccgcccgggcaggtctgggccaggggcaccaacacgtcctctctcaccaggaagcccacgggctcctgtttgacctggagttccattttcaccaggggcaccaggttcacccttcacaccaggagcaccgggctgtcccttcaatccatccagaccattgtgncccctaatgcctttgaagccaggaagtccaggagttccagggaaaccacgagcaccctgtggtccaacaactcctctctcaccaggtcgtccgggttttccagggtgaccatcttcaccagccttgccaggagggccagacctcggccgcgaccacgct

SEQ ID NO: 275 - DNA, 97 bp, Homo sapiens
  misc_feature 2, 35, 72: n = A,T,C or G
ancgtggtcgcggccgaggtcctcaccagaggtgncacctacaacatcatagtggaggcactgaaagaccancagaggcataaggttcgggaagagg

SEQ ID NO: 276 - DNA, 610 bp, Homo sapiens
  misc_feature 358, 360, 363, 382, 424, 433, 464, 468, 477, 491, 499,511, 558, 584, 588, 590: n = A,T,C or G
tcgagcggccgcccgggcaggtccattttctccctgacggtcccacttctctccaatcttgtagttcacaccattgtcatggcaccatctagatgaatcacatctgaaatgaccacttccaaagcctaagcactggcacaacagtttaaagcctgattcagacattcgttcccactcatctccaacggcataatgggaaactgtgtaggggtcaaagcacgagtcatccgtaggttggttcaagccttcgttgacagagttgtccacggtaacaacctcttcccgaaccttatgcctctgctggtctttcagtgcctccactatgatgttgtaggtggcacctctggtgaggacctcngnccngaacaacgcttaagcccgnattctgcagaataatcccatcacacttggcggccgcttcgancatgcatcntaaaaggggccccaatttcccccttataagngaanccgtatttnccaatttcactggncccgccgnttttacaaacgncggtgaactggggaaaaaccctggcggttacccaactttaatcgccnttggcagcacaatccccccttttcgnccancntgggcgtaaataaccgaaaa

SEQ ID NO: 277 - DNA, 38 bp, Homo sapiens
  misc_feature 2, 5, 18, 21, 31: n = A,T,C or G
ancgnggtcgcggccgangtnttttttcttnttttttt

SEQ ID NO: 278 - DNA, 443 bp, Homo sapiens
  misc_feature 156, 212, 233, 245, 327, 331, 336, 361, 364, 381, 391,397, 419, 437: n = A,T,C or G
agcgtggtcgcggccgaggtctgaggttacatgcgtggtggtggacgtgagccacgaagaccctgaggtcaagttcaactggtacgtggacggcgtggaggtgcataatgccaagacaaagccgcgggaggagcagtacaacagcacgtaccgggnggtcagcgtcctcaccgtcctgcaccagaattggttgaatggcaaggagtacaagngcaaggtttccaacaaagccntcccagcccccntcgaaaaaaccatttccaaagccaaagggcagccccgagaaccacaggtgtacaccctgcccccatcccgggaggaaaagancaanaaccnggttcagccttaacttgcttggtcnaangctttttatcccaacgnacttcccccntggaantgggaaaaaccaatgggccaanccgaaaaacaattacaanaacccc

SEQ ID NO: 279 - DNA, 348 bp, Homo sapiens
  misc_feature 219, 256, 291, 297, 307, 314, 317: n = A,T,C or G
tcgagcggccgcccgggcaggtgtcggagtccagcacgggaggcgtggtcttgtagttgttctccggctgcccattgctctcccactccacggcgatgtcgctgggatagaagcctttgaccaggcaggtcaggctgacctggttcttggtcatctcctcccgggatgggggcagggtgaacacctggggttctcggggcttgccctttggttttgaanatggttttctcgatgggggctggaagggctttgttgnaaaccttgcacttgactccttgccattcacccagncctggngcaggacggngaggacnctnaccacacggaaccgggctggtggactgctcc

SEQ ID NO: 280 - DNA, 149 bp, Homo sapiens
  misc_feature 18, 34, 51, 118, 120, 140: n = A,T,C or G
agcgtggtcgcggacgangtcctgtcagagtggnactggtagaagttccangaaccctgaactgtaagggttcttcatcagtgccaacaggatgacatgaaatgatgtactcagaagngncctggaatggggcccatganatggttgcc

SEQ ID NO: 281 - DNA, 404 bp, Homo sapiens
  misc_feature 383, 386, 388, 393: n = A,T,C or G
tcgagcggccgcccgggcaggtccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcggcaccccccctgggtatgaacctgggaaaanggnanttaanctttcctggca

SEQ ID NO: 282 - DNA, 507 bp, Homo sapiens
  misc_feature 320, 341, 424, 450, 459, 487, 498: n = A,T,C or G
agcgtggtcgcggccgaggtctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaaggtnccctggtactgggttacagantaaccaccactcccaaaaatggaccaggaaccacaaaaacttaaactgcagggtccagatcaaaacagaaatgactattgaangcttgcagcccacagtgggagtatgngggtagtgnctatgcttcagaatccaagcggaaaaangtcaagccttntgggttcaa

SEQ ID NO: 283 - DNA, 325 bp, Homo sapiens
  misc_feature 216, 292, 303, 304: n = A,T,C or G
tcgagcggccgcccgggcaggtccttgcagctctgcagtgtcttcttcaccatcaggtgcagggaatagctcatggattccatcctcagggctcgagtaggtcaccctgtacctggaaacttgcccctgtgggctttcccaagcaattttgatggaatcgacatccacatcagtgaatgccagtcctttagggcgatcaatgttggttactgcagnctgaaccagaggctgactctctccgcttggattctgagcatagacactaaccacatactccactgtgggctgcaanccttcaataanncatttctgtttgatctggacc

SEQ ID NO: 284 - DNA, 331 bp, Homo sapiens
  misc_feature 54, 59, 63, 121, 312, 327: n = A,T,C or G
tcgagcggccgcccgggcaggtctggtggggtcctggcacacgcacatgggggngttgntctnatccagctgcccagcccccattggcgagtttgagaaggtgtgcagcaatgacaacaanaccttcgactcttcctgccacttctttgccacaaagtgcaccctggagggcaccaagaagggccacaagctccacctggactacatcgggccttgcaaatacatccccccttgcctggactctgagctgaccgaattcccccttgcgcatgcgggactggctcaagaaccgtcctggcacccttgtatganagggatgaagacacnaccc

SEQ ID NO: 285 - DNA, 509 bp, Homo sapiens
  misc_feature 316, 319, 327, 329, 339, 344, 357, 384, 398, 427, 443,450, 478: n = A,T,C or G
agcgtggtcgcggccgaggtctgtcctacagtcctcaggactctactccctcagcagcgtggtgaccgtgccctccagcaacttcggcacccagacctacacctgcaacgtagatcacaagcccagcaacaccaaggtggacaagagagttgagcccaaatcttgtgacaaaactcacacatgcccaccgtgcccagcacctgaactcctggggggaccgtcagtcttcctcttcccccgcatcccccttccaaacctgcccgggcggccgctcgaaagccgaattccagcacactggcggccggtactagtgganccnaacttggnanccaacctggnggaantaatgggcataanctgtttctggggggaaattggtatccngtttacaattcccncacaacatacgagccggaagcataaaagngtaaaagcctgggggnggcctantgaagtgaagctaaactcacattaattngcgttgccgctcactggcccgcttttccagc

SEQ ID NO: 286 - DNA, 336 bp, Homo sapiens
  misc_feature 188, 251, 267: n = A,T,C or G
tcgagcggccgcccgggcaggtttggaagggggatgcgggggaagaggaagactgacggtccccccaggagttcaggtgctgggcacggtgggcatgtgtgagttttgtcacaagatttgggctcaactctcttgtccaccttggtgttgctgggcttgtgatctacgttgcaggtgtaggtctgggngccgaagttgctggagggcacggtcaccacgctgctgagggagtagagtcctgaggactgtangacagacctcggccgngaccacgctaagccgaattctgcagatatccatcacactggcggccgctccgagcatgcattttagagg

SEQ ID NO: 287 - DNA, 30 bp, Homo sapiens
  misc_feature 8, 18: n = A,T,C or G
agcgtggncgcggacgangacaacaacccc

SEQ ID NO: 288 - DNA, 316 bp, Homo sapiens
  misc_feature 22, 130: n = A,T,C or G
tcgagcggccgcccgggcaggnccacatcggcagggtcggagccctggccgccatactcgaactggaatccatcggtcatgctcttgccgaaccagacatgcctcttgtccttggggttcttgctgatgnaccagttcttctgggccacactgggctgagtggggtacacgcaggtctcaccagtctccatgttgcagaagactttgatggcatccaggttgcagccttggttggggtcaatccagtactctccactcttccagtcagagtggcacatcttgaggtcacggcaggtgcgggcggggttcttgacct

SEQ ID NO: 289 - DNA, 308 bp, Homo sapiens
  misc_feature 36, 165, 191, 195, 218, 235: n = A,T,C or G
agcgtggtcgcggccgaggtccagcctggagataanggtgaaggtggtgcccccggacttccaggtatagctggacctcgtggtagccctggtgagagaggtgaaactggccctccaggacctgctggtttccctggtgctcctggacagaatggtgaacctggnggtaaaggagaaagaggggctccggntganaaaggtgaaggaggccctcctgnattggcaggggccccangacttagaggtggagctggcccccctggccccgaaggaggaaagggtgctgctggtcctcctgggccacctgg

SEQ ID NO: 290 - DNA, 324 bp, Homo sapiens
  misc_feature 184: n = A,T,C or G
tcgagcggccgcccgggcaggtctgggccaggaggaccaataggaccagtaggaccccttgggccatctttccctgggacaccatcagcacctggaccgcctggttcacccttgtcaccctttggaccaggacttccaagacctcctctttctccaggcattccttgcagaccaggagtaccancagcaccaggtggcccaggaggaccagcagcaccctttcctccttcgggaccagggggaccagctccacctctaagtcctggggcccctgccaatccaggagggcctccttcacctttctcacccggagcccctctttct

SEQ ID NO: 291 - DNA, 278 bp, Homo sapiens
  misc_feature 249, 267: n = A,T,C or G
tcgagcggccgcccgggcaggtccaccgggatattcgggggtctggcaggaatgggaggcatccagaacgagaaggagaccatgcaaagcctgaacgaccgcctggcctcttacctggacagagtgaggagcctggagaccgacaaccggaggctggagagcaaaatccgggagcacttggagaagaagggaccccaggtcagagactggagccattacttcaagatcatcgaggacctgagggctcanatcttcgcaaatactgcngacaatgcccg

SEQ ID NO: 292 - DNA, 299 bp, Homo sapiens
  misc_feature 6, 19, 25, 51, 53, 61, 63, 70, 109, 136, 157, 241, 276: n = A,T,C or G
atgcgnggtcgcggccgangaccanctctggctcatacttgactctaaagncntcaccagnanttacggncattgccaatctgcagaacgatgcgggcattgtccgcantatttgcgaagatctgagccctcaggncctcgatgatcttgaagtaanggctccagtctctgacctggggtcccttcttctccaagtgctcccggattttgctctccagcctccggttctcggtctccaagncttctcactctgtccaggaaaagaggccaggcggncgatcagggcttttgcatggact

SEQ ID NO: 293 - DNA, 101 bp, Homo sapiens
agcgtggtcgcggccgaggttgtacaagctttttttttttttttttttttttttttttttttttttttttttttttttttttttttttttttttttttttt

SEQ ID NO: 294 - DNA, 285 bp, Homo sapiens
  misc_feature 64, 103, 110, 237, 282: n = A,T,C or G
tcgagcggccgcccgggcaggtctgccaacaccaagattggcccccgccgcatccacacagttngtgtgcggggaggtaacaagaaataccgtgccctgaggntggacgnggggaatttctcctggggctcagagtgttgtactcgtaaaacaaggatcatcgatgttgtctacaatgcatctaataacgagctggttcgtaccaagaccctggtgaagaattgcatcgtgctcatngacagcacaccgtaccgacagtgggtaccgaagtcccactatgcncct

SEQ ID NO: 295 - DNA, 216 bp, Homo sapiens
tcgagcggccgcccgggcaggtccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaag

SEQ ID NO: 296 - DNA, 414 bp, Homo sapiens
  misc_feature 7, 10, 33, 61, 62, 63, 88, 109, 122, 255, 298, 307, 340,355, 386, 393: n = A,T,C or G
agcgtgntcncggccgaggatggggaagctcgnctgtctttttccttccaatcaggggctnnntcttctgattattcttcagggcaangacataaattgtatattcggntcccggttccagnccagtaatagtagcctctgtgacaccagggcggggccgagggaccacttctctgggaggagacccaggcttctcatacttgatgatgaagccggtaatcctggcacgtgggcggctgccatgataccaccaangaattgggtgtggtggacctgcccgggcgggccgctcgaaaanccgaattcntgcaagaatatccatcacacttgggcgggccgntcgaaccatgcatcntaaaagggccccaatttcccccctattaggngaagccncatttaacaaattccacttgg

SEQ ID NO: 297 - DNA, 376 bp, Homo sapiens
  misc_feature 312, 326, 335, 361: n = A,T,C or G
tcgagcggccgcccgggcaggtctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagccttgagccagcagaatcgaaaacattcggaacccaagaagggcaagcccgcaaagaaaccccgcccgcacctggccgngaacctccaagaangtgcccacntcttgactgggaaaaaaagggaaaantacttggaattggac

SEQ ID NO: 298 - DNA, 357 bp, Homo sapiens
  misc_feature 345, 346: n = A,T,C or G
agcgtggtcgcggccgaggtccacatcggcagggtcggagccctggccgccatactcgaactggaatccatcggtcatgctctcgccgaaccagacatgcctcttgtccttggggttcttgctgatgtaccagttcttctgggccacactgggctgagtggggtacacgcaggtctcaccagtctccatgttgcagaagactttgatggcatccaggttgcagccttggttggggtcaatccagtactctccactcttccagtcagaagtggcacatcttgaggtcacggcagggtgcgggcggggttcttgcgggctgcccttctgggctcccggaatgttctnngaacttgctgg

SEQ ID NO: 299 - DNA, 307 bp, Homo sapiens
  misc_feature 281, 285, 306: n = A,T,C or G
agcgtggtcgcggccgaggtccactagaggtctgtgtgccattgcccaggcagagtctctgcgttacaaactcctaggagggcttgctgtgcggagggcctgctatggtgtgctgcggttcatcatggagagtggggccaaaggctgcgaggttgtggtgtctgggaaactccgaggacagagggctaaatccatgaagtttgtggatggcctgatgatccacagcggagaccctgttaactactacgttgacacttgcttgtgcgccacgtgttgctcanacangggtgggctgggcatcaaggng

SEQ ID NO: 300 - DNA, 351 bp, Homo sapiens
tcgagcggccgcccgggcaggtctgccaaggagaccctgttatgctgtggggactggctggggcatggcaggcggctctggcttcccacccttctgttctgagatgggggtggtgggcagtatctcatctttgggttccacaatgctcacgtggtcaggcaggggcttcttagggccaatcttaccagttgggtcccagggcagcatgatcttcaccttgatgcccagcacaccctgtctgagcaacacgtggcgcacagcaagtgtcaacgtaagtaagttaacagggtctccgctgtggatcatcaggccatccacaaacttcatggatttaaccctctgtcctcggag

SEQ ID NO: 301 - DNA, 330 bp, Homo sapiens
tcgagcggccgcccgggcaggtgtttcagaggttccaaggtccactgtggaggtcccaggagtgctggtggtgggcacagaggtccgatgggtgaaaccattgacatagagactgttcctgtccagggtgtaggggcccagctctttgatgccattggccagttggctcagctcccagtacagccgctctctgttgagtccagggcttttggggtcaagatgatggatgcagatggcatccactccagtggctgctccatccttctcggacctgagagaggtcagtctgcagccagagtacagagggccaacactggtgttctttgaata

SEQ ID NO: 302 - DNA, 317 bp, Homo sapiens
  misc_feature 129, 295: n = A,T,C or G
agcgtggtcgcggccgaggtctgtactgggagctaagcaaactgaccaatgacattgaagagctgggcccctacaccctggacaggaacagtctctatgtcaatggtttcacccatcagagctctgtgnccaccaccagcactcctgggacctccacagtggatttcagaacctcagggactccatcctccctctccagccccacaattatggctgctggccctctcctggtaccattcaccctcaacttcaccatcaccaacctgcagtatggggaggacatgggtcaccctgnctccaggaagttcaacaccaca

SEQ ID NO: 303 - DNA, 283 bp, Homo sapiens
  misc_feature 139, 146, 195: n = A,T,C or G
tcgagcggccgcccggacaggtctgggcggatagcaccgggcatattttggaatggatgaggtctggcaccctgagcagtccagcgaggacttggtcttagttgagcaatttggctaggaggatagtatgcagcacggntctgagnctgtgggatagctgccatgaagtaacctgaaggaggtgctggctggtangggttgattacagggttgggaacagctcgtacacttgccattctctgcatatactggttagtgaggtgagcctggccctcttcttttg

SEQ ID NO: 304 - DNA, 72 bp, Homo sapiens
  misc_feature 59: n = A,T,C or G
agcgtggtcgcggccgaggtgagccacaggtgaccggggctgaagctggggctgctggncctgctggtcctg

SEQ ID NO: 305 - DNA, 245 bp, Homo sapiens
  misc_feature 5, 11, 22, 98, 102: n = A,T,C or G
cagcngctccnacggggcctgngggaccaacaacaccgttttcacccttaggccctttggctcctctttctcctttagcaccaggttgaccagcagcnccancaggaccagcaaatccattggggccagcaggaccgacctcaccacgttcaccagggcttccccgaggaccagcaggaccagcaggaccagcagccccagcttcgccccggtcacctgtggctcacctcggccgcgaccacgct

SEQ ID NO: 306 - DNA, 246 bp, Homo sapiens
  misc_feature 144, 159: n = A,T,C or G
tcgagcggtcgcccgggcaggtccaccgggatagccgggggtctggcaggaatgggaggcatccagaacgagaaggagaccatgcaaagcctgaacgaccgcctggcctcttacctggacagagtgaggagcctggagaccganaaccggaggctgganagcaaaatccgggagcacttggagaagaagggaccccaggtcaagagactggagccattacttcaagatcatcgagggacctggagg

SEQ ID NO: 307 - DNA, 333 bp, Homo sapiens
  misc_feature 5: n = A,T,C or G
agcgnggtcgcggccgaggtccagctctgtctcatacttgactctaaagtcatcagcagcaagacgggcattgtcaatctgcagaacgatgcgggcattgtccgcagtatttgcgaagatctgagccctcaggtcctcgatgatcttgaagtaatggctccagtctctgacctggggtcccttcttctccaagtgctcccggattttgctctccagcctccggttctcggtctccaggctcctcactctgtccaggtaagaaggcccaggcggtcgttcaggctttgcatggtctccttctcgttctggatgcctcccattcctgccagaccc

SEQ ID NO: 308 - DNA, 310 bp, Homo sapiens; identical to SEQ ID NO: 81

SEQ ID NO: 309 - DNA, 429 bp, Homo sapiens
agcgtggtcgcggccgaggtccacatcggcagggtcggagccctggccgccatactcgaactggaatccatcggtcatgctctcgccgaaccagacatgcctcttgtccttggggttcttgctgatgtaccagttcttctgggccacactgggctgagtggggtacaccgcaggtctcaccagtctccatgttgcagaagactttgatggcatccaggttgcagccttggttggggtcaatccagtactctccactcttccagtcagaagtgggcacatcttgaggtcaccggcaggtgccgggccgggggttcttgcggcttgccctctgggctccggatgttctcgatctgcttggctcaggctcttgagggtgggtgtccacctcgaggtcacggtcaccgaaacctgcccgggcggcccgctcga

SEQ ID NO: 310 - DNA, 430 bp, Homo sapiens
  misc_feature 342: n = A,T,C or G
tcgagcggtcgcccgggcaggtttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtgggcccagaagaaactggtacatcagcaaggaaccccaaggacaagaggcattgtcttggttcggcgagnagcatgacccgatggattccagtttcgagtattggcggccagggcttcccgacccttgccgatgtggacctcggccgcgaccaccgct

SEQ ID NO: 311 - DNA, 2996 bp, Homo sapiens
cagccaccggagtggatgccatctgcacccaccgccctgaccccacaggccctgggctggacagagagcagctgtatttggagctgagccagctgacccacagcatcactgagctgggcccctacaccctggacagggacagtctctatgtcaatggtttcacacagcggagctctgtgcccaccactagcattcctgggacccccacagtggacctgggaacatctgggactccagtttctaaacctggtccctcggctgccagccctctcctggtgctattcactctcaacttcaccatcaccaacctgcggtatgaggagaacatgcagcaccctggctccaggaagttcaacaccacggagagggtccttcagggcctggtccctgttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggcccctatgccctggacaacgacagcctctttgtcaatggtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctgcaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatggtttcacccatcggagctctgtacccaccaccagcaccggggtggtcagcgaggagccattcacactgaacttcaccatcaacaacctgcgctacatggcggacatgggccaacccggctccctcaagttcaacatcacagacaacgtcatgaagcacctgctcagtcctttgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacggttacaatgaacctggtccagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatggctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctacctgaatttcaccatcaccaacctaccatattcccaggacaaagcccagccaggcaccaccaattaccagaggaacaaaaggaatattgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattttcccaacagaaatgagcccttaactgggaattctgaccttcccttctgggctgtcatcctcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcctggggtgcctttcccccagccagggtccaaagaagcttggctggggcagaaataaaccatattggtcggacacaaaaaaaaaaaaa

SEQ ID NO: 312 - protein, 914 aa, Homo sapiens
MSMVSHSGALCPPLAFLGPPQWTWEHLGLQFLNLVPRLPALSWCYSLSTSPSPTCGMRRTCSTLAPGSSTPRRGSFRAWSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYFPNRNEPLTGNSDLPFWAVILIGLAGLLGLITCLICGVLVTTRRRKKEGEYNVQQQCPGYYQSHLDLEDLQ

SEQ ID NO: 313 - DNA, 656 bp, Homo sapiens
acagccagtcggagctgcaagtgttctgggtggatcgcgyatatgcactcaaaatgctctttgtaaaggaaagccacaacatgtccaagggacctgaggcgacttggaggctgagcaaagtgcagtttgtctacgactcctcggagaaaacccacttcaaagacgcagtcagtgctgggaagcacacagccaactcgcaccacctctctgccttggtcacccccgctgggaagtcctatgagtgtcaagctcaacaaaccatttcactggcctctagtgatccgcagaagacggtcaccatgatcctgtctgcggtccacatccaaccttttgacattatctcagattttgtcttcagtgaagagcataaatgcccagtggatgagcgggagcaactggaagaaaccttgcccctgattttggggctcatcttgggcctcgtcatcatggtaacactcgcgatttaccacgtccaccacaaaatgactgccaaccaggtgcagatccctcgggacagatcccagtataagcacatgggctagaggccgttaggcaggcaccccctattcctgctcccccaactggatcaggtagaacaacaaaagcacttttccatcttgtacacgagatacaccaacatagctacaatcaaacag

SEQ ID NO: 314 - DNA, 519 bp, Homo sapiens
tgtgcgtggaccagtcagcttccgggtgtgactggagcagggcttgtcgtcttcttcagagtcactttgcaggggttggtgaagctgctcccatccatgtacagctcccagtctactgatgtttaaggatggtctcggtggttaggcccactagaataaactgagtccaatacctctacacagttatgtttaactgggctctctgacaccgggaggaaggtggcggggtttaggtgttgcaaacttcaatggttatgcggggatgttcacagagcaagctttggtatctagctagtctagcattcattagctaatggtgtcctttggtatttattaaaatcaccacagcatagggggactttatgtttaggttttgtctaagagttagcttatctgcttcttgtgctaacagggctattgctaccagggactttggacatgggggccagcgtttggaaacctcatctagtttttttgagagataggccactggccttggacctcggccgcgaccacgct

SEQ ID NO: 315 - DNA, 441 bp, Homo sapiens
cacagagcgtttattgacaccaccactcctgaaaattgggatttcttattaggttcccctaaaagttcccatgttgattacatgtaaatagtcacatatatacaatgaaggcagtttcttcagaggcaaccagggtttatagtgctaggtaaatgtcatctcttttgtgctactgactcattgtcaaacgtctctgcactgttttcagcctctccacgttgcctctgtcctgcttcttagttccttctttgtgacaaaccaaaagaataagaggatttagaacaggactgcttttcccctatgatttaaaaattccaatgactttcgcccttgggagaaatttccaaggaaatctctctcgctcgctctctccgttttcctttgtgagcttctgggggagggttagtggtgactttttgatacgaaaaaatgcattttgtg

SEQ ID NO: 316 - DNA, 247 bp, Homo sapiens
tggcgcggctgctggatttcaccttcttgcacctgccggtgagcgcctggggtctaaaggggcgggatactccattatggcccctcgccctgtagggctggaatagttagaaaaggcaacccagtctagcttggtaagaagagagacatgcccccaacctcggcgccctttttcctcacgatctgctgtccttacttcagcgactgcaggagcttcacctgcaagaaaacagcattgagctgctgac

SEQ ID NO: 317 - DNA, 409 bp, Homo sapiens
tgacagggctcctggagttgttaagtcaccaagtagctgcaggggatggacactgccccacacgatgtgggatgaacagcagccttggtttgtagcccagggtgtccatggatttgacccgaatgctccctggaggccctgtggcgaggacaggcactggatggtccagaccctctggctggaggagtggtggagccaggactgggccttcagccatgagggctagaataacctgacctcttgcattctaacactgggtcattaatgacacctttccagtggatgttgcaaaaaccaacactgtcaggaacctggccctgggagggctcaggtgagctcacaaggagaggtcaagccaagccaaagggtaggkaacacacaacaccaggggaaaccagcccccaaacca

SEQ ID NO: 318 - DNA, 320 bp, Homo sapiens
  misc_feature 6, 17, 24, 271: n = A,T,C or G
caaggnagatcttaagnggggtcntatgtaagtgtgctcctggctccagggttcctggagcctcacgaggtcaggggaacccttgtagaactccaccagcagcatcatctcgtgaaggatgtcattggtcaggaagctgtcctggacgtaggccatctccacatccatggggatgccatagtcactgggcctttgctcgggaggaggcatcacccagaaaggcgagatcttggactcggggcctgggttgccagaatagtaaggggagcanagcagggcgaggcagggctggaagccattgctggagccctgcagccgca

SEQ ID NO: 319 - DNA, 212 bp, Homo sapiens
  misc_feature 172: n = A,T,C or G
tgaagcaatagcgcccccattttacaggcggagcatggaagccagagaggtgggtgggggagggggtccttccctggctcaggcagatgggaagatgaggaagccgctgaagacgctgtcggcctcagagccctggtaaatgtgaccctttttggggtctttttcaacccanacctggtcaccctgctgcagacctcggccgcgaccacgct

SEQ ID NO: 320 - DNA, 769 bp, Homo sapiens
tggaggtgtagcagtgagaggagatytcaggcaagagtgtcacagcagagccctaaascctccaactcaccagtgagagatgagactgcccagtactcagccttcatctcctgggccacctggagggcgtctttctccatcagcgcatactgagcaggggtactcagatccttcttggaacctacaaggaagagaagcacactggaagggtcattctccttcagggcatcggccagccactgcctgccatgggaggtggaaagtaagggatgagtgagtctgcagggcccctcccactgacattcataggcccaattaccccctctctggtcctacatgcattcttcttcttcctgaccacccctctgttctgaaccctctcttcccggagcctcccattatattgcaggatgctcacttacttggtatgttccagagatgccacatcattcaggttgaagacaatgatgatggcttggaagagtggcagaaacagccccaggttgacagggaagacactactgctcatttccccaatccttccagctccatatgagaaagccatgtgcactctgagacccacctaccccacttcacccagccccttaccttgagctcctctatagtaggttgatgcaatgcatttgaacctctcctgcccagcggtatcccaactggaaggaaggaagagtgaagcacaggtatgtatcttggggggtgtgggtgctggggagaagggatagctggaaggggtgtggaagcactcaca

SEQ ID NO: 321 - DNA, 690 bp, Homo sapiens
  misc_feature 633, 666: n = A,T,C or G
tgggctgtgggcggcacctgtgctctgcaggccagacagcgatagaagcctttgtctgtgcctactcccccggaggcaactgggaggtcaacgggaagacaatcatcccctataagaagggtgcctggtgttcgctctgcacagccagtgtctcaggctgcttcaaagcctgggaccatgcaggggggctctgtgaggtccccaggaatccttgtcgcatgagctgccagaaccatggacgtctcaacatcagcacctgccactgccactgtccccctggctacacgggcagatactgccaagtgaggtgcagcctgcagtgtgtgcacggccggttccgggaggaggagtgctcgtgcgtctgtgacatcggctacgggggagcccagtgtgccaccaaggtgcattttcccttccacacctgtgacctgaggatcgacggagactgcttcatggtgtcttcagaggcagacacctattacagaagccaggatgaaatgtcagaggaatggcggggtgctggcccagatcaagagccagaaagtgcaggacatcctcgccttctatctgggccgcctggagaccaccaacgaggtgactgacagtgactttgagaccaggaacttctggatngggctcacctacaagaccgccaaggactccttncgctgggccacaggggagcaccag

SEQ ID NO: 322 - DNA, 104 bp, Homo sapiens
gtcgcaagccggagcaccaccatgtagcctttcccgaagtaccggaccttctcctcctccacgctcacatcacggacatcatggagcaggaccaccacctggtc

SEQ ID NO: 323 - DNA, 118 bp, Homo sapiens
gggccctgggcgcttccaaatgacccaggaggtggtctgcgacgaatgccctaatgtcaaactagtgaatgaagaacgaacactggaagtagaaatagagcctggggtgagagacgga

SEQ ID NO: 324 - DNA, 354 bp, Homo sapiens
tgctctccgggagcttgaagaagaaactggctacaaaggggacattgccgaatgttctccagcggtctgtatggacccaggcttgtcaaactgtactatacacatcgtgacagtcaccattaacggagatgatgccgaaaacgcaaggccgaagccaaagccaggggatggagagtttgtggaagtcatttctttacccaagaatgacctgctgcagagacttgatgctctggtagctgaagaacatctcacagtggacgccagggtctattcctacgctctagcgctgaaacatgcaaatgcaaagccatttgaagtgcccttcttgaaattttaagcccaaatatgacactg

SEQ ID NO: 325 - DNA, 642 bp, Homo sapiens
  misc_feature 1: n = A,T,C or G
ncatgcttgaatgggctcctggtgagagattgccccctggtggtgaaacaatcgtgtgtgcccactgataccaagaccaatgaaagagacacagttaagcagcaatccatctcatttccaggcacttcaataggtcgctgattggtccttgcaccagcagtggtagtcgtacctatttcagagaggtctgaaattcaggttcttagtttgccagggacaggccctaccttatatttttttccatcttcatcatccacttctgcttacagtttgctgcttacaataacttaatgatggattgagttatctgggtggtctctagccatctgggcagtgtggttctgtctaaccaaagggcattggcctcaaaccctgcatttggtttaggggctaacagagctcctcagataatcttcacacacatgtaactgctggagatcttattctattatgaataagaaacgagaagtttttccaaagtgttagtcaggatctgaaggctgtcattcagataacccagcttttccttttggcttttagcccattcagactttgccagagtcaagccaaggattgcttttttgctacagttttctgccaaatggcctagttcctgagtacctggaaaccagagagaaagag

SEQ ID NO: 326 - DNA, 455 bp, Homo sapiens
tccgtgaggatgagcttcgagtccttcaccaggcactgcaggggcacagtcacgtcaatcaccttcaccttctcgctcttcctgctcttgtcattgacaaacttcccgtaccaggcattgacgatgatgaggcccattctggactcttctgcctcaattatccttcggacagattcctgcatcagccggacagcggactccgcctcttgcttcttctgcagcacatcggtggcggcgctttccctctgcttctccaattccttctctttctgagccctgaggtatggtttgatgatcagacggtgcatggcaaagtagaccactagaggccccacggtggcatagaacatggcgctgggcagaagctggtccgtcaagtgaatagggaagaagtatgtctgactggccctgttgagcttgactttgagagaaacgccctgtggaactccaacgct

SEQ ID NO: 327 - DNA, 321 bp, Homo sapiens
ttcactgtgaactcgcagtcctcgatgaactcgcacagatgtgacagccctgtctccttgctctctgagttctcttcaatgatgctgatgatgcagtccacgatagcgcgcttatactcaaagccaccctcttcccgcagcatggtgaacaggaagttcataaggacggcgtgtttgcgaggatatttctgacacagggcactgatggcctggacaaccaccaccttgaattcatccgagatttctgacatgaaggaggagatctgcttcatgaggcggtcgatgctgctctcgctgcccgtcttaaggagggtggtgatg

SEQ ID NO: 328 - DNA, 476 bp, Homo sapiens
  misc_feature 302, 311: n = A,T,C or G
tgcaggaggggccatgggggctgtgaatgggatgcagccccatggtgtccctgataaatccagtgtgcagtctgatgaagtctgggtgggtgtggtctacgggctggcagctaccatgatccaagaggtaatgcactccttttcccatctctccaccatctgtatcctggccmagaaaaacttcccttcaaaccaaccaaaatttcctttcaaaggcataacccaaatgccatccttggtccggtctaataaagcctcccccatttttcccctggtatgcattcccaggctccctggccttncagggcttnctgtctgtgggtcatagtttatctcctcccacttgctgggagctccttgaaggcaaagactctactgcctccatctatccagtggaagtggctcttcagagggtgccaagttagtatgtatgactgtcatctctcccaacagggcctgacttggsagggcttcca

SEQ ID NO: 329 - DNA, 340 bp, Homo sapiens
cgagggagattgccagcaccctgatggagagtgagatgatggagatcttgtcagtgctagctaagggtgaccacagccctgtcacaagggctgctgcagcctgcctggacaaagcagtggaatatgggcttatccaacccaaccaagatggagagtgagggggttgtccctgggcccaaggctcatgcacacgctacctattgtggcacggagagtaaggacggaagcagctttggctggtggtggctggcatgcccaatactcttgcccatcctcgcttgctgccctaggatgtcctctgttctgagtcagcggccacgttcagtcacacagccctgct

SEQ ID NO: 330 - DNA, 277 bp, Homo sapiens
tgtcaccatcacattggtgccaaatacccagaagacatcgtagatgaagagtccgcccagcaggatgcagccagtgctgacattgttgaggtgcaggagctctactccattaagggagaaggccaggccaaaaaggttgttggcaatccagtgcttcctcagcaggtaccagacgccaacgatgctgctcaggcccaggcacaccaggtccttggtgtcaaattcataattgatgatctcctccttgttttcccagaaccctgtgtgaagagcagac

SEQ ID NO: 331 - DNA, 136 bp, Homo sapiens
ttgcttcccacctcctttctctgtcctctcctgaggttctgccttacaatggggacactgatacaaaccacacacacaatgaggatgaaaacagataacaggtaaaatgacctcacctgcccgggcggccgctcga

SEQ ID NO: 332 - DNA, 184 bp, Homo sapiens
ttgtgagataaacgcagatactgcaatgcattaaaacgcttgaaatactcatcagggatgttgctgatcttattgttgtctaagtagagagttagaagagagacagggagaccagaaggcagtctggctatctgattgaagctcaagtcaaggtattcgagtgatttaagacctttaaaagcag

SEQ ID NO: 333 - DNA, 384 bp, Homo sapiens
cggaaaacttcgaggaattgctcaaagtgctgggggtgaatgtgatgctgaggaagattgctgtggctgcagcgtccaagccagcagtggagatcaaacaggagggagacactttctacatcaaaacctccaccaccgtgcgcaccacagagattaacttcaaggttggggaggagtttgaggagcagactgtggatgggaggccctgtaagagcctggtgaaatgggagagtgagaataaaatggtctgtgagcagaagctcctgaagggagagggccccaagacctcgtggaccagagaactgaccaacgatggggaactgatcctgaccatgacggcggatgacgttgtgtgcaccagggtctacgtccgagagtgagcgg

SEQ ID NO: 334 - DNA, 169 bp, Homo sapiens
  misc_feature 2, 165: n = A,T,C or G
cnacaaacagagcagacaccctggatccggtcctgctactggccaggacggctggaccgtaaaattgaatttccacttcctgaccgccgccagaagagattgattttctccactatcactagcaagatgaacctctctgaggaggttgacttggaagactatgtngccc

SEQ ID NO: 335 - DNA, 185 bp, Homo sapiens
ccaggtttgcagcccaggctgcacatcaggggactgcctcgcaatacttcatgctgttgctgctgactgatggtgctgtgacggatgtggaagccacacgtgaggctgtggtgcgtgcctcgaacctgcccatgtcagtgatcattgtgggtgtgggtggtgctgactttgaggccatggagcag

SEQ ID NO: 336 - DNA, 358 bp, Homo sapiens
  misc_feature 26: n = A,T,C or G
ctgcccctgccttacggcggccaganacacacccaggatggcattggccccaaacttggatttgttctcagtcccatccaactccagcatcaggttgtccagtttctcttgctccaccacagagagacctgagctgatgagggctggcgcgatggtggagttgatgtggtccactgccttcaggacacctttgcctaagtaacgctgtttgtctccatccctcagctccagggcctcatagatgcccgtagaggctccactgggcactgcagcccggaaaagacctttggcagtatagagatccacctccactgtggggttcccgcgggagtccaggatctcccgggcccagatcttc

SEQ ID NO: 337 - DNA, 271 bp, Homo sapiens
  misc_feature 17: n = A,T,C or G
cacaaagccaccagccngggaaatcagaatttacttgatgcaactgacttgtaatagccagaaatcctgcccagcatgggattcagaacctggtctgcaaccaaatccaccgtcaaagttcatacaggataaaacaaattcaattgccttttccacattaatagcatcaagcttccccaacaaagccaaagttgccaccgcacaaaaagagaatcttgtgtcaatttctccctactttataaaagtagatttttcacatcccatgaagcag

SEQ ID NO: 338 - DNA, 326 bp, Homo sapiens
  misc_feature 15, 17, 18: n = A,T,C or G
ctgtgctcccgactngnncatctcaggtaccaccgactgcactgggcggggccctctggggggaaaggctccacggggcagggatacatctcgaggccagtcatcctctggaggcagcccaatcaggtcaaagattttgcccaactggtcggcttcagagtttccacagaagagaggctttcgacgaaacatctctgcaaagatacagccaacactccacatgtccacaggtgttgcatatgtggactgcagaagaacttcgggagctcggtaccagagtgtaacaaccacgggtgtaagtgccatctggtagctgtagattctgg

SEQ ID NO: 339 - DNA, 260 bp, Homo sapiens
  misc_feature 47, 54, 60, 69, 90, 91, 96, 113, 117, 119, 195: n = A,T,C or G
ttcacctgaggactcatttcgtgccctttgttgacttcaagcaaagnccttcanggtctncaaggacgncacatttccacttgcgaatgnnctcanggctcatcttgaagaanaagnancccaagtgctggatcccagactcgggggtaaccttgtgggtaagagctcatccagtttatgctttaggacgtccanctactcgggggagctggaagcctgcgtggatgcggccctgctggacctcggccgcgaccacgcta

SEQ ID NO: 340 - DNA, 220 bp, Homo sapiens
  misc_feature 15, 18: n = A,T,C or G
ctggaagcccggctnggnctggcagcggaaggagccaggcaggttcacgcagcggtgctggcagtagcggtagcggcactcgtctatgtccacacactcgggcccgatcttgcggtaaccatcagggcaggtgcactgataggagccaggcaagttatggcagtcctggctggggcgacagtcgtgcagggcctgggcacactcgtccacatccacacag

SEQ ID NO: 341 - DNA, 384 bp, Homo sapiens
ctgctaccaggggagcgagagctgactatcccagcctcggctaatgtattctacgccatggatggagcttcacacgatttcctcctgcggcagcggcgaaggtcctctactgctacaccgggcgtcaccagtggcccgtctgcctcaggaactcctccgagtgagggaggagggggctcctttcccaggatcaaggccacagggaggaagattgcacgggcactgttctgaggaggaagccccgttggcttacagaagtcatggtgttcataccagatgtgggtagccatcctgaatggtggcaattatatcacattgagacagaaattcagaaagggagccagccaccctggggcagtgaagtgccactggtttaccagacag

SEQ ID NO: 342 - DNA, 245 bp, Homo sapiens
ctggctaagctcatcattgttactggtgggcaccatgtccttgaagcttcaggcaagcaatgtaaccaacaagaatgaccccaagtccatcaactctcgagtcttcattggaaacctcaacacagctctggtgaagaaatcagatgtggagaccatcttctctaagtatggccgtgtggccggctgttctgtgcacaagggctatgcctttgttcagtactccaatgagcgccatgcccgggcag

SEQ ID NO: 343 - DNA, 611 bp, Homo sapiens
ccaaaaaaatcaagatttaatttttttatttgcactgaaaaactaatcataactgttaattctcagccatctttgaagcttgaaagaagagtctttggtattttgtaaacgttagcagactttcctgccagtgtcagaaaatcctatttatgaatcctgtcggtattccttggtatctgaaaaaaataccaaatagtaccatacatgagttatttctaagtttgaaaaataaaaagaaattgcatcacactaattacaaaatacaagttctggaaaaaatatttttcttcattttaaaactttttttaactaataatggctttgaaagaagaggcttaatttgggggtggtaactaaaatcaaaagaaatgattgacttgagggtctctgtttggtaagaatacatcattagcttaaataagcagcagaaggttagttttaattatgtagcttctgttaatattaagtgttttttgtctgttttacctcaatttgaacagataagtttgcctgcatgctggacatgcctcagaaccatgaatagcccgtactagatcttgggaacatggatcttagagtcctttggaataagttcttatataaataccccc

SEQ ID NO: 344 - DNA, 311 bp, Homo sapiens
  misc_feature 1, 275, 284, 296, 297, 300: n = A,T,C or G
nctcgaaaaagcccaagacagcagaagcagacacctccagtgaactagcaaagaaaagcaaagaagtattcagaaaagagatgtcccagttcatcgtccagtgcctgaacccttaccggaaacctgactgcaaagtgggaagaattaccacaactgaagactttaaacatctggctcgcaagctgactcacggtgttatgaataaggagctgaagtactgtaagaatcctgaggacctggagtgcaatgagaatgtgaaacacaaaaccaaggantacattaanaagtacatgcannaantttggggcttg

SEQ ID NO: 345 - DNA, 201 bp, Homo sapiens
cacacggtcatcccgactgccaacctggaggcccaggccctgtggaaggagccgggcagcaatgtcaccatgagtgtggatgctgagtgtgtgcccatggtcagggaccttctcaggtacttctactcccgaaggattgacatcaccctgtcgtcagtcaagtgcttccacaagctggcctctgcctatggggccaggcag

SEQ ID NO: 346 - DNA, 370 bp, Homo sapiens
ctgctccagggcgtggtgtgccttcgtggcctctgcctcctccgaggagccaggctgtgttctcttcagaatgttctggagcagcagtttgaggcgggtgatgcgttggaagggcagaatcagaaaggacttgagggaaaggcgctggcagacggggtcgctctccagcttctccaagacctcccggaaattgctgttgctattcatcaggctctggaaggtgcgttcctgataggtctggttggtgacataaggcaggtagacccggcggaagtctggggcgtggttcaggactacgtcacatacttggaaggagaagatattgttctcaaagttctcttccaggtctgaaaggaacgtggcgctgacg

SEQ ID NO: 347 - DNA, 416 bp, Homo sapiens
  misc_feature 416: n = A,T,C or G
ctgttgtgctgtgtatggacgtgggctttaccatgagtaactccattcctggtatagaatccccatttgaacaagcaaagaaggtgataaccatgtttgtacagcgacaggtgtttgctgagaacaaggatgagattgctttagtcctgtttggtacagatggcactgacaatcccctttctggtggggatcagtatcagaacatcacagtgcacagacatctgatgctaccagattttgatttgctggaggacattgaaagcaaaatccaaccaggttctcaacaggctgacttcctggatgcactaatcgtgagcatggatgtgattcaacatgaaacaataggaaagaagtttggagaagaggcatattgaaatattcactgacctcaagcagcccgattcagcaaaagtcan

SEQ ID NO: 348 - DNA, 351 bp, Homo sapiens
gtacaggagaggatggcaggtgcagagcgggcactgagctctgcaggtgaaagggctcggcagttggatgctctcctggaggctctgaaattgaaacgggcaggaaatagtctggcagcctctacagcagaagaaacggcaggcagtgcccagggacgagcaggagacagatgccttcctcttgtctcaactgcaaagaggcgttccttcctctttcactaatcctcctcagcacagaccctttacgggtgtcaggctgggggacagtaaggtctttcccttcccacaaggccatatctcaggctgtctcagtggggggaaaccttggacaatacccgggctttcttgggc

SEQ ID NO: 349 - DNA, 207 bp, Homo sapiens
  misc_feature 1: n = A,T,C or G
nccgggacatctccaccctcaacagtggcaagaagagcctggagactgaacacaaggccttgaccagtgagattgcactgctgcagtccaggctgaagacagagggctctgatctgtgcgacagagtgagcgaaatgcagaagctggatgcacaggtcaaggagctggtgctgaagtcggcggtggaggctgagcgcctggtggctg

SEQ ID NO: 350 - DNA, 323 bp, Homo sapiens
ccatacagggctgttgcccaggccctagaggtcattcctcgtaccctgatccagaactgtggggccagcaccatccgtctacttacctcccttcgggccaagcacacccaggagaactgtgagacctggggtgtaaatggtgagacgggtactttggtggacatgaaggaactgggcatatgggagccattggctgtgaagctgcagacttataagacagcagtggagacggcagttctgctactgcgaattgatgacatcgtttcaggccacgaaaagaaaggcgatgaccagagccggcaaggcggggctcctgatgctgg

SEQ ID NO: 351 - DNA, 353 bp, Homo sapiens
  misc_feature 12, 25, 39, 42: n = A,T,C or G
cgccgcatcccntggtcccttccantcccttttcctttntcngggaacgtgtatgcggtttgtttttgttttgtagggtttttttccttctccacctctccctgtctcttttgctccatgttgtccgtttctgtggggttaggtttatgtttttaatcatctgaggtcacgtctatttcctccggactcgcctgcttggtggcgattctccaccggttaatatggtgcgtcccttttttcttttgttgcgaatctgagccttcttcctccagcttctgccttttgaactttgttcttcggttctgaaaccatacttttacctgagtttccgtgaggctgaggctgtgtgccaa

SEQ ID NO: 352 - DNA, 467 bp, Homo sapiens
ctgcccacactgatcacttgcgagatgtccttagggtacaagaacaggaattgaagtctgaatttgagcagaacctgtctgagaaactctctgaacaagaattacaatttcgtcgtctcagtcaagagcaagttgacaactttactctggatataaatactgcctatgccagactcagaggaatcgaacaggctgttcagagccatgcagttgctgaagaggaagccagaaaagcccaccaactctggctttcagtggaggcattaaagtacagcatgaagacctcatctgcagaaacacctactatcccgctgggtagtgcagttgaggccatcaaagccaactgttctgataatgaattcacccaagctttaaccgcagctatccctccagagtccctgacccgtggggtgtacagtgaagagacccttagagcccgtttctatgctgttcaaaaactggcccga

SEQ ID NO: 353 - DNA, 350 bp, Homo sapiens
ctgctgcagccacagtagttcctcccatggtgggtggccctcctggtcctgctggcccaggaaatctgtccccaccaggaacagcccctggaaaacggccccgtcctctaccaccttgtggaaatgctgcacgggaactgcctcctggaggaccagctttaccttccccagacatttgtcctgattgtgtagttttcctggactgcatttcaaattgactcaggaactgtttattgcatggagttacaacaggattctgaccatgaagttctcttttaggtaacagatccattaacttttttgaagatgcttcagatccaacaccaacaagggcaaacccctttgactgg

SEQ ID NO: 354 - DNA, 351 bp, Homo sapiens
atttagatgagatctgaggcatggagacatggagacagtatacagactcctagatttaagttttaggttttttgcttttctaatcaccaattcttatatacaatgtatattttagactcgagcagatgatcatcttcatcttaagtcattccttttgactgagtatggcaggattagagggaatggcagtatagatcaatgtctttttctgtaaagtataggaaaaaccagagaggaaaaaaagagctgacaattggaaggtagtagaaaattgacgataatttcttcttaacaaataatagttgtatatacaaggaggctagtcaaccagattttatttgttgagggcga

SEQ ID NO: 355 - DNA, 308 bp, Homo sapiens
ttttggcgcaagttttacagattttattaaagtcgaagctattggtcttggaagatgaaaatgcaaatgttgatgaggtggaattgaagccagataccttaataaaattatatcttggttataaaaataagaaattaagggttaacatcaatgtgccaatgaaaaccgaacagaagcaggaacaagaaaccacacacaaaaacatcgaggaagaccgcaaactactgattcaggcggccatcgtgagaatcatgaagatgaggaaggttctgaaacaccagcagttacttggcgaggtcctcactcag

SEQ ID NO: 356 - DNA, 207 bp, Homo sapiens
ctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggagg

SEQ ID NO: 357 - DNA, 188 bp, Homo sapiens
  misc_feature 25, 29: n = A,T,C or G
tcgaccacgccctcgtagcgcatgngctncaggacgatgctcagagtgatgaacaccccggtgcggcccacgccagcactgcagtgcaccgtgataggcccatcctgtccaaactgctccttggtcttatgcacctgcccgatgaagtcaatgaatccctcgcctgtcttgggcacgccctgctctgg

SEQ ID NO: 358 - DNA, 291 bp, Homo sapiens
ctgggagcatcggcaagctactgccttaaaatccgatctccccgagtgcacaatttctgtcccttttaagggttcacaacactaaagatttcacatgaaagggttgtgattgatttgagcaggcaggcggtacgtgacaggggctgcatgcaccggtggtcagagagaaacagaacagggcagggaatttcacaatgttcttctatacaatggctggaatctatgaataacatcagtttctaagttatgggttgatttttaactactgggtttaggccaggcaggcccagg

SEQ ID NO: 359 - DNA, 117 bp, Homo sapiens
  misc_feature 79, 98, 100: n = A,T,C or G
gccaccacactccagcctgggcaatacagcaagactgtctcaaaaaaaaaaaaaaaaaaacccaaaaaaactcaaaaangtaatgaatgatacccaangngccttttctagaaaaag

SEQ ID NO: 360 - DNA, 394 bp, Homo sapiens
ctgttcctctggggtggtccagttctagagtgggagaaagggagtcaggcgcattgggaatcgtggttccagtctggttgcagaatctgcacatttgccaagaaattttccctgtttggaaagtttgccccagctttcccgggcacaccaccttttgtcccaagtgtctgccggtcgaccaatctgcctgccacacattgaccaagccagacccggttcacccagctcgaggatcccaggttgaagagtggccccttgaggccctggaaagaccaatcactggacttcttcccttgagagtcagaggtcacccgtgattctgcctgcaccttatcattgatctgcagtgatttctgcaaatcaagagaaactctgcagggcactcccctgtttc

SEQ ID NO: 361 - DNA, 394 bp, Homo sapiens
  misc_feature 28, 31: n = A,T,C or G
ctgggcggatagcaccgggcatattttnttnatggatgaggtctggcaccctgagcagtccagcgaggacttggtcttagttgagcaatttggctaggaggatagtatgcagcacggttctgagtctgtgggatagctgccatgaagtaacctgaaggaggtgctggctggtaggggttgattacagggttgggaacagctcgtacacttgccattctctgcatatactggttagtgaggtgagcctggcgctcttctttgcgctgagctaaagctacatacaatggctttgtggacctcggccgcgaccacgctaagccgaattccagcacactggcggccgttactagtggatccgagctcggtaccaagcttggcgtaatcatggtcatag

SEQ ID NO: 362 - DNA, 268 bp, Homo sapiens
ctgcgcgtggaccagtcagcttccgggtgtgactggagcagggcttgtcgtcttcttcagagtcactttgcaggggttggtgaagctgctcccatccatgtacagctcccagtctactgatgtttaaggatggtctcggtggttaggcccactagaataaactgagtccaatacctctacacagttatgtttaactgggctctctgacaccgggaggaaggtggcggggtttaggtgttgcaaacttcaatggttatgcggggatgtt

SEQ ID NO: 363 - DNA, 323 bp, Homo sapiens
ccttgaccttttcagcaagtgggaaggtgtaatccgtctccacagacaaggccaggactcgtttgtacccgttgatgatagaatggggtactgatgcaacagttgggtagccaatctgcagacagacactggcaacattgcggacaccctccaggaagcgagaatgcagagtttcctctgtgatatcaagcacttcagggttgtagatgctgccattgtcgaacacctgctggatgaccagcccaaaggagaagggggagatgttgagcatgttcagcagcgtggcttcgctggctcccactttgtctccagtcttgatcaga

SEQ ID NO: 364 - DNA, 393 bp, Homo sapiens
  misc_feature 29: n = A,T,C or G
ccaagctctccatcgtccccgtgcgcagnggctactgggggaacaagatcggcaagccccacactgtcccttgcaaggtgacaggccgctgcggctctgtgctggtacgcctcatcactgcacccaggggcactggcatcgtctccgcacctgtgcctaagaagctgctcatgatggctggcatcgatgactgctacacctcagcccggggctgcactgccaccctgggcaacttcgccaaggccacctttgatgccatttctaagacctacagctacctgacccccgacctctggaaggagactgtattcaccaagtctccctatcaggagttcactgaccacctcgtcaagacccacaccagagtctccgtgcagcggactcaggctccag

SEQ ID NO: 365 - DNA, 371 bp, Homo sapiens
cctcctcagagcggtagctgttcttattgccccggcagcctccatagatgaagttattgcaggagttcctctccacgtcaaagtaccagcgtgggaaggatgcacggcaaggcccagtgactgcgttggcggtgcagtattcttcatagttgaacatatcgctggagtggtcttcagaatcctgccttctgggagcacttgggacagaggaatccgctgcattcctgctggtggacctcggccgcgaccacgctaagccgaattccagcacactggcggccgttactagtggatccgagctcggtaccaagcttggcgtaatcatggtcatagctgtttcctgtgtgaaattgttatccgctcacaattcc

SEQ ID NO: 366 - DNA, 393 bp, Homo sapiens
atttcttgccagatgggagctctttggtgaagactcctttcgggaaaagttttttggcttcttcttcagggatggttggaaggaccatcacactatccccatccttccaatcaactggggtggcaacccttttttctgctgtcagctggagagagatgactaccctgagaatctcatcaaagttcctgccagtggtagctgggtagaggatagacagcttcagcttcttatcaggaccaaaaacaaacaccacacgagctgccacaggcatgcccttttcatccttctctgctggatccagcatgcccaacaggatggcaagctcccgattcctatcatcgatgatgggaaaaggtaacttttctgtgggctcttcacaattgtaagcattga

SEQ ID NO: 367 - DNA, 327 bp, Homo sapiens
  misc_feature 34, 54, 55: n = A,T,C or G
ccagctctgtctcatacttgactctaaagtcttnagcagcaagacgggcattgnnaatctgcagaacgatgcgggcattgtccacagtatttgcgaagatctgagccctcaggtcctcgatgatcttgaagtaatggctccagtctctgacctggggtcccttcttctccaagtgctcccggattttgctctccagcctccggttctcggtctccaggctcctcactctgtccaggtaagaggccaggcggtcgttcaggctttgcatggtctccttctcgttctggatgcctcccattcctgccagacccccggctatcccggtgg

SEQ ID NO: 368 - DNA, 306 bp, Homo sapiens
  misc_feature 24: n = A,T,C or G
ctggagaaggacttcagcagtttnaagaagtactgccaagtcatccgtgtcattgcccacacccagatgcgcctgcttcctctgcgccagaagaaggcccacctgatggagatccaggtgaacggaggcactgtggccgagaagctggactgggcccgcgagaggcttgagcagcaggtacctgtgaaccaagtgtttgggcaggatgagatgatcgacgtcatcggggtgaccaagggcaaaggctacaaaggggtcaccagtcgttggcacaccaagaagctgccccgcaagacccaccgagga

SEQ ID NO: 369 - DNA, 394 bp, Homo sapiens
tcgacccacaccggaacacggagagctgggccagcattggcacttgataggatttcccgtcggctgccacgaaagtgcgtttctttgtgttctcgggttggaaccgtgatttccacagacccttgaaatacactgcgttgacgaggaccagtctggtgagcacaccatcaataagatctggggacagcagattgtcaatcatatccctggtttcatttttaacccatgcattgatggaatcacaggcagaggctggatcctcaaagttcacattccggacctcacactggaacacatctttgttccttgtaacaaaaggcacttcaatttcagaggcattcttaacaaacacggcgttagccactgtcacaatgtctttattcttcttggagac

SEQ ID NO: 370 - DNA, 653 bp, Homo sapiens
ccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggacaagaagctctctctcagacaaccatctcatgggccccattccaggacacttctgagtacatcatttcatgtcatcctgttggcactgatgaagaacccttacagttcagggttcctggaacttctaccagtgccactctgacagga

SEQ ID NO: 371 - DNA, 268 bp, Homo sapiens
ctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgact

SEQ ID NO: 372 - DNA, 392 bp, Homo sapiens
gctggtgcccctggtgaacgtggacctcctggattggcaggggccccaggacttagaggtggaactggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaacctcggccgcgac

SEQ ID NO: 373 - DNA, 388 bp, Homo sapiens
  misc_feature 30: n = A,T,C or G
ccaagcgctcagatcggcaaggggcaccanttttgatctgcccagtgcacagccccacaaccaggtcagcgatgaaggtatcttcagtctcccccgaacgatgagacaccatgacgccccaaccattggcctgggccagcttgcacgcctgaagagactcggtcacggagccaatctggttgactttgagcaggaggcagttgcaggacttctcgttcacggccttggcgatcctctttgggttggtcactgtgagatcatcccccactacctggattcctgcactggctgtgaacttctgccaagctccccagtcatcctggtcaaagggatcttcgatagacaccactgggtagtccttgatgaaggacttgtacaggtcagccag

SEQ ID NO: 374 - DNA, 393 bp, Homo sapiens
ctgacgaccgcgtgaacccctgcattgggggtgtcatcctcttccatgagacactctaccagaaggcggatgatgggcgtcccttcccccaagttatcaaatccaagggcggtgttgtgggcatcaaggtagacaagggcgtggtccccctggcagggacaaatggcgagactaccacccaagggttggatgggctgtctgagcgctgtgcccagtacaagaaggacggagctgacttcgccaagtggcgttgtgtgctgaagattggggaacacaccccctcagccctcgccatcatggaaaatgccaatgttctggcccgttatgccagtatctgccagcagaatggcattgtgcccatcgtggagcctgagatcctccctgatggggacc

SEQ ID NO: 375 - DNA, 394 bp, Homo sapiens
  misc_feature 30, 33: n = A,T,C or G
ccacaaatggcgtggtccatgtcatcaccnttnttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaaca

SEQ ID NO: 376 - DNA, 392 bp, Homo sapiens
  misc_feature 30: n = A,T,C or G
ctgcccagcccccattggcgagtttgattnggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctg

SEQ ID NO: 377 - DNA, 292 bp, Homo sapiens
caatgtttgatgcttaacccccccaatttctgtgagatggatggccagtgcaagcgtgacttgaagtgttgcatgggcatgtgtgggaaatcctgcgtttcccctgtgaaagcttgattcctgccatatggaggaggctctggagtcctgctctgtgtggtccaggtcctttccaccctgagacttggctccaccactgatatcctcctttggggaaaggcttggcacacagcaggctttcaagaagtgccagttgatcaatgaataaataaacgagcctatttctctttgc

SEQ ID NO: 378 - DNA, 395 bp, Homo sapiens
ctgctgcttcagcgaagggtttctggcatatccaatgataaggctgccaaagactgttccaataccagcaccagaaccagccactcctactgttgcagcacctgcaccaataaatttggcagcagtatcaatgtctctgctgattgcactggtctgaaactccctttggattagctgagacacaccattctgggccctgattttcctaagatagaactccaactctttgccctctagcacatagccatctgctcggccacactgtcccggccttgaagcgatgcacgcaagaagcttgccctgctggaactgctcctccaggagactgctgattttggcattctttttcctttcatcatatttcttctgaattttttagatcgttttttgtttaa

SEQ ID NO: 379 - DNA, 223 bp, Homo sapiens
ccagatgaaatgctgccgcaatggctgtgggaaggtgtcctgtgtcactcccaatttctgagctccagccaccaccaggctgagcagtgaggagagaaagtttctgcctggccctgcatctggttccagcccacctgccctcccctttttcgggactctgtattccctcttgggctgaccacagcttctccctttcccaaccaataaagtaaccactttcagc

SEQ ID NO: 380 - DNA, 317 bp, Homo sapiens
  misc_feature 30, 32: n = A,T,C or G
tcgaccacagtattccaaccctcctgtgcntngagaagtgatggagggtgctgacaaccagggtgcaggagaacaaggtagaccagtgaggcagaatatgtatcggggatatagaccacgattccgcaggggccctcctcgccaaagacagcctagagaggacggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccag

SEQ ID NO: 381 - DNA, 392 bp, Homo sapiens
  misc_feature 29, 30, 31: n = A,T,C or G
cctgaaggaagagctggcctacctgaatnnnaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagacctcggccgcgaccacgctaagccgaattccagcacactggcggccgttactagtggatccgagctcgg

SEQ ID NO: 382 - DNA, 234 bp, Homo sapiens
cctcgatgtctaaatgagcgtggtaaaggatggtgcctgctggggtctcgtagatacctcgggacttcattccaatgaagcggttctccacgatgtcaatacggcccacgccatgcttgcccgcgacttcgttcaggtacatgaagagctccaaggaggtctggtgggtggtgccatccttgacgttggtcaccttcacagggaccccttttttgaactccatctccagaatgt

SEQ ID NO: 383 - DNA, 396 bp, Homo sapiens
  misc_feature 66: n = A,T,C or G
ccttgaccttttcagcaagtgggaaggtgttttccgtctccacagacaaggccaggactcgtttgnacccgttgatgatagaatggggtactgatgcaacagttgggtagccaatctgcagacagacactggcaacattgcggacacccaggatttcaatggtgcccctggagattttagtggtgatacctaaagcctggaaaaaggaggtcttctcgggcccgagaccagtgttctgggctggcacagtgacttcacatggggcaatggcaccagcacgggcagcagacctgcccgggcggccgctcgaaagccgaattccagcacactggcggccgttactagtggatccgagctcggtaccaagcttggcgtaatcatggtcatagctgtttc

SEQ ID NO: 384 - DNA, 396 bp, Homo sapiens
gctgaataggcacagagggcacctgtacaccttcagaccagtctgcaacctcaggctgagtagcagtgaactcaggagcgggagcagtccattcaccctgaaattcctccttggtcactgccttctcagcagcagcctgctcttctttttcaatctcttcaggatctctgtagaagtacagatcaggcatgacctcccatgggtgttcacgggaaatggtgccacgcatgcgcagaacttcccgagccagcatccaccacatcaaacccactgagtgagctcccttgttgttgcatgggatggcaatgtccacatagcgcagaggagaatctgtgttacacagcgcaatggtaggtaggttaacataagatgcctccgtgagaggctggtggtcag

SEQ ID NO: 385 - DNA, 2943 bp, Homo sapiens
cagccaccggagtggatgccatctgcacccaccgccctgaccccacaggccctgggctggacagagagcagctgtatttggagctgagccagctgacccacagcatcactgagctgggcccctacaccctggacagggacagtctctatgtcaatggtttcacacagcggagctctgtgcccaccactagcattcctgggacccccacagtggacctgggaacatctgggactccagtttctaaacctggtccctcggctgccagccctctcctggtgctattcactctcaacttcaccatcaccaacctgcggtatgaggagaacatgcagcaccctggctccaggaagttcaacaccacggagagggtccttcagggcctggtccctgttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggcccctatgccctggacaacgacagcctctttgtcaatggtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctgcaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatggtttcacccatcggagctctgtacccaccaccagcaccggggtggtcagcgaggagccattcacactgaacttcaccatcaacaacctgcgctacatggcggacatgggccaacccggctccctcaagttcaacatcacagacaacgtcatgaagcacctgctcagtcctttgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacggttacaatgaacctggtccagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatggctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctacctgaatttcaccatcaccaacctaccatattcccaggacaaagcccagccaggcaccaccaattaccagaggaacaaaaggaatattgaggatgcggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattttcccaacagaaatgagcccttaactgggaattctgaccttcccttctgggctgtcatcctcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcctggggtgcctttcccccagccagggtccaaagaagcttggctggggcagaaataaaccatattggtcggaaaaaaaaaaaaaaaaaaaaaaaaaaaaaaaaaaaaaaaaaaaaaaa

SEQ ID NO: 386 - DNA, 2608 bp, Homo sapiens
gttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggcccctatgccctggacaacgacagcctctttgtcaatggtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctgcaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatggtttcacccatcggagctctgtacccaccaccagcaccggggtggtcagcgaggagccattcacactgaacttcaccatcaacaacctgcgctacatggcggacatgggccaacccggctccctcaagttcaacatcacagacaacgtcatgaagcacctgctcagtcctttgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacggttacaatgaacctggtccagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatggctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctacctgaatttcaccatcaccaacctaccatattcccaggacaaagcccagccaggcaccaccaattaccagaggaacaaaaggaatattgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattttcccaacagaaatgagcccttaactgggaattctgaccttcccttctgggctgtcatcctcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcctggggtgcctttcccccagccagggtccaaagaagcttggctggggcagaaataaaccatattggtcggacacaaaaaaaaaaaaaa

SEQ ID NO: 387 - DNA, 1761 bp, Homo sapiens
ctgaacttcaccatcaacaacctgcgctacatggcggacatgggccaacccggctccctcaagttcaacatcacagacaacgtcatgaagcacctgctcagtcctttgttccagaggagcagcctgggtgcacggtacacaggctgcagggtcatcgcactaaggtctgtgaagaacggtgctgagacacgggtggacctcctctgcaggtaggtgcagaggaggtccacggcatcacccggctgggcccctactctctggacaaagacagcctctaccttaacgctcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatggctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctacctgaatttcaccatcaccaacctaccatattcccaggacaaagcccagccaggcaccaccaattaccagaggaacaaaaggaatattgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattttcccaacagaaatgagcccttaactgggaattctgaccttcccttctgggctgtcatcctcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcctggggtgcctttcccccagccagggtccaaagaagcttggctggggcagaaataaaccatattggtcggacacaaaaaaaaaaaaaaa

SEQ ID NO: 388 - protein, 772 aa, Homo sapiens
MSMVSHSGALCPPLAFLGPPQWTWEHLGLQFLNLVPRLPALSWCYSLSTSPSPTCGMRRTCSTLAPGSSTPRRGSFRAWSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDAAPHRGGLPV

SEQ ID NO: 389 - protein, 833 aa, Homo sapiens
FKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYFPNRNEPLTGNSDLPFWAVILIGLAGLLGLITCLICGVLVTTRRRKKEGEYNVQQQCPGYYQSHLDLEDLQ

SEQ ID NO: 390 - protein, 438 aa, Homo sapiens
MGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYFPNRNEPLTGNSDLPFWAVILIGLAGLLGLITCLICGVLVTTRRRKKEGEYNVQQQCPGYYQSHLDLEDLQ

SEQ ID NO: 391 - DNA, 2627 bp, Homo sapiens
ccacgcgtccgcccacgcgtccggaaggcagcggcagctccactcagccagtacccagatacgctgggaaccttccccagccatggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaataatgtgccttggccacaaaaaagcatgcaaagtcattgttacaacagggatctacagaactatttcaccaccagatatgacctagttttatatttctgggaggaaatgaattcatatctagaagtctggagtgagcaaacaagagcaagaaacaaaaagaagccaaaagcagaaggctccaatatgaacaagataaatctatcttcaaagacatattagaagttgggaaaataattcatgtgaactagacaagtgtgttaagagtgataagtaaaatgcacgtggagacaagtgcatccccagatctcagggacctccccctgcctgtcacctggggagtgagaggacaggatagtgcatgttctttgtctctgaatttttagttatatgtgctgtaatgttgctctgaggaagcccctggaaagtctatcccaacatatccacatcttatattccacaaattaagctgtagtatgtaccctaagacgctgctaattgactgccacttcgcaactcaggggcggctgcattttagtaatgggtcaaatgattcactttttatgatgcttccaaaggtgccttggcttctcttcccaactgacaaatgccaaagttgagaaaaatgatcataattttagcataaacagagcagtcggcgacaccgattttataaataaactgagcaccttctttttaaacaaacaaatgcgggtttatttctcagatgatgttcatccgtgaatggtccagggaaggacctttcaccttgactatatggcattatgtcatcacaagctctgaggcttctcctttccatcctgcgtggacagctaagacctcagttttcaatagcatctagagcagtgggactcagctggggtgatttcgccccccatctccgggggaatgtctgaagacaattttggttacctcaatgagggagtggaggaggatacagtgctactaccaactagtggataaaggccagggatgctgctcaacctcctaccatgtacaggacgtctccccattacaactacccaatccgaagtgtcaactgtgtcaggactaagaaaccctggttttgagtagaaaagggcctggaaagaggggagccaacaaatctgtctgcttcctcacattagtcattggcaaataagcattctgtctctttggctgctgcctcagcacagagagccagaactctatcgggcaccaggataacatctctcagtgaacagagttgacaaggcctatgggaaatgcctgatgggattatcttcagcttgttgagcttctaagtttctttcccttcattctaccctgcaagccaagttctgtaagagaaatgcctgagttctagctcaggttttcttactctgaatttagatctccagacccttcctggccacaattcaaattaaggcaacaaacatataccttccatgaagcacacacagacttttgaaagcaaggacaatgactgcttgaattgaggccttgaggaatgaagctttgaaggaaaagaatactttgtttccagcccccttcccacactcttcatgtgttaaccactgccttcctggaccttggagccacggtgactgtattacatgttgttatagaaaactgattttagagttctgatcgttcaagagaatgattaaatatacatttcctacaccaaaaaaaaaaaaa

SEQ ID NO: 392 - protein, 309 aa, Homo sapiens
HASAHASGRQRQLHSASTQIRWEPSPAMASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 393 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 394 - protein, 20 aa, Homo sapiens
MASLGQILFWSIISIIIILA

SEQ ID NO: 395 - protein, 20 aa, Homo sapiens
IIILAGAIALIIGFGISGRH

SEQ ID NO: 396 - protein, 20 aa, Homo sapiens
ISGRHSITVTTVASAGNIGE

SEQ ID NO: 397 - protein, 20 aa, Homo sapiens
GNIGEDGILSCTFEPDIKLS

SEQ ID NO: 398 - protein, 20 aa, Homo sapiens
DIKLSDIVIQWLKEGVLGLV

SEQ ID NO: 399 - protein, 20 aa, Homo sapiens
VLGLVHEFKEGKDELSEQDE

SEQ ID NO: 400 - protein, 20 aa, Homo sapiens
SEQDEMFRGRTAVFADQVIV

SEQ ID NO: 401 - protein, 20 aa, Homo sapiens
DQVIVGNASLRLKNVQLTDA

SEQ ID NO: 402 - protein, 21 aa, Homo sapiens
VQLTDAGTYKCYIITSKGKGN

SEQ ID NO: 403 - protein, 20 aa, Homo sapiens
KGKGNANLEYKTGAFSMPEV

SEQ ID NO: 404 - protein, 20 aa, Homo sapiens
SMPEVNVDYNASSETLRCEA

SEQ ID NO: 405 - protein, 20 aa, Homo sapiens
LRCEAPRWFPQPTVVWASQV

SEQ ID NO: 406 - protein, 20 aa, Homo sapiens
WASQVDQGANFSEVSNTSFE

SEQ ID NO: 407 - protein, 20 aa, Homo sapiens
NTSFELNSENVTMKVVSVLY

SEQ ID NO: 408 - protein, 20 aa, Homo sapiens
VSVLYNVTINNTYSCMIEND

SEQ ID NO: 409 - protein, 20 aa, Homo sapiens
MIENDIAKATGDIKVTESEI

SEQ ID NO: 410 - protein, 20 aa, Homo sapiens
TESEIKRRSHLQLLNSKASL

SEQ ID NO: 411 - protein, 20 aa, Homo sapiens
SKASLCVSSFFAISWALLPL

SEQ ID NO: 412 - protein, 20 aa, Homo sapiens
SSFFAISWALLPLSPYLMLK

SEQ ID NO: 413 - protein, 35 aa, Homo sapiens
ISGRHSITVTTVASAGNIGEDGILSCTFEPDIKLS

SEQ ID NO: 414 - protein, 35 aa, Homo sapiens
VLGLVHEFKEGKDELSEQDEMFRGRTAVFADQVIV

SEQ ID NO: 415 - protein, 65 aa, Homo sapiens
KGKGNANLEYKTGAFSMPEVNVDYNASSETLRCEAPRWFPQPTVVWASQVDQGANFSEVSNTSFE

SEQ ID NO: 416 - protein, 10 aa, Homo sapiens
KLSDIVIQWL

SEQ ID NO: 417 - protein, 10 aa, Homo sapiens
SLGQILFWSI

SEQ ID NO: 418 - protein, 10 aa, Homo sapiens
LLNSKASLCV

SEQ ID NO: 419 - protein, 10 aa, Homo sapiens
SLCVSSFFAI

SEQ ID NO: 420 - protein, 10 aa, Homo sapiens
VLYNVTINNT

SEQ ID NO: 421 - protein, 10 aa, Homo sapiens
ILFWSIISII

SEQ ID NO: 422 - protein, 10 aa, Homo sapiens
LLPLSPYLML

SEQ ID NO: 423 - protein, 10 aa, Homo sapiens
CMIENDIAKA

SEQ ID NO: 424 - protein, 10 aa, Homo sapiens
KTGAFSMPEV

SEQ ID NO: 425 - protein, 10 aa, Homo sapiens
WALLPLSPYL

SEQ ID NO: 426 - protein, 10 aa, Homo sapiens
IILAGAIALI

SEQ ID NO: 427 - protein, 10 aa, Homo sapiens
QLTDAGTYKC

SEQ ID NO: 428 - protein, 10 aa, Homo sapiens
ALLPLSPYLM

SEQ ID NO: 429 - protein, 10 aa, Homo sapiens
QLLNSKASLC

SEQ ID NO: 430 - protein, 10 aa, Homo sapiens
ILSCTFEPDI

SEQ ID NO: 431 - protein, 10 aa, Homo sapiens
WLKEGVLGLV

SEQ ID NO: 432 - protein, 10 aa, Homo sapiens
LQLLNSKASL

SEQ ID NO: 433 - protein, 10 aa, Homo sapiens
QILFWSIISI

SEQ ID NO: 434 - protein, 10 aa, Homo sapiens
GISGRHSITV

SEQ ID NO: 435 - protein, 10 aa, Homo sapiens
FEPDIKLSDI

SEQ ID NO: 436 - protein, 9 aa, Homo sapiens
ALLPLSPYL

SEQ ID NO: 437 - protein, 9 aa, Homo sapiens
SLCVSSFFA

SEQ ID NO: 438 - protein, 9 aa, Homo sapiens
ILFWSIISI

SEQ ID NO: 439 - protein, 9 aa, Homo sapiens
QLLNSKASL

SEQ ID NO: 440 - protein, 9 aa, Homo sapiens
KVVSVLYNV

SEQ ID NO: 441 - protein, 9 aa, Homo sapiens
ILAGAIALI

SEQ ID NO: 442 - protein, 9 aa, Homo sapiens
WLKEGVLGL

SEQ ID NO: 443 - protein, 9 aa, Homo sapiens
IILAGAIAL

SEQ ID NO: 444 - protein, 9 aa, Homo sapiens
NVTMKVVSV

SEQ ID NO: 445 - protein, 9 aa, Homo sapiens
EMFRGRTAV

SEQ ID NO: 446 - protein, 9 aa, Homo sapiens
AVFADQVIV

SEQ ID NO: 447 - protein, 9 aa, Homo sapiens
LLPLSPYLM

SEQ ID NO: 448 - protein, 9 aa, Homo sapiens
LLNSKASLC

SEQ ID NO: 449 - protein, 9 aa, Homo sapiens
VIQWLKEGV

SEQ ID NO: 450 - protein, 9 aa, Homo sapiens
AISWALLPL

SEQ ID NO: 451 - protein, 9 aa, Homo sapiens
SLGQILFWS

SEQ ID NO: 452 - protein, 9 aa, Homo sapiens
IALIIGFGI

SEQ ID NO: 453 - protein, 9 aa, Homo sapiens
CTFEPDIKL

SEQ ID NO: 454 - protein, 9 aa, Homo sapiens
IVGNASLRL

SEQ ID NO: 455 - protein, 9 aa, Homo sapiens
GQILFWSII

SEQ ID NO: 456 - DNA, 3447 bp, Homo sapiens
atgcccttgttcaagaacaccagtgtcagctctctgtactctggttgcagactgaccttgctcaggcctgagaaggatggggcagccaccagagtggatgctgtctgcacccatcgtcctgaccccaaaagccctggactggacagagagcggctgtactggaagctgagccagctgacccacggcatcactgagctgggcccctacaccctggacaggcacagtctctatgtcaatggtttcacccatcagagctctatgacgaccaccagaactcctgatacctccacaatgcacctggcaacctcgagaactccagcctccctgtctggacctacgaccgccagccctctcctggtgctattcacaattaacttcaccatcactaacctgcggtatgaggagaacatgcatcaccctggctctagaaagtttaacaccacggagagagtccttcagggtctgctcaggcctgtgttcaagaacaccagtgttggccctctgtactctggctgcagactgaccttgctcaggcccaagaaggatggggcagccaccaaagtggatgccatctgcacctaccgccctgatcccaaaagccctggactggacagagagcagctatactgggagctgagccagctaacccacagcatcactgagctgggcccctacaccctggacagggacagtctctatgtcaatggtttcacacagcggagctctgtgcccaccactagcattcctgggacccccacagtggacctgggaacatctgggactccagtttctaaacctggtccctcggctgccagccctctcctggtgctattcactctcaacttcaccatcaccaacctgcggtatgaggagaacatgcagcaccctggctccaggaagttcaacaccacggagagggtccttcagggcctgctcaggtccctgttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggccactatgccctggacaacgacagcctctttgtcaatggtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctccaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatggtttcacccatcggagctctgtacccaccaccagcaccggggtggtcagcgaggagccattcacactgaacttcaccatcaacaacctgcgctacatggcggacatgggccaacccggctccctcaagttcaacatcacagacaacgtcatgaagcacctgctcagtcctttgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacggttacaatgaacctggtctagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatggctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctacccgaatttcaccatcaccaacctaccatattcccaggacaaagcccagccaggcaccaccaattaccagaggaacaaaaggaatattgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattctcccaacagaaatgagcccttaactgggaattctgaccttcccttctgggctgtcatcttcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatga

SEQ ID NO: 457 - DNA, 3557 bp, Homo sapiens
gagagggtccttcagggtctgcttatgcccttgttcaagaacaccagtgtcagctctctgtactctggttgcagactgaccttgctcaggcctgagaaggatggggcagccaccagagtggatgctgtctgcacccatcgtcctgaccccaaaagccctggactggacagagagcggctgtactggaagctgagccagctgacccacggcatcactgagctgggcccctacaccctggacaggcacagtctctatgtcaatggtttcacccatcagagctctatgacgaccaccagaactcctgatacctccacaatgcacctggcaacctcgagaactccagcctccctgtctggacctacgaccgccagccctctcctggtgctattcacaattaacttcaccatcactaacctgcggtatgaggagaacatgcatcaccctggctctagaaagtttaacaccacggagagagtccttcagggtctgctcaggcctgtgttcaagaacaccagtgttggccctctgtactctggctgcagactgaccttgctcaggcccaagaaggatggggcagccaccaaagtggatgccatctgcacctaccgccctgatcccaaaagccctggactggacagagagcagctatactgggagctgagccagctaacccacagcatcactgagctgggcccctacaccctggacagggacagtctctatgtcaatggtttcacacagcggagctctgtgcccaccactagcattcctgggacccccacagtggacctgggaacatctgggactccagtttctaaacctggtccctcggctgccagccctctcctggtgctattcactctcaacttcaccatcaccaacctgcggtatgaggagaacatgcagcaccctggctccaggaagttcaacaccacggagagggtccttcagggcctgctcaggtccctgttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggccactatgccctggacaacgacagcctctttgtcaatggtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctccaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatggtttcacccatcggagctctgtacccaccaccagcaccggggtggtcagcgaggagccattcacactgaacttcaccatcaacaacctgcgctacatggcggacatgggccaacccggctccctcaagttcaacatcacagacaacgtcatgaagcacctgctcagtcctttgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacggttacaatgaacctggtctagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatggctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctacccgaatttcaccatcaccaacctaccatattcccaggacaaagcccagccaggcaccaccaattaccagaggaacaaaaggaatattgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattctcccaacagaaatgagcccttaactgggaattctgaccttcccttctgggctgtcatcttcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcctggggtgcctttcccccagccagggtccaaagaagcttggctggggcagaaataaaccatattggtcg

SEQ ID NO: 458 - protein, 1148 aa, Homo sapiens
MPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGHYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGSRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGLDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYPNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVIFIGLAGLLGLITCLICGVLVTTRRRKKEGEYNVQQQCPGYYQSHLDLEDLQ

SEQ ID NO: 459 - protein, 1156 aa, Homo sapiens
ERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGHYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGSRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGLDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYPNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVIFIGLAGLLGLITCLICGVLVTTRRRKKEGEYNVQQQCPGYYQSHLDLEDLQ

SEQ ID NO: 460 - protein, 79 aa, Homo sapiens
MSMVSHSGALCPPLAFLGPPQWTWEHLGLQFLNLVPRLPALSWCYSLSTSPSPTCGMRRTCSTLAPGSSTPRRGSFRAW

SEQ ID NO: 461 - protein, 313 aa, Homo sapiens
MPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLR

SEQ ID NO: 462 - DNA, 2996 bp, Homo sapiens; identical to SEQ ID NO: 311

SEQ ID NO: 463 - DNA, 3557 bp, Homo sapiens; identical to SEQ ID NO: 457

SEQ ID NO: 464 - DNA, 2712 bp, Homo sapiens
aggacatgcgtcaccctggctccaggaagttcaacaccacagagagggtcctgcagggtctgcttggtcccttgttcaagaactccagtgtcggccctctgtactctggctgcagactgatctctctcaggtctgagaaggatggggcagccactggagtggatgccatctgcacccaccaccttaaccctcaaagcctggactggacagggagcagctgtactggcagctgagccagatgaccaatggcatcaaagagctgggcccctacaccctggaccggaacagtctctacgtcaatggtttcacccatcggagctctgggctcaccaccagcactccttggacttccacagttgaccttggaacctcagggactccatcccccgtccccagccccacaactgctggccctctcctggtgccattcaccctaaacttcaccatcaccaacctgcagtatgaggaggacatgcatcgccctggatctaggaagttcaacgccacagagagggtcctgcagggtctgcttagtcccatattcaagaactccagtgttggccctctgtactctggctgcagactgacctctctcaggcccgagaaggatggggcagcaactggaatggatgctgtctgcctctaccaccctaatcccaaaagacctgggctggacagagagcagctgtactgggagctaagccagctgacccacaacatcactgagctgggcccctacagcctggacagggacagtctctatgtcaatggtttcacccatcagaactctgtgcccaccaccagtactcctgggacctccacagtgtactgggcaaccactgggactccatcctccttccccggccacacagagcctggccctctcctgataccattcacattcaactttaccatcaccaacctgcattatgaggaaaacatgcaacaccctggttccaggaagttcaacgccacagagagggtcctgcagggtctgcttagtcccatattcaagaactccagtgttggccctctgtactctggctgcagactgacctctctcaggcccgagaaggatggggcagcaactggaatggatgctgtctgtctctaccgaccctaatcccatcggacctgggctggacagagagcagctgtactgggagctgagccagctgacccacgacatcactgagctgggcccctacagccctggacagggacagtctctatgtcaatggtttcacccatcagaactctgtgcccaccaccagtactcctgggacctccacagtgtactgggcaaccactgggactccatcctccttccccggccacacagagcctggccctctcctgataccattcactttcaactttaccatcaccaacctgcattatgaggaaaacatgcaacacctggttccaggaagttcaacaccacggagagggttctgcagggtctgctcacgcccttgttcaagaacaccagtgttggccctctgtactctggctgcagactgaccttgctcagacctgagaagcaggaggcagccactggagtggacaccatctgcactcaccgccttgaccctctaaaccctggactggacagagagcagctatactgggagctgagcaaactgacccgtggcatcatcgagctgggcccctacctcctggacagaggcagtctctatgtcaatggtttcacccatcggaactttgtgcccatcaccagcactcctgggacctccacagtacacctaggaacctctgaaactccatcctccctacctagacccatagtgcctggccctctcctggtgccattcaccctcaacttcaccatcaccaacttgcagtatgaggaggccatgcgacaccctggctccaggaagttcaataccacggagagggtcctacagggtctgctcaggcccttgttcaagaataccagtatcggccctctgtactccagctgcagactgaccttgctcaggccagagaaggacaaggcagccaccagagtggatgccatctgtacccaccaccctgaccctcaaagccctggactgaacagagagcagctgtactgggagctgagccagctgacccacggcatcactgagctgggcccctacaccctggacaggcacagtctctatgtcaatggtttcacccatcagagccccataccaaccaccagcactcctgatacctccacaatgcacctgggaacctcgagaactccagcctccctgtctggacctacgaccgccagccctctcctggtgctattcacaattaacttcaccatcactaacctgcggtatgaggagaacatgcatcaccgctggctctagaaagtttaacaccacggagagagtccttcagggtctgctcaggcctgtgttcaaagaacaccagtgttggccctctgtactctggctgcagactgaccttgctcaggcccgagaaggatggggcagccacgcaaagtggatgccatctgcacctaccgccctgatcccaaaagccctggactggacagagagcagctatactgggagctgagccagggtgatgcatgttctcctcatatcgcaggttagtgatggtgaagttaattgtgaatagcaccaggagagggctggcggtcatgggtccagacagggagcctggagttctcgaggttgccaggtgcatgtc

SEQ ID NO: 465 - DNA, 1175 bp, Homo sapiens
gaggtatgctaactactactattatttagtaggctttgttagaaacttctgttgttatagtcaagggacgcatggaaactttttatattattctctctttaaatcctgttgcatatgtttagaagtaggccttttggaaatatataaagttctccacttttgaacatgttgtttctttcccacctccacgacagctgccagccctctcctggtgctattcactctcaacttcaccatcaccaacctgcggtatgaggagaacatgcagcaccctggctccaggaagttcaacactacagagagggtccttcagggcctgctaaggcccttgttcaagaacaccagtgttggccctctgtactctggctgcaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatggtttcacccatcggagctctgtacccaccaccagcaccggggtggtcagcgaggagccattcacactgaacttcaccatcaacaacctgcgctacatggcggacatgggccaacccggctccctcaagttcaacatcacagacaacgtcatgaagcacctgctcagtcctttgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacggttacaatgaacctggtccagatgagcctcctacaactcccaagccagccaccacattcctgcctcctctgtcagaagccacaacagccatggggtaccacctgaagaccctcacactcaattcacatctccaatctccagtattcaccagatatgggcaagggctcaaggtacattcaatccaccgaggggggtccttcagcaactggtcag

SEQ ID NO: 466 - DNA, 1959 bp, Homo sapiens
catccccagctcgaacagcagccacagtcccattcatggtgccattcaccctcaacttcaactcatcaccaacctgcagtacgaggaggacatgcggcacctggttccaggaagttcaacgcgcacagagagagaactgcagggtcgtgctcaaaccctagatcaggaatagcagtctggaatacctctattcaggctgcagactagcctcactcaggccagagaaggatagctcagccacggcagtggatgccatctgcacacatcgccctgaccctgaagacctcggactggacagagagcgactgtactgggagctgagcaatctgacaaatggcatccaggagctgggcccctacaccctggaccggaacagtctctatgtcaatggtttcacccatcgaagctctatgcccaccaccagcactcctgggacctccacagtggatgtgggaacctcagggactccatcctccagccccagccccacgactgctggccctctcctgatgccgttcaccctcaacttcaccatcaccaacctgcagtacgaggaggacatgcgtcgcactggctccaggaagttcaacaccatggagagtgtcctgcagggtctgctcaagcccttgttcaagaacaccagtgttggccctctgtactctggctgcagattgaccttgctcaggcccaagaaagatggggcagccactggagtggatgccatctgcacccaccgccttgaccccaaaagccctggactcaacagggagcagctgtactgggagctaagcaaactgaccaatgacattgaagagctgggcccctacaccctggacaggaacagtctctatgtcaatggtttcacccatcagagctctgtgtccaccaccagcactcctgggacctccacagtggatctcagaacctcagtggactccatcctccctctccagccccacaattatggctgctggccctctcctggtaccattcaccctcaacttcaccatcaccaacctgcagtatggggaggacatgggtcaccctggctccaggaagttcaacaccacagagagggtcctgcagggtctgcttggtcccatattcaagaacaccagtgttggccctctgtactctggctgcagactgacctctctcaggtccaagaaggatggagcagccactggagtggatgccatctgcatccatcatcttgaccccaaaagccctggactcaacagagagcggctgtactgggagctgagccaactgaccaatggcatcaaagagctgggcccctacaccctggacaggaacagtctctatgtcaatggtttcacccatcggacctctgtgcccaccaccagtactcctgggacctccacagtgtactgggcaaccactgggactccatcctccctccccgccacacagagcctggccctctcctgataccattcacattcaactttaccatcacctacctgcattatagaggaaaacatgcaacacccgtggttccaggaacgatgtcaacaccacaggagagggttctgcagggtcttcgctcacgcccattgttacaagaacaccagtagttggccctctgtactctggctgcagaatgaccttgctcagacctgagaagcaggaggcaacacactggaatggacaccatctgtatccaccagcgttagatcccatcaggacctggactggacagagagcaggctatactgggagctagagccagctgacccacagcatcacagagctgggaccctacagccctggatagggacagtctctatgtcaatggcttcaacccttggagctctgtgccaaccaccagcactcctgggacctccacagtgcacctggcaacctctgggactccatcctccctgcctggccacacagcccctgtccctctcttgataccattcaccctcaacttac

SEQ ID NO: 467 - DNA, 1636 bp, Homo sapiens
gacctcctctgcacctacctgcagcccctcagcggcccaggtctgcctatcaagcaggtgttccatgagctgagccagcagacccatggcatcacccggctgggcccctactctctggacaaagacagcctctaccttaacggttacaatgaacctggtccagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatggctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctacctgaatttcaccatcaccaacctaccatattcccaggacaaagcccagccaggcaccaccaattaccagaggaacaaaaggaatattgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattctcccaacagaaatgagcccttaactgggaattctgaccttcccttctgggctgtcatcctcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcctggggtgcctttcccccagccagggtccaaagaagcttggctggggcagaaataaaccatattggtcgga

SEQ ID NO: 468 - DNA, 231 bp, Homo sapiens
actacatgacacattccgcttctgcctggtcaccaacttgacaaatggagtcatcagtttatcaaccaacaagcagctccagcacccagcacttctacctgaatttcaccatcaccaacctaccatattcccaggacaaagcccagccaggcaccaccaattaccagaggaacaaaaggaatattgaggatgcgctcaaccaactcttccgaaacagcagcatcgagagtt

SEQ ID NO: 469 - DNA, 607 bp, Homo sapiens
atgaagagctatcgctgtccaggacatagaagcccagttgggtgacaccatgggtcagctgactcagctcccagtaaagctgctgtatgtccagcccggggcccacagggtcagggtggtaggtgcaggtggtgtccacaccagtggctgccccatccttctcaggccaggtgctgaaggaccccctcggtggagttgaatgtagctgagcccttgcccatatctggtgaatactggagattggagatggtgaagttgagtgtgagggtcttcaggtggtaccccatggctgttgtggcttctgacagaggaggcaggaatgtggtggctggcttgggagttgtaggaggctcatctggaccaggttcattgtaaccgttaaggtagaggctgtctttgtccagagagtaggggcccagccgggtgatgccatgggtctgctggctcagctcatggaacacctgcttgataggcagacctgggccgctgaggggctgcaggtaggtgcagaggaggtccacccgtgtctcagcaccgttcttcacagaccttagtgcgatgaccctgcagcctgtgtaccgtgcacccaggctgctcctctggaaca

SEQ ID NO: 470 - DNA, 981 bp, Homo sapiens
ggtaaccacagctgacccatggcatcaaagagctgggcccctacaccctggacaggaacagtctctatgtcaatggtttcacccatcggagctctgtggcccccaccagcactcctgggacctccacagtggaccttgggacctcagggactccatcctccctccccagccccacaacagctgttcctctcctggtgccgttcaccctcaactttaccatcaccaatctgcagtatggggaggacatgcgtcaccctggctccaggaagttcaacaccacagagagggtcctgcagggtctgcttggtcccttgttcaagaactccagtgtcggccctctgtactctggctgcagactgatctctctcaggtctgagaaggatggggcagccactggagtggatgccatctgcacccaccaccttaaccctcaaagccctggactggacagggagcagctgtactggcagctgagccagagaccacaacctcatttatcacctattctgagacacacacaagttcagccattccaactctccctgtctccccctggtgcatcaaagatgctgacctcactggtcatcagttctgggacagacagcactacaactttcccaacactgacggagaccccatatgaaccagagacaacagccatacagctcattcatcctgcagagaccaacacaatggttcccaggacaactcccaagttttcccatagtaagtcagacaccacactcccagtagccatcaccagtcctgggccagaagccagttcagctgtttcaacgacaactatctcacctgatatgtcagatctggtgacctcactggtccctagttctgggacagacaccagtacaaccttcccaacattgagtgagaccccatatgaaccagagactacagccacgtggctcactcatcctgcagaaaccagaacaacggtttctgggacaattcccaacttttccc

SEQ ID NO: 471 - DNA, 959 bp, Homo sapiens
cagatggcatccactccggtggcttccccatctttctctggcctgagcaaggtcagcctgcagccagagtacagagggccaacactggtgttcttgaacaagggccttagcaggccctgaaggaccctctctgtagtgttgaacttcctggagccaggccacatgttctcctcataccgcaggttagtgatggtgaagttgagggtgaatagtatcaggagatggctggcagctgaagggccaaatatcgaggctggagtcttagatgctcccagatacactgtgggggtcccaggagtgctggtggtggacacagagctccgatgagtgaaaccattgacaaagaggctgtcgttgtccagggcataggggcccagctcagtgatattgtgggtcagctggctcagctcccaatacagctgctctctgtccagcctagggcttttggggtcagggtggtgggtgcagatggcatccactccagtggctgtcccatccttttcaggcctgagcaaagtcagtctgcagccagagtacagagggccaacactggtgctcttgaacagggacctgagcaggccctgaaggaccctctccgtggtgttgaacttcctggagccagggtgctgcatgttctcctcataccgcaggttggtgatggtgaagttgagagtgaatagcaccaggagagggctggcagccgagggaccaggtttagaaactggagtcccagatgttcccaggtccactgtgggggtcccaggaatgctagtggtgggcacagagctccgctgtgtgaaaccattgacatagagactgtccctgtccagggtgtaggggcccagctcagtgatgctgtgggttagctggctcagctcccagtatagctgctctctgtccagtccagggcttttgggatcagggcggtaggtgcagatggcatccactttggtggctgc

SEQ ID NO: 472 - DNA, 1315 bp, Homo sapiens
ccaccgtcttgaccccaaaagccctggagtggacagggagcagctatactgggagctgagccagctgaccaatggcatcaaagagctgggcccctacacctggacaggaacagtctctatgtcaatggtttcacccatcggacctctgtgcccaccaccagcactcctgggacctccacagtggaccttggaacctcagggactccattctccctcccaagccccgcaactgctggccctctcctggtgctgttcaccctcaacttcaccatcaccaacctgaagtatgaggaggacatgcatcgccctggctccaggaagttcaacaccactgagagggtcctgcagactctgcttggtcctatgttcaagaacaccagtgttggccttctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatggcatcaaagagctgggcccctacaccctggacaggaacagtctctatgtcaatggtttcacccattggatccctgtgcccaccagcagcactcctgggacctccacagtggaccttgggtcagggactccatcctccctccccagccccacaactgctggccctctcctggtgccgttcaccctcaacttcaccatcaccaacctgaagtacgaggaggacatgcattgccctggctccaggaagttcaacaccacagagagagtcctgcagagtctgcttggtcccatgttcaagaacaccagtgttggccctctgtactctggctgcagactgaccttgctcaggtccgagaaggatggagcagccactggagtggatgccatctgcacccaccgtcttgaccccaaaagcctggagtggacagggagcagctatactgggagctgagccagctgaccaatgccatcaaagagctgggtccctacaccctggacagcaacagtcttctatgtcaatggtttcacccatcagacctctgcgcccaacaccagcactcctgggacctccacagtggaccttgggacctcagggactccatcctccctccccagccctacatctgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacactacga

SEQ ID NO: 473 - DNA, 689 bp, Homo sapiens
acggcatcaggagagggccagcagtcgtggggctggggctggaggatggagtccctgaggttcccacatccactgtggaggtcccaggagtgctggtggtgggcatagagcttcgatgggtgaaaccattgacatagagactgttccggtccagggtgtaggggcccagctcagtgatgccgtgggtcagctggctcagctcccagtacagctgctctctgttcagtccagggctttgagggtcagggtggtgggtacagatggcatccactctggtggctgccttgtccttctctggccttgagcaaggtcagtctgcagcctgagagctaacagaggtccgataactggtattcttgaacaagggcctagagcagaaccctgtaggaccatcgccgtggtatatgaacttcctagagccaggatttcgcacggccatcactcatactgcaacttgctgatggcaaagttgaggataaacggcaccaggagagggccagccacttatgggtctaggtagggaggatggagtttcagaggttctcgagatccactgtggaggtcccaggagtgctggtggtggacacagagctctgatgggtgaaaccattgacatagagactgttcctgtccagggtgtaggggcccagctcttcaatgtcattggtcagtttgcttagctcccagta

SEQ ID NO: 474 - DNA, 495 bp, Homo sapiens
gtggatatgagttgaatactcactgctggtggtggacacagagctctgatgggtgaaacctgcatagagaaggagggaggagagtgggtaagagacaaggagaggtgggggaccaaatggaggtcaatgctaccctggtgcaatgaaccgagtttcatggtacagggacaattgaagattttctatcagcatcctcacatcaggaaagaatgccctgagggaacacagtccatgatggtaaggaaaccatgaagtccagaccttagtcatcccatgtagagcacatgacagaattttcaaaggccaggcagggagtgtgacctctagttagagattagaggctgcccagcaagggggaagagatttcaaccacatcacagccactcaccattgacatagagactgttcctgtccagggtgtaggggcccagctcttcaatgtcattggtcagtttgcttagctcccagtacagctgctccctgttgagtccaggg

SEQ ID NO: 475 - DNA, 192 bp, Homo sapiens
agtgcccaggctactaccagtcacacctagacctggaggatctgcaatgactggaacttgccggtgcctggggatagcctcttcatcaatggctatgcaccccagaatttatcaatccggggcgagtaccagataaatttccacattgtcaactggaacctcagtaatccagaccccacatcctcagagtac

SEQ ID NO: 476 - DNA, 500 bp, Homo sapiens
ccggtggctgccccacgtttttcaggcctgagcaaggtcagtctgcagccagagtacagagggccaacactggtgctcttgaacaagggcttgagcagaccctgcaggactctctccgtggtgttgaacttcctggaaccagggtgacgcatgtcctcctcatactgcaggttggtgatagtgaagttgagggtgaatggcaccaggagagggccagggctgtgtggccagggagggaggctggagtcccagaggtttccaggtgcactgcagaggtcccaggaatactggtggttggcacagagctccgatgggtgaagccattgacatagagactgtccctgtccaggtgtaggggcccagctctgtaacgctgttggtcagctggctcagctcccagtatagccgctctctgtccagtccaggaccagtgggatcaaggcggagggtgcagatggcgtccactccagtggctgccccatgtttctcaggtctgagca

SEQ ID NO: 477 - DNA, 191 bp, Homo sapiens
gaggtatgctaactactactattatttagtaggctttgttagaaacttctgttgttatagtcaagggacgcatggaaactttttatattattctctctttaaatcctgttgcatatgtttagaagtaggccttttggaaatatataaagttctccacttttgaacatgttgtttctttcccacctccacga

SEQ ID NO: 478 - protein, 914 aa, Homo sapiens; identical to SEQ ID NO: 312

SEQ ID NO: 479 - protein, 1148 aa, Homo sapiens; identical to SEQ ID NO: 458

SEQ ID NO: 480 - protein, 230 aa, Homo sapiens
MHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSLEWTGSSYTGS

SEQ ID NO: 481 - protein, 210 aa, Homo sapiens
MQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAI

SEQ ID NO: 482 - protein, 97 aa, Homo sapiens
MSMVSHSGALCPPLAFLGPPQWTWEHLGLQFLNLVPRLPALSWCYSLSTSPSPTCGMRRTCSTLAPGSSTPRRGSFRACSGPCSRAPVLALCTLAAD

SEQ ID NO: 483 - protein, 438 aa, Homo sapiens [sequence omitted]

SEQ ID NO: 484 - protein, 216 aa, Homo sapiens
MTLKSWAPTPWTGTVSMSMVSPIRALCPPPALLGPPQWISEPQWTPSSLSSPTIMAAGPLLVPFTLNFTITNLQYGEDMGHPGSRKFNTTERVLQGLLGPIFKNTSVGPLYSGCRLTSLRSKKDGAATGVDAICIHHLDPKSPGLNRERLYWELSQLTNGIKELGPYTLDRNSLYVNGFTHRTSVPTTSTPGTSTVYWATTGTPSSLPATQSLALS

SEQ ID NO: 485 - protein, 268 aa, Homo sapiens
MPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPKKDGAATGVDAICTHRLDPKSPGLNREQLYWELSKLTNDIEELGPYTLDRNSLYVNGFTHQSSVSTTSTPGTSTVDLRTSVDSILPLQPHNYGCWPSPGTIHPQLHHHQPAVWGGHGSPWLQEVQHHREGPAGSAWSHIQEHQCWPSVLWLQTDLSQVQEGWSSHWSGCHLHPSS

SEQ ID NO: 486 - protein, 304 aa, Homo sapiens
MQHPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNSHLQSPVFTRYGQGLKVHSIHRGGSFSNWS

SEQ ID NO: 487 - protein, 294 aa, Homo sapiens
MTNGIKELGPYTLDRNSLYVNGFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNATERVLQGLLSPIFKNSSVGPLYSGCRLTSLRPEKDGAATGMDAVCLYRP

SEQ ID NO: 488 - protein, 233 aa, Homo sapiens
SLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYFPNRNEPLTGNSDLPFWAVILIGLAGLLGLITCLICGVLVTTRRRKKEGEYNVQQQCPGYYQSHLDLEDLQ

SEQ ID NO: 489 - protein, 178 aa, Homo sapiens
SLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYFPNRNEPLTGNSDLPF

SEQ ID NO: 490 - protein, 15 aa, Homo sapiens
TCGMRRTCSTLAPGS

SEQ ID NO: 491 - protein, 15 aa, Homo sapiens
CRLTLLRPEKDGTAT

SEQ ID NO: 492 - protein, 15 aa, Homo sapiens
DGTATGVDAICTHHP

SEQ ID NO: 493 - protein, 15 aa, Homo sapiens
CTHHPDPKSPRLDRE

SEQ ID NO: 494 - protein, 15 aa, Homo sapiens
RLDREQLYWELSQLT

SEQ ID NO: 495 - protein, 15 aa, Homo sapiens
LGPYALDNDSLFVNG

SEQ ID NO: 496 - protein, 15 aa, Homo sapiens
SVSTTSTPGTPTYVL

SEQ ID NO: 497 - protein, 15 aa, Homo sapiens
LRPEKDGEATGVDAI

SEQ ID NO: 498 - protein, 15 aa, Homo sapiens
DPTGPGLDREQLYLE

SEQ ID NO: 499 - protein, 15 aa, Homo sapiens
LDRDSLYVNGFTHRS

SEQ ID NO: 500 - protein, 15 aa, Homo sapiens
GPYSLDKDSLYLNGY

SEQ ID NO: 501 - protein, 15 aa, Homo sapiens
YLNGYNEPGPDEPPT

SEQ ID NO: 502 - protein, 15 aa, Homo sapiens
ATFNSTEGVLQHLLR

SEQ ID NO: 503 - protein, 15 aa, Homo sapiens
QLISLRPEKDGAATG

SEQ ID NO: 504 - protein, 15 aa, Homo sapiens
GAATGVDTTCTYHPD

SEQ ID NO: 505 - protein, 15 aa, Homo sapiens
TYHPDPVGPGLDIQQ

SEQ ID NO: 506 - protein, 15 aa, Homo sapiens
LDIQQLYWELSQLTH

SEQ ID NO: 507 - protein, 15 aa, Homo sapiens
HIVNWNLSNPDPTSS

SEQ ID NO: 508 - protein, 15 aa, Homo sapiens
DPTSSEYITLLRDIQ

SEQ ID NO: 509 - protein, 15 aa, Homo sapiens
LRDIQDKVTTLYKGS

SEQ ID NO: 510 - protein, 15 aa, Homo sapiens
LYKGSQLHDTFRFCL

SEQ ID NO: 511 - protein, 15 aa, Homo sapiens
DKAQPGTTNYQRNKR

SEQ ID NO: 512 - DNA, 450 bp, Homo sapiens
gttacaatgaacctggtccagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatg

SEQ ID NO: 513 - DNA, 402 bp, Homo sapiens
gtttcacccatcggagctctgtacccaccaccagcaccggggtggtcagcgaggagccattcacactgaacttcaccatcaacaacctgcgctacatggcggacatgggccaacccggctccctcaagttcaacatcacagacaacgtcatgaagcacctgctcagtcctttgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacg

SEQ ID NO: 514 - DNA, 465 bp, Homo sapiens
gtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctgcaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatg

SEQ ID NO: 515 - DNA, 463 bp, Homo sapiens
gtttcacacagcggagctctgtgcccaccactagcattcctgggacccccacagtggacctgggaacatctgggactccagtttctaaacctggtccctcggctgccagccctctcctggtgctattcactctcaacttcaccatcaccaacctgcggtatgaggagaacatgcagcaccctggctccaggaagttcaacaccacggagagggtccttcagggcctggtccctgttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggcccctatgccctggacaacgacagcctctttgtcaatg

SEQ ID NO: 516 - DNA, 156 bp, Homo sapiens
cagccaccggagtggatgccatctgcacccaccgccctgaccccacaggccctgggctggacagagagcagctgtatttggagctgagccagctgacccacagcatcactgagctgggcccctacaccctggacagggacagtctctatgtcaatg

SEQ ID NO: 517 - DNA, 450 bp, Homo sapiens
gttacaatgaacctggtctagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatg

SEQ ID NO: 518 - DNA, 402 bp, Homo sapiens; identical to SEQ ID NO: 513

SEQ ID NO: 519 - DNA, 465 bp, Homo sapiens
gtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctccaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatg

SEQ ID NO: 520 - DNA, 468 bp, Homo sapiens
gtttcacacagcggagctctgtgcccaccactagcattcctgggacccccacagtggacctgggaacatctgggactccagtttctaaacctggtccctcggctgccagccctctcctggtgctattcactctcaacttcaccatcaccaacctgcggtatgaggagaacatgcagcaccctggctccaggaagttcaacaccacggagagggtccttcagggcctgctcaggtccctgttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggccactatgccctggacaacgacagcctctttgtcaatg

SEQ ID NO: 521 - DNA, 468 bp, Homo sapiens
gtttcacccatcagagctctatgacgaccaccagaactcctgatacctccacaatgcacctggcaacctcgagaactccagcctccctgtctggacctacgaccgccagccctctcctggtgctattcacaattaacttcaccatcactaacctgcggtatgaggagaacatgcatcaccctggctctagaaagtttaacaccacggagagagtccttcagggtctgctcaggcctgtgttcaagaacaccagtgttggccctctgtactctggctgcagactgaccttgctcaggcccaagaaggatggggcagccaccaaagtggatgccatctgcacctaccgccctgatcccaaaagccctggactggacagagagcagctatactgggagctgagccagctaacccacagcatcactgagctgggcccctacaccctggacagggacagtctctatgtcaatg

SEQ ID NO: 522 - DNA, 262 bp, Homo sapiens
gagagggtccttcagggtctgcttatgcccttgttcaagaacaccagtgtcagctctctgtactctggttgcagactgaccttgctcaggcctgagaaggatggggcagccaccagagtggatgctgtctgcacccatcgtcctgaccccaaaagccctggactggacagagagcggctgtactggaagctgagccagctgacccacggcatcactgagctgggcccctacaccctggacaggcacagtctctatgtcaatg

SEQ ID NO: 523 - DNA, 302 bp, Homo sapiens
aggacatgcgtcaccctggctccaggaagttcaacaccacagagagggtcctgcagggtctgcttggtcccttgttcaagaactccagtgtcggccctctgtactctggctgcagactgatctctctcaggtctgagaaggatggggcagccactggagtggatgccatctgcacccaccaccttaaccctcaaagcctggactggacagggagcagctgtactggcagctgagccagatgaccaatggcatcaaagagctgggcccctacaccctggaccggaacagtctctacgtcaatg

SEQ ID NO: 524 - DNA, 468 bp, Homo sapiens
gtttcacccatcggagctctgggctcaccaccagcactccttggacttccacagttgaccttggaacctcagggactccatcccccgtccccagccccacaactgctggccctctcctggtgccattcaccctaaacttcaccatcaccaacctgcagtatgaggaggacatgcatcgccctggatctaggaagttcaacgccacagagagggtcctgcagggtctgcttagtcccatattcaagaactccagtgttggccctctgtactctggctgcagactgacctctctcaggcccgagaaggatggggcagcaactggaatggatgctgtctgcctctaccaccctaatcccaaaagacctgggctggacagagagcagctgtactgggagctaagccagctgacccacaacatcactgagctgggcccctacagcctggacagggacagtctctatgtcaatg

SEQ ID NO: 525 - DNA, 470 bp, Homo sapiens
gtttcacccatcagaactctgtgcccaccaccagtactcctgggacctccacagtgtactgggcaaccactgggactccatcctccttccccggccacacagagcctggccctctcctgataccattcacattcaactttaccatcaccaacctgcattatgaggaaaacatgcaacaccctggttccaggaagttcaacgccacagagagggtcctgcagggtctgcttagtcccatattcaagaactccagtgttggccctctgtactctggctgcagactgacctctctcaggcccgagaaggatggggcagcaactggaatggatgctgtctgtctctaccgaccctaatcccatcggacctgggctggacagagagcagctgtactgggagctgagccagctgacccacgacatcactgagctgggcccctacagccctggacagggacagtctctatgtcaatg

SEQ ID NO: 526 - DNA, 467 bp, Homo sapiens
gtttcacccatcagaactctgtgcccaccaccagtactcctgggacctccacagtgtactgggcaaccactgggactccatcctccttccccggccacacagagcctggccctctcctgataccattcactttcaactttaccatcaccaacctgcattatgaggaaaacatgcaacacctggttccaggaagttcaacaccacggagagggttctgcagggtctgctcacgcccttgttcaagaacaccagtgttggccctctgtactctggctgcagactgaccttgctcagacctgagaagcaggaggcagccactggagtggacaccatctgcactcaccgccttgaccctctaaaccctggactggacagagagcagctatactgggagctgagcaaactgacccgtggcatcatcgagctgggcccctacctcctggacagaggcagtctctatgtcaatg

SEQ ID NO: 527 - DNA, 468 bp, Homo sapiens
gtttcacccatcggaactttgtgcccatcaccagcactcctgggacctccacagtacacctaggaacctctgaaactccatcctccctacctagacccatagtgcctggccctctcctggtgccattcaccctcaacttcaccatcaccaacttgcagtatgaggaggccatgcgacaccctggctccaggaagttcaataccacggagagggtcctacagggtctgctcaggcccttgttcaagaataccagtatcggccctctgtactccagctgcagactgaccttgctcaggccagagaaggacaaggcagccaccagagtggatgccatctgtacccaccaccctgaccctcaaagccctggactgaacagagagcagctgtactgggagctgagccagctgacccacggcatcactgagctgggcccctacaccctggacaggcacagtctctatgtcaatg

SEQ ID NO: 528 - DNA, 537 bp, Homo sapiens
gtttcacccatcagagccccataccaaccaccagcactcctgatacctccacaatgcacctgggaacctcgagaactccagcctccctgtctggacctacgaccgccagccctctcctggtgctattcacaattaacttcaccatcactaacctgcggtatgaggagaacatgcatcaccgctggctctagaaagtttaacaccacggagagagtccttcagggtctgctcaggcctgtgttcaaagaacaccagtgttggccctctgtactctggctgcagactgaccttgctcaggcccgagaaggatggggcagccacgcaaagtggatgccatctgcacctaccgccctgatcccaaaagccctggactggacagagagcagctatactgggagctgagccagggtgatgcatgttctcctcatatcgcaggttagtgatggtgaagttaattgtgaatagcaccaggagagggctggcggtcatgggtccagacagggagcctggagttctcgaggttgccaggtgcatgtc

SEQ ID NO: 529 - DNA, 231 bp, Homo sapiens
tgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacg

SEQ ID NO: 530 - DNA, 376 bp, Homo sapiens
gttacaatgaacctggtccagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagcgatagctcttcat

SEQ ID NO: 531 - DNA, 75 bp, Homo sapiens
ggtaaccacagctgacccatggcatcaaagagctgggcccctacaccctggacaggaacagtctctatgtcaatg

SEQ ID NO: 532 - DNA, 906 bp, Homo sapiens
gtttcacccatcggagctctgtggcccccaccagcactcctgggacctccacagtggaccttgggacctcagggactccatcctccctccccagccccacaacagctgttcctctcctggtgccgttcaccctcaactttaccatcaccaatctgcagtatggggaggacatgcgtcaccctggctccaggaagttcaacaccacagagagggtcctgcagggtctgcttggtcccttgttcaagaactccagtgtcggccctctgtactctggctgcagactgatctctctcaggtctgagaaggatggggcagccactggagtggatgccatctgcacccaccaccttaaccctcaaagccctggactggacagggagcagctgtactggcagctgagccagagaccacaacctcatttatcacctattctgagacacacacaagttcagccattccaactctccctgtctccccctggtgcatcaaagatgctgacctcactggtcatcagttctgggacagacagcactacaactttcccaacactgacggagaccccatatgaaccagagacaacagccatacagctcattcatcctgcagagaccaacacaatggttcccaggacaactcccaagttttcccatagtaagtcagacaccacactcccagtagccatcaccagtcctgggccagaagccagttcagctgtttcaacgacaactatctcacctgatatgtcagatctggtgacctcactggtccctagttctgggacagacaccagtacaaccttcccaacattgagtgagaccccatatgaaccagagactacagccacgtggctcactcatcctgcagaaaccagaacaacggtttctgggacaattcccaacttttccc

SEQ ID NO: 533 - DNA, 404 bp, Homo sapiens
gtttcacccatcggagctctgtggcccccaccagcactcctgggacctccacagtggaccttgggacctcagggactccatcctccctccccagccccacaacagctgttcctctcctggtgccgttcaccctcaactttaccatcaccaatctgcagtatggggaggacatgcgtcaccctggctccaggaagttcaacaccacagagagggtcctgcagggtctgcttggtcccttgttcaagaactccagtgtcggccctctgtactctggctgcagactgatctctctcaggtctgagaaggatggggcagccactggagtggatgccatctgcacccaccaccttaaccctcaaagccctggactggacagggagcagctgtactggcagctgagccag

SEQ ID NO: 534 - DNA, 157 bp, Homo sapiens
gcagccaccaaagtggatgccatctgcacctaccgccctgatcccaaaagccctggactggacagagagcagctatactgggagctgagccagctaacccacagcatcactgagctgggcccctacaccctggacagggacagtctctatgtcaatg

SEQ ID NO: 535 - DNA, 468 bp, Homo sapiens
gtttcacacagcggagctctgtgcccaccactagcattcctgggacccccacagtggacctgggaacatctgggactccagtttctaaacctggtccctcggctgccagccctctcctggtgctattcactctcaacttcaccatcaccaacctgcggtatgaggagaacatgcagcaccctggctccaggaagttcaacaccacggagagggtccttcagggcctgctcaggtccctgttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggcccctatgccctggacaacgacagcctctttgtcaatg

SEQ ID NO: 536 - DNA, 334 bp, Homo sapiens
gtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctgcaggctgaccttgctcaggccagagaaagatggggaagccaccggagtggatgccatctg

SEQ ID NO: 537 - DNA, 127 bp, Homo sapiens
ccaccgtcttgaccccaaaagccctggagtggacagggagcagctatactgggagctgagccagctgaccaatggcatcaaagagctgggcccctacacctggacaggaacagtctctatgtcaatg

SEQ ID NO: 538 - DNA, 468 bp, Homo sapiens
gtttcacccatcggacctctgtgcccaccaccagcactcctgggacctccacagtggaccttggaacctcagggactccattctccctcccaagccccgcaactgctggccctctcctggtgctgttcaccctcaacttcaccatcaccaacctgaagtatgaggaggacatgcatcgccctggctccaggaagttcaacaccactgagagggtcctgcagactctgcttggtcctatgttcaagaacaccagtgttggccttctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatggcatcaaagagctgggcccctacaccctggacaggaacagtctctatgtcaatg

SEQ ID NO: 539 - DNA, 465 bp, Homo sapiens
gtttcacccattggatccctgtgcccaccagcagcactcctgggacctccacagtggaccttgggtcagggactccatcctccctccccagccccacaactgctggccctctcctggtgccgttcaccctcaacttcaccatcaccaacctgaagtacgaggaggacatgcattgccctggctccaggaagttcaacaccacagagagagtcctgcagagtctgcttggtcccatgttcaagaacaccagtgttggccctctgtactctggctgcagactgaccttgctcaggtccgagaaggatggagcagccactggagtggatgccatctgcacccaccgtcttgaccccaaaagcctggagtggacagggagcagctatactgggagctgagccagctgaccaatgccatcaaagagctgggtccctacaccctggacagcaacagtcttctatgtcaatg

SEQ ID NO: 540 - DNA, 255 bp, Homo sapiens
gtttcacccatcagacctctgcgcccaacaccagcactcctgggacctccacagtggaccttgggacctcagggactccatcctccctccccagccctacatctgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacactacga

SEQ ID NO: 541 - DNA, 390 bp, Homo sapiens
catccccagctcgaacagcagccacagtcccattcatggtgccattcaccctcaacttcaactcatcaccaacctgcagtacgaggaggacatgcggcacctggttccaggaagttcaacgcgcacagagagagaactgcagggtcgtgctcaaaccctagatcaggaatagcagtctggaatacctctattcaggctgcagactagcctcactcaggccagagaaggatagctcagccacggcagtggatgccatctgcacacatcgccctgaccctgaagacctcggactggacagagagcgactgtactgggagctgagcaatctgacaaatggcatccaggagctgggcccctacaccctggaccggaacagtctctatgtcaatg

SEQ ID NO: 542 - DNA, 468 bp, Homo sapiens
gtttcacccatcgaagctctatgcccaccaccagcactcctgggacctccacagtggatgtgggaacctcagggactccatcctccagccccagccccacgactgctggccctctcctgatgccgttcaccctcaacttcaccatcaccaacctgcagtacgaggaggacatgcgtcgcactggctccaggaagttcaacaccatggagagtgtcctgcagggtctgctcaagcccttgttcaagaacaccagtgttggccctctgtactctggctgcagattgaccttgctcaggcccaagaaagatggggcagccactggagtggatgccatctgcacccaccgccttgaccccaaaagccctggactcaacagggagcagctgtactgggagctaagcaaactgaccaatgacattgaagagctgggcccctacaccctggacaggaacagtctctatgtcaatg

SEQ ID NO: 543 - DNA, 475 bp, Homo sapiens
gtttcacccatcagagctctgtgtccaccaccagcactcctgggacctccacagtggatctcagaacctcagtggactccatcctccctctccagccccacaattatggctgctggccctctcctggtaccattcaccctcaacttcaccatcaccaacctgcagtatggggaggacatgggtcaccctggctccaggaagttcaacaccacagagagggtcctgcagggtctgcttggtcccatattcaagaacaccagtgttggccctctgtactctggctgcagactgacctctctcaggtccaagaaggatggagcagccactggagtggatgccatctgcatccatcatcttgaccccaaaagccctggactcaacagagagcggctgtactgggagctgagccaactgaccaatggcatcaaagagctgggcccctacaccctggacaggaacagtctctatgtcaatg

SEQ ID NO: 544 - DNA, 485 bp, Homo sapiens
gtttcacccatcggacctctgtgcccaccaccagtactcctgggacctccacagtgtactgggcaaccactgggactccatcctccctccccgccacacagagcctggccctctcctgataccattcacattcaactttaccatcacctacctgcattatagaggaaaacatgcaacacccgtggttccaggaacgatgtcaacaccacaggagagggttctgcagggtcttcgctcacgcccattgttacaagaacaccagtagttggccctctgtactctggctgcagaatgaccttgctcagacctgagaagcaggaggcaacacactggaatggacaccatctgtatccaccagcgttagatcccatcaggacctggactggacagagagcaggctatactgggagctagagccagctgacccacagcatcacagagctgggaccctacagccctggatagggacagtctctatgtcaatg

SEQ ID NO: 545 - DNA, 141 bp, Homo sapiens
gcttcaacccttggagctctgtgccaaccaccagcactcctgggacctccacagtgcacctggcaacctctgggactccatcctccctgcctggccacacagcccctgtccctctcttgataccattcaccctcaacttac

SEQ ID NO: 546 - DNA, 142 bp, Homo sapiens
gacctcctctgcacctacctgcagcccctcagcggcccaggtctgcctatcaagcaggtgttccatgagctgagccagcagacccatggcatcacccggctgggcccctactctctggacaaagacagcctctaccttaacg

SEQ ID NO: 547 - DNA, 185 bp, Homo sapiens
gttcttacccaggccagagaaggatagctcagccacggcagtggatgccatctgcacacatcgccctgaccctgaagacctcggactggacagagagcgactgtactgggagctgagcaatctgacaaatggcatccaggagctgggcccctacaccctggaccggaacagtctctatgtcaatg

SEQ ID NO: 548 - DNA, 462 bp, Homo sapiens
gtttcacccatcgaagctctatgcccaccaccagcactcctgggacctccacagtggatgtgggaacctcagggactccatcctccagccccagccccacgactgctggccctctcctgatgccgttcaccctcaacttcaccatcaccaacctgcagtacgaggaggacatgcgtcgcactggctccaggaagttcaacaccatggagagtgtcctgcagggtctgcccttgttcaagaacaccagtgttggccctctgtactctggctgcagattgaccttgctcaggcccgagaaagatggggcagccactggagtggatgccatctgcacccaccgccttgaccccaaaagccctggactcaacagggagcagctgtactgggagctaagcaaactgaccaatgacattgaagagctgggcccctacaccctggacaggaacagtctctatgtcaatg

SEQ ID NO: 549 - DNA, 400 bp, Homo sapiens
actccatcctccctctccagccccacaattatggctgctggccctctcctggtaccattcaccctcaacttcaccatcaccaacctgcagtatggggaggacatgggtcaccctggctccaggaagttcaacaccacagagagggtcctgcagggtctgcttggtcccatattcaagaacaccagtgttggccctctgtactctggctgcagactgacctctctcaggtctgagaaggatggagcagccactggagtggatgccatctgcatccatcatcttgaccccaaaagccctggactcaacagagagcggctgtactgggagctgagccaactgaccaatggcatcaaagagctgggcccctacaccctggacaggaacagtctctatgtcaatg

SEQ ID NO: 550 - DNA, 468 bp, Homo sapiens
gtttcacccatcggacctctgtgcccaccagcagcactcctgggacctccacagtggaccttggaacctcagggactccattctccctcccaagccccgcaactgctggccctctcctggtgctgttcaccctcaacttcaccatcaccaacctgaagtatgaggaggacatgcatcgccctggctccaggaagttcaacaccactgagagggtcctgcagactctgcttggtcctatgttcaagaacaccagtgttggccttctgtactctggctgcagactgaccttgctcaggtccgagaaggatggagcagtcactggagtggatgccatctgcacccaccgtcttgaccccaaaagccctggagtggacagggagcagctatactgggagctgagccagctgaccaatggcatcaaagagctgggcccctacaccctggacaggcacagtctctatgtcaatg

SEQ ID NO: 551 - DNA, 366 bp, Homo sapiens
ctgctggccctctcctggtgctgttcaccctcaacttcaccatcaccaacctgaagtatgaggaggacatgcatcgccctggctccaggaagttcaacaccactgagagggtcctgcagactctgcgtggtcctatgttcaagaacaccagtggtggccttctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatggcatcaaagagctgggcccctacaccctggacaggaacagtctctatgtcaatg

SEQ ID NO: 552 - DNA, 465 bp, Homo sapiens
gtttcacccattggatccctgtgcccaccagcagcactcctgggacctccacagtggaccttgggtcagggactccatcctccctccccagccccacaactgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacaccagtgtcggccttctgtactctggctgcagactgaccttgctcaggcctgagaagaatggggcagccactggaatggatgccatctgcagccaccgtcttgaccccaaaagccctggactcaacagagagcagctgtactgggagctgagccagctgacccatggcatcaaagagctgggcccctacaccctggacaggcacagtctctatgtcaatg

SEQ ID NO: 553 - DNA, 401 bp, Homo sapiens
aacgattcgaccttctcctgggacctccacagtggaccttgggtcagggactccatcctccctccccagccccacaactgctggccctctcctggtgccgttcaccctcaacttcaccatcaccaacctggagtacgaggaggacatgcattgccctggctccaggaagttcaacaccacagagagagtcctgcagagtctgcttggtcccatgttcaagaacaccagtgttggccctctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatggcatcaaagaaa

SEQ ID NO: 554 - DNA, 385 bp, Homo sapiens
tcctgggacctccacagtggaccttgggtcctcagggactccatcctccctccccagccctacatctgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacaccagtgtcggccttctgtactctggctgcagactgaccttgctcaggcctgagaagaatggggcagccactggaatggatgccatctgcagccaccgtcttgaccccaaaagccctggactcaacagagagcagctgtactgggagctgagccagctgacccatggcatcaaa

SEQ ID NO: 555 - DNA, 173 bp, Homo sapiens
gtctgagaaggatggggcagccactggagtggatgccatctgcacccaccaccttaaccctcaaagccctggactggacagggagcagctgtactggcagctgagccagatgaccaatggcatcaaagagctgggcccctacaccctggaccggaacagtctctacgtcaatg

SEQ ID NO: 556 - DNA, 468 bp, Homo sapiens
gtttcacccatcggagctctgggctcaccaccagcactccttggacttccacagttgaccttggaacctcagggactccatcccccgtccccagccccacaactgctggccctctcctggtgccattcaccctaaacttcaccatcaccaacctgcagtatgaggaggacatgcatcgccctggatctaggaagttcaacgccacagagagggtcctgcagggtctgcttagtcccatattcaagaactccagtgttggccctctgtactctggctgcagactgacctctctcaggcccgagaaggatggggcagcaactggaatggatgctgtctgcctctaccaccctaatcccaaaagacctgggctggacagagagcagctgtactgggagctaagccagctgacccacaacatcactgagctgggcccctacagcctggacaggcacagtctctatgtcaatg

SEQ ID NO: 557 - DNA, 468 bp, Homo sapiens
gtttcacccatcagaactctgtgcccaccaccagtactcctgggacctccacagtgtactgggcaaccactgggactccatcctccttccccggccacacagagcctggccctctcctgataccattcactttcaactttaccatcaccaacctgcattatgaggaaaacatgcaacaccctggttccaggaagttcaacaccacggagagggttctgcagggtctgctcacgcccttgttcaagaacaccagtgttggccctctgtactctggctgcagactgaccttgctcagacctgagaagcaggaggcagccactggagtggacaccatctgtacccaccgcgttgatcccatcggacctggactggacagagagcggctatactgggagctgagccagctgaccaacagcatcacagagctgggaccctacaccctggatagggacagtctctatgtcaatg

SEQ ID NO: 558 - DNA, 468 bp, Homo sapiens
gcttcaacccttggagctctgtgccaaccaccagcactcctgggacctccacagtgcacctggcaacctctgggactccatcctccctgcctggccacacagcccctgtccctctcttgataccattcaccctcaactttaccatcaccaacctgcattatgaagaaaacatgcaacaccctggttccaggaagttcaacaccacggagagggttctgcagggtctgctcaagcccttgttcaagagcaccagcgttggccctctgtactctggctgcagactgaccttgctcagacctgagaaacatggggcagccactggagtggacgccatctgcaccctccgccttgatcccactggtcctggactggacagagagcggctatactgggagctgagccagctgaccaacagcgttacagagctgggcccctacaccctggacagggacagtctctatgtcaatg

SEQ ID NO: 559 - DNA, 468 bp, Homo sapiens
gcttcacccatcggagctctgtgccaaccaccagtattcctgggacctctgcagtgcacctggaaacctctgggactccagcctccctccctggccacacagcccctggccctctcctggtgccattcaccctcaacttcactatcaccaacctgcagtatgaggaggacatgcgtcaccctggttccaggaagttcaacaccacggagagagtcctgcagggtctgctcaagcccttgttcaagagcaccagtgttggccctctgtactctggctgcagactgaccttgctcaggcctgaaaaacgtggggcagccaccggcgtggacaccatctgcactcaccgccttgaccctctaaaccctggactggacagagagcagctatactgggagctgagcaaactgacctgtggcatcatcgagctgggcccctacctcctggacagaggcagtctctatgtcaatg

SEQ ID NO: 560 - DNA, 468 bp, Homo sapiens
gtttcacccatcggaactttgtgcccatcaccagcactcctgggacctccacagtacacctaggaacctctgaaactccatcctccctacctagacccatagtgcctggccctctcctggtgccattcaccctcaacttcaccatcaccaacttgcagtatgaggaggccatgcgacaccctggctccaggaagttcaataccacggagagggtcctacagggtctgctcaggcccttgttcaagaataccagtatcggccctctgtactccagctgcagactgaccttgctcaggccagagaaggacaaggcagccaccagagtggatgccatctgtacccaccaccctgaccctcaaagccctggactgaacagagagcagctgtactgggagctgagccagctgacccacggcatcactgagctgggcccctacaccctggacagggacagtctctatgtcgatg

SEQ ID NO: 561 - DNA, 468 bp, Homo sapiens
gtttcactcattggagccccataccaaccaccagcactcctgggacctccatagtgaacctgggaacctctgggatcccaccttccctccctgaaactacagccaccggccctctcctggtgccattcacactcaacttcaccatcactaacctacagtatgaggagaacatgggtcaccctggctccaggaagttcaacatcacggagagtgttctgcagggtctgctcaagcccttgttcaagagcaccagtgttggccctctgtattctggctgcagactgaccttgctcaggcctgagaaggacggagtagccaccagagtggacgccatctgcacccaccgccctgaccccaaaatccctgggctagacagacagcagctatactgggagctgagccagctgacccacagcatcactgagctgggaccctacaccctggatagggacagtctctatgtcaatg

SEQ ID NO: 562 - DNA, 407 bp, Homo sapiens
gtttcacccagcggagctctgtgcccaccaccagcaccactggccctgtcctgctgccattcaccctcaattttaccatcattaacctgcagtatgaggaggacatgcatcgccctggctccaggaagttcaacaccacggagagggtccttcagggtctgcttatgcccttgttcaagaacaccagtgtcagctctctgtactctggttgcagactgaccttgctcaggcctgagaaggatggggcagccaccagagtggatgctgtctgcacccatcgtcctgaccccaaaagccctggactggacagagagcggctgtactggaagctgagccagctgacccacggcatcactgagctgggcccctacaccctggacaggcacagtctctatgtcaatggtttc

SEQ ID NO: 563 - DNA, 468 bp, Homo sapiens; identical to SEQ ID NO: 521

SEQ ID NO: 564 - DNA, 468 bp, Homo sapiens; identical to SEQ ID NO: 520

SEQ ID NO: 565 - DNA, 465 bp, Homo sapiens; identical to SEQ ID NO: 514

SEQ ID NO: 566 - DNA, 402 bp, Homo sapiens; identical to SEQ ID NO: 513

SEQ ID NO: 567 - DNA, 450 bp, Homo sapiens; identical to SEQ ID NO: 517

SEQ ID NO: 568 - DNA, 1060 bp, Homo sapiens
  misc_feature 406,742,801: n = A,T,C or G
gctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctaccygaatttcaccatcaccaacctaccatattcccaggacaaagcccagccaggcaccaccaattaccagaggaacaaaaggaatattgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattytcccaacagaaatgagcccttaactgggaattctgaccttcccttctgggctgtcatcytcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcctggggtgcctttcccccagccagggtccaaagaagcttggctggggcagaaataaaccatattggtcggacacaaaaaaaaaaaaa

SEQ ID NO: 569 - DNA, 10622 bp, Homo sapiens
  misc_feature 1,691,1164,1165,1730,2015,2149,2785,3044,3163,4483,4632,4825,4841,4849,4883,4915,4932,4947,6355,6370,7716,8210,9131,9968,10304,10363: n = A,T,C or G
ncatccccagctcgaacagcagccacagtcccattcatggtgccattcaccctcaacttcaactcatcaccaacctgcagtacgaggaggacatgcggcacctggttccaggaagttcaacgcgcacagagagagaactgcagggtcgtgctcaaaccctagatcaggaatagcagtctggaatacctctattcaggctgcagactagcctcactcaggccagagaaggatagctcagccacggcagtggatgccatctgcacacatcgccctgaccctgaagacctcggactggacagagagcgactgtactgggagctgagcaatctgacaaatggcatccaggagctgggcccctacaccctggaccggaacagtctctatgtcaatggtttcacccatcgaagctctatgcccaccaccagcactcctgggacctccacagtggatgtgggaacctcagggactccatcctccagccccagccccacgactgctggccctctcctgatgccgttcaccctcaacttcaccatcaccaacctgcagtacgaggaggacatgcgtcgcactggctccaggaagttcaacaccatggagagtgtcctgcagggtctgctcaagcccttgttcaagaacaccagtgttggccctctgtactctggctgcagattgaccttgctcaggcccragaaagatggggcagccactggagtggatgccatctgcacccaccgccttgaccccaaaagccctggactcaacagggagcagctgtactgggagctaagcaaactgaccaatgacattgaagagctgggcccctacaccctggacaggaacagtctctatgtcaatggtttcacccatcagagctctgtgtccaccaccagcactcctgggacctccacagtggatctcagaacctcagtgactccatcctccctctccagccccacaattatggctgctggccctctcctggtaccattcaccctcaacttcaccatcaccaacctgcagtatggggaggacatgggtcaccctggctccaggaagttcaacaccacagagagggtcctgcagggtctgcttggtcccatattcaagaacaccagtgttggccctctgtactctggctgcagactgacctctctcaggtcyragaaggatggagcagccactggagtggatgccatctgcatccatcatcttgaccccaaaagccctggactcaacagagagcggctgtactgggagctgagccaactgaccaatggcatcaaagagctgggcccctacaccctggacaggaacagtctctatgtcaatgctgctggccctctcctggtgctgttcaccctcaacttcaccatcaccaacctgaagtatgaggaggacatgcatcgccctggctccaggaagttcaacaccactgagagggtcctgcagactctgcgtggtcctatgttcaagaacaccagtggtggccttctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatggcatcaaagagctgggcccctacaccctggacaggaacagtctctatgtcaatggtttcacccatcggacctctgtgcccaccascagcactcctgggacctccacagtggaccttggaacctcagggactccattctccctcccaagccccgcaactgctggccctctcctggtgctgttcaccctcaacttcaccatcaccaacctgaagtatgaggaggacatgcatcgccctggctccaggaagttcaacaccactgagagggtcctgcagactctgcttggtcctatgttcaagaacaccagtgttggccttctgtactctggctgcagactgaccttgctcaggtccgagaaggatggagcagycactggagtggatgccatctgcacccaccgtcttgaccccaaaagccctggagtggacagggagcagctatactgggagctgagccagctgaccaatggcatcaaagagctgggcccctacaccctggacaggmacagtctctatgtcaatggtttcacccattggatccctgtgcccaccagcagcactcctgggacctccacagtggaccttgggtcagggactccatcctccctccccagccccacaactgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacaccagtgtcggccttctgtactctggctgcagactgaccttgctcaggcctgagaagaatggggcagccactggaatggatgccatctgcagccaccgtcttgaccccaaaagccctggactcaacagagagcagctgtactgggagctgagccagctgacccatggcatcaaagagctgggcccctacaccctggacaggcacagtctctatgtcaatggtttcacccattggatccctgtgcccaccagcagcactcctgggacctccacagtggaccttgggtcagggactccatcctccctccccagccccacaactgctggccctctcctggtgccgttcaccctcaacttcaccatcaccaacctgragtacgaggaggacatgcattgccctggctccaggaagttcaacaccacagagagagtcctgcagagtctgcttggtcccatgttcaagaacaccagtgttggccctctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatgscatcaaagagctgggtccctacaccctggacagcaacagtctctatgtcaatggtttcacccatcagacctctgcgcccaacaccagcactcctgggacctccacagtggaccttgggwcctcagggactccatcctccctccccagccctacatctgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacaccagtgtcggccttctgtactctggctgcagactgaccttgctcaggcctgagaagaatggggcagccactggaatggatgccatctgcagccaccgtcttgaccccaaaagccctggactcaacagagagcagctgtactgggagctgagccagctgacccatggcatcaaagagctgggcccctacaccctggacaggaacagtctctatgtcaatggtttcacccatcggagctctgtggcccccaccagcactcctgggacctccacagtggaccttgggacctcagggactccatcctccctccccagccccacaacagctgttcctctcctggtgccgttcaccctcaactttaccatcaccaatctgcagtatggggaggacatgcgtcaccctggctccaggaagttcaacaccacagagagggtcctgcagggtctgcttggtcccttgttcaagaactccagtgtcggccctctgtactctggctgcagactgatctctctcaggtctgagaaggatggggcagccactggagtggatgccatctgcacccaccaccttaaccctcaaagccctggactggacagggagcagctgtactggcagctgagccagatgaccaatggcatcaaagagctgggcccctacaccctggaccggaacagtctctacgtcaatggtttcacccatcggagctctgggctcaccaccagcactccttggacttccacagttgaccttggaacctcagggactccatcccccgtccccagccccacaactgctggccctctcctggtgccattcaccctaaacttcaccatcaccaacctgcagtatgaggaggacatgcatcgccctggatctaggaagttcaacgccacagagagggtcctgcagggtctgcttagtcccatattcaagaactccagtgttggccctctgtactctggctgcagactgacctctctcaggcccgagaaggatggggcagcaactggaatggatgctgtctgcctctaccaccctaatcccaaaagacctgggctggacagagagcagctgtactgggagctaagccagctgacccacaacatcactgagctgggcccctacagcctggacaggsacagtctctatgtcaatggtttcacccatcagaactctgtgcccaccaccagtactcctgggacctccacagtgtactgggcaaccactgggactccatcctccttccccggccacacagagcctggccctctcctgataccattcacwttcaactttaccatcaccaacctgcattatgaggaaaacatgcaacaccctggttccaggaagttcaacaccacggagagggttctgcagggtctgctcacgcccttgttcaagaacaccagtgttggccctctgtactctggctgcagactgaccttgctcagacctgagaagcaggaggcagccactggartggacaccatctgtayccaccgcsttgatcccatcggacctggactggacagagagcrgctatactgggagctgagccagctgacccacrgcatcacagagctgggmccctacaccctggayagggacagtctctatgtcaatggcttcaacccttggagctctgtgccaaccaccagcactcctgggacctccacagtgcacctggcaacctctgggactccatcctccctgcctggccacacagcccctgtccctctcttgataccattcaccctcaactttaccatcaccaacctgcattatgaagaaaacatgcaacaccctggttccaggaagttcaacaccacggagagggttctgcagggtctgctcaagcccttgttcaagagcaccagcgttggccctctgtactctggctgcagactgaccttgctcagacctgagaaacatggggcagccactggagtggacgccatctgcaccctccgccttgatcccactggtcctggactggacagagagcggctatactgggagctgagccagctgaccaacagcgttacagagctgggcccctacaccctggacagggacagtctctatgtcaatggcttcacccatcggagctctgtgccaaccaccagtattcctgggacctctgcagtgcacctggaaacctctgggactccagcctccctccctggccacacagcccctggccctctcctggtgccattcaccctcaacttcactatcaccaacctgcagtatgaggaggacatgcgtcaccctggttccaggaagttcaacaccacggagagagtcctgcagggtctgctcaagcccttgttcaagagcaccagtgttggccctctgtactctggctgcagactgaccttgctcaggcctgaaaaacgtggggcagccaccggcgtggacaccatctgcactcaccgccttgaccctctaaaccctggactggacagagagcagctatactgggagctgagcaaactgacctgtggcatcatcgagctgggcccctacctcctggacagaggcagtctctatgtcaatggtttcacccatcggaactttgtgcccatcaccagcactcctgggacctccacagtacacctaggaacctctgaaactccatcctccctacctagacccatagtgcctggccctctcctggtgccattcaccctcaacttcaccatcaccaacttgcagtatgaggaggccatgcgacaccctggctccaggaagttcaataccacggagagggtcctacagggtctgctcaggcccttgttcaagaataccagtatcggccctctgtactccagctgcagactgaccttgctcaggccagagaaggacaaggcagccaccagagtggatgccatctgtacccaccaccctgaccctcaaagccctggactgaacagagagcagctgtactgggagctgagccagctgacccacggcatcactgagctgggcccctacaccctggacaggsacagtctctatgtcratggtttcactcattggagccccataccaaccaccagcactcctgggacctccatagtgaacctgggaacctctgggatcccaccttccctccctgaaactacagccaccggccctctcctggtgccattcacactcaacttcaccatcactaacctacagtatgaggagaacatgggtcaccctggctccaggaagttcaacatcacggagagtgttctgcagggtctgctcaagcccttgttcaagagcaccagtgttggccctctgtattctggctgcagactgaccttgctcaggcctgagaaggacggagtagccaccagagtggacgccatctgcacccaccgccctgaccccaaaatccctgggctagacagacagcagctatactgggagctgagccagctgacccacagcatcactgagctgggaccctacaccctggatagggacagtctctatgtcaatggtttcacccagcggagctctgtgcccaccaccagcactcctgggactttcacagtacagccggaaacctctgagactccatcatccctccctggccccacagccactggccctgtcctgctgccattcaccctcaattttaccatcattaacctgcagtatgaggaggacatgcatcgccctggctccaggaagttcaacaccacggagagggtccttcagggtctgcttatgcccttgttcaagaacaccagtgtcagctctctgtactctggttgcagactgaccttgctcaggcctgagaaggatggggcagccaccagagtggatgctgtctgcacccatcgtcctgaccccaaaagccctggactggacagagagcggctgtactggaagctgagccagctgacccacggcatcactgagctgggcccctacaccctggacaggcacagtctctatgtcaatggtttcacccatcagagctctatgacgaccaccagaactcctgatacctccacaatgcacctggcaacctcgagaactccagcctccctgtctggacctacgaccgccagccctctcctggtgctattcacaattaacttcaccatcactaacctgcggtatgaggagaacatgcatcaccctggctctagaaagtttaacaccacggagagagtccttcagggtctgctcaggcctgtgttcaagaacaccagtgttggccctctgtactctggctgcagactgaccttgctcaggcccaagaaggatggggcagccaccaaagtggatgccatctgcacctaccgccctgatcccaaaagccctggactggacagagagcagctatactgggagctgagccagctracccacagcatcactgagctgggcccctacaccctggacagggacagtctctatgtcaatggtttcacacagcggagctctgtgcccaccactagcattcctgggacccccacagtggacctgggaacatctgggactccagtttctaaacctggtccctcggctgccagccctctcctggtgctattcactctcaacttcaccatcaccaacctgcggtatgaggagaacatgcagcaccctggctccaggaagttcaacaccacggagagggtccttcagggcctgctcaggtccctgttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggccmctatgccctggacaacgacagcctctttgtcaatggtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctgcaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatggtttcacccatcggagctctgtacccaccaccagcaccggggtggtcagcgaggagccattcacactgaacttcaccatcaacaacctgcgctacatggcggacatgggccaacccggctccctcaagttcaacatcacagacaacgtcatgaagcacctgctcagtcctttgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacggttacaatgaacctggtcyagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatggctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctaccygaatttcaccatcaccaacctaccatattcccaggacaaagcccagccaggcaccaccaattaccagaggaacaaaaggaatattgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattytcccaacagaaatgagcccttaactgggaattctgaccttcccttctgggctgtcatcytcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcctggggtgcctttcccccagccagggtccaaagaagcttggctggggcagaaataaaccatattggtcggacacaaaaaaaaaaaaa

SEQ ID NO: 570 - DNA, 469 bp, Homo sapiens
  misc_feature 71,92,93,120,124,168,178,218,230,300,321,350,387,412,414,415,422,423,451: n = A,T,C or G
gtttcacccatcggagctctgtgcccaccaccagcactcctgggacctccacagtggacctgggaacctcwgggactccatcctccctcccyrgccccacagctgctggccctctcctgrtgcyattcaccctcaacttcaccatcaccaacctgcagtatgaggagracatgcatcrccctggctccaggaagttcaacaccacggagagggtcctkcagggtctgctyaggtcccttgttcaagaacaccagtgttggccctctgtactctggctgcagactgaccttgctcaggccygagaaggatggggcagccacyggagtggatgccatctgcacccaccgccytgaccccaaaagccctggactggacagagagcagctrtactgggagctgagccagctgaccmayrgcatcamwgagctgggcccctacaccctggacaggracagtctctatgtcaatg

SEQ ID NO: 571 - protein, 130 aa, Homo sapiens
  variant 69,107,110: Xaa = Any amino acid
HPQLEQQPQSHSWCHSPSTSTHHQPAVRGGHAAPGSRKFNAHRERTAGSCSNPRSGIAVWNTSIQAADXPHSGQRRIAQPRQWMPSAHIALTLKTSDWTESDCTGSXAIXQMASRSWAPTPWTGTVSMSM

SEQ ID NO: 572 - protein, 130 aa, Homo sapiens
  variant 1,58,78,92,94: Xaa = Any amino acid
XIPSSNSSHSPIHGAIHPQLQLITNLQYEEDMRHLVPGSSTRTERELQGRAQTLDQEXQSGIPLFRLQTSLTQAREGXLSHGSGCHLHTSPXPXRPRTGQRATVLGAEQSDKWHPGAGPLHPGPEQSLCQ

SEQ ID NO: 573 - protein, 130 aa, Homo sapiens
  variant 1,54: Xaa = Any amino acid
XSPARTAATVPFMVPFTLNFNSSPTCSTRRTCGTWFQEVQRAQRENCRVVLKPXIRNSSLEYLYSGCRLASLRPEKDSSATAVDAICTHRPDPEDLGLDRERLYWELSNLTNGIQELGPYTLDRNSLYVN

SEQ ID NO: 574 - protein, 156 aa, Homo sapiens
  variant 101: Xaa = Any amino acid
GFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPXKDGAATGVDAICTHRLDPKSPGLNREQLYWELSKLTNDIEELGPYTLDRNSLYVN

SEQ ID NO: 575 - protein, 158 aa, Homo sapiens
  variant 103: Xaa = Any amino acid
GFTHQSSVSTTSTPGTSTVDLRTSVTPSSLSSPTIMAAGPLLVPFTLNFTITNLQYGEDMGHPGSRKFNTTERVLQGLLGPIFKNTSVGPLYSGCRLTSLRSXKDGAATGVDAICIHHLDPKSPGLNRERLYWELSQLTNGIKELGPYTLDRNSLYVN

SEQ ID NO: 576 - protein, 122 aa, Homo sapiens
AAGPLLVLFTLNFTITNLKYEEDMHRPGSRKFNTTERVLQTLRGPMFKNTSGGLLYSGCRLTLLRSEKDGAATGVDAICTHRLDPKSPGVDREQLYWELSQLTNGIKELGPYTLDRNSLYVN

SEQ ID NO: 577 - protein, 156 aa, Homo sapiens
  variant 11,106,151: Xaa = Any amino acid
GFTHRTSVPTXSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAXTGVDAICTHRLDPKSPGVDREQLYWELSQLTNGIKELGPYTLDRXSLYVN

SEQ ID NO: 578 - protein, 155 aa, Homo sapiens
GFTHWIPVPTSSTPGTSTVDLGSGTPSSLPSPTTAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRHSLYVN

SEQ ID NO: 579 - protein, 155 aa, Homo sapiens
  variant 52,138: Xaa = Any amino acid
GFTHWIPVPTSSTPGTSTVDLGSGTPSSLPSPTTAGPLLVPFTLNFTITNLXYEEDMHCPGSRKFNTTERVLQSLLGPMFKNTSVGPLYSGCRLTLLRSEKDGAATGVDAICTHRLDPKSPGVDREQLYWELSQLTNXIKELGPYTLDSNSLYVN

SEQ ID NO: 580 - protein, 156 aa, Homo sapiens
  variant 23: Xaa = Any amino acid
GFTHQTSAPNTSTPGTSTVDLGXSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVN

SEQ ID NO: 581 - protein, 156 aa, Homo sapiens
GFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVN

SEQ ID NO: 582 - protein, 156 aa, Homo sapiens
  variant 151: Xaa = Any amino acid
GFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRXSLYVN

SEQ ID NO: 583 - protein, 156 aa, Homo sapiens
  variant 109,114,117,128,139: Xaa = Any amino acid
GFTHQNSVPTTSTPGTSTVYWATTGTPSSFPGHTEPGPLLIPFTFNFTITNLHYEENMQHPGSRKFNTTERVLQGLLTPLFKNTSVGPLYSGCRLTLLRPEKQEAATGXDTICXHRXDPIGPGLDREXLYWELSQLTHXITELGPYTLDRDSLYVN

SEQ ID NO: 584 - protein, 156 aa, Homo sapiens
GFNPWSSVPTTSTPGTSTVHLATSGTPSSLPGHTAPVPLLIPFTLNFTITNLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVN

SEQ ID NO: 585 - protein, 156 aa, Homo sapiens
GFTHRSSVPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLDREQLYWELSKLTCGIIELGPYLLDRGSLYVN

SEQ ID NO: 586 - protein, 156 aa, Homo sapiens
  variant 151,156: Xaa = Any amino acid
GFTHRNFVPITSTPGTSTVHLGTSETPSSLPRPIVPGPLLVPFTLNFTITNLQYEEAMRHPGSRKFNTTERVLQGLLRPLFKNTSIGPLYSSCRLTLLRPEKDKAATRVDAICTHHPDPQSPGLNREQLYWELSQLTHGITELGPYTLDRXSLYVX

SEQ ID NO: 587 - protein, 156 aa, Homo sapiens
GFTHWSPIPTTSTPGTSIVNLGTSGIPPSLPETTATGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVN

SEQ ID NO: 588 - protein, 156 aa, Homo sapiens
GFTQRSSVPTTSTPGTFTVQPETSETPSSLPGPTATGPVLLPFTLNFTIINLQYEEDMHRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVN

SEQ ID NO: 589 - protein, 156 aa, Homo sapiens
GFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVN

SEQ ID NO: 590 - protein, 156 aa, Homo sapiens
  variant 145: Xaa = Any amino acid
GFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGXYALDNDSLFVN

SEQ ID NO: 591 - protein, 155 aa, Homo sapiens
GFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVN

SEQ ID NO: 592 - protein, 134 aa, Homo sapiens
GFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLN

SEQ ID NO: 593 - protein, 150 aa, Homo sapiens
  variant 7: Xaa = Any amino acid
GYNEPGXDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFIN

SEQ ID NO: 594 - protein, 318 aa, Homo sapiens
  variant 136,248,268: Xaa = Any amino acid
GYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYXNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYXPNRNEPLTGNSDLPFWAVIXIGLAGLLGLITCLICGVLVTTRRRKKEGEYNVQQQCPGYYQSHLDLEDLQ

SEQ ID NO: 595 - protein, 3451 aa, Homo sapiens
  VARIANT 177, 335, 523, 618, 663, 875, 961, 1001, 1441, 1555,1560, 1563, 1574, 1585, 2065, 2070, 2683, 2990, 3269, 3381, 3401: Xaa = Any Amino Acid
IRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPXKDGAATGVDAICTHRLDPKSPGLNREQLYWELSKLTNDIEELGPYTLDRNSLYVNGFTHQSSVSTTSTPGTSTVDLRTSVTPSSLSSPTIMAAGPLLVPFTLNFTITNLQYGEDMGHPGSRKFNTTERVLQGLLGPIFKNTSVGPLYSGCRLTSLRSXKDGAATGVDAICIHHLDPKSPGLNRERLYWELSQLTNGIKELGPYTLDRNSLYVNAAGPLLVLFTLNFTITNLKYEEDMHRPGSRKFNTTERVLQTLRGPMFKNTSGGLLYSGCRLTLLRSEKDGAATGVDAICTHRLDPKSPGVDREQLYWELSQLTNGIKELGPYTLDRNSLYVNGFTHRTSVPTXSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAXTGVDAICTHRLDPKSPGVDREQLYWELSQLTNGIKELGPYTLDRXSLYVNGFTHWIPVPTSSTPGTSTVDLGSGTPSSLPSPTTAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRHSLYVNGFTHWIPVPTSSTPGTSTVDLGSGTPSSLPSPTTAGPLLVPFTLNFTITNLXYEEDMHCPGSRKFNTTERVLQSLLGPMFKNTSVGPLYSGCRLTLLRSEKDGAATGVDAICTHRLDPKSPGVDREQLYWELSQLTNXIKELGPYTLDSNSLYVNGFTHQTSAPNTSTPGTSTVDLGXSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRXSLYVNGFTHQNSVPTTSTPGTSTVYWATTGTPSSFPGHTEPGPLLIPFTFNFTITNLHYEENMQHPGSRKFNTTERVLQGLLTPLFKNTSVGPLYSGCRLTLLRPEKQEAATGXDTICXHRXDPIGPGLDREXLYWELSQLTHXITELGPYTLDRDSLYVNGFNPWSSVPTTSTPGTSTVHLATSGTPSSLPGHTAPVPLLIPFTLNFTITNLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTHRSSVPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLDREQLYWELSKLTCGIIELGPYLLDRGSLYVNGFTHRNFVPITSTPGTSTVHLGTSETPSSLPRPIVPGPLLVPFTLNFTITNLQYEEAMRHPGSRKFNTTERVLQGLLRPLFKNTSIGPLYSSCRLTLLRPEKDKAATRVDAICTHHPDPQSPGLNREQLYWELSQLTHGITELGPYTLDRXSLYVXGFTHWSPIPTTSTPGTSIVNLGTSGIPPSLPETTATGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTIINLQYEEDMHRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGXYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGXDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYXNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYXPNRNEPLTGNSDLPFWAVIXIGLAGLLGLITCLICGVLVTTRRRKKEGEYNVQQQCPGYYQSHLDLEDLQ

SEQ ID NO: 596 - protein, 156 aa, Homo sapiens
GFTHRSSVPTTSTPGTSTVDLGTSGTPSSLPSPTAAGPLLVPFTLNFTITNLQYEEDMHHPGSRKFNTTERVLQGLLGPLFKNTSVGPLYSGCRLTLLRPEKDGAATGVDAICTHRLDPKSPGLDREQLYWELSQLTHGITELGPYTLDRDSLYVN

SEQ ID NO: 597 - protein, 28 aa, Artificial Sequence
  Peptide that correspond to the extracellular domain of O772P molecule.
KSYFSDCQVSTFRSVPNRHHTGVDSLCN

SEQ ID NO: 598 - protein, 28 aa, Artificial Sequence
  Peptide that correspond to the extracellular domain of O772P molecule.
FSPLARRVDRVAIYEEFLRMTRNGTQLQ

SEQ ID NO: 599 - protein, 28 aa, Artificial Sequence
  Peptide that correspond to the extracellular domain of O772P molecule.
TLDRSSVLVDGYFPNRNEPLTGNSDLPF

SEQ ID NO: 600 - protein, 20 aa, Homo sapiens
MASLGQILFWSIISIIIILA

SEQ ID NO: 601 - protein, 20 aa, Homo sapiens
IIILAGAIALIIGFGISGRH

SEQ ID NO: 602 - protein, 20 aa, Homo sapiens
ISGRHSITVTTVASAGNIGE

SEQ ID NO: 603 - protein, 20 aa, Homo sapiens
GNIGEDGILSCTFEPDIKLS

SEQ ID NO: 604 - protein, 20 aa, Homo sapiens
DIKLSDIVIQWLKEGVLGLV

SEQ ID NO: 605 - protein, 20 aa, Homo sapiens
VLGLVHEFKEGKDELSEQDE

SEQ ID NO: 606 - protein, 20 aa, Homo sapiens
SEQDEMFRGRTAVFADQVIV

SEQ ID NO: 607 - protein, 20 aa, Homo sapiens
DQVIVGNASLRLKNVQLTDA

SEQ ID NO: 608 - protein, 21 aa, Homo sapiens
VQLTDAGTYKCYIITSKGKGN

SEQ ID NO: 609 - protein, 20 aa, Homo sapiens
KGKGNANLEYKTGAFSMPEV

SEQ ID NO: 610 - protein, 20 aa, Homo sapiens
SMPEVNVDYNASSETLRCEA

SEQ ID NO: 611 - protein, 20 aa, Homo sapiens
LRCEAPRWFPQPTVVWASQV

SEQ ID NO: 612 - protein, 20 aa, Homo sapiens
WASQVDQGANFSEVSNTSFE

SEQ ID NO: 613 - protein, 20 aa, Homo sapiens
NTSFELNSENVTMKVVSVLY

SEQ ID NO: 614 - protein, 20 aa, Homo sapiens
VSVLYNVTINNTYSCMIEND

SEQ ID NO: 615 - protein, 20 aa, Homo sapiens
MIENDIAKATGDIKVTESEI

SEQ ID NO: 616 - protein, 20 aa, Homo sapiens
TESEIKRRSHLQLLNSKASL

SEQ ID NO: 617 - protein, 20 aa, Homo sapiens
SKASLCVSSFFAISWALLPI

SEQ ID NO: 618 - protein, 20 aa, Homo sapiens
SSFFAISWALLPLSPYLMLK

SEQ ID NO: 619 - DNA, 30 bp, Artificial Sequence
  PCR primer
cagaagcttatggcttccctggggcagact

SEQ ID NO: 620 - DNA, 31 bp, Artificial Sequence
  PCR primer
cagcggccgcttattttagcatcaggtaagg

SEQ ID NO: 621 - DNA, 849 bp, Rhesus Monkey
atggcttccctggggcagatcctcttctggagcataattagcatcatctttattctggctggagcaattgcactcatcattggctttggtatttcagggagacactccatcacagtcactactgttgcctcagctgggaacattggggaggatggaatcctgagctgcacttttgaacctgacatcaaactttctgatatcgtgatacaatggctgaaggaaggtgttataggcttggtccatgagttcaaagaaggcaaagatgagctgtcggagcaggatgaaatgttcagaggccggacagcagtgtttgctgatcaagtgatagttggcaatgcctctctgcggctgaaaaatgtacaacttacagacgctggcacctacaaatgttacatcatcacttctaaaggcaaggggaatgctaaccttgagtataaaactggagccttcagcatgccagaggtgaatgtggactataacgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctatacaatgttacgatcaacaacacatactcctgtatgattgaaaatgacattgccaaagcaacaggggatatcaaagtgacagaatctgagatcaaaagacggagtcacctacagctgctaaactcaaaggcttctctgtgtgtctcttctttccttgccatcagctgggcacttctgcctctcgccccttacctgatgctaaaataa

SEQ ID NO: 622 - DNA, 1399 bp, Mus musculus
  misc_feature 1093,1130,1134,1233: n = A,T,C or G
cccgcgtccgcggacgcgtggggcagcaggcaggcagctccactcaccaaaatctggccccacacacagcaggactgtgggaaggaactccctctccatggcttccttggggcagatcatcttttggagtattattaacatcatcatcatcctggctggggccatcgcactcatcattggctttggcatttcaggcaagcacttcatcacggtcacgaccttcacctcagctggaaacattggagaggacgggaccctgagctgcacttttgaacctgacatcaaactcaacggcatcgtcatccagtggctgaaagaaggcatcaaaggtttggtccacgagttcaaagaaggcaaagacgacctctcacagcagcatgagatgttcagaggccgcacagcagtgtttgctgatcaggtggtagttggcaatgcttccctgagactgaaaaacgtgcagctcacggatgctggcacctacacatgttacatccgcacctcaaaaggcaaagggaatgcaaaccttgagtataagaccggagccttcagtatgccagagataaatgtggactataatgccagttcagagagtttacgctgcgaggctcctcggtggttcccccagcccacagtggcctgggcatctcaagtcgaccaaggagccaatttctcagaagtctccaacaccagctttgagttgaactctgagaatgtgaccatgaaggtcgtatctgtgctctacaatgtcacaatcaacaacacatactcctgtatgattgaaaacgacattgccaaagccaccggggacatcaaagtgacagattcagaggtcaaaaggcgaagtcagctgcagttgctgaactctgggccttccccgtgtgttttttcttctgcctttgtggctggctgggcactcctatctctctcctgttgcctgatgctaagatgaggggccctggctacacaaaagcatgcaacgttgctggtccaacagaatcccggagaactacagaaatattttcctcaagacatgacctagttttatatttttagaagaagatgaaatcatgtctagaagtctggagagagcagncaggaccaagatgtggaaggaaaacaaaagtaacccncagncccccccgatcggaacaagatggacctagaaaataattcaaccaaactagagtatactaagtgtgctgttacaatgtgtgtagggtaggtgtcctcccmcatctcaggggcctcccctggtcccccagctcctgagttaggatgggctgttatgatgtccctctgaaggttcctggatggttcctactgccatatactcattttatattcagcccattaaaccatagtgaatgctaaaaaaaaaaaaaaaaaaaaaaaaaaaaaa

SEQ ID NO: 623 - protein, 282 aa, Rhesus Monkey
MASLGQILFWSIISIIFILAGAIALIIGFGISGRHSITVTTVASAGNIGEDGILSCTFEPDIKLSDIVIQWLKEGVIGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFLAISWALLPLAPYLMLK

SEQ ID NO: 624 - protein, 283 aa, Mus musculus
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGPSPCVFSSAFVAGWALLSLSCCLMLR

(19) United States
(12) Patent Application Publication
(10) Pub. No.: US 2011/0177082 A1
(43) Pub. Date: Jul. 21, 2011

(54) COMPOSITIONS AND METHODS FOR THE THERAPY AND DIAGNOSIS OF OVARIAN CANCER

(75) Inventors: Chaitanya S. Bangur, Seattle, WA (US); Marc W. Retter, Carnation, WA (US); Gary R. Fanger, Mill Creek, WA (US); Paul Hill, Everett, WA (US)

(73) Assignee: CORIXA CORPORATION, Seattle, WA (US)

(21) Appl. No.: 13/009,413

(22) Filed: Jan. 19, 2011

Related U.S. Application Data
(63) Continuation of application No. 10/860,790, filed on Jun. 2, 2004, now Pat. No. 7,888,477.
Application No. 10/860,790 is a continuation of application No. 10/198,053, filed on Jul. 17, 2002, now Pat. No. 6,858,710.
Application No. 10/198,053 is a continuation-in-part of application No. 09/907,969, filed on Jul. 17, 2001, now abandoned.
Application No. 09/907,969 is a continuation-in-part of application No. 09/884,441, filed on Jun. 18, 2001, now abandoned.
Application No. 09/884,441 is a continuation-in-part of application No. 09/827,271, filed on Apr. 4, 2001, now Pat. No. 6,962,980.
Application No. 09/827,271 is a continuation-in-part of application No. 09/667,857, filed on Sep. 20, 2000, now Pat. No. 6,699,664.
Application No. 09/667,857 is a continuation-in-part of application No. 09/636,801, filed on Aug. 10, 2000, now Pat. No. 7,202,334.
Application No. 09/636,801 is a continuation-in-part of application No. 09/617,747, filed on Jul. 17, 2000, now abandoned.

(51) Int. Cl. A61K 39/395; C07H 21/00; C07K 14/00; C07K 7/00; C07K 16/00; A61K 31/7088; A61K 38/00; A61K 35/12; A61K 39/00; A61P 35/00; C12N 15/63; G01N 33/68; C12N 5/0783; G01N 33/53; G01N 33/566; C12N 1/00; C12N 5/10

(52) U.S. Cl. 424/139.1; 536/23.1; 530/350; 530/326; 530/387.9; 514/44 R; 514/1.1; 424/93.71; 424/185.1; 514/19.3; 435/320.1; 436/64; 435/375; 435/325; 436/501; 435/243; 435/419

(57) ABSTRACT

Compositions and methods for the therapy and diagnosis of cancer, particularly ovarian cancer, are disclosed. Illustrative compositions comprise one or more ovarian tumor polypeptides, immunogenic portions thereof, polynucleotides that encode such polypeptides, antigen presenting cell that expresses such polypeptides, and T cells that are specific for cells expressing such polypeptides. The disclosed compositions are useful, for example, in the diagnosis, prevention and/or treatment of diseases, particularly ovarian cancer.

### CROSS-REFERENCES TO RELATED APPLICATIONS

This application is a continuation of U.S. patent application Ser. No. 10/860,790, filed Jun. 2, 2004, now allowed, which is a continuation of U.S. patent application Ser. No. 10/198,053, filed Jul. 17, 2002, now issued, which is a continuation-in-part of U.S. patent application Ser. No. 09/907,969, filed Jul. 17, 2001, now abandoned, which is a continuation-in-part of U.S. patent application Ser. No. 09/884,441, filed Jun. 18, 2001, now abandoned, which is a continuation-in-part of U.S. patent application Ser. No. 09/827,271, filed Apr. 4, 2001, now issued, which is a continuation-in-part of U.S. patent application Ser. No. 09/667,857, filed Sep. 20, 2000, now issued, which is a continuation-in-part of U.S. patent application Ser. No. 09/636,801, filed Aug. 10, 2000, now issued, which is a continuation-in-part of U.S. patent application Ser. No. 09/617,747, filed Jul. 17, 2000, now abandoned, which applications are incorporated herein by reference in their entireties.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 210121_{—}462C12 SEQUENCE_LISTING.txt. The text file is 524 KB, was created on Jan. 19, 2011, and is being submitted electronically via EFS-Web, concurrent with the filing of the specification.

### BACKGROUND OF THE INVENTION

1. Field of the Invention

The present invention relates generally to therapy and diagnosis of cancer, such as ovarian cancer. The invention is more specifically related to polypeptides, comprising at least a portion of an ovarian tumor protein, and to polynucleotides encoding such polypeptides. Such polypeptides and polynucleotides are useful in pharmaceutical compositions, e.g., vaccines, and other compositions for the diagnosis and treatment of ovarian cancer.

2. Description of the Related Art

Cancer is a significant health problem throughout the world. Although advances have been made in detection and therapy of cancer, no vaccine or other universally successful method for prevention and/or treatment is currently available. Current therapies, which are generally based on a combination of chemotherapy or surgery and radiation, continue to prove inadequate in many patients.

Ovarian cancer is a significant health problem for women in the United States and throughout the world. Although advances have been made in detection and therapy of this cancer, no vaccine or other universally successful method for prevention or treatment is currently available. Management of the disease currently relies on a combination of early diagnosis and aggressive treatment, which may include one or more of a variety of treatments such as surgery, radiotherapy, chemotherapy and hormone therapy. The course of treatment for a particular cancer is often selected based on a variety of prognostic parameters, including an analysis of specific tumor markers. However, the use of established markers often leads to a result that is difficult to interpret, and high mortality continues to be observed in many cancer patients.

Immunotherapies have the potential to substantially improve cancer treatment and survival. Such therapies may involve the generation or enhancement of an immune response to an ovarian carcinoma antigen. However, to date, relatively few ovarian carcinoma antigens are known and the generation of an immune response against such antigens has not been shown to be therapeutically beneficial.

Accordingly, there is a need in the art for improved methods for identifying ovarian tumor antigens and for using such antigens in the therapy of ovarian cancer. The present invention fulfills these needs and further provides other related advantages.

In spite of considerable research into therapies for these and other cancers, ovarian cancer remains difficult to diagnose and treat effectively. Accordingly, there is a need in the art for improved methods for detecting and treating such cancers. The present invention fulfills these needs and further provides other related advantages.

### BRIEF SUMMARY OF THE INVENTION

In one aspect, the present invention provides polynucleotide compositions comprising a sequence selected from the group consisting of:

(a) sequences provided in SEQ ID NOs: 1-311, 313-387, 391, 457, 460-477, 512-570 and 619-622;

(b) complements of the sequences provided in SEQ ID NOs: 1-311, 313-387, 391, 457, 460-477, 512-570 and 619-622;

(c) sequences consisting of at least 20, 25, 30, 35, 40, 45, 50, 75 and 100 contiguous residues of a sequence provided in SEQ ID NOs: 1-311, 313-387, 391, 457, 460-477, 512-570 and 619-622;

(d) sequences that hybridize to a sequence provided in SEQ ID NOs: 1-311, 313-387, 391, 457, 460-477, 512-570 and 619-622, under moderate or highly stringent conditions;

(e) sequences having at least 75%, 80%, 85%, 90%, 95%, 96%, 97%, 98% or 99% identity to a sequence of SEQ ID NOs: 1-311, 313-387, 391, 457, 460-477, 512-570 and 619-622;

(f) degenerate variants of a sequence provided in SEQ ID NOs: 1-311, 313-387, 391, 457, 460-477, 512-570 and 619-622.

In one preferred embodiment, the polynucleotide compositions of the invention are expressed in at least about 20%, more preferably in at least about 30%, and most preferably in at least about 50% of ovarian tumors samples tested, at a level that is at least about 2-fold, preferably at least about 5-fold, and most preferably at least about 10-fold higher than that for normal tissues.

The present invention, in another aspect, provides polypeptide compositions comprising an amino acid sequence that is encoded by a polynucleotide sequence described above.

The present invention further provides polypeptide compositions comprising an amino acid sequence selected from the group consisting of sequences recited in SEQ ID NOs: 312, 388-389, 392-455, 458-459, 478-511, 571-618 and 623-624.

In certain preferred embodiments, the polypeptides and/or polynucleotides of the present invention are immunogenic, i.e., they are capable of eliciting an immune response, particularly a humoral and/or cellular immune response, as further described herein.

The present invention further provides fragments, variants and/or derivatives of the disclosed polypeptide and/or polynucleotide sequences, wherein the fragments, variants and/or derivatives preferably have a level of immunogenic activity of at least about 50%, preferably at least about 70% and more preferably at least about 90% of the level of immunogenic activity of a polypeptide sequence set forth in SEQ ID NOs: 312, 388-389, 392-455, 458-459, 478-511, and 571-618 and 623-624 or a polypeptide sequence encoded by a polynucleotide sequence set forth in SEQ ID NOs: 1-311, 313-387, 391, 457, 460-477, 512-570 and 619-622.

The present invention further provides polynucleotides that encode a polypeptide described above, expression vectors comprising such polynucleotides and host cells transformed or transfected with such expression vectors.

Within other aspects, the present invention provides pharmaceutical compositions comprising a polypeptide or polynucleotide as described above and a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof; and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Illustrative antigen presenting cells include dendritic cells, macrophages, monocytes, fibroblasts and B cells.

Within related aspects, pharmaceutical compositions are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant. The fusions proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with ovarian cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with ovarian cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4⁺ and/or CD8⁺ T cells isolated from a patient with one or more of: (i) a polypeptide comprising at least an immunogenic portion of polypeptide disclosed herein; (ii) a polynucleotide encoding such a polypeptide; and (iii) an antigen-presenting cell that expressed such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer, preferably an ovarian cancer, in a patient comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody.

The present invention also provides, within other aspects, methods for monitoring the progression of a cancer in a patient. Such methods comprise the steps of: (a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample, e.g., tumor sample, serum sample, etc., obtained from a patient with an oligonucleotide that hybridizes to a polynucleotide that encodes a polypeptide of the present invention; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a polypeptide of the present invention; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE SEVERAL VIEWS OF THE DRAWINGS

FIGS. 1A-1S (SEQ ID NO:1-71) depict partial sequences of polynucleotides encoding representative secreted ovarian carcinoma antigens.

FIGS. 2A-2C depict full insert sequences for three of the clones of FIG. 1. FIG. 2A shows the sequence designated O7E (11731; SEQ ID NO:72), FIG. 2B shows the sequence designated O9E (11785; SEQ ID NO:73) and FIG. 2C shows the sequence designated O8E (13695; SEQ ID NO:74).

FIG. 3 presents results of microarray expression analysis of the ovarian carcinoma sequence designated O8E.

FIG. 4 presents a partial sequence of a polynucleotide (designated 3g; SEQ ID NO:75) encoding an ovarian carcinoma sequence that is a splice fusion between the human T-cell leukemia virus type I oncoprotein TAX and osteonectin.

FIG. 5 presents the ovarian carcinoma polynucleotide designated 3f (SEQ ID NO:76).

FIG. 6 presents the ovarian carcinoma polynucleotide designated 6b (SEQ ID NO:77).

FIGS. 7A and 7B present the ovarian carcinoma polynucleotides designated 8e (SEQ ID NO:78) and 8h (SEQ ID NO:79).

FIG. 8 presents the ovarian carcinoma polynucleotide designated 12c (SEQ ID NO:80).

FIG. 9 presents the ovarian carcinoma polynucleotide designated 12h (SEQ ID NO:81).

FIG. 10 depicts results of microarray expression analysis of the ovarian carcinoma sequence designated 3f.

FIG. 11 depicts results of microarray expression analysis of the ovarian carcinoma sequence designated 6b.

FIG. 12 depicts results of microarray expression analysis of the ovarian carcinoma sequence designated 8e.

FIG. 13 depicts results of microarray expression analysis of the ovarian carcinoma sequence designated 12c.

FIG. 14 depicts results of microarray expression analysis of the ovarian carcinoma sequence designated 12h.

FIGS. 15A-15EEE depict partial sequences of additional polynucleotides encoding representative secreted ovarian carcinoma antigens (SEQ ID NO:82-310).

FIG. 16 is a diagram illustrating the location of various partial O8E sequences within the full length sequence.

FIG. 17 is a graph illustrating the results of epitope mapping studies on O8E protein.

FIG. 18 is graph of a fluorescence activated cell sorting (FACS) analysis of O8E cell surface expression.

FIG. 19 is graph of a FACS analysis of O8E cell surface expression.

FIG. 20 shows FACS analysis results for O8E transfected HEK293 cells demonstrating cell surface expression of O8E.

FIG. 21 shows FACS analysis results for SKBR3 breast tumor cells demonstrating cell surface expression of O8E.

FIG. 22 shows O8E expression in HEK 293 cells. The cells were probed with anti-O8E rabbit polyclonal antisera #2333L.

FIG. 23 shows the ELISA analysis of anti-O8E rabbit sera.

FIG. 24 shows the ELISA analysis of affinity purified rabbit anti-O8E polyclonal antibody.

FIG. 25 is a graph determining antibody internalization of anti-O8E mAb showing that mAbs against amino acids 61-80 induces ligand internalization.

### BRIEF DESCRIPTION OF THE SEQUENCE IDENTIFIERS

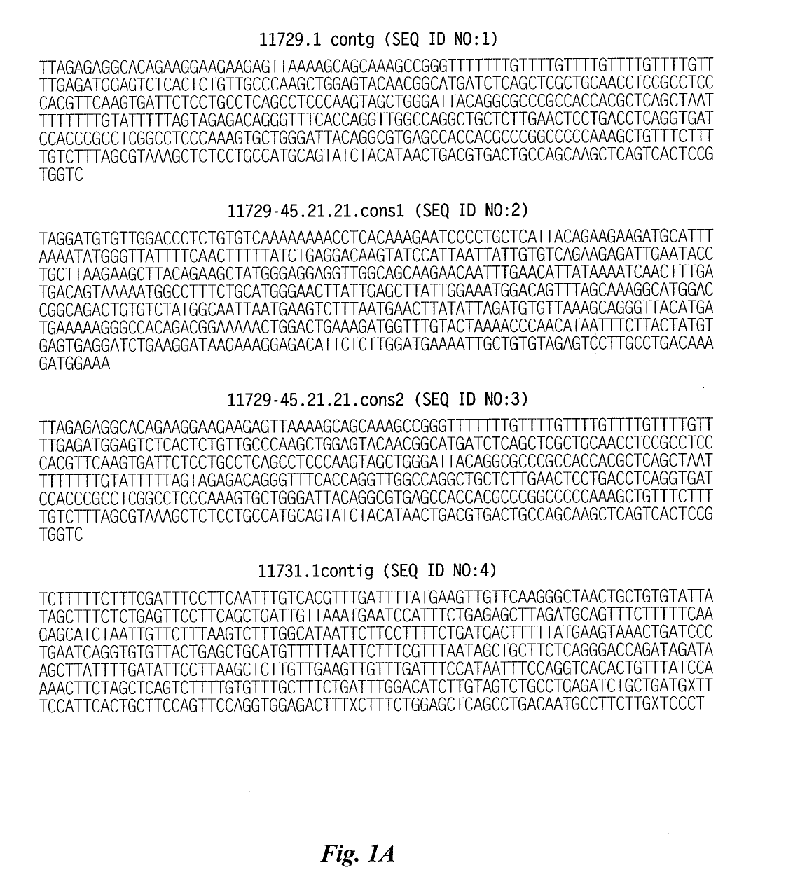

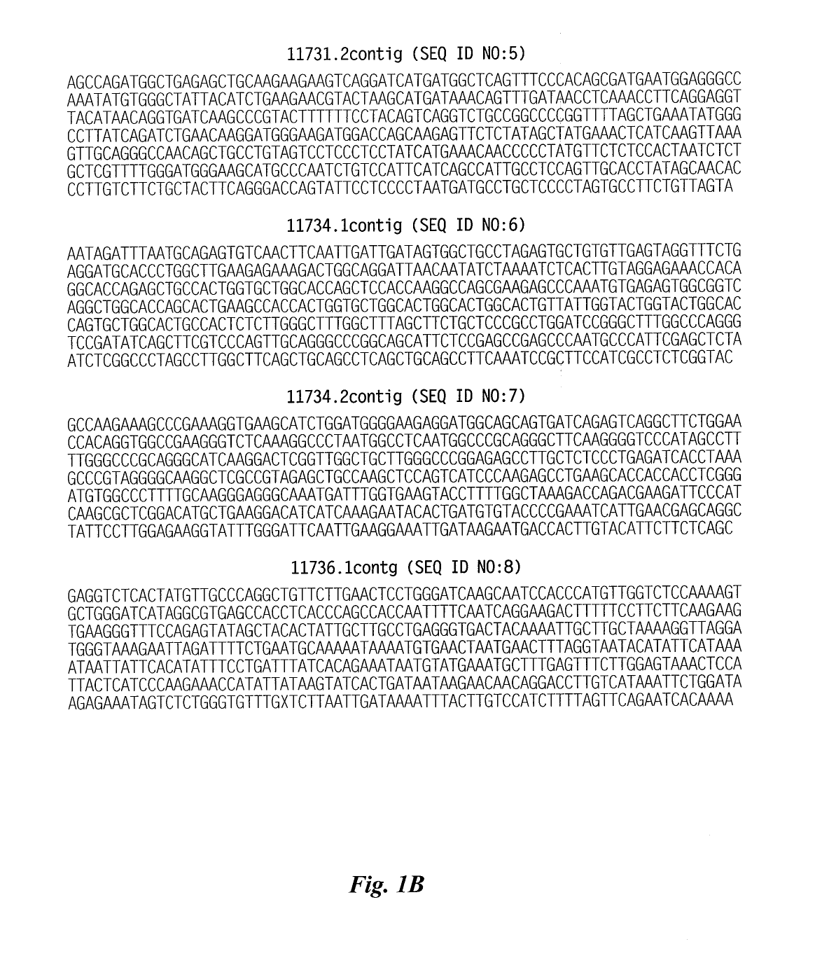

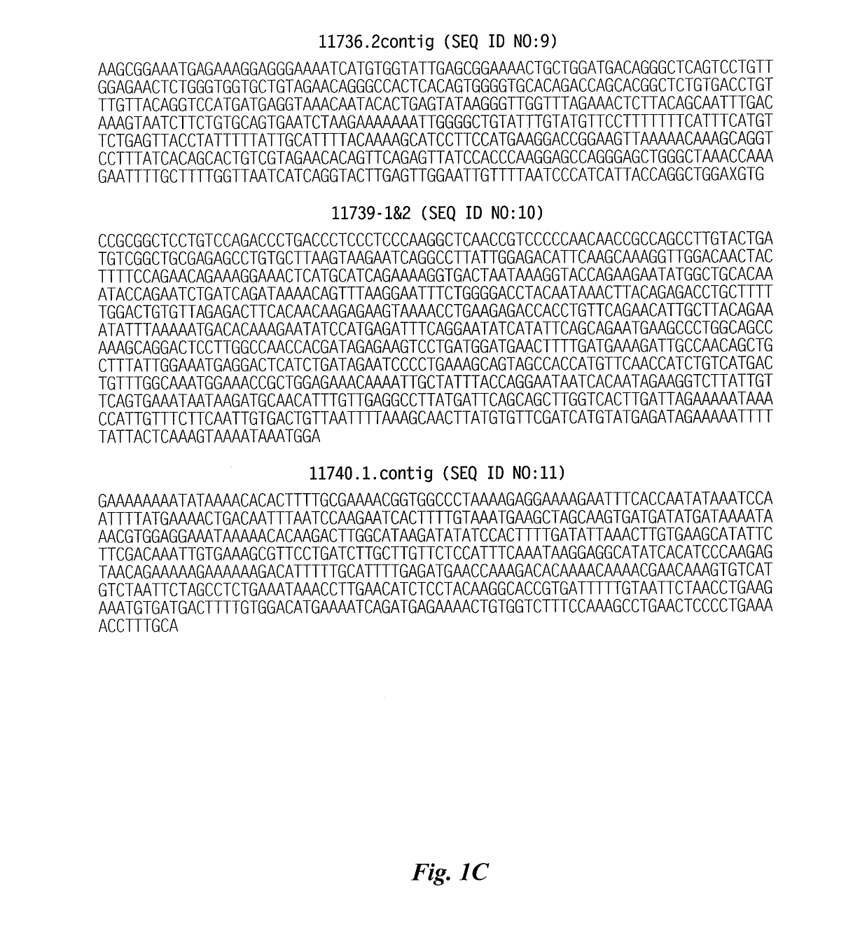

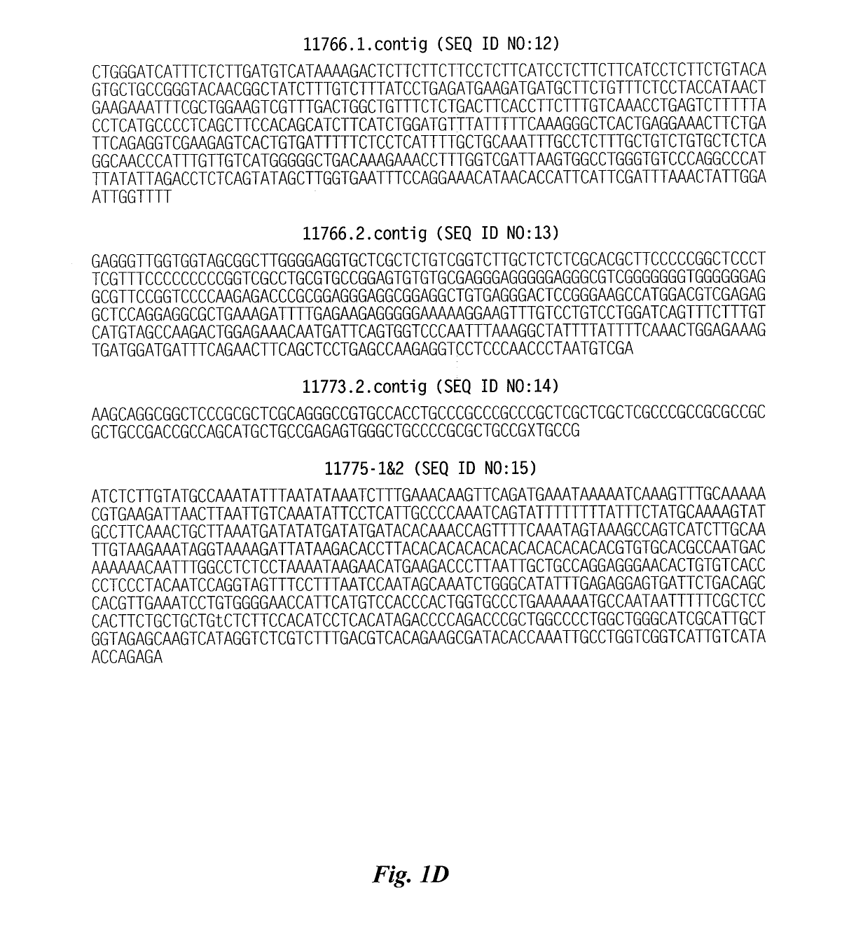

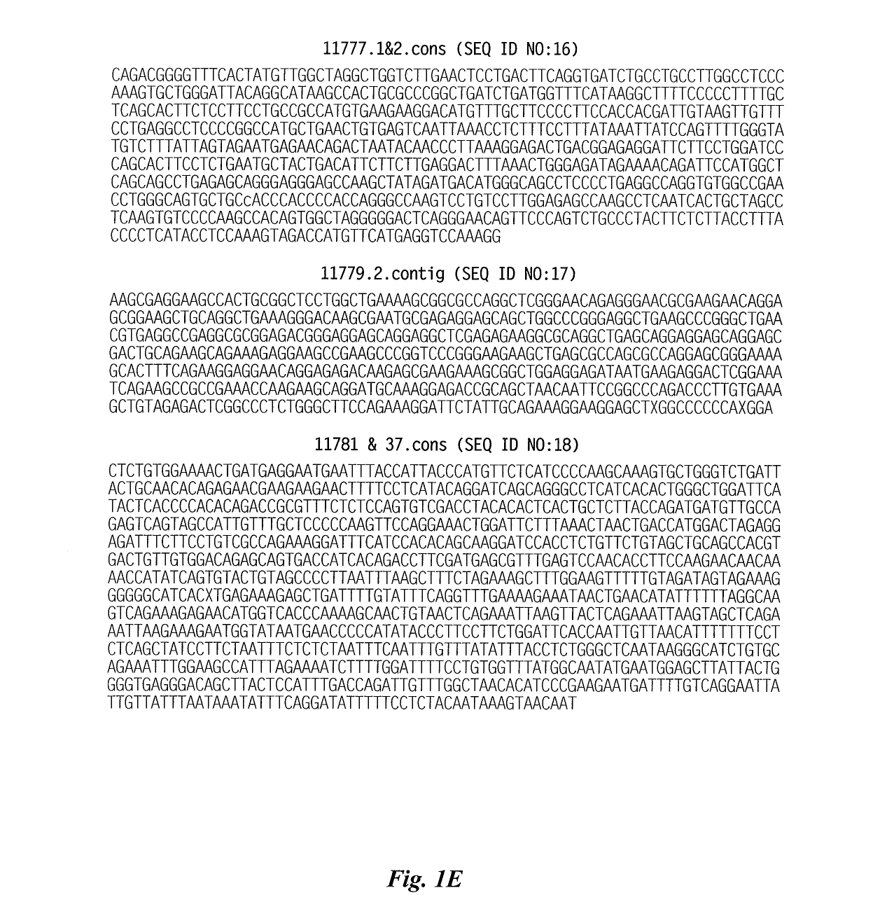

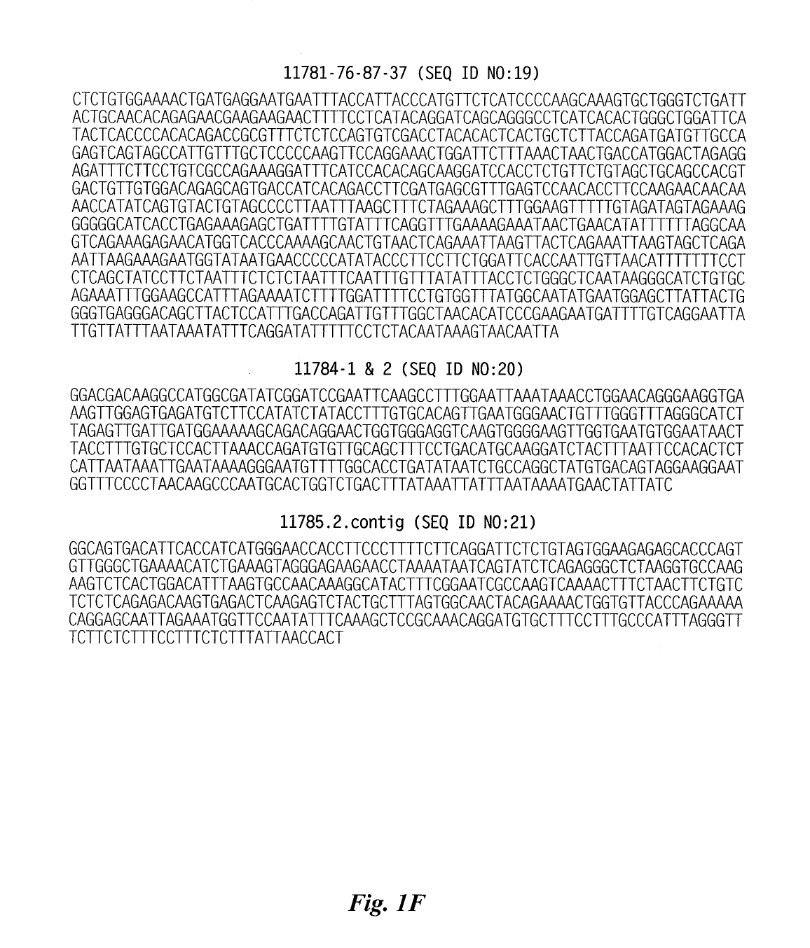

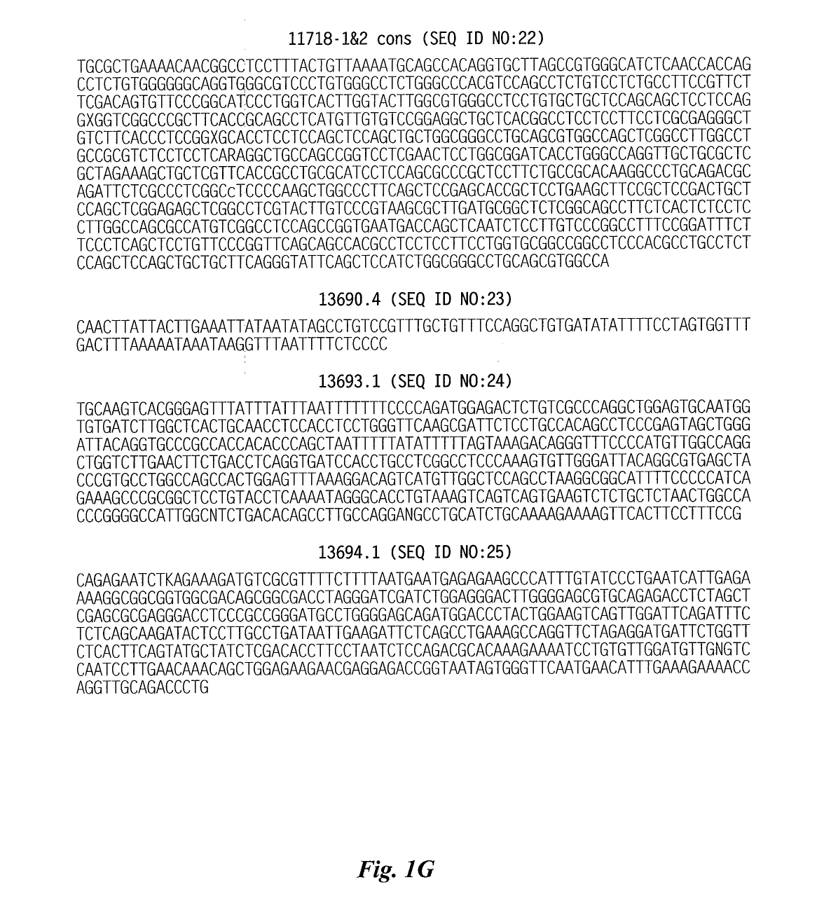

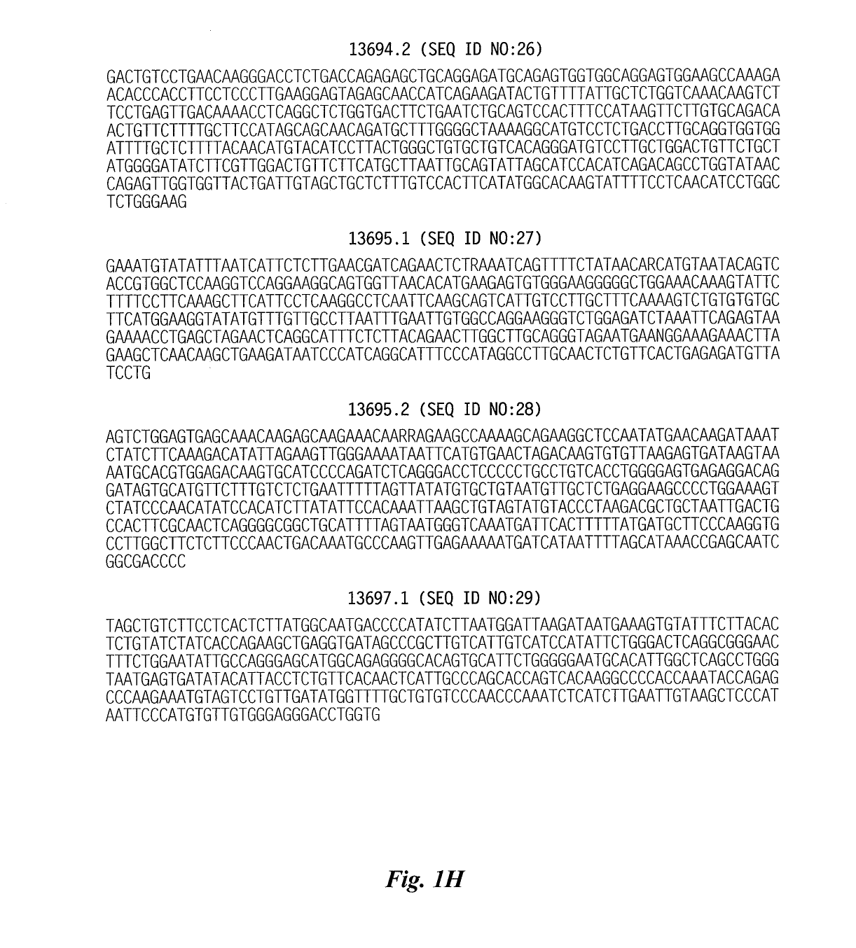

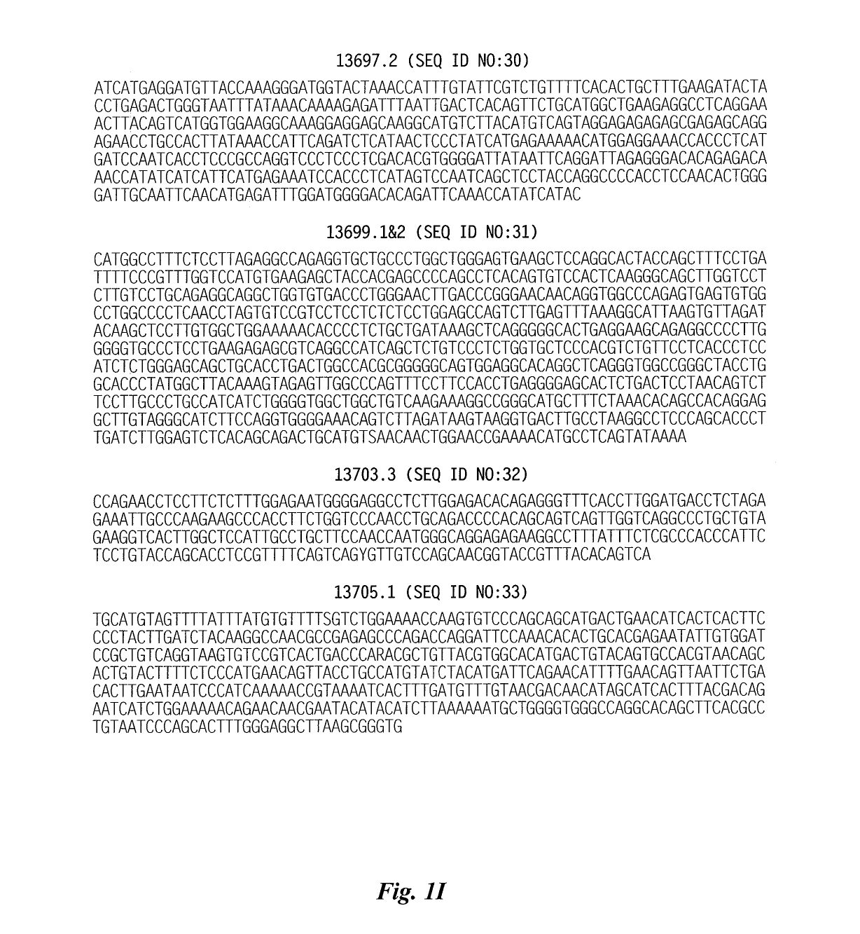

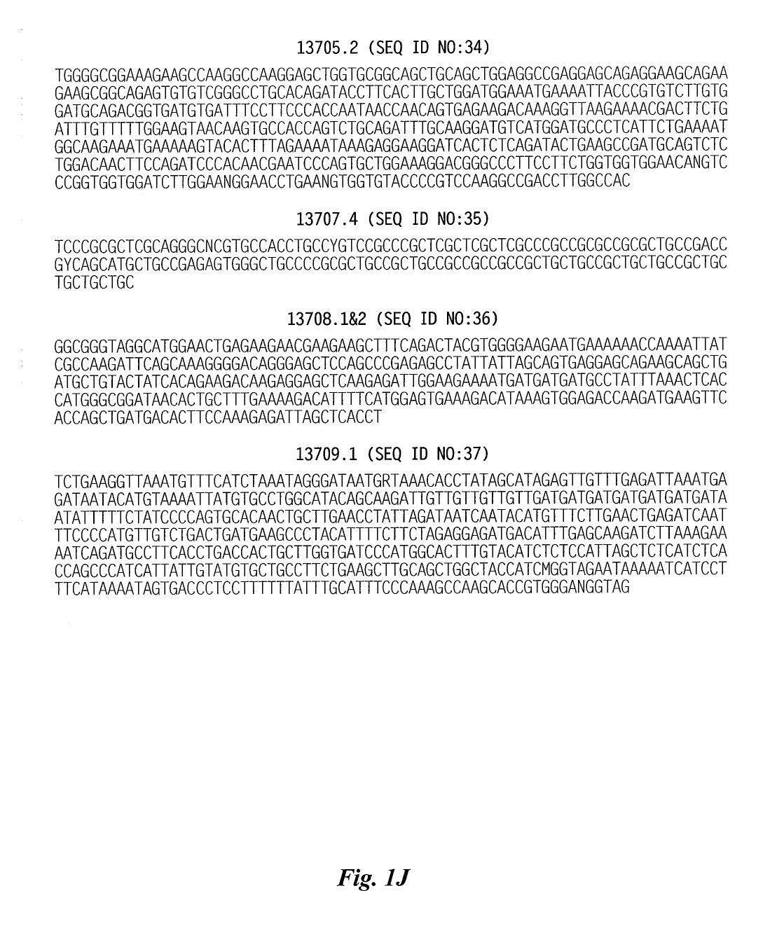

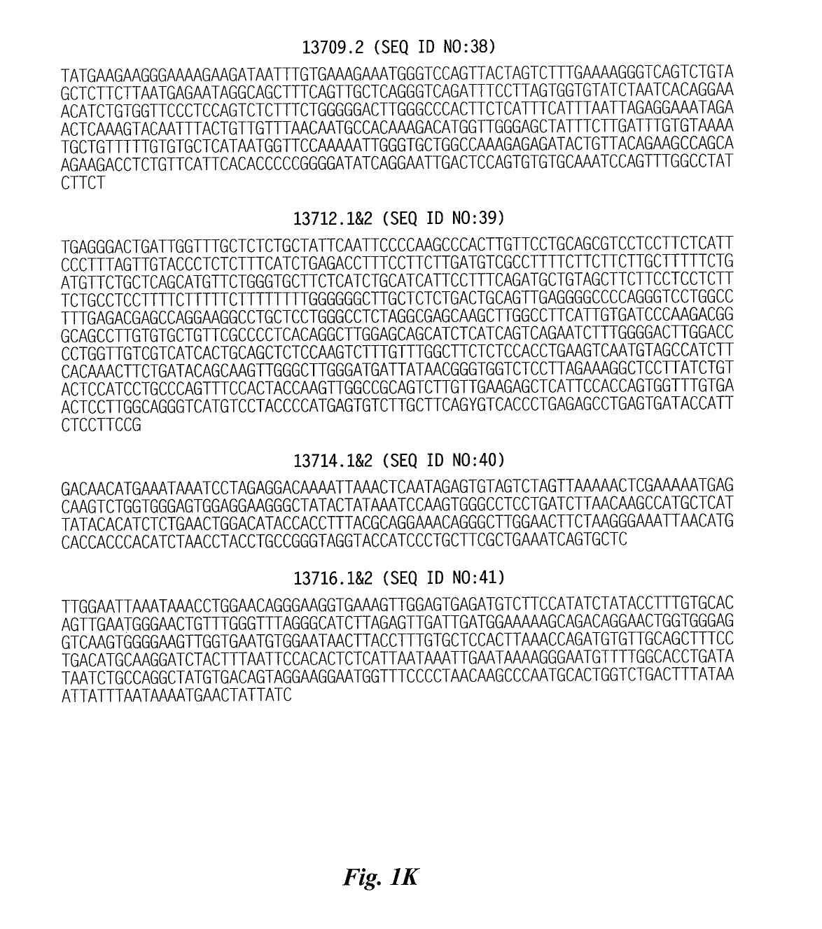

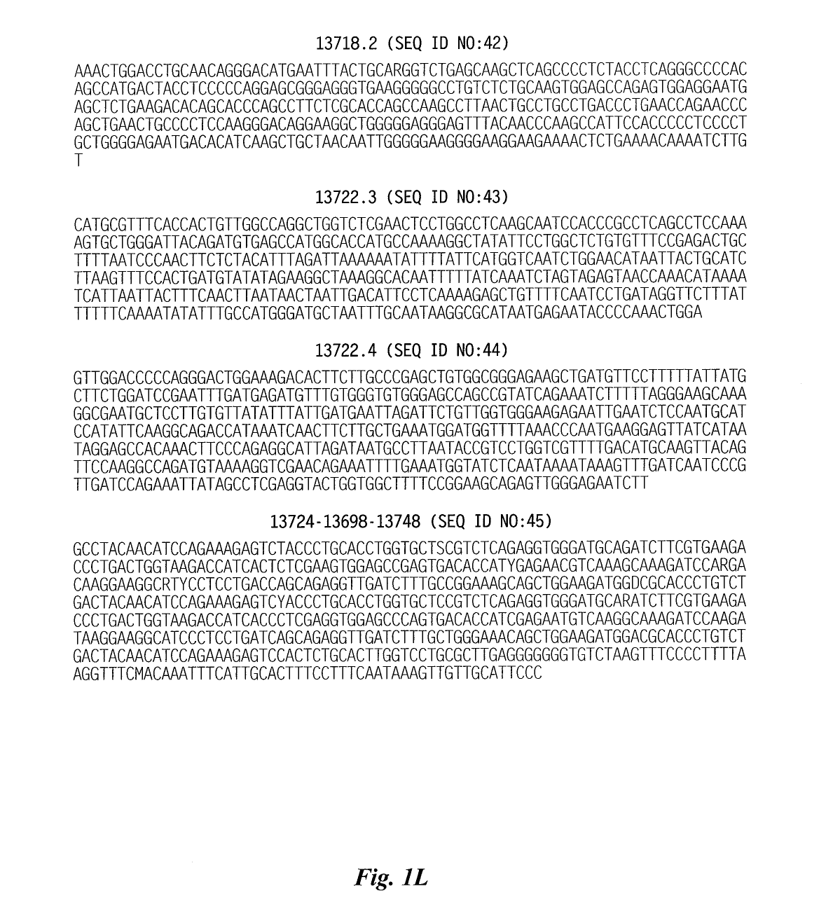

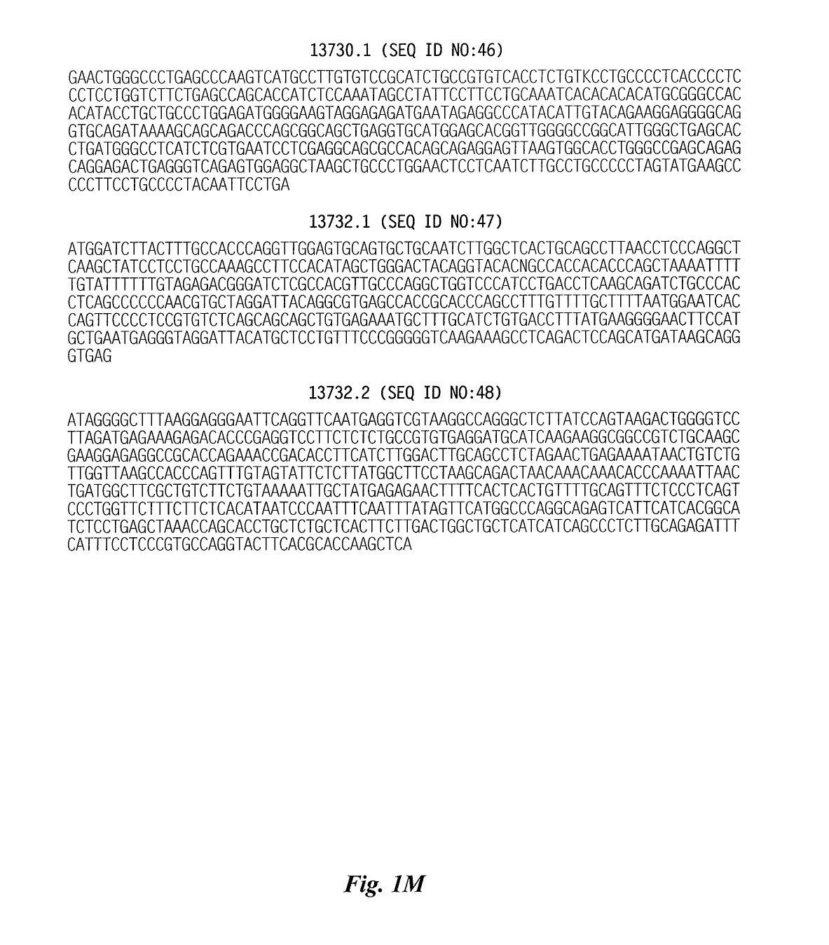

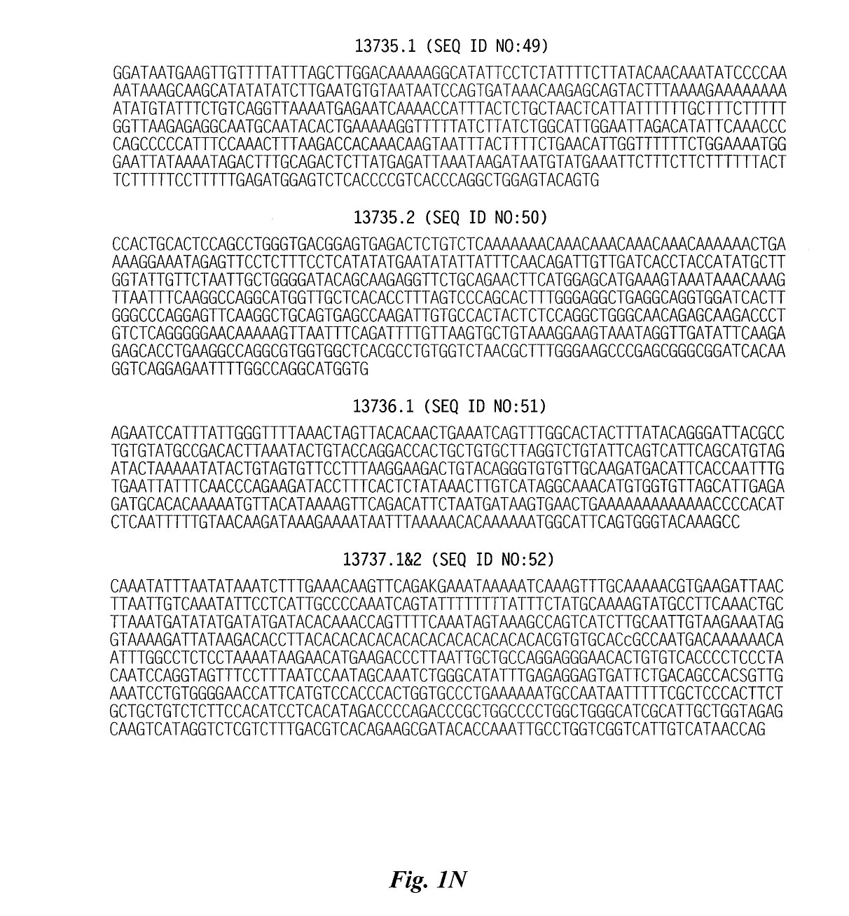

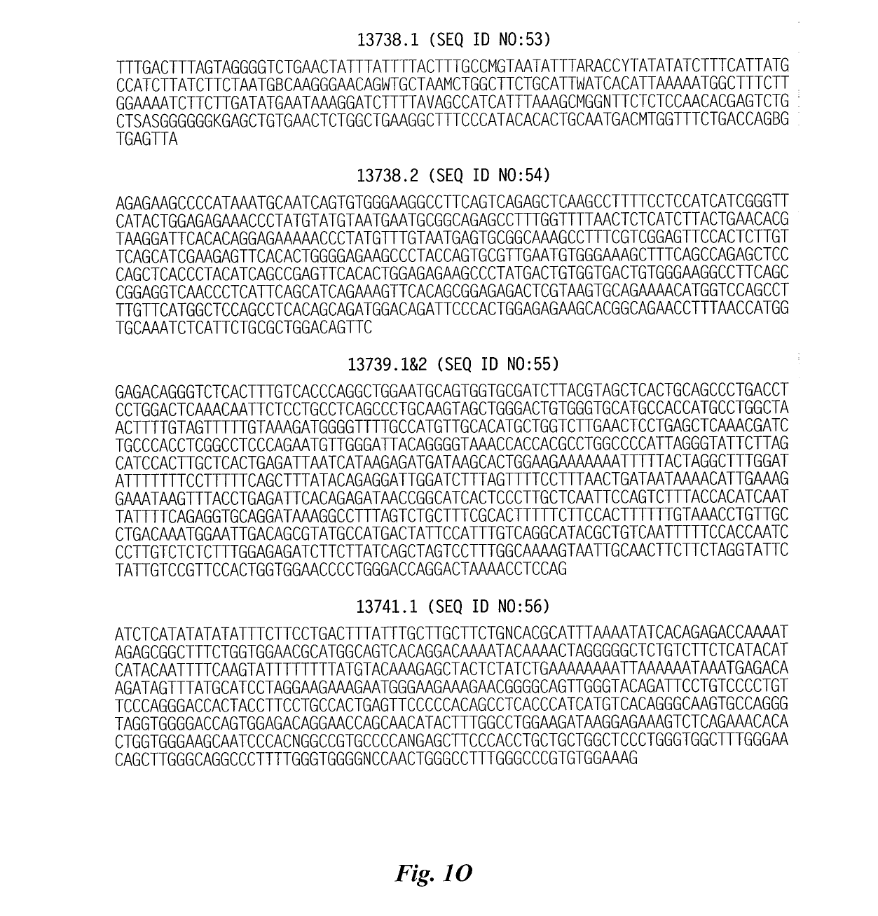

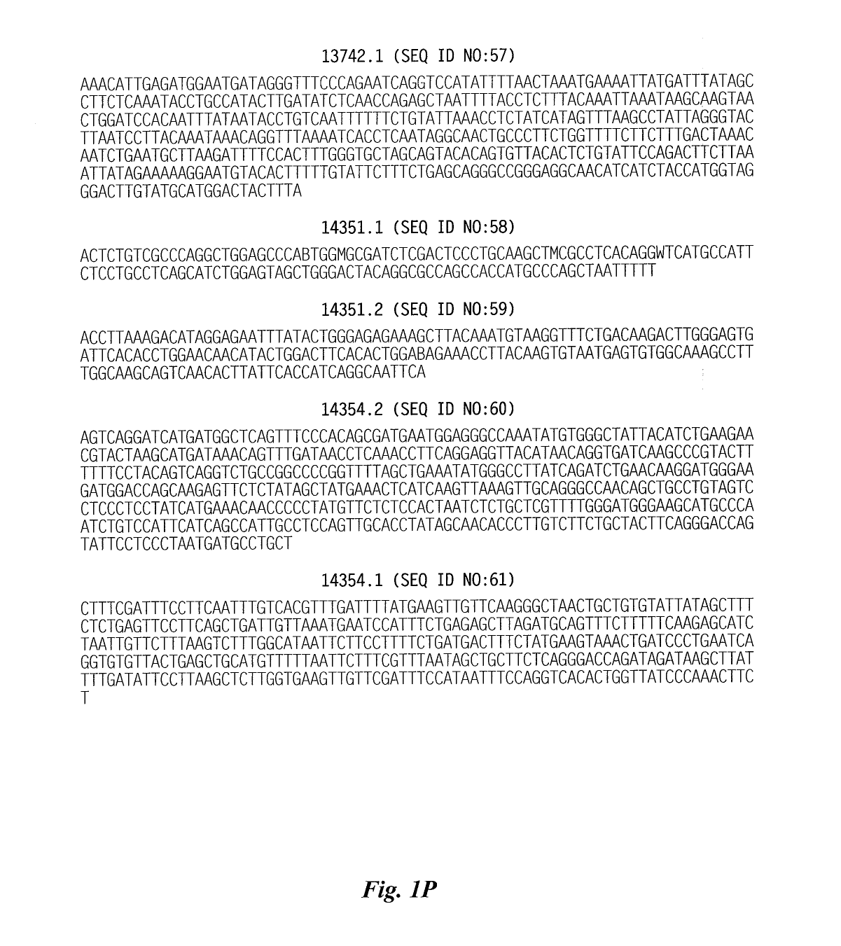

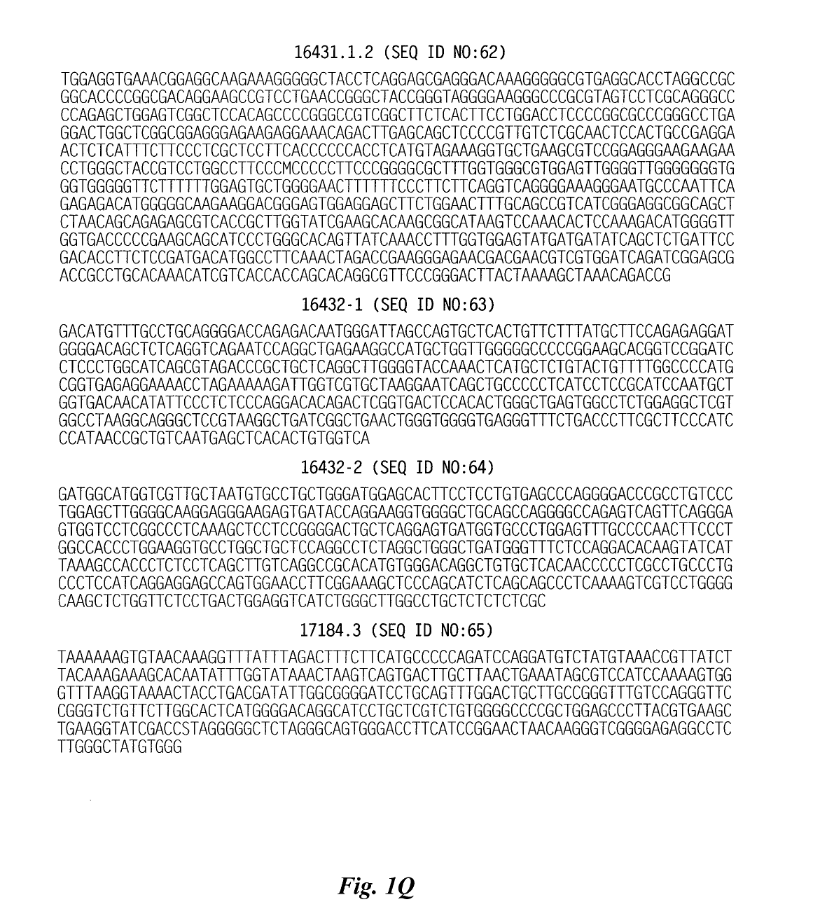

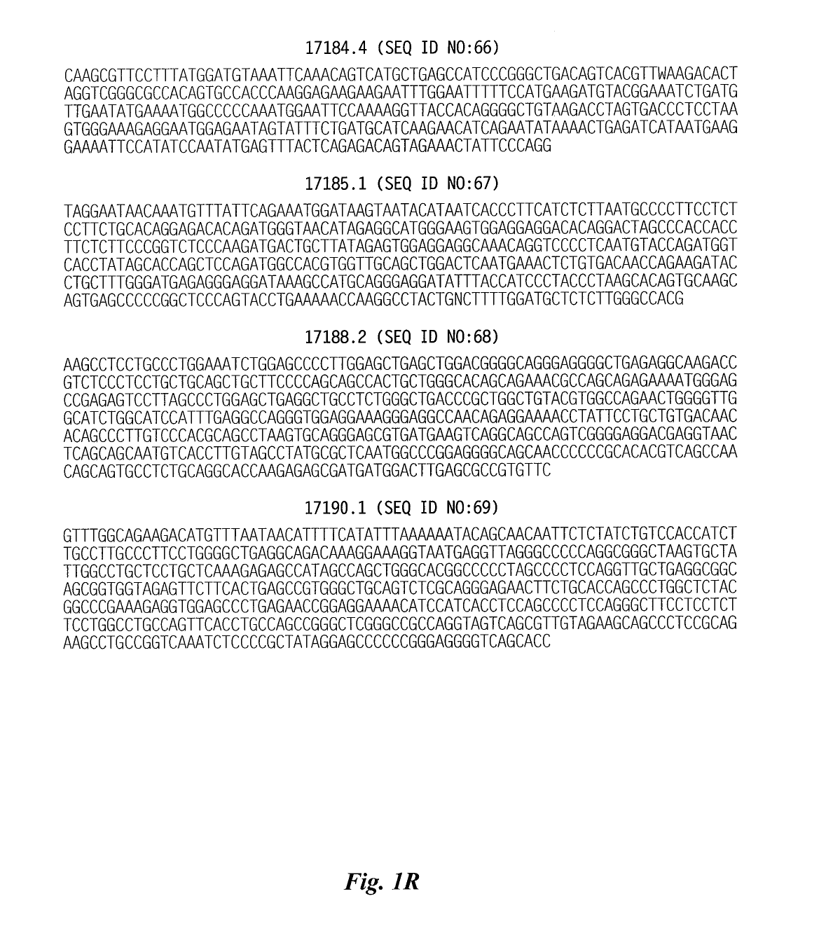

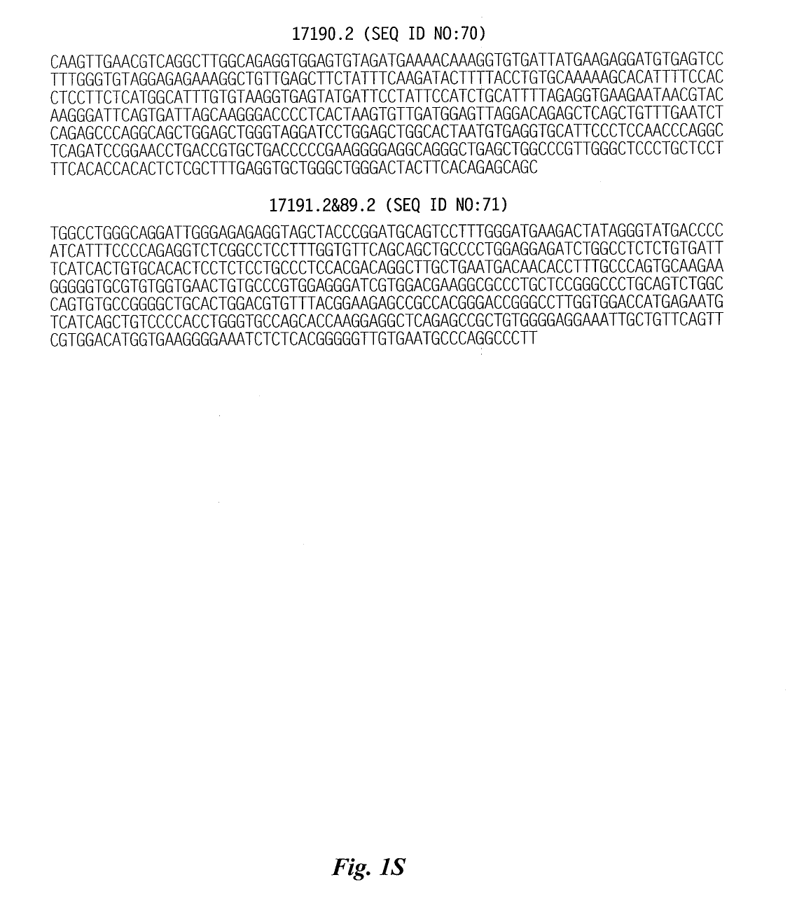

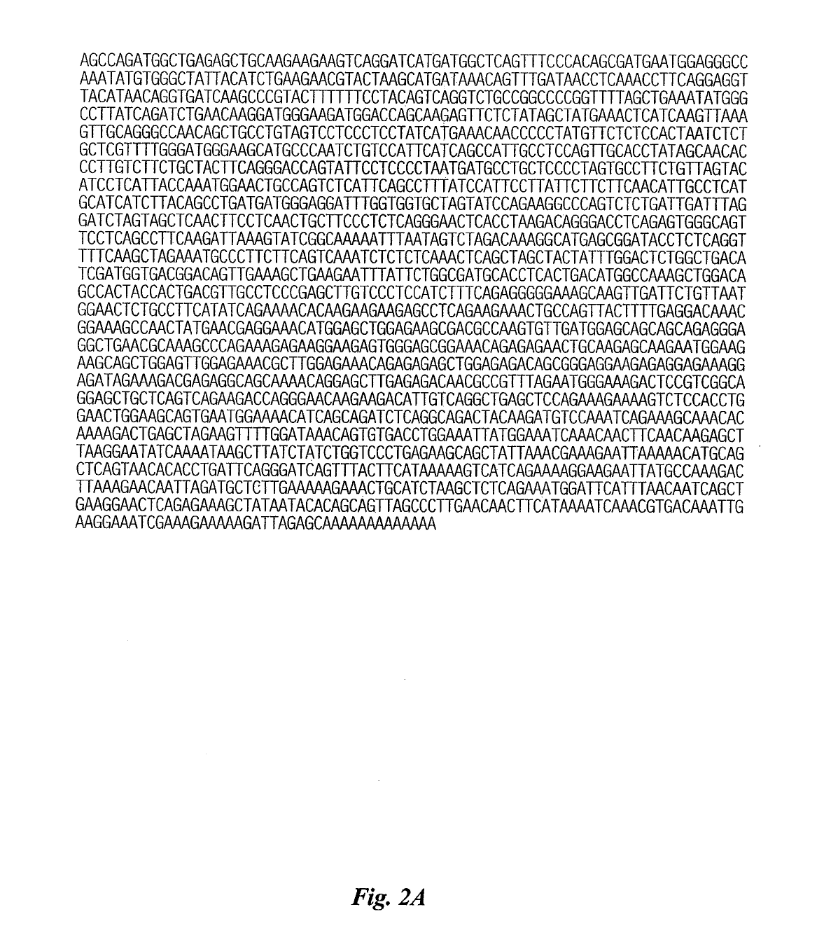

SEQ ID NO:1-71 are ovarian carcinoma antigen polynucleotides shown in FIGS. 1A-1S.

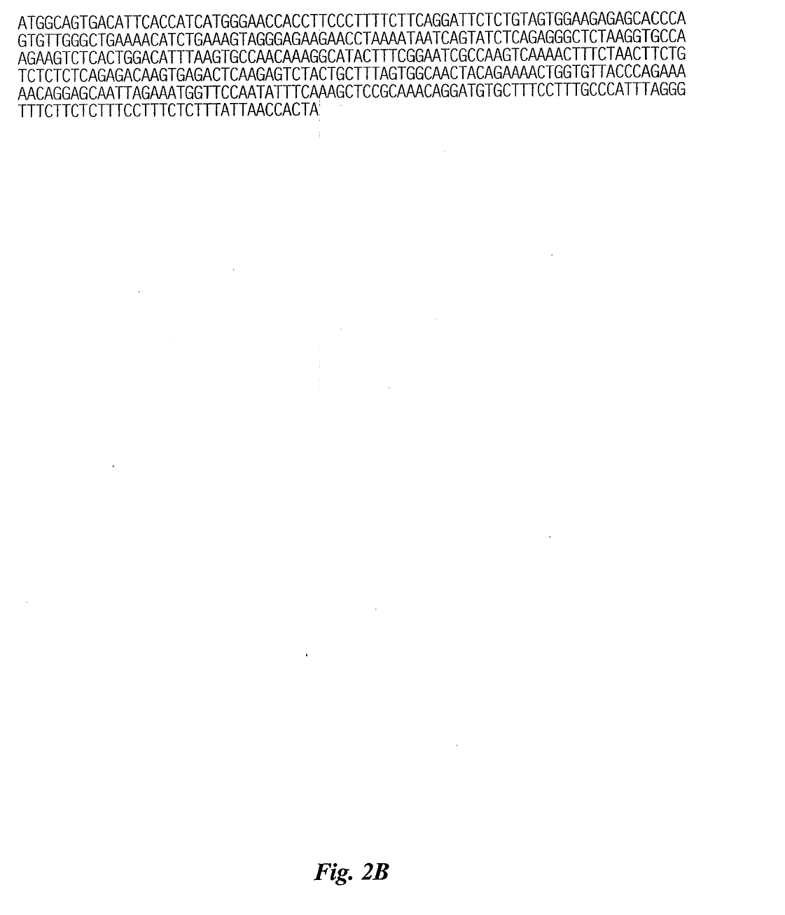

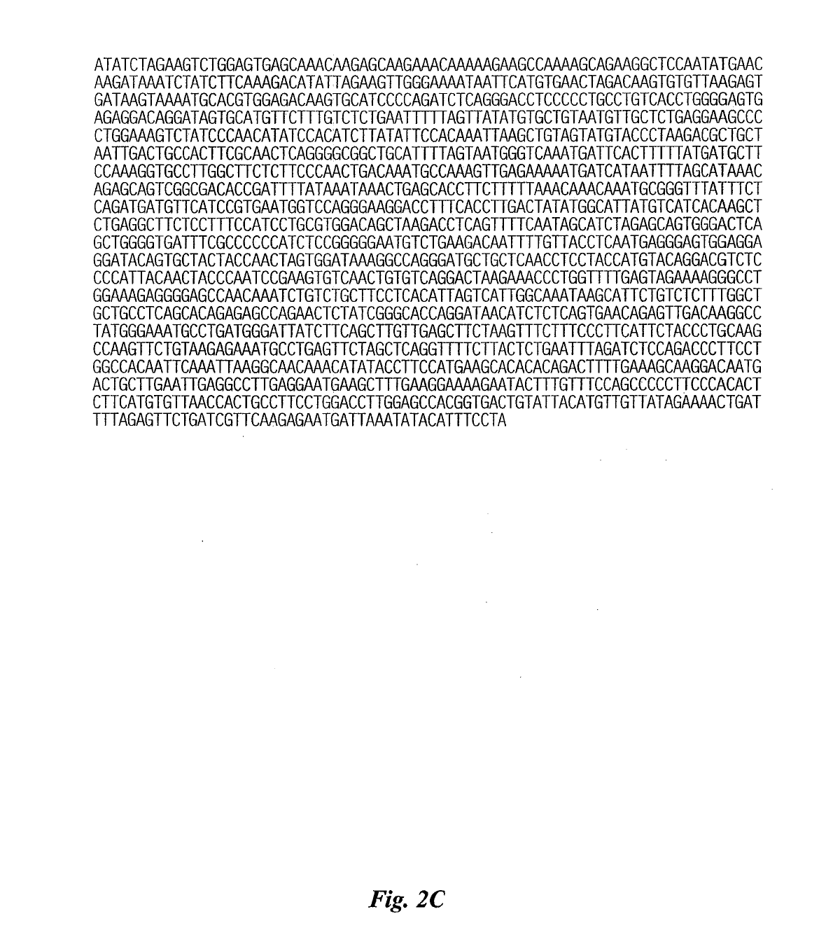

SEQ ID NO:72-74 are ovarian carcinoma antigen polynucleotides shown in FIGS. 2A-2C.

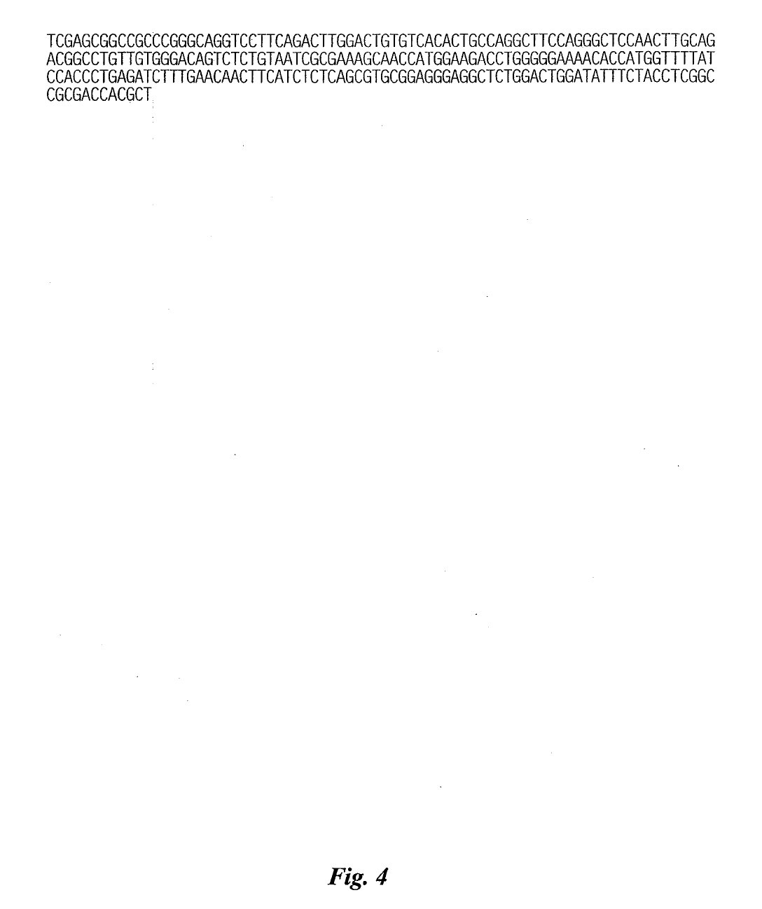

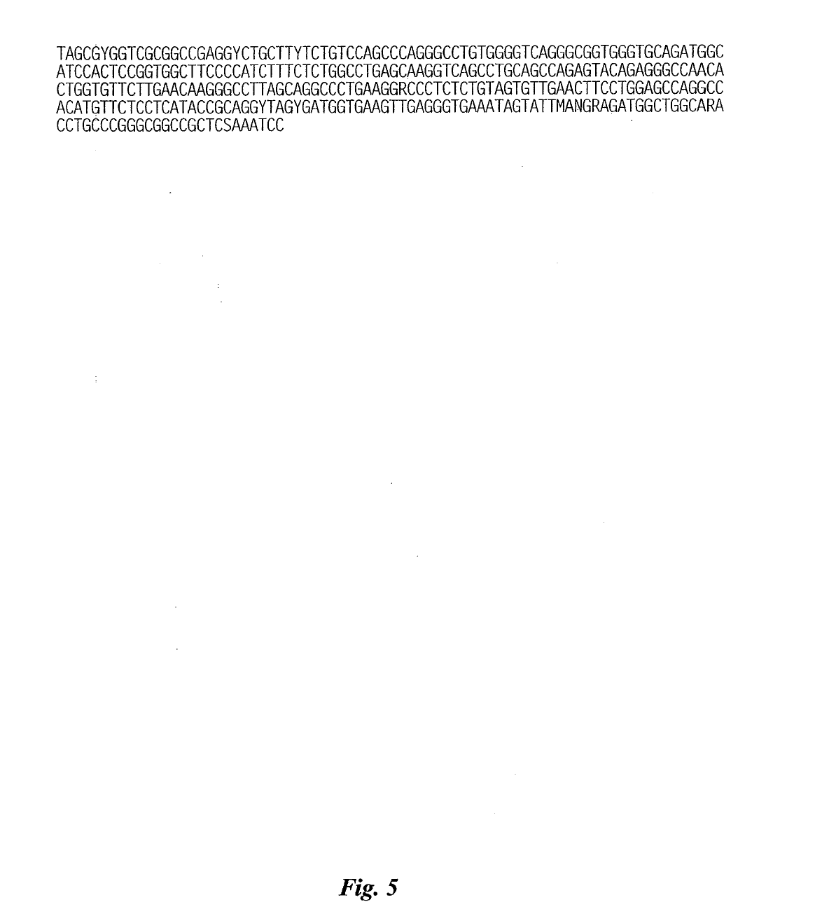

SEQ ID NO:75 is the ovarian carcinoma polynucleotide 3g (FIG. 4).

SEQ ID NO:76 is the ovarian carcinoma polynucleotide 3f (FIG. 5).

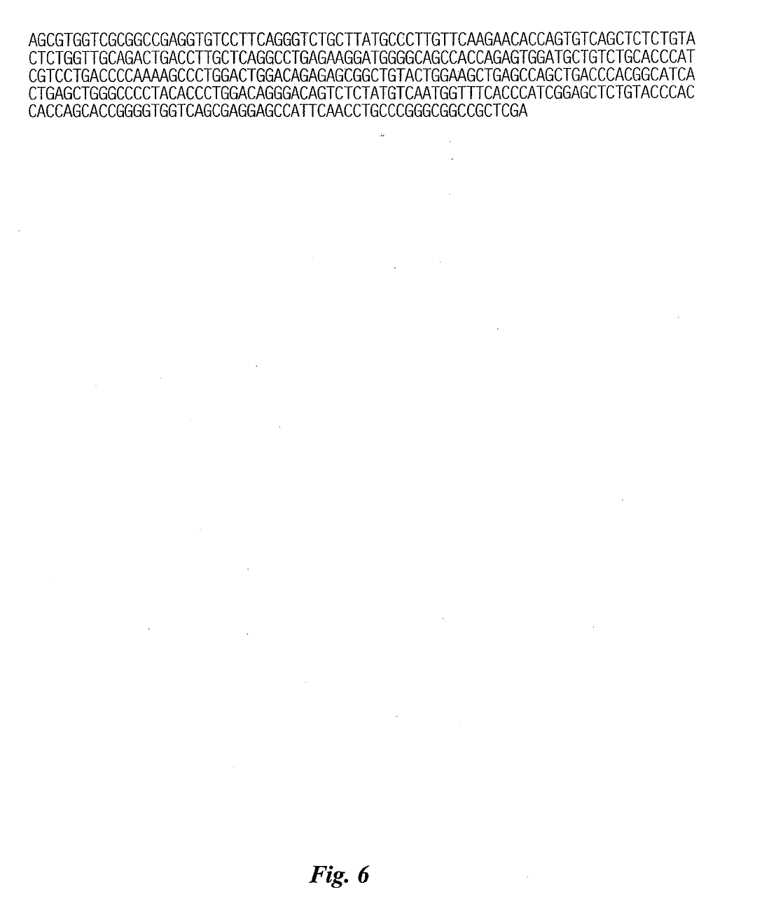

SEQ ID NO:77 is the ovarian carcinoma polynucleotide 6b (FIG. 6).

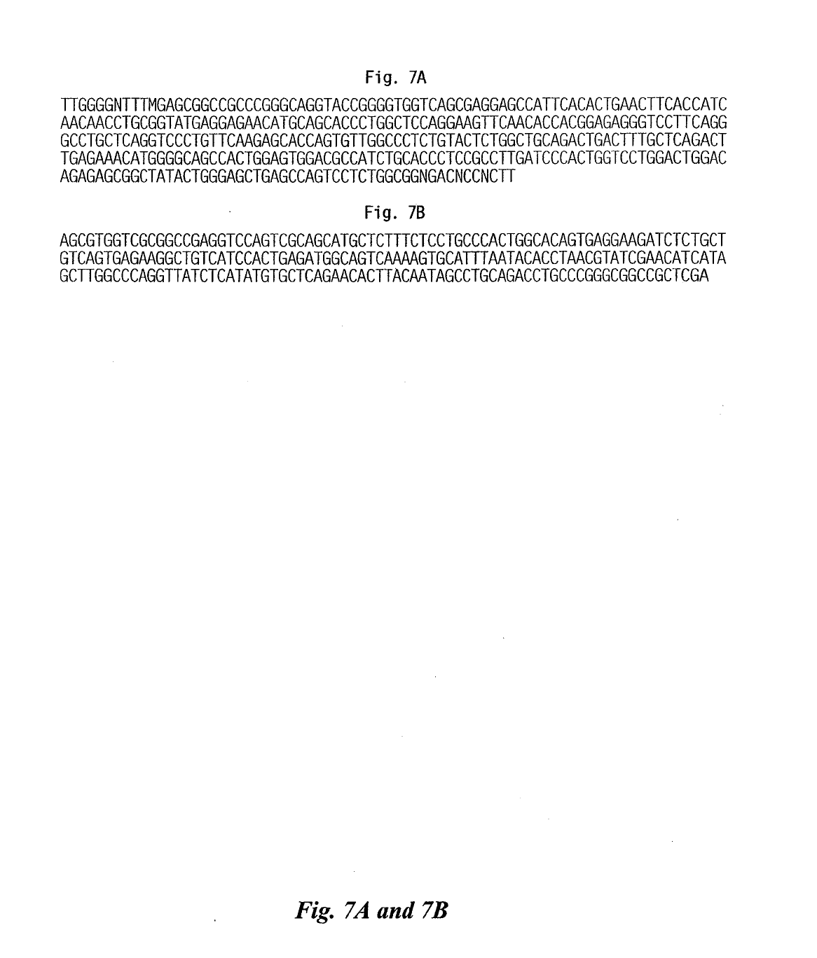

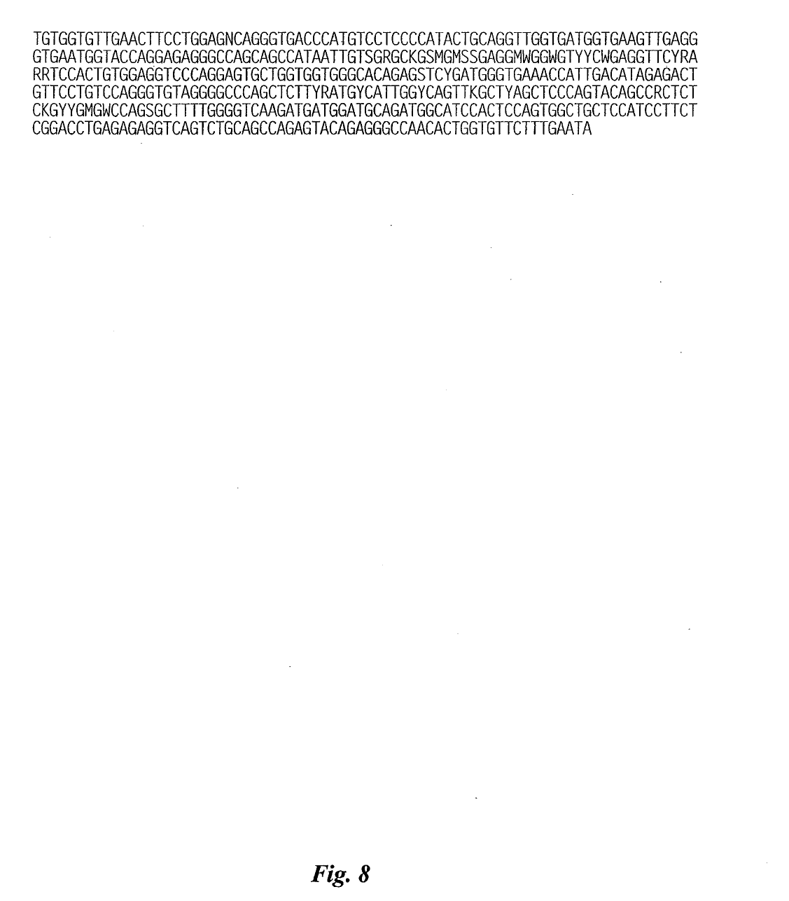

SEQ ID NO:78 is the ovarian carcinoma polynucleotide 8e (FIG. 7A).

SEQ ID NO:79 is the ovarian carcinoma polynucleotide 8h (FIG. 7B).

SEQ ID NO:80 is the ovarian carcinoma polynucleotide 12e (FIG. 8).

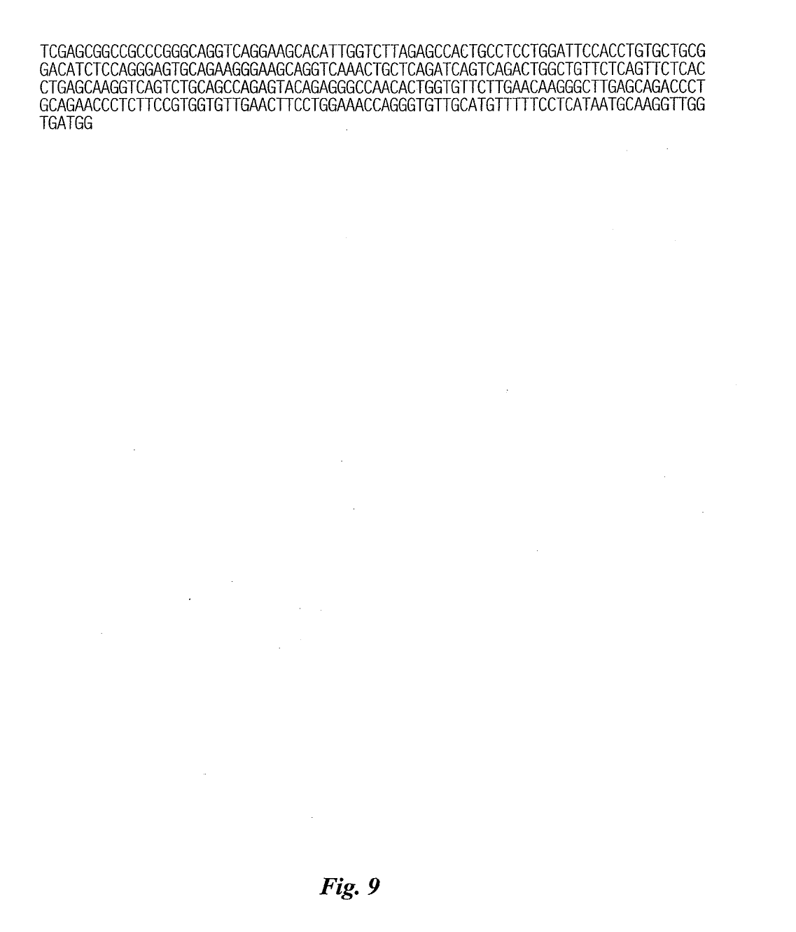

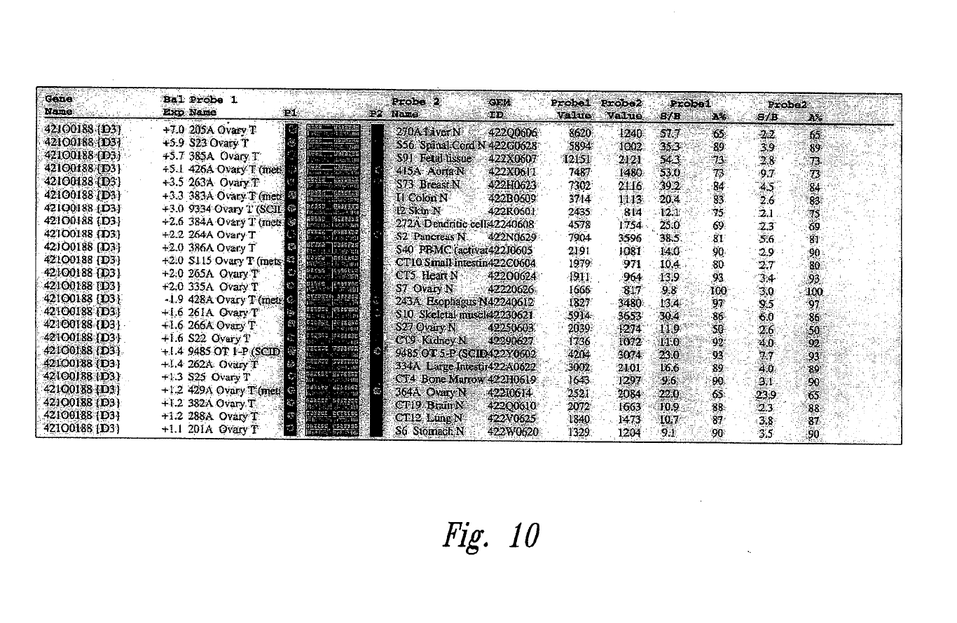

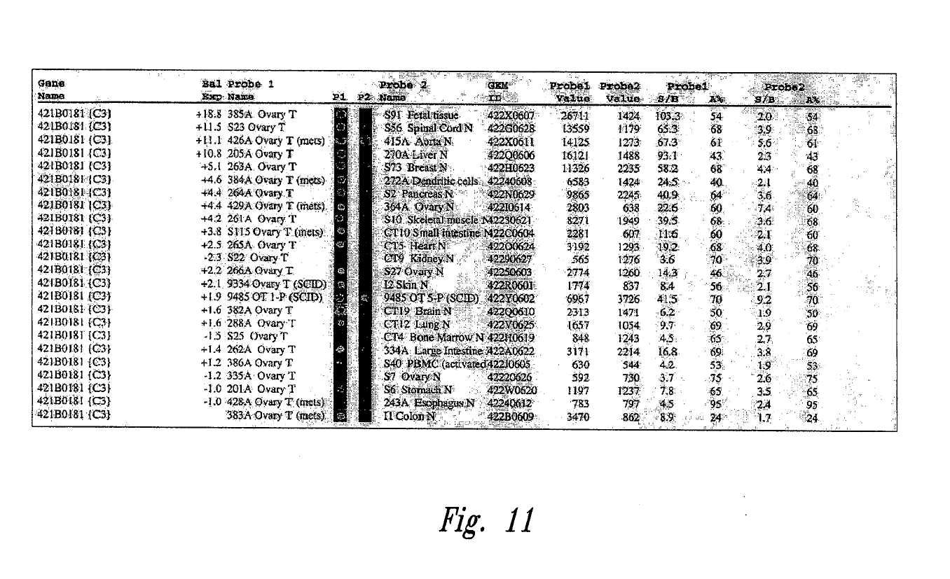

SEQ ID NO:81 is the ovarian carcinoma polynucleotide 12h (FIG. 9).

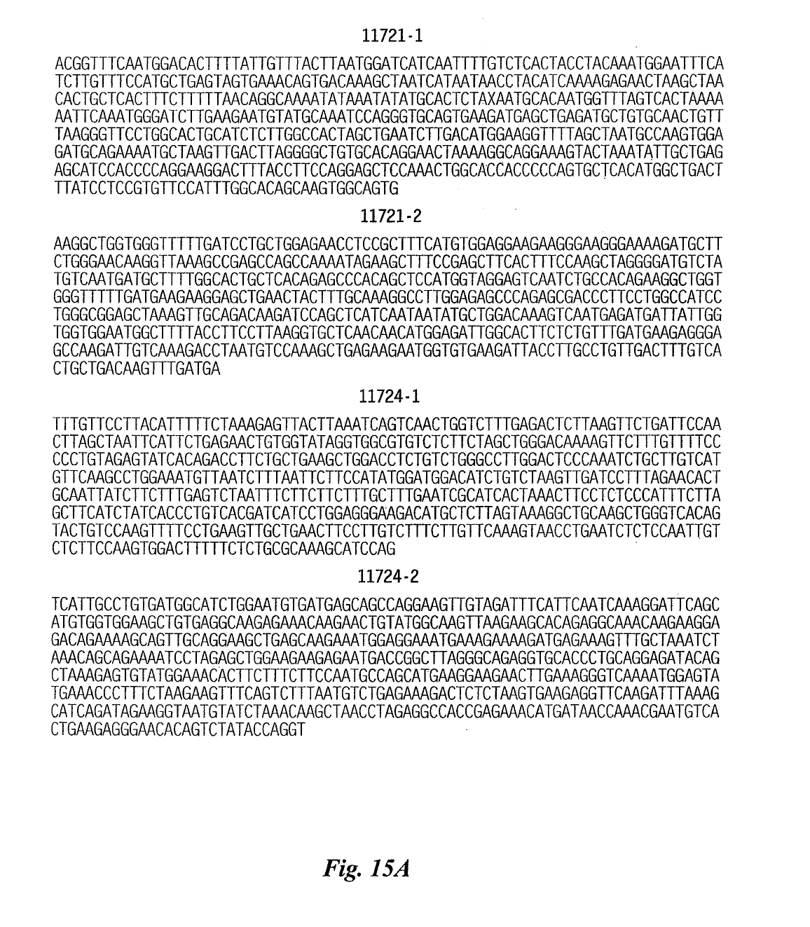

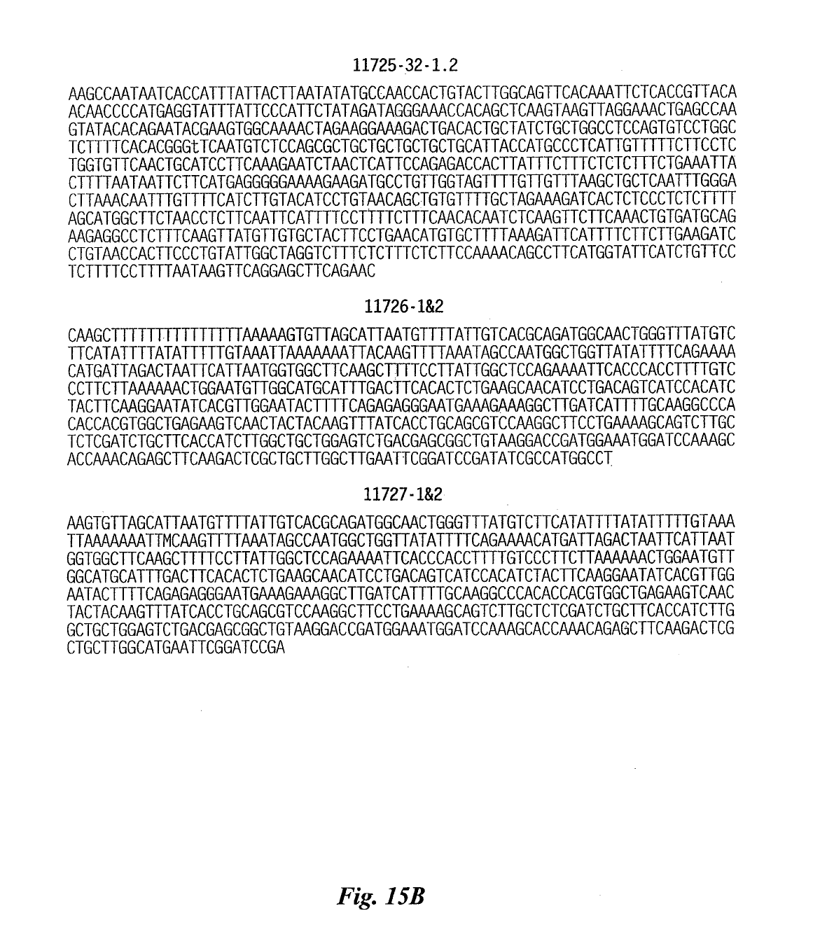

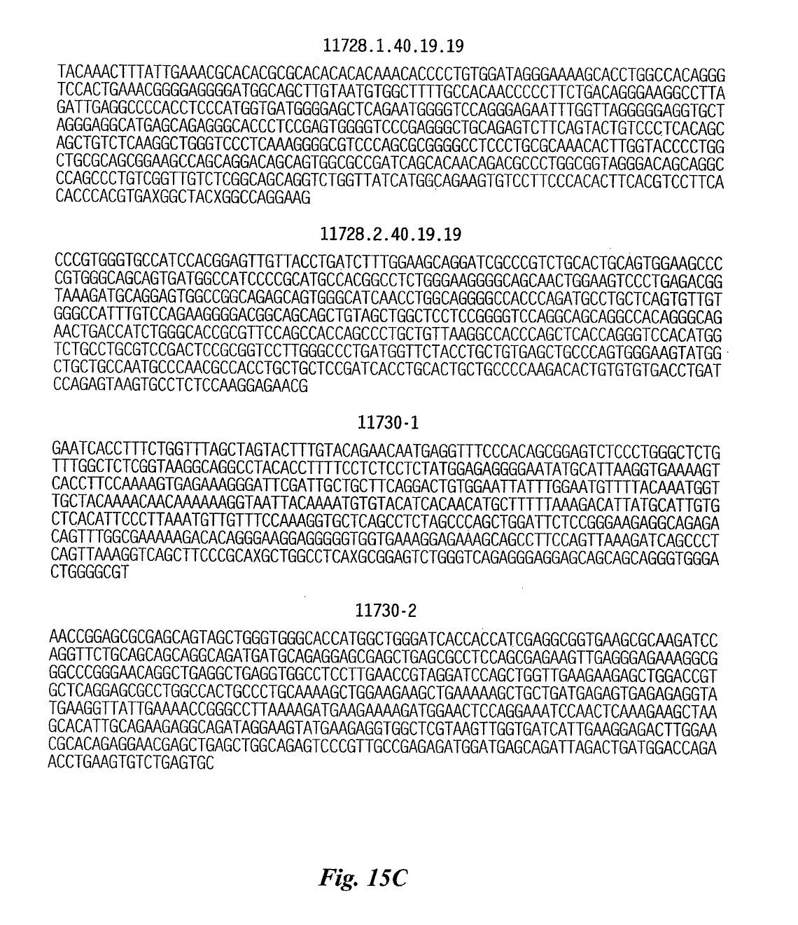

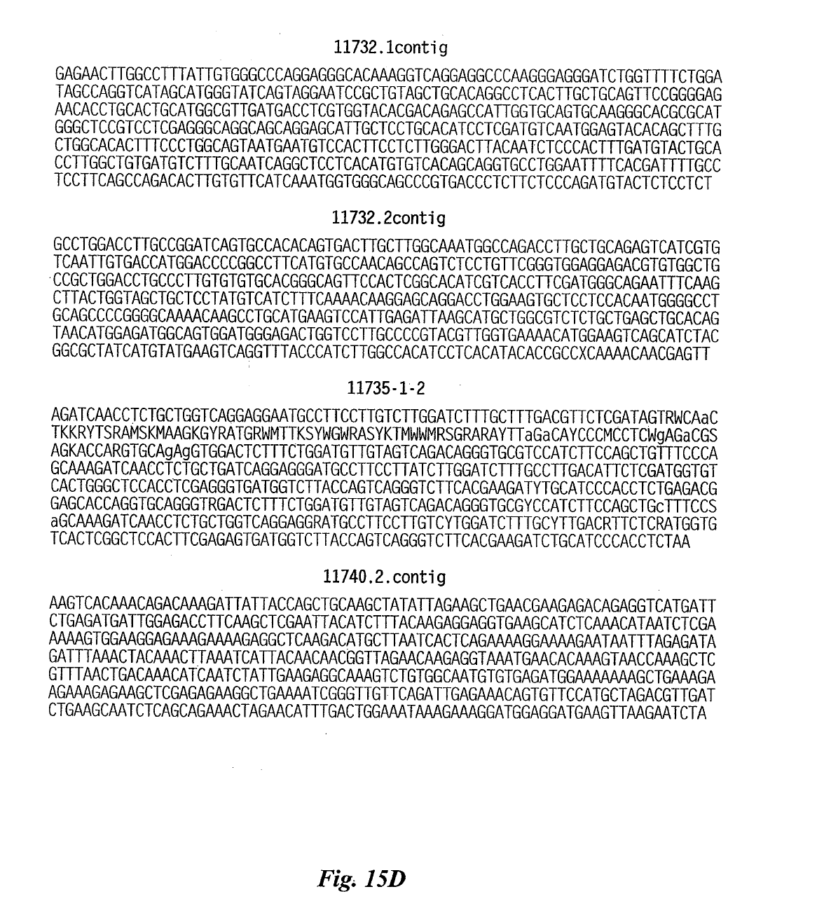

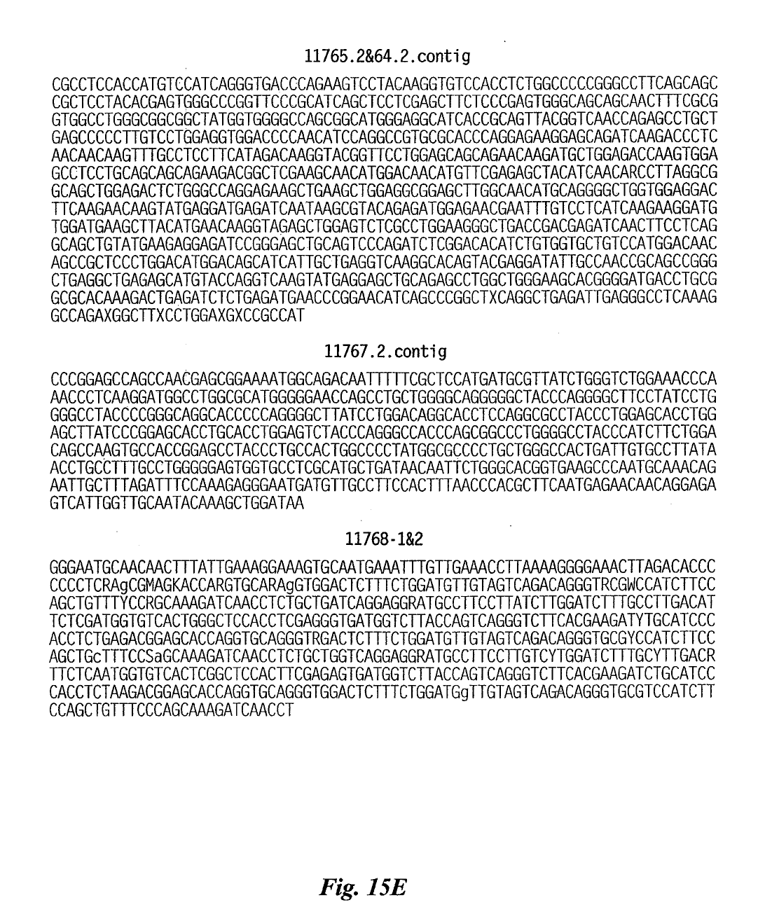

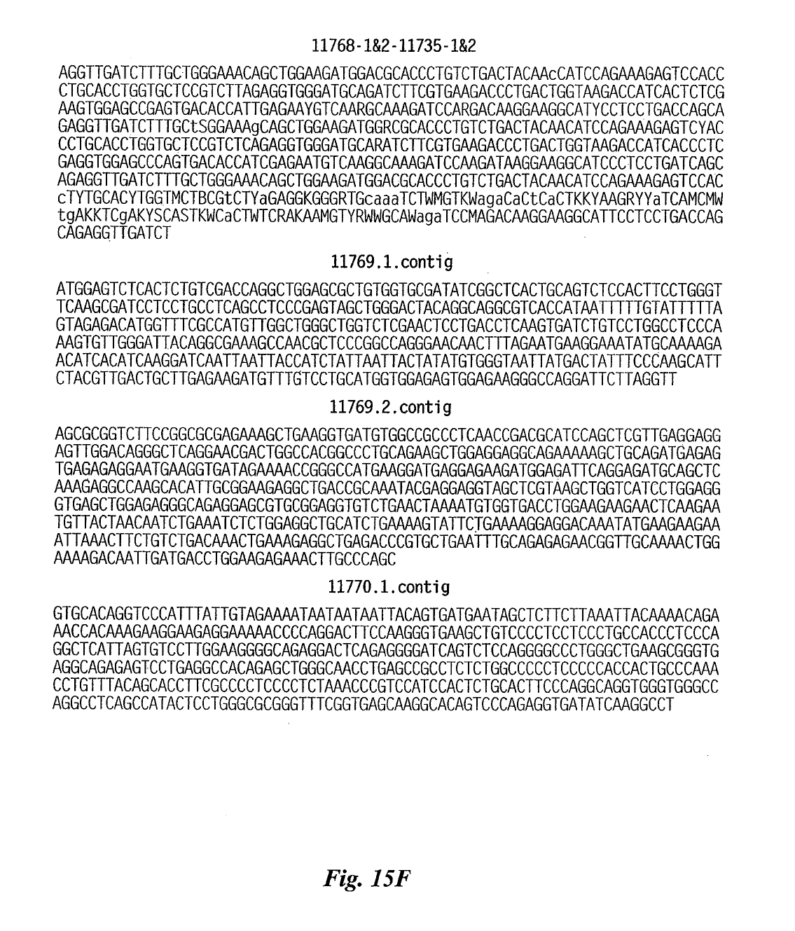

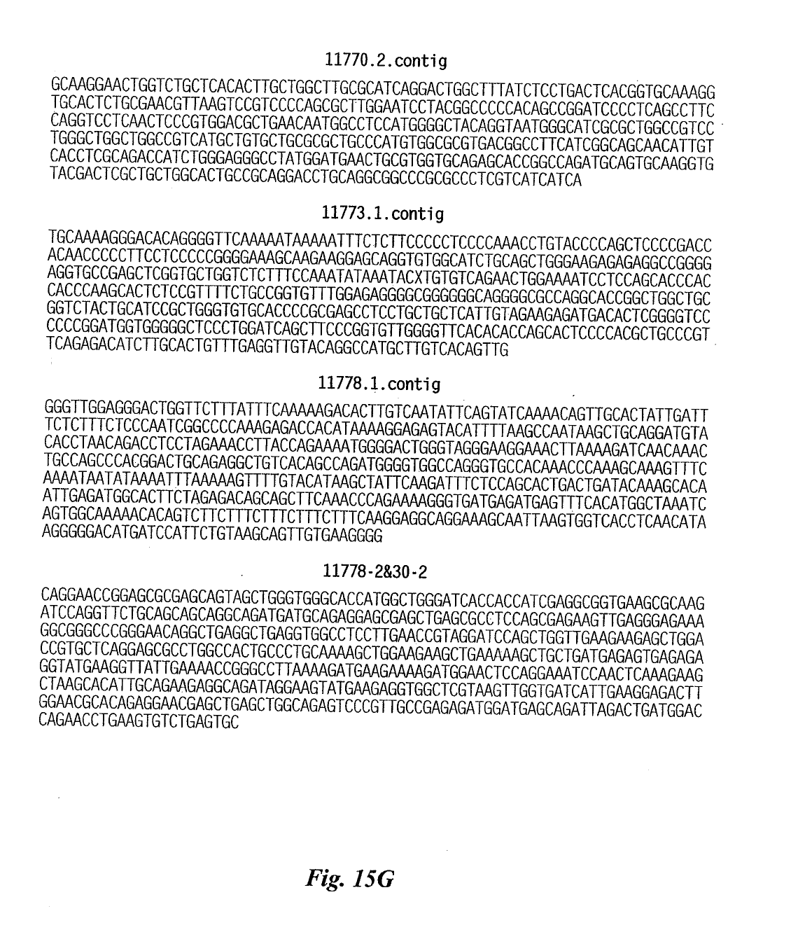

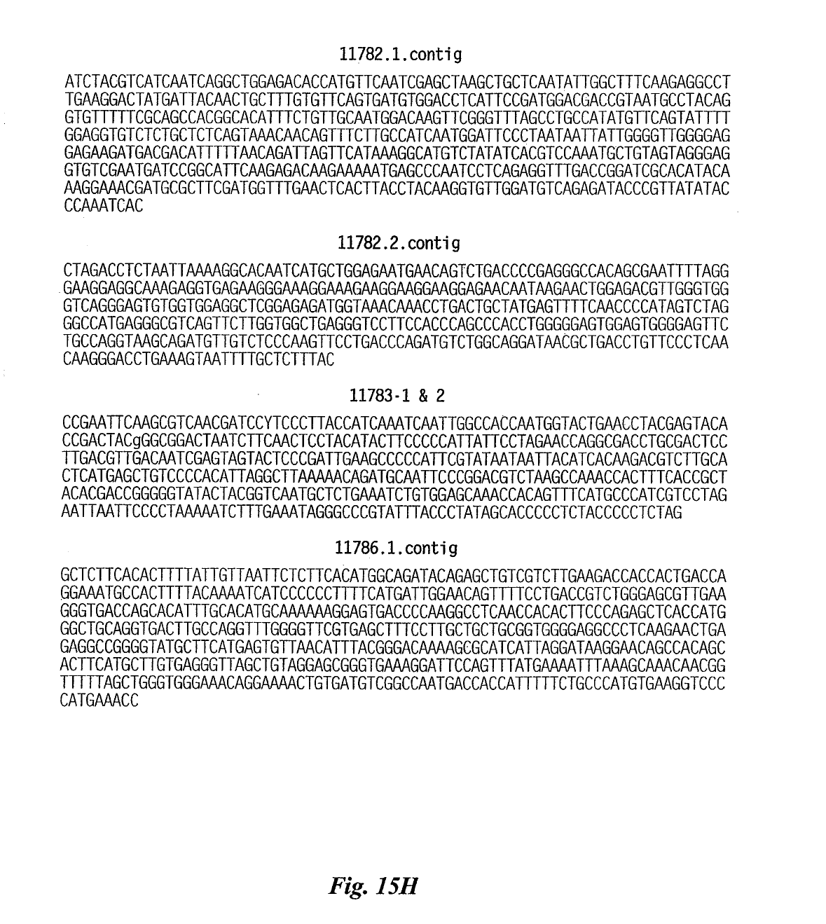

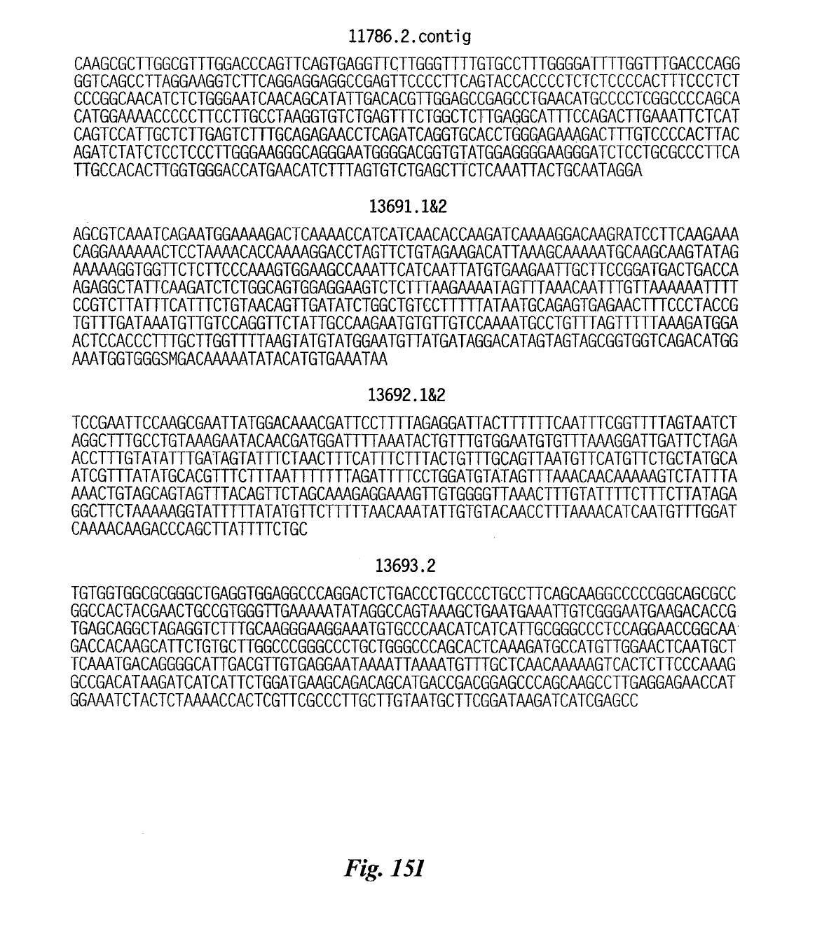

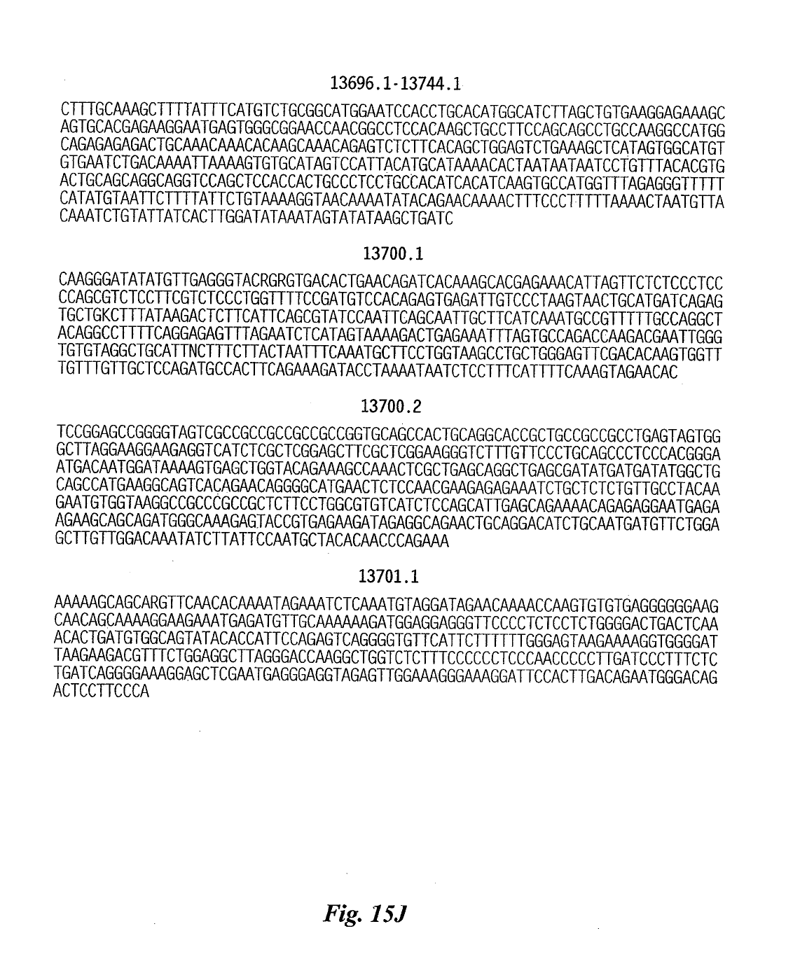

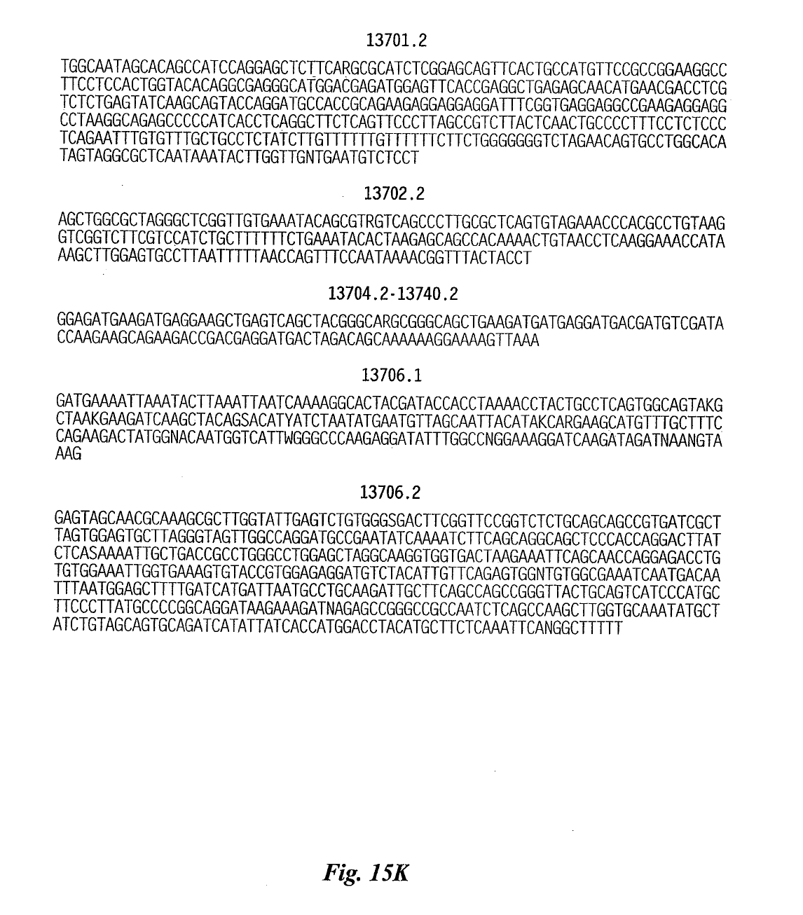

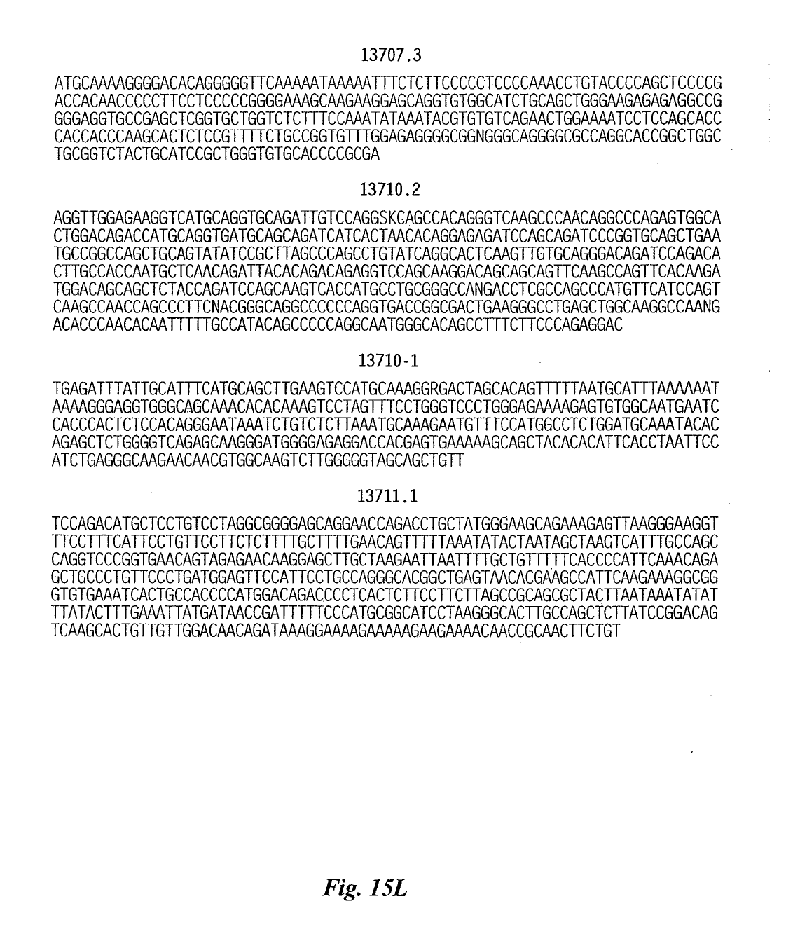

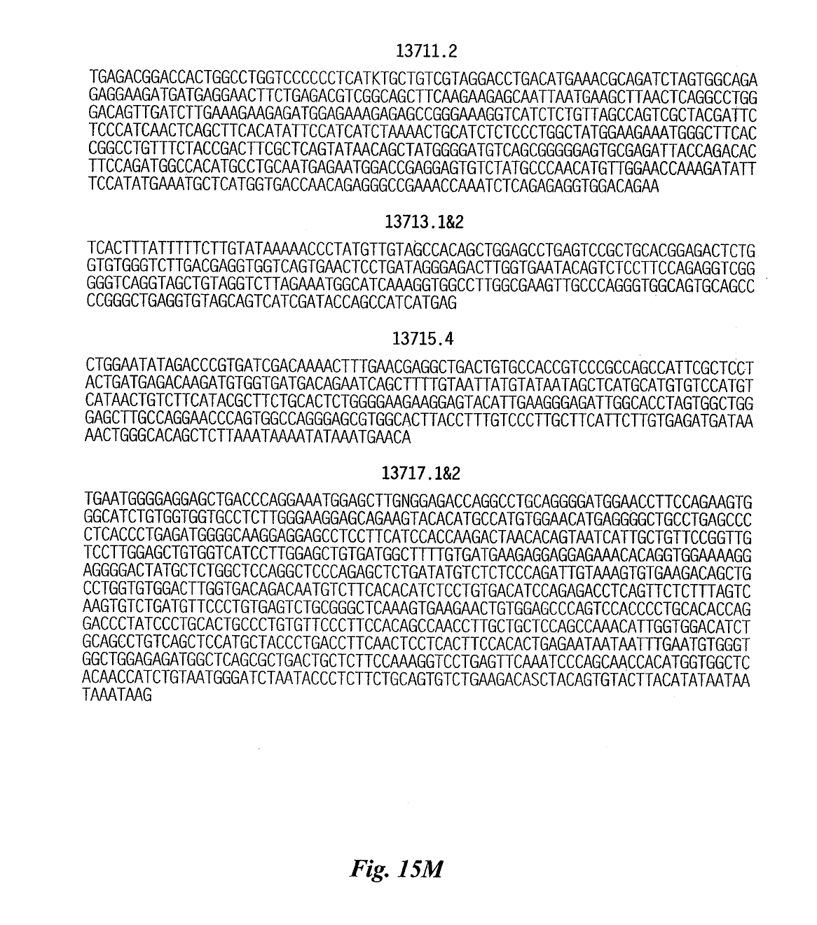

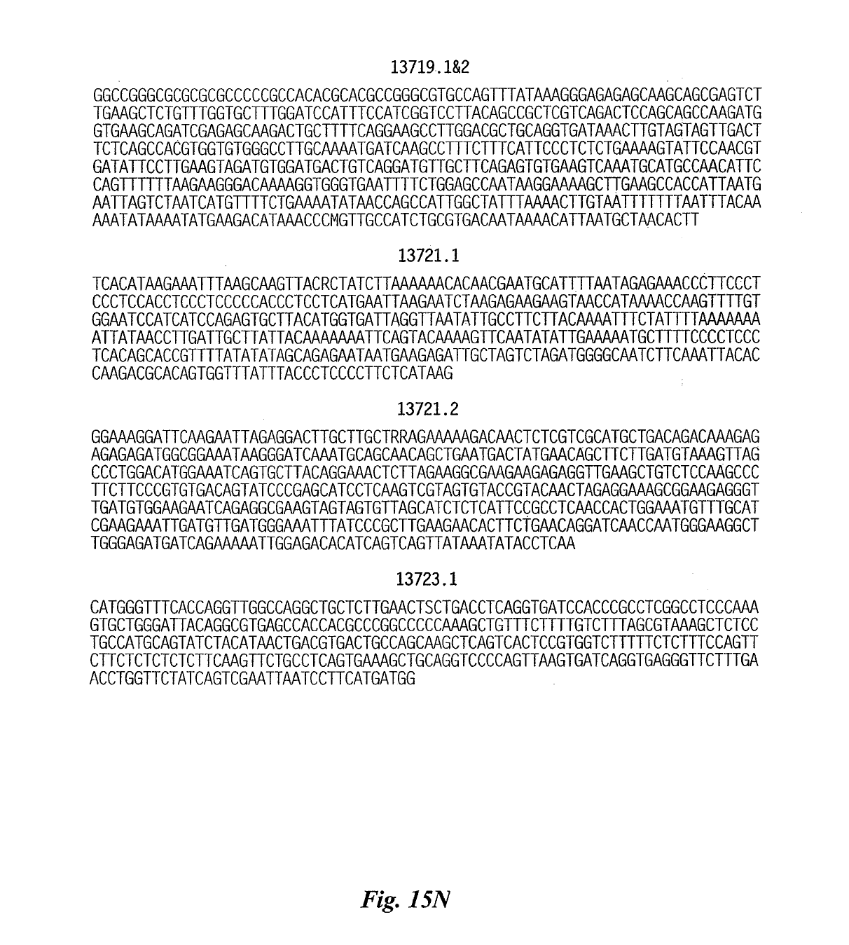

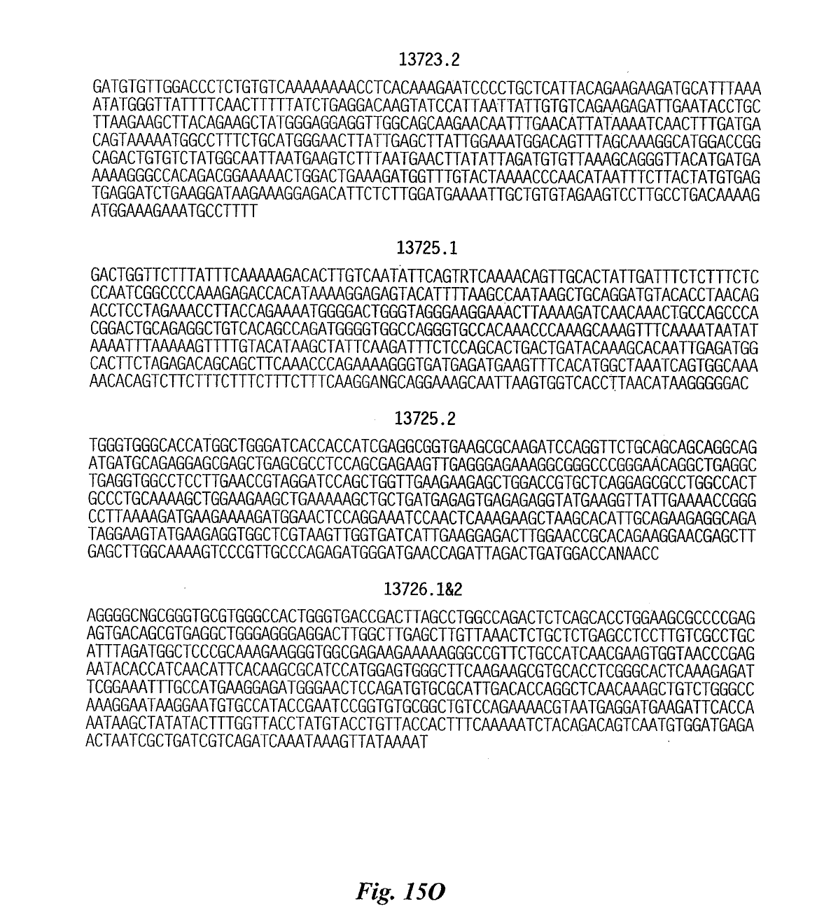

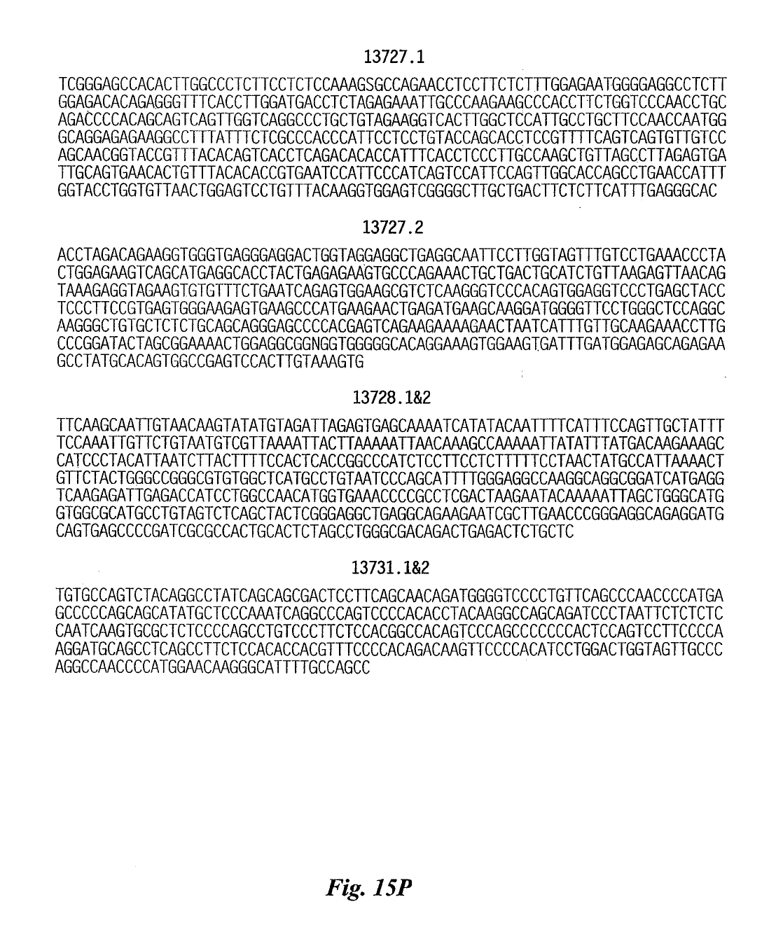

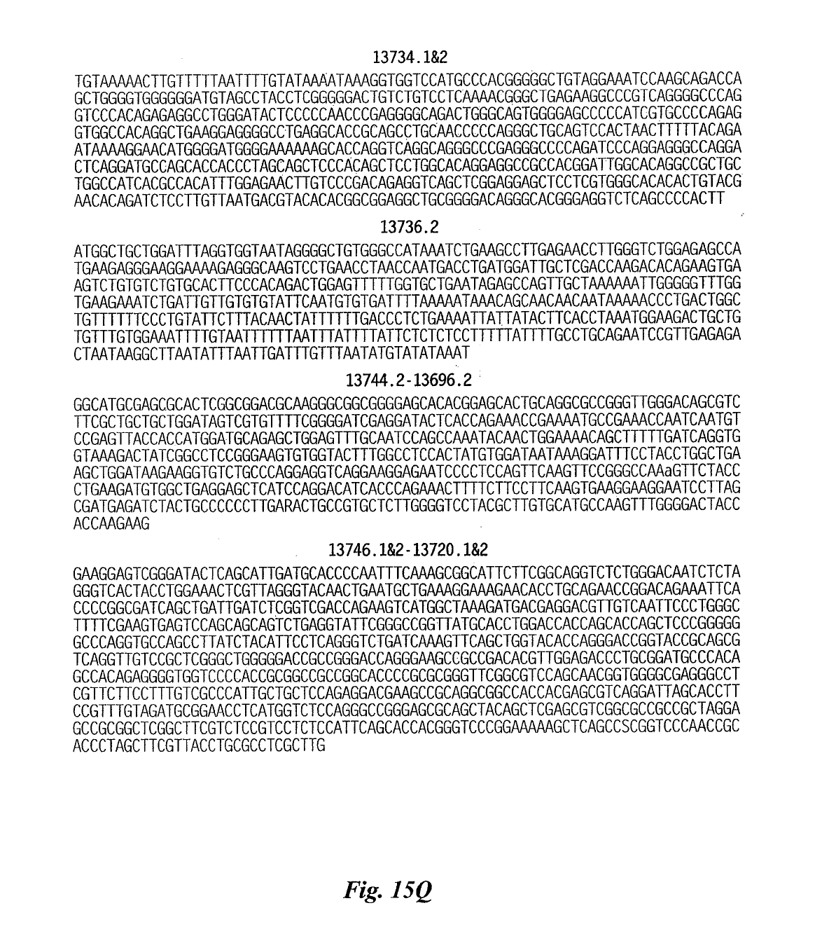

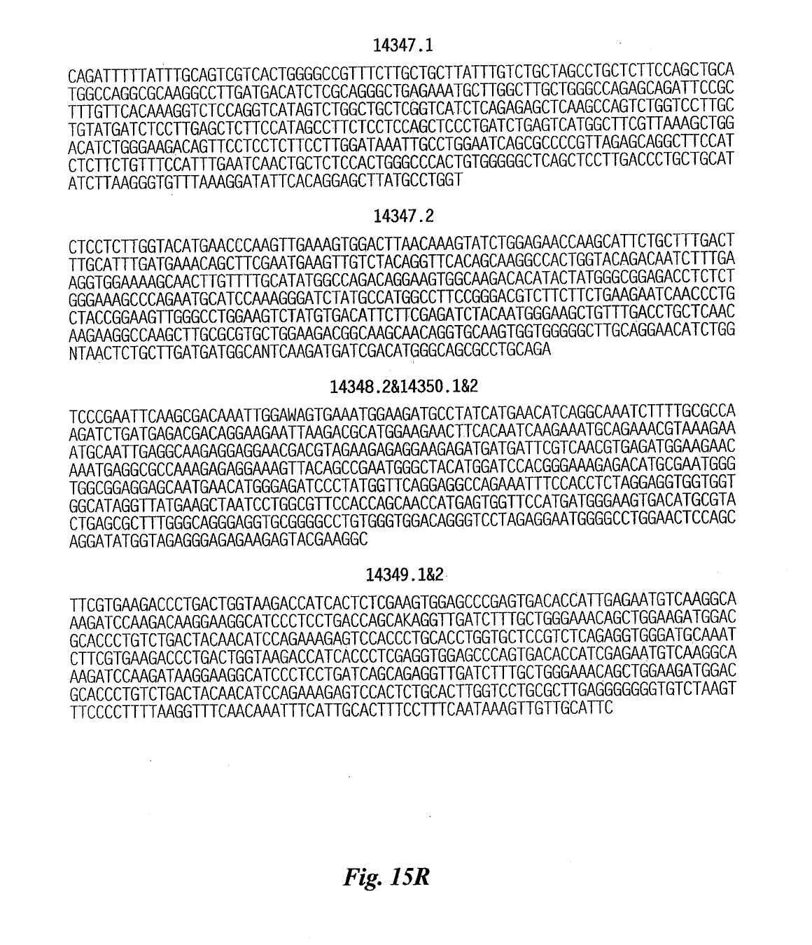

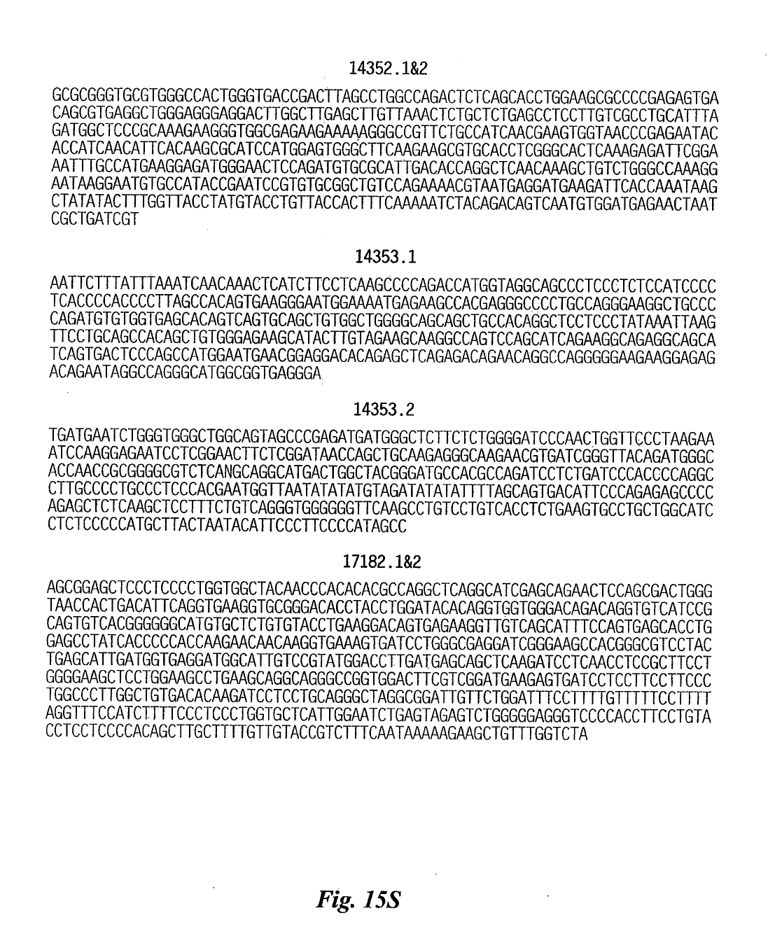

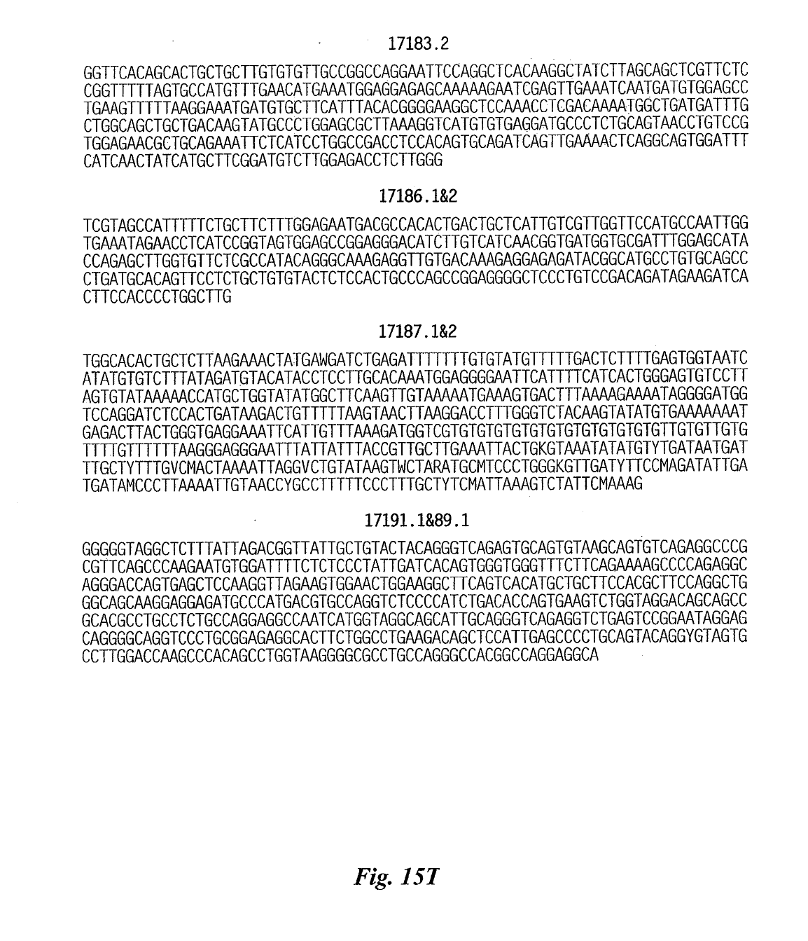

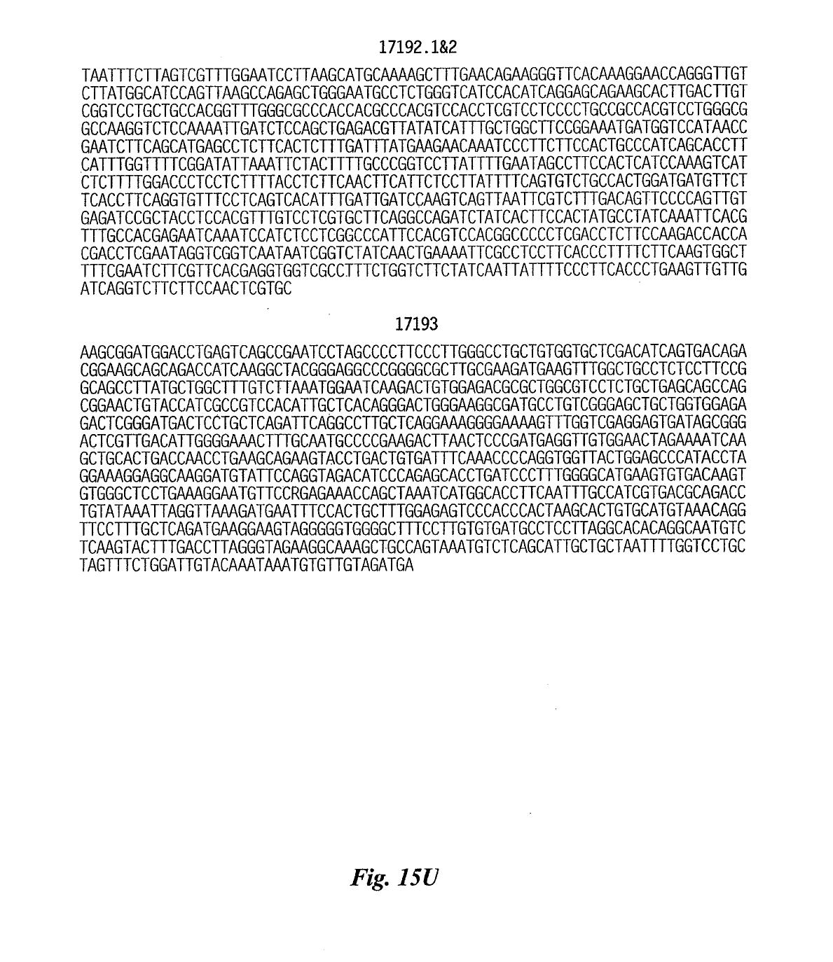

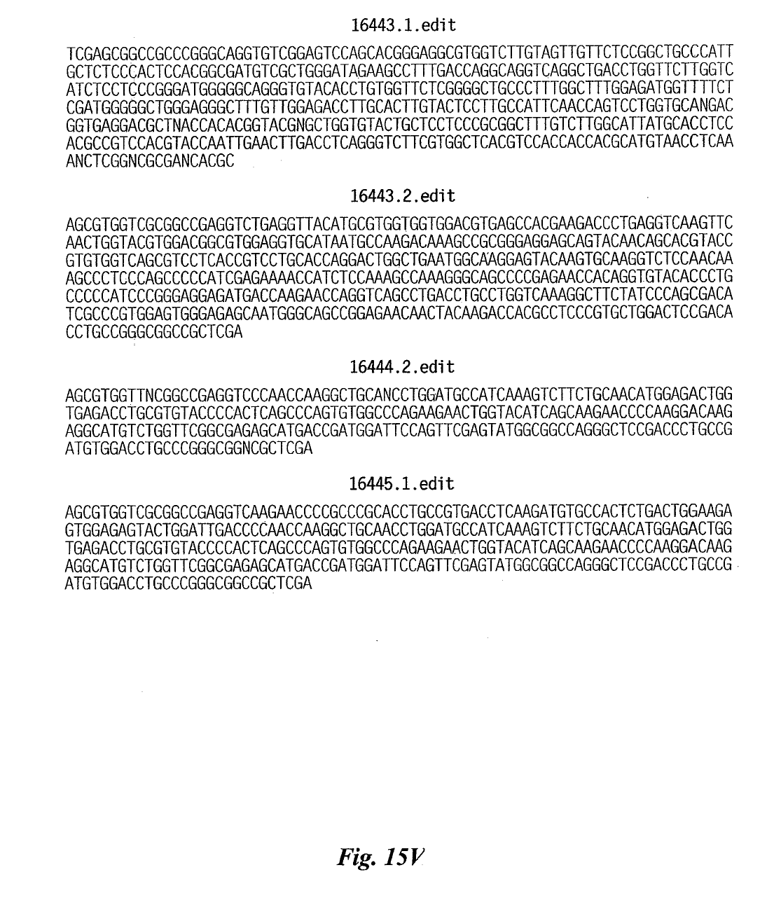

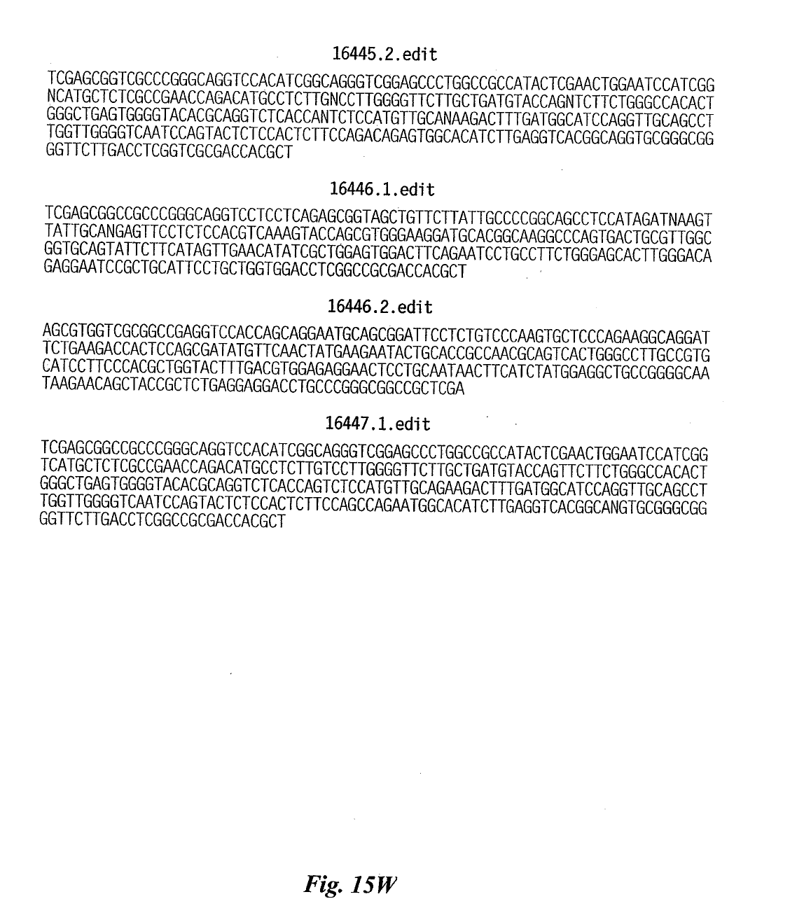

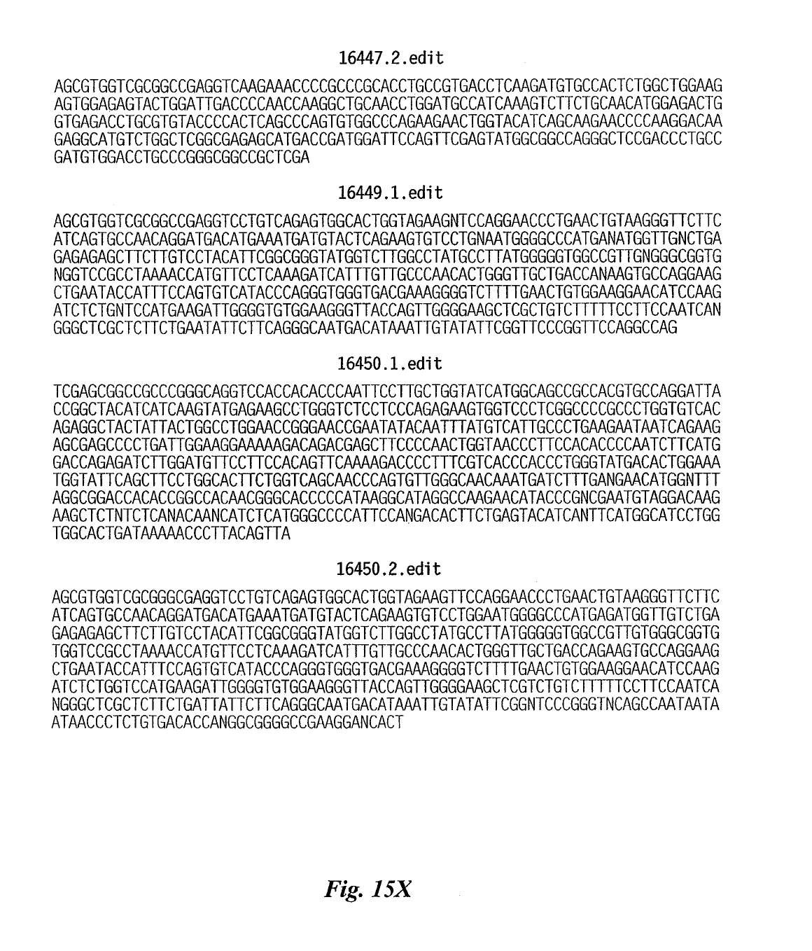

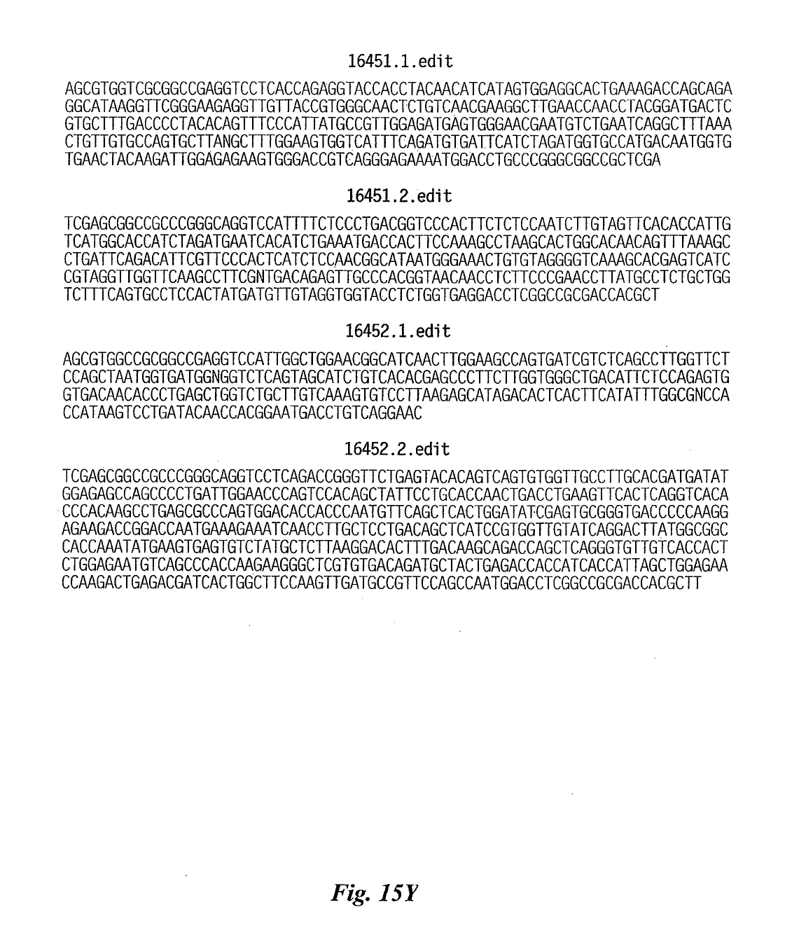

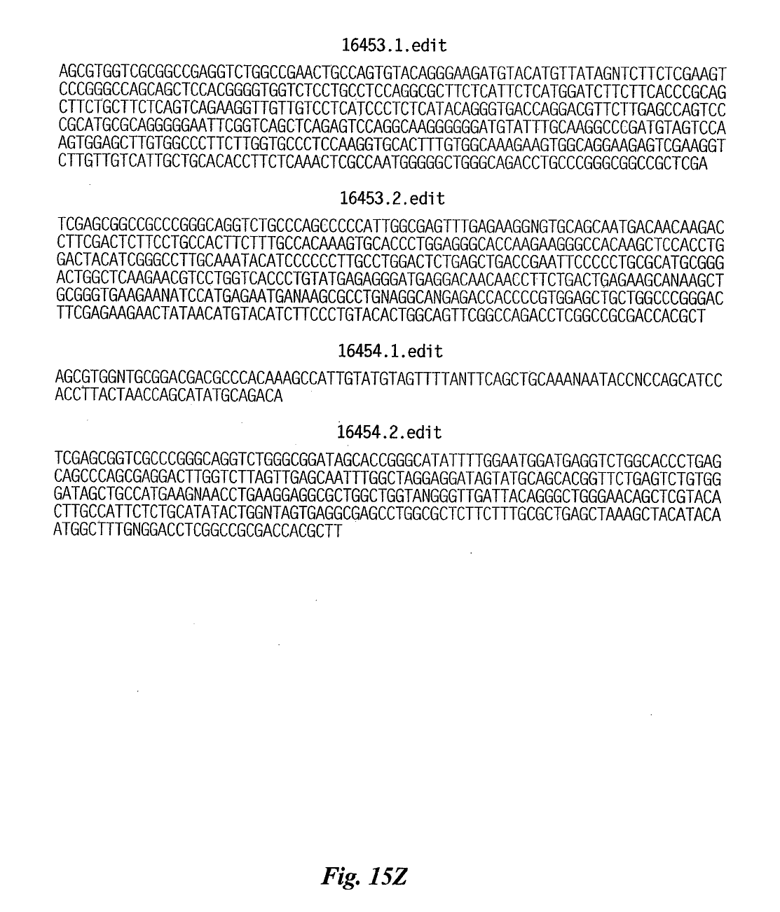

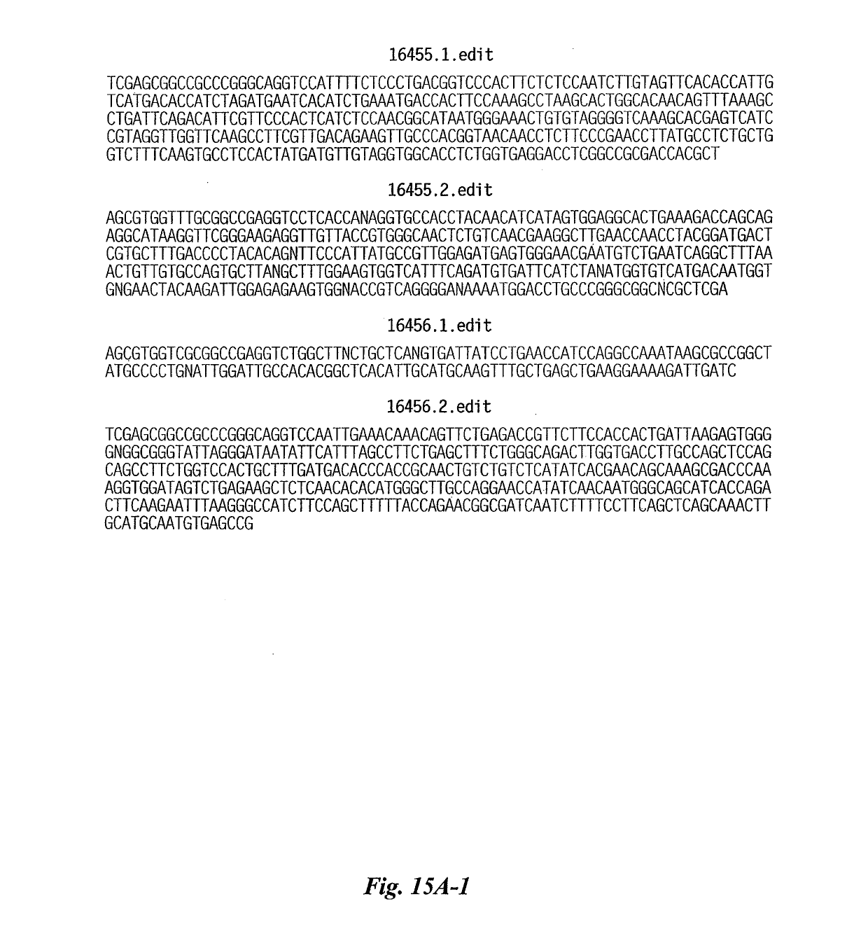

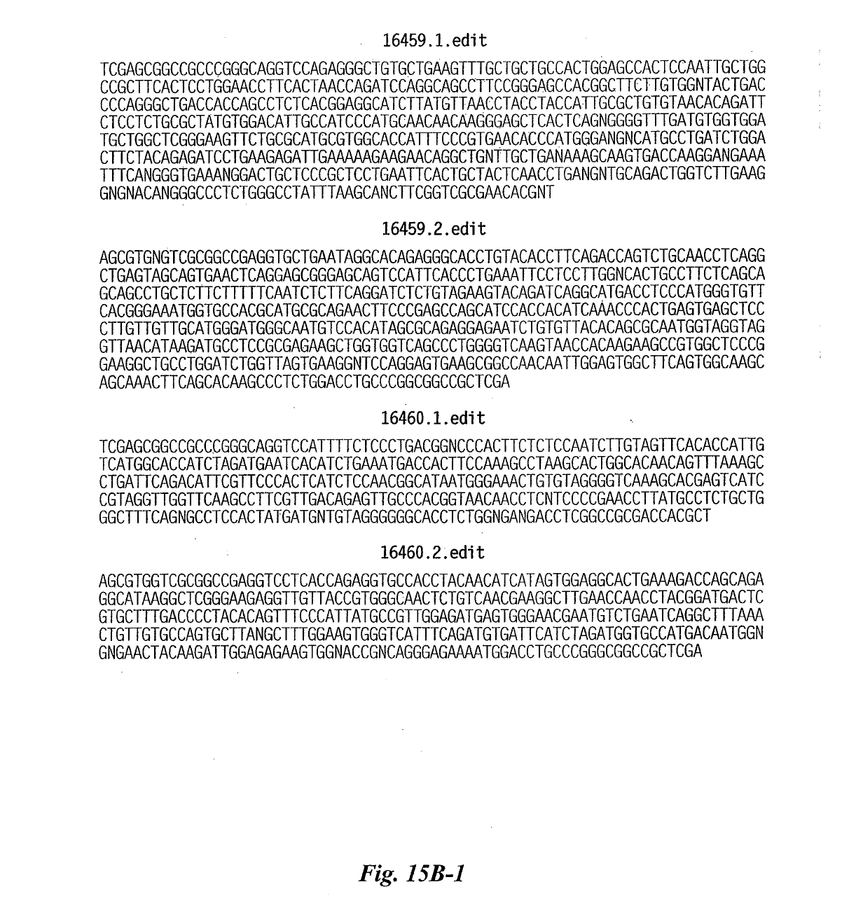

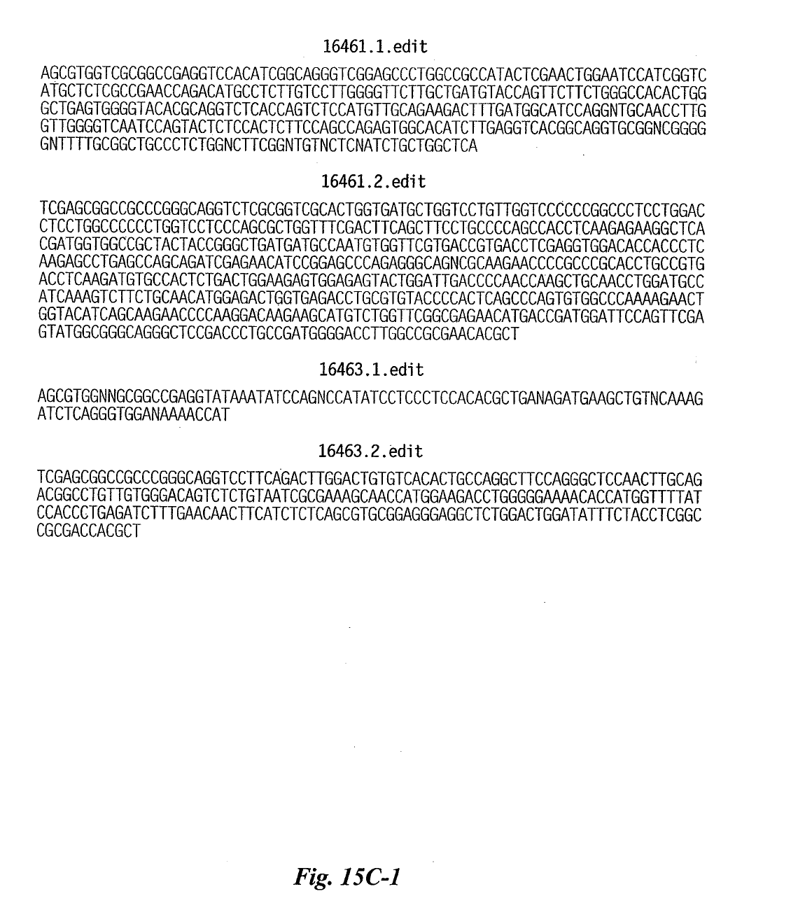

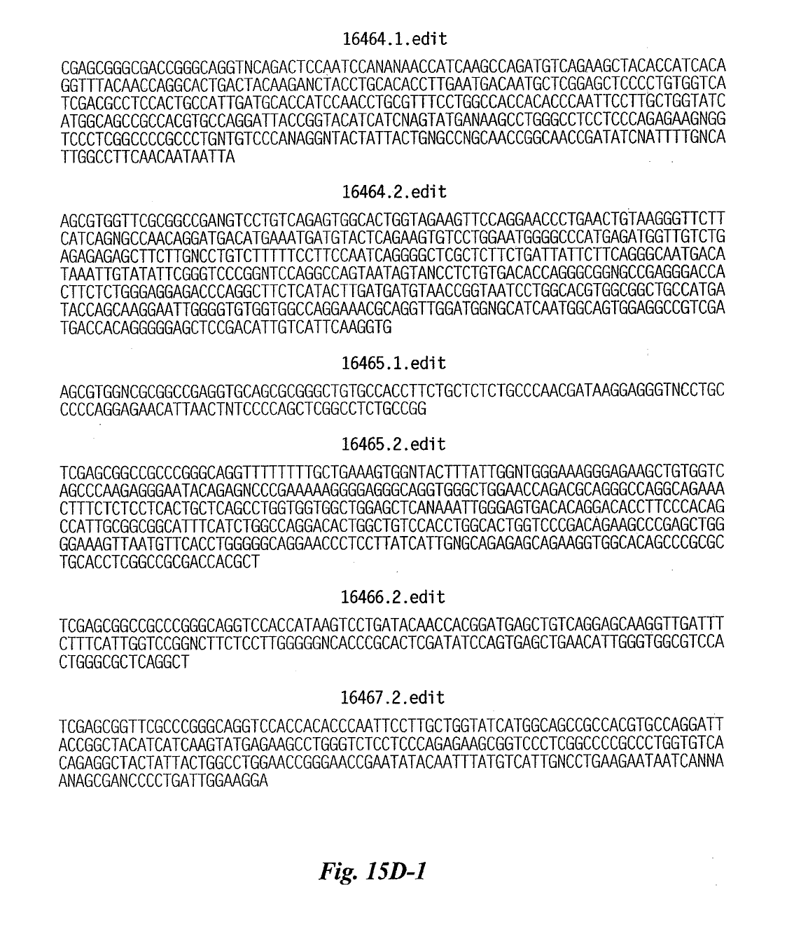

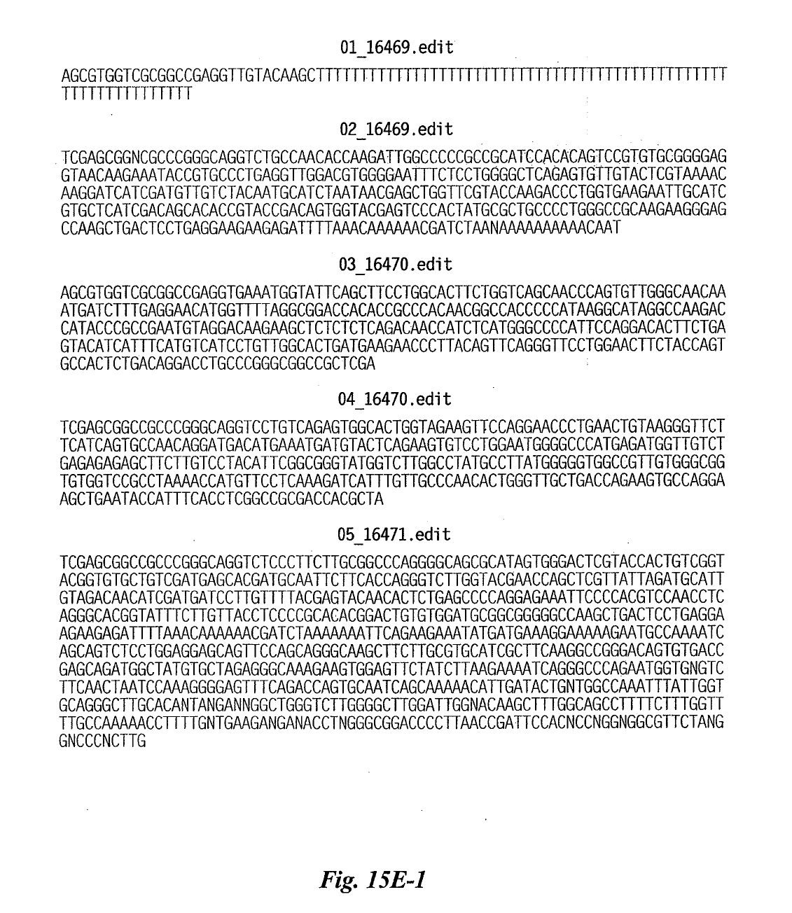

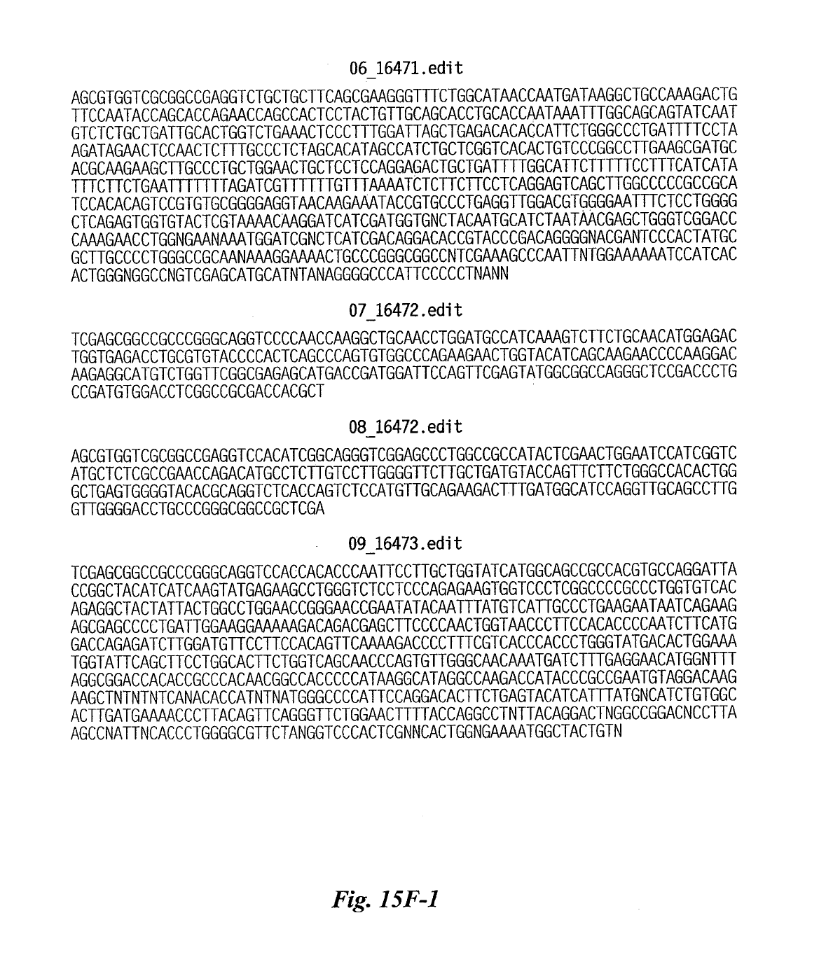

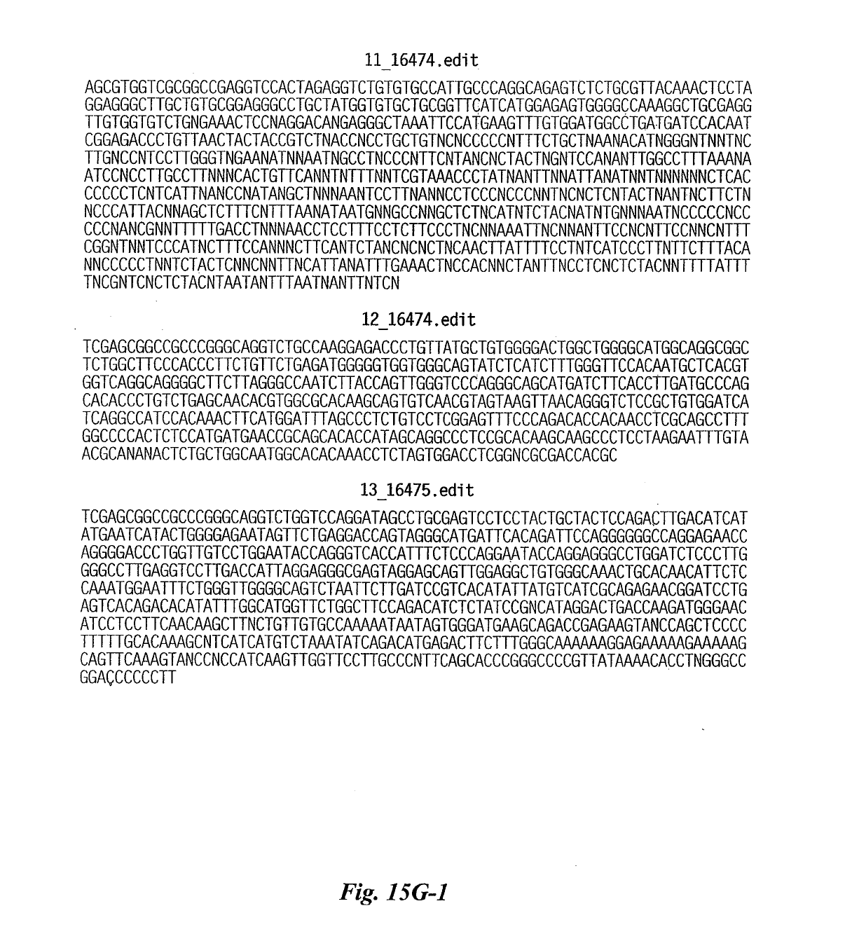

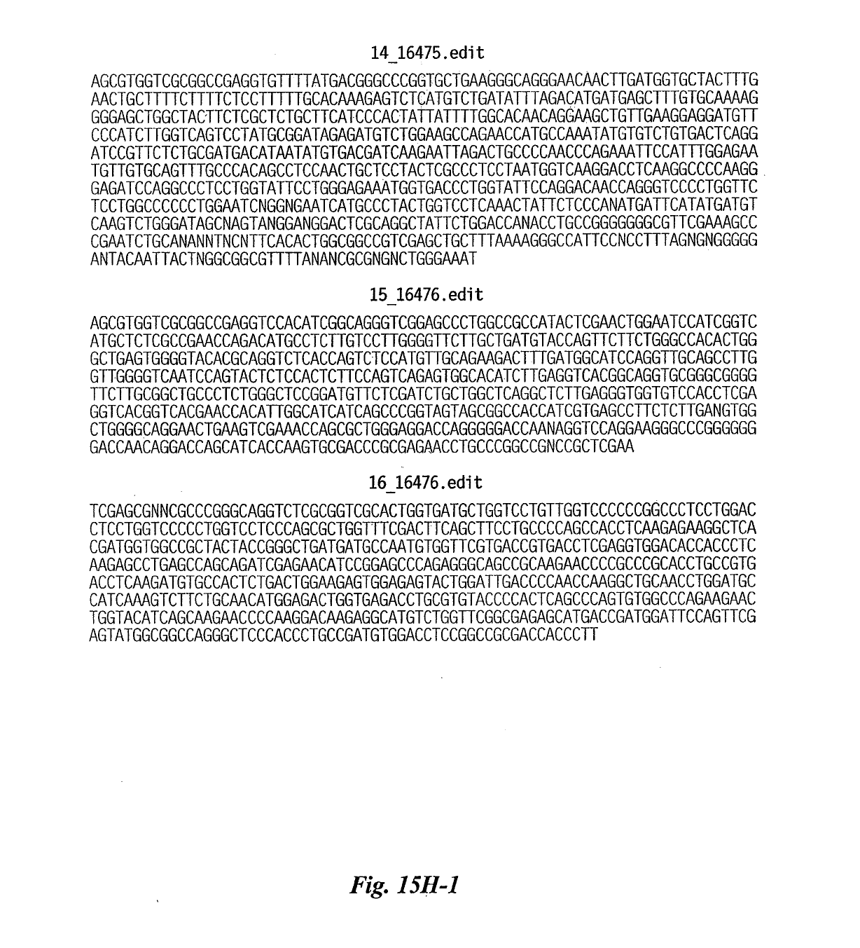

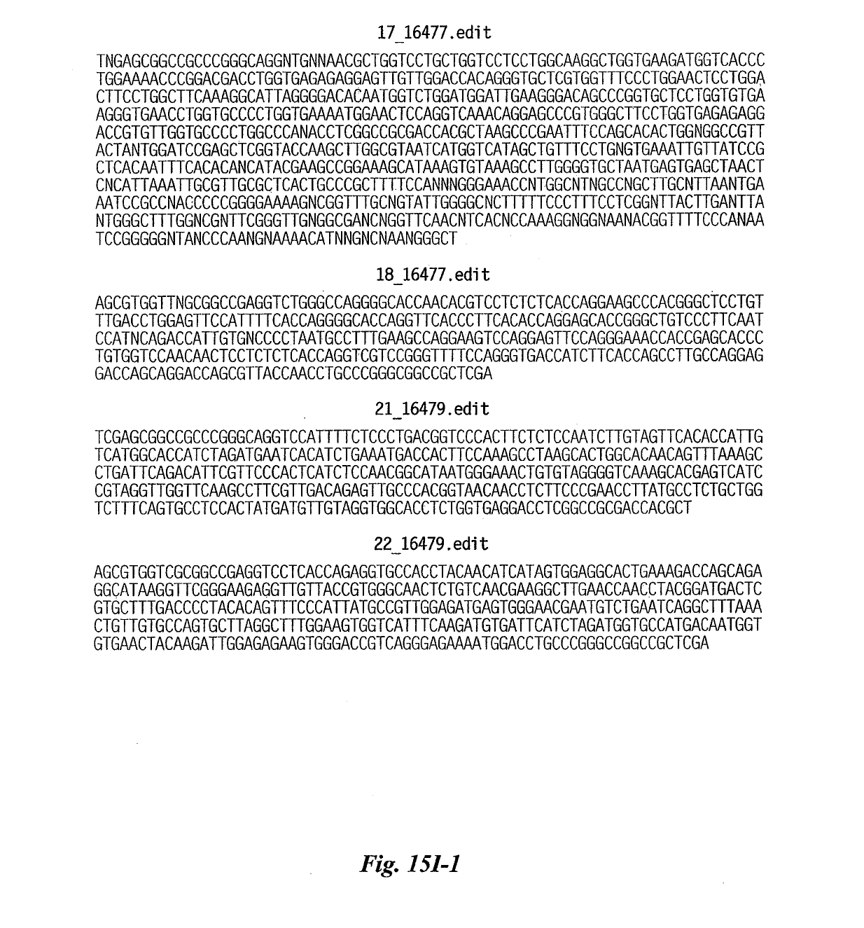

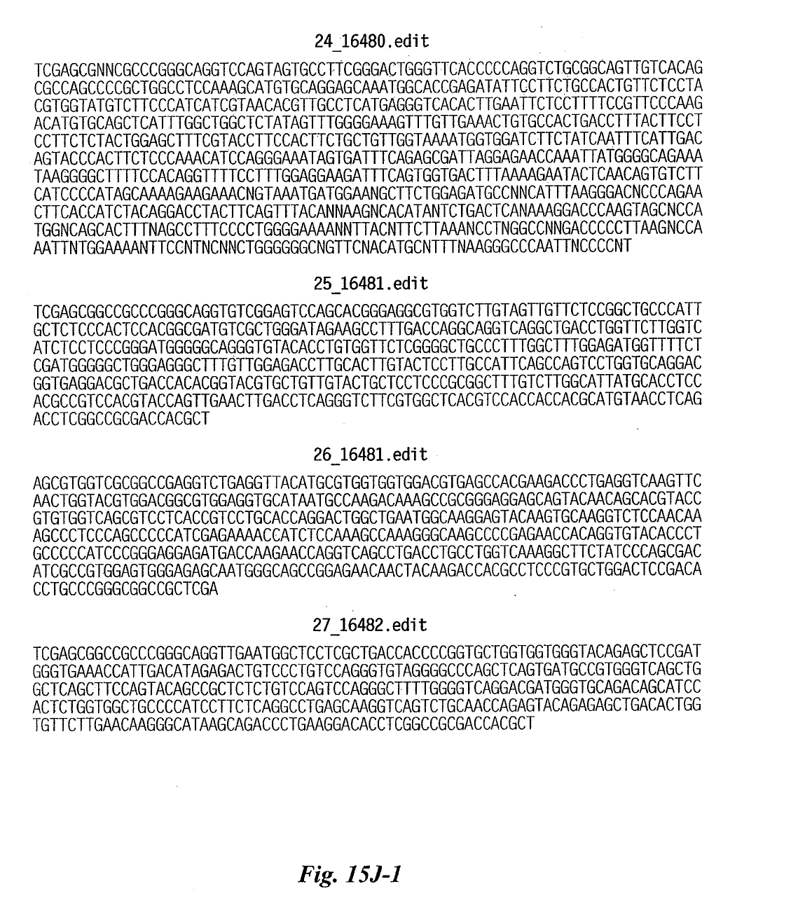

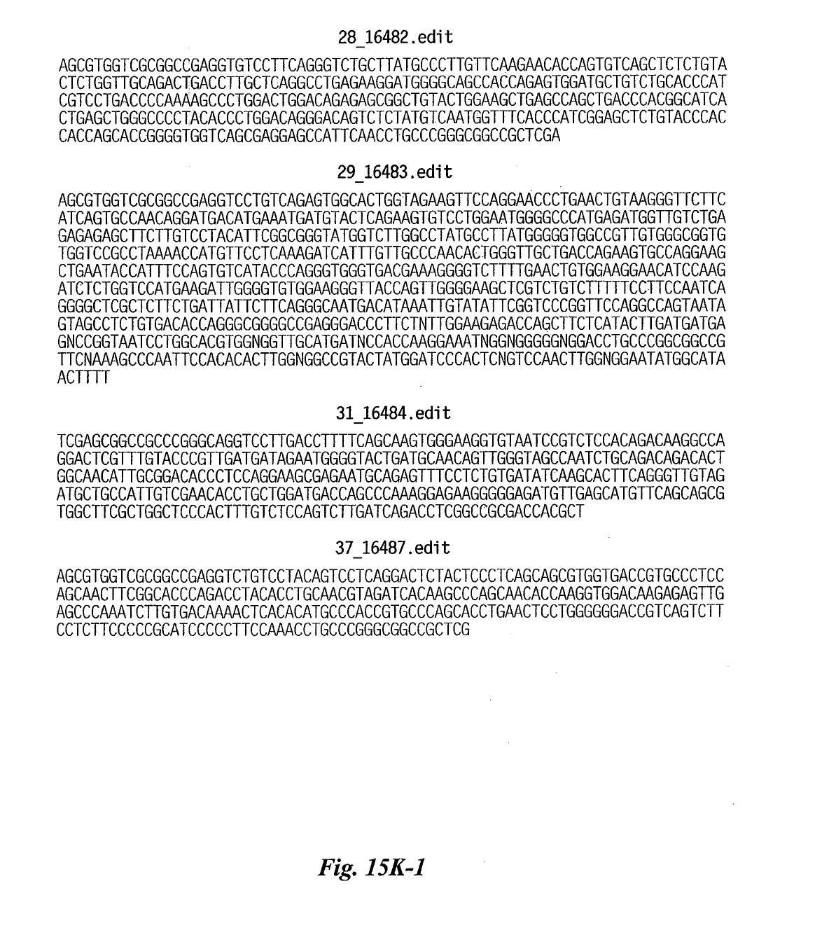

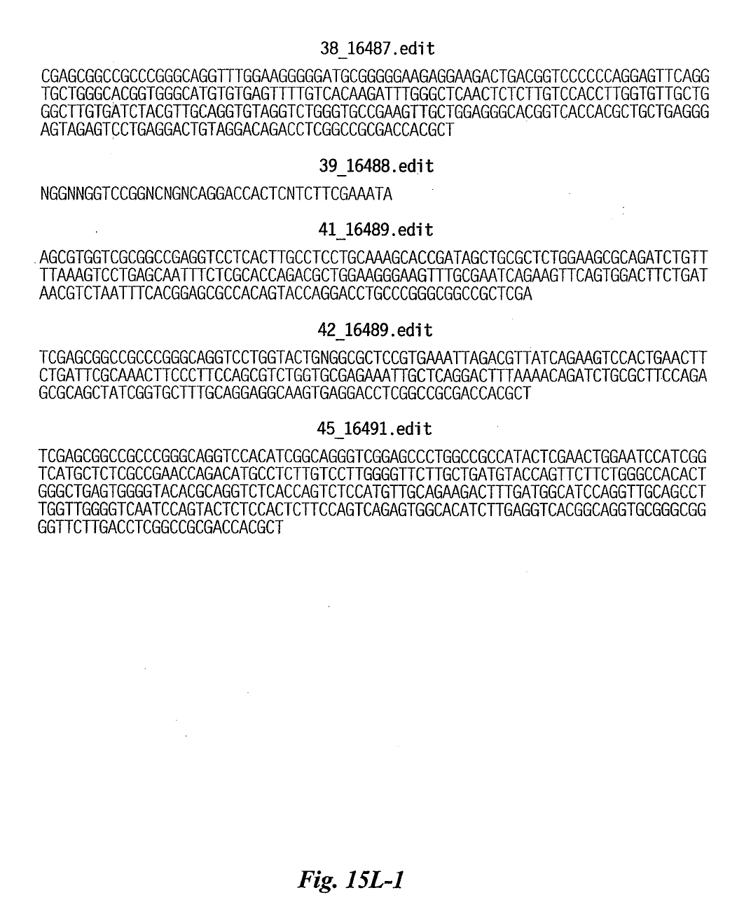

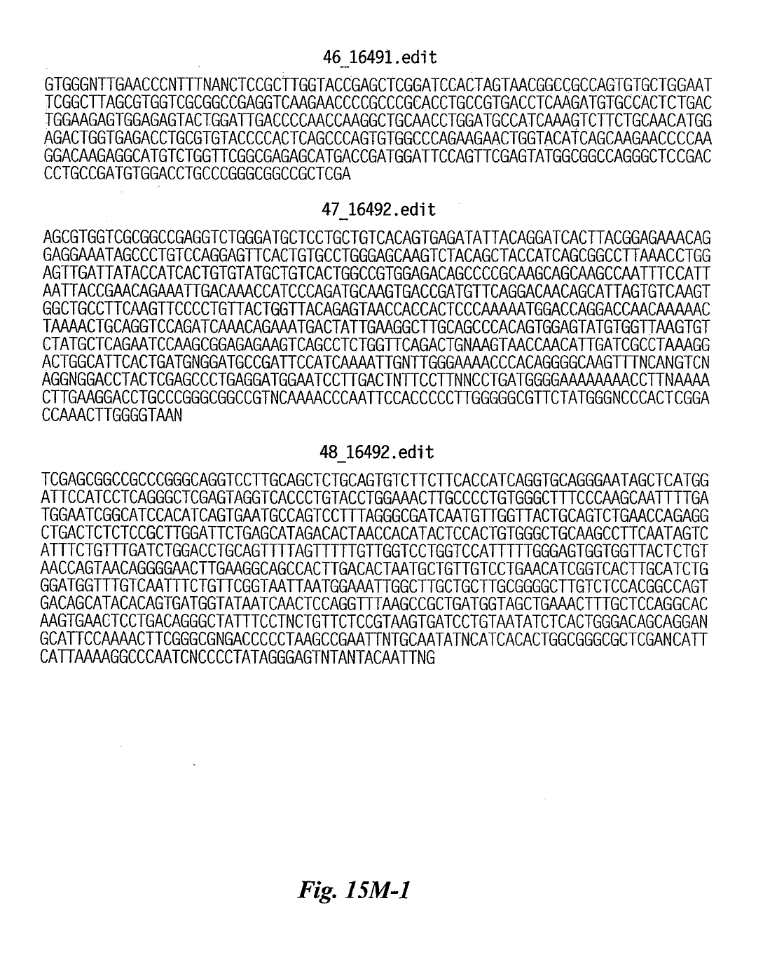

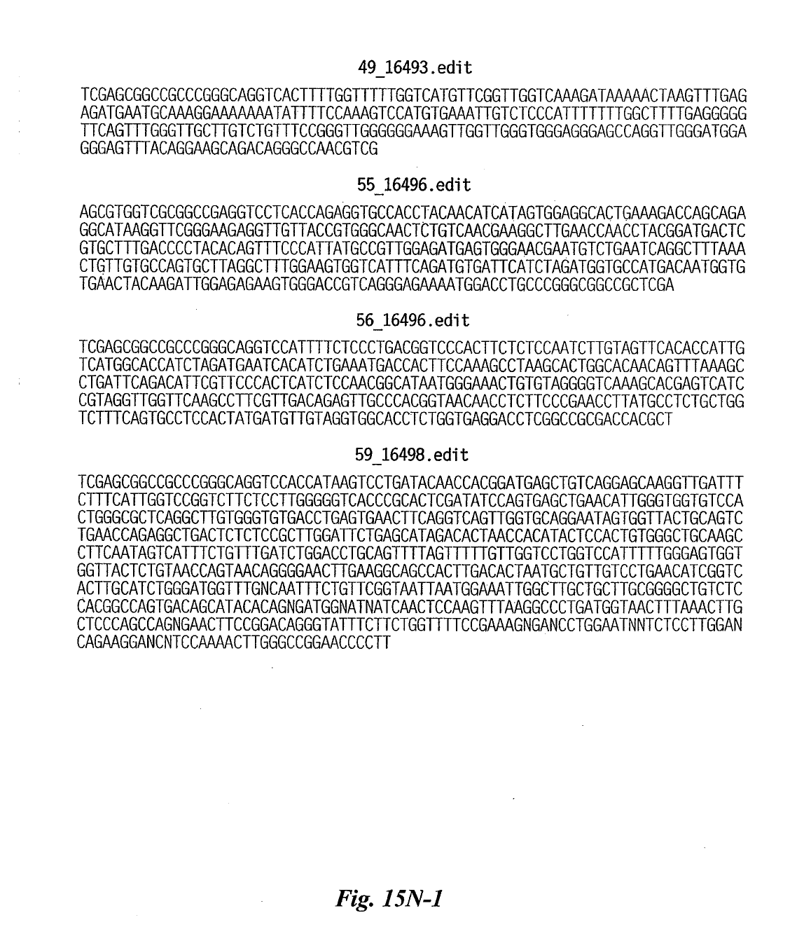

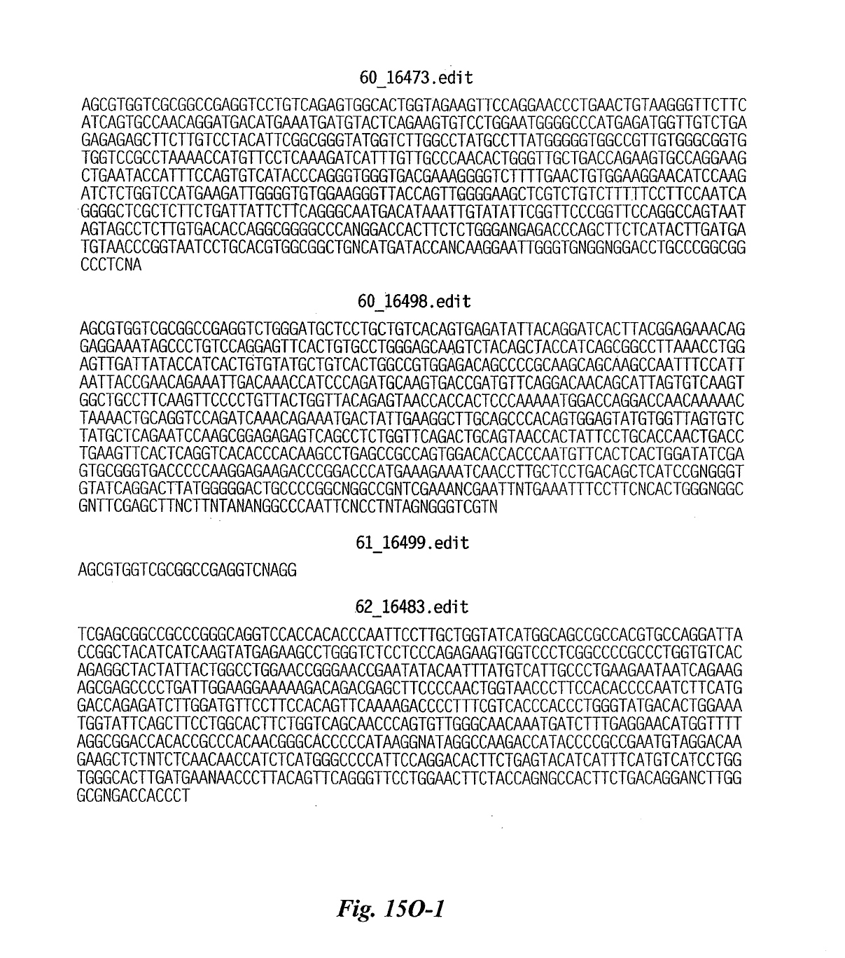

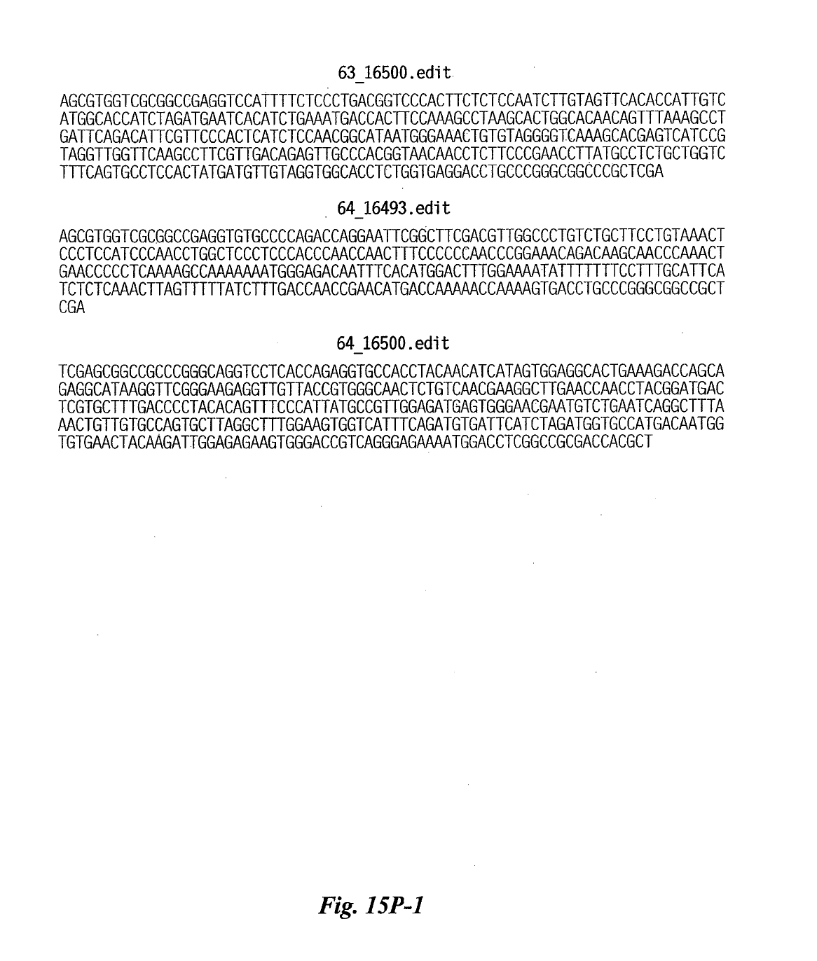

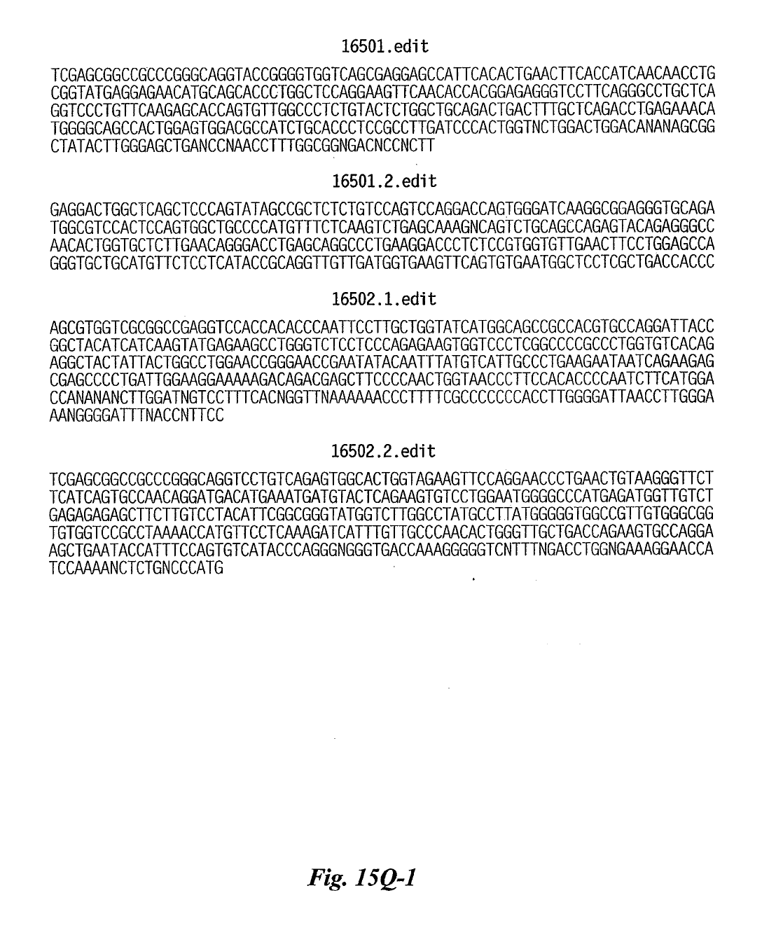

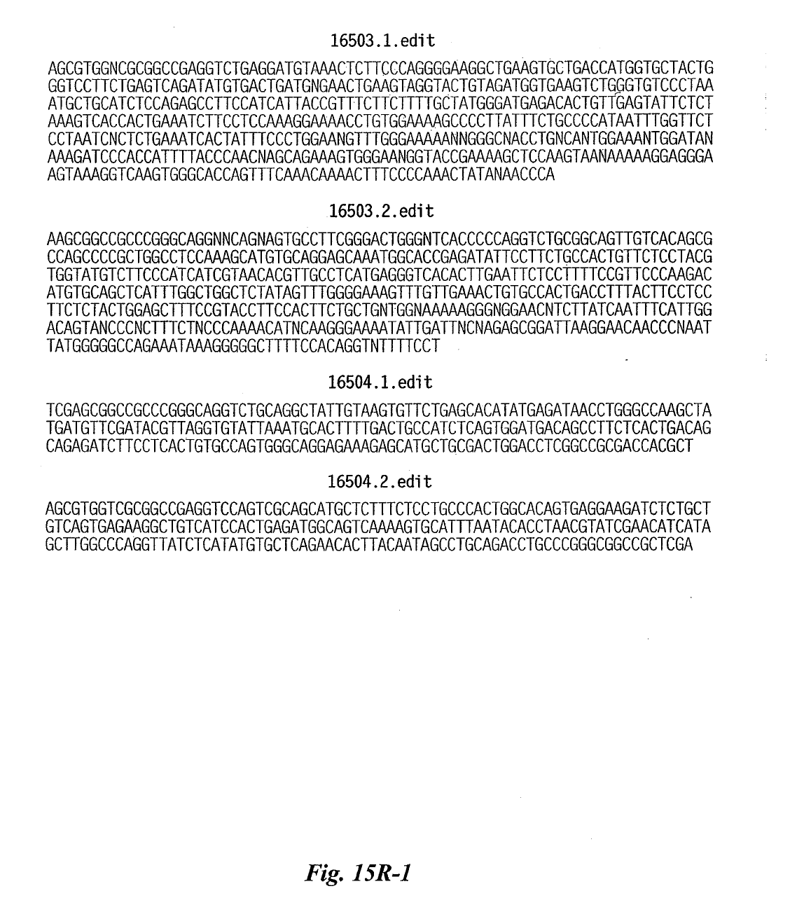

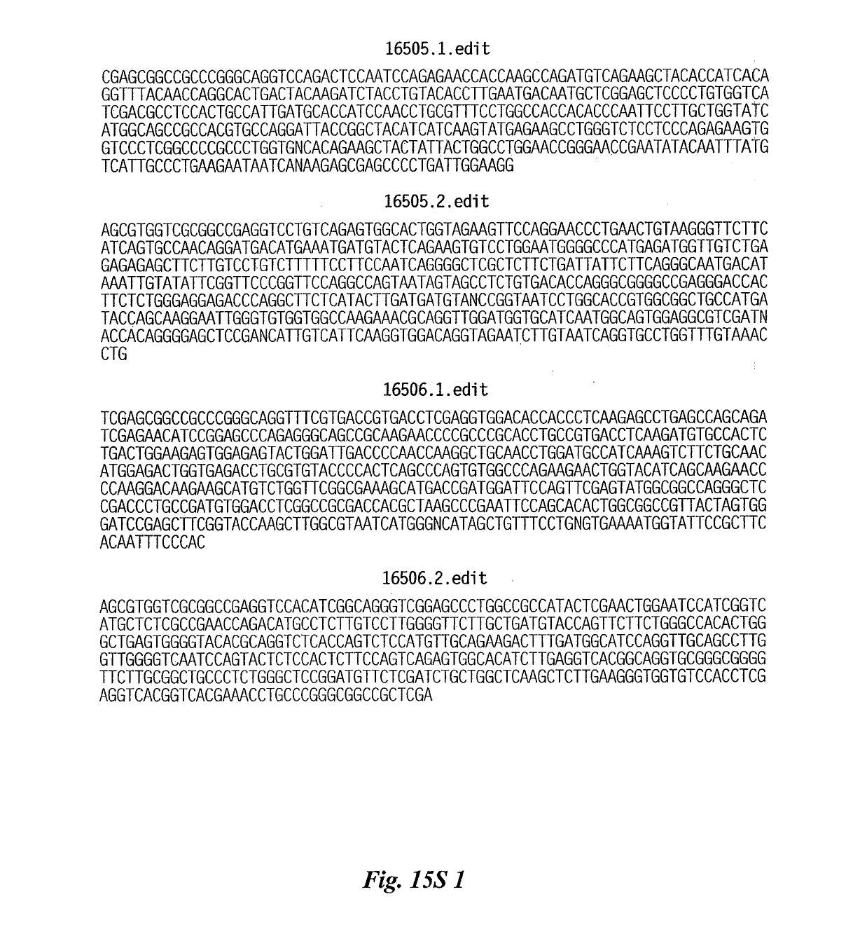

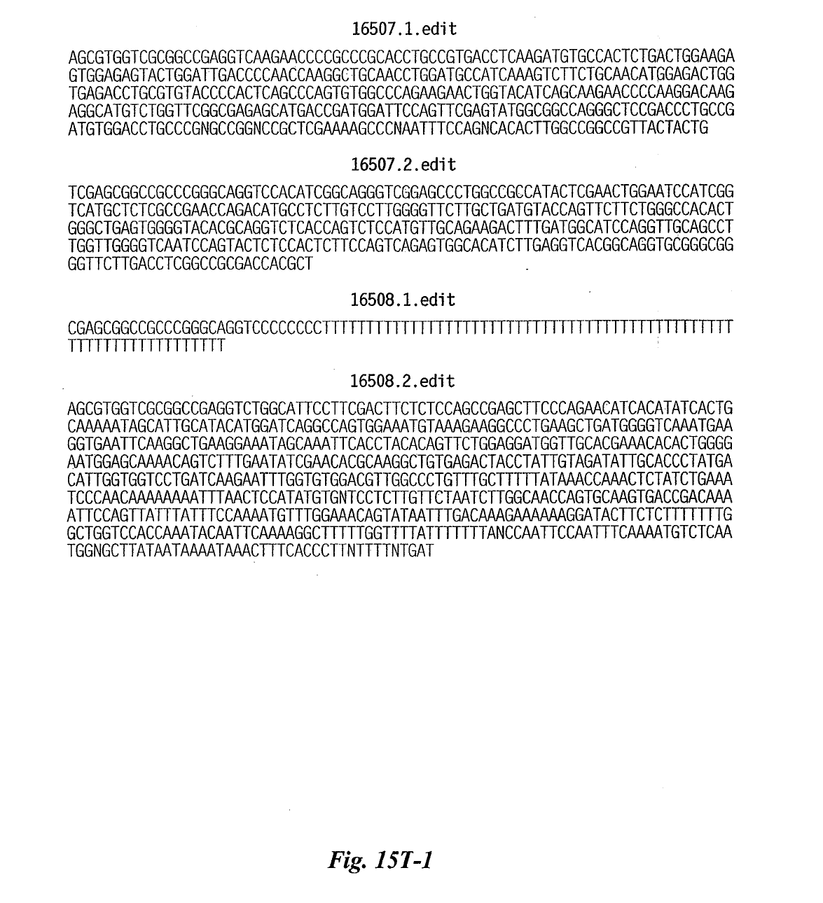

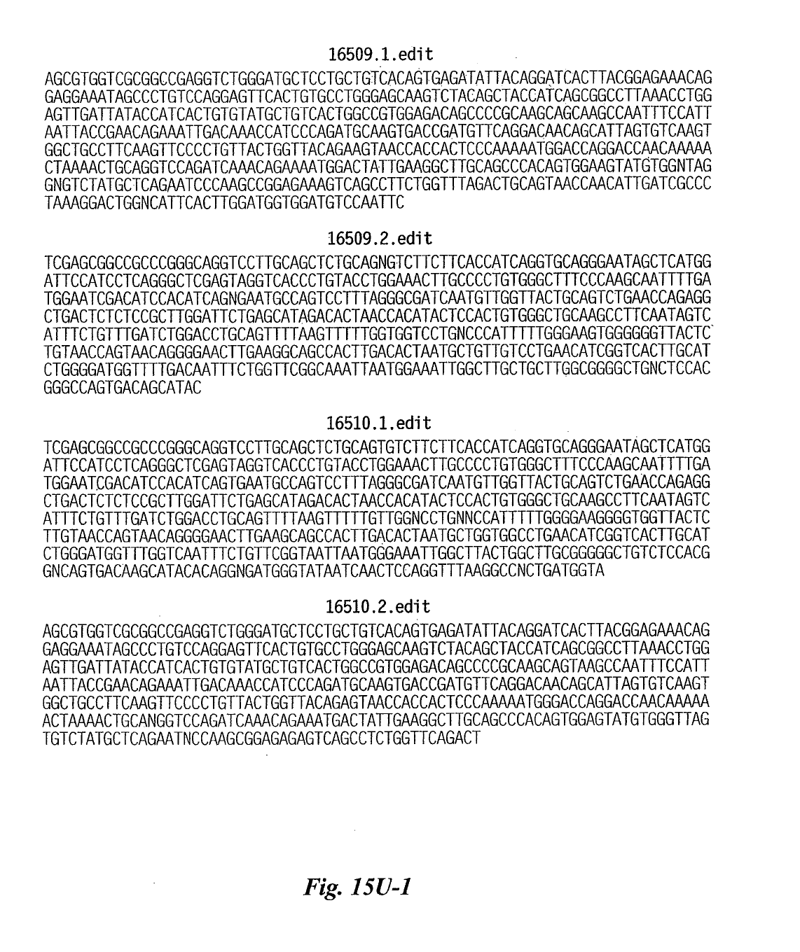

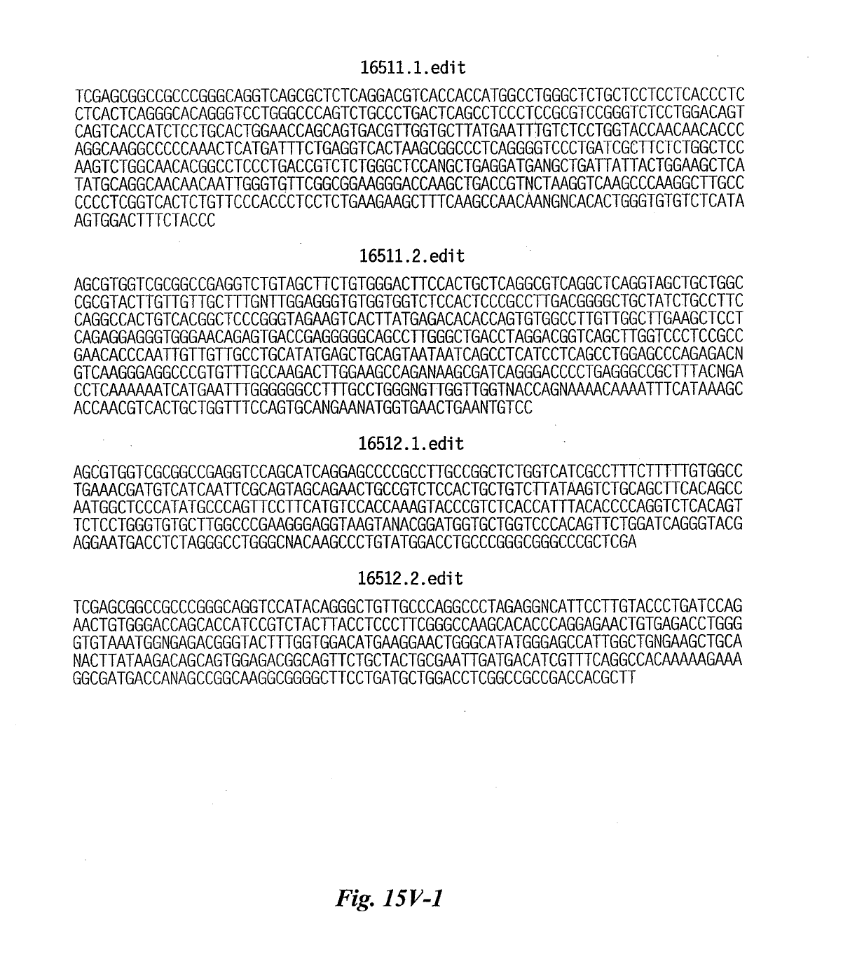

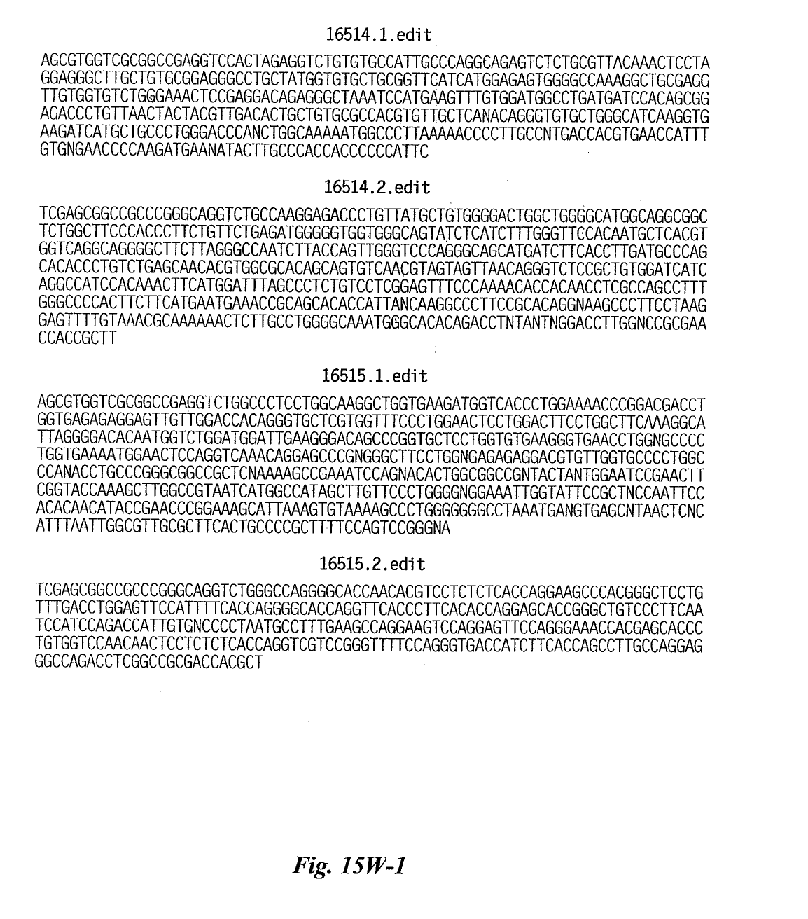

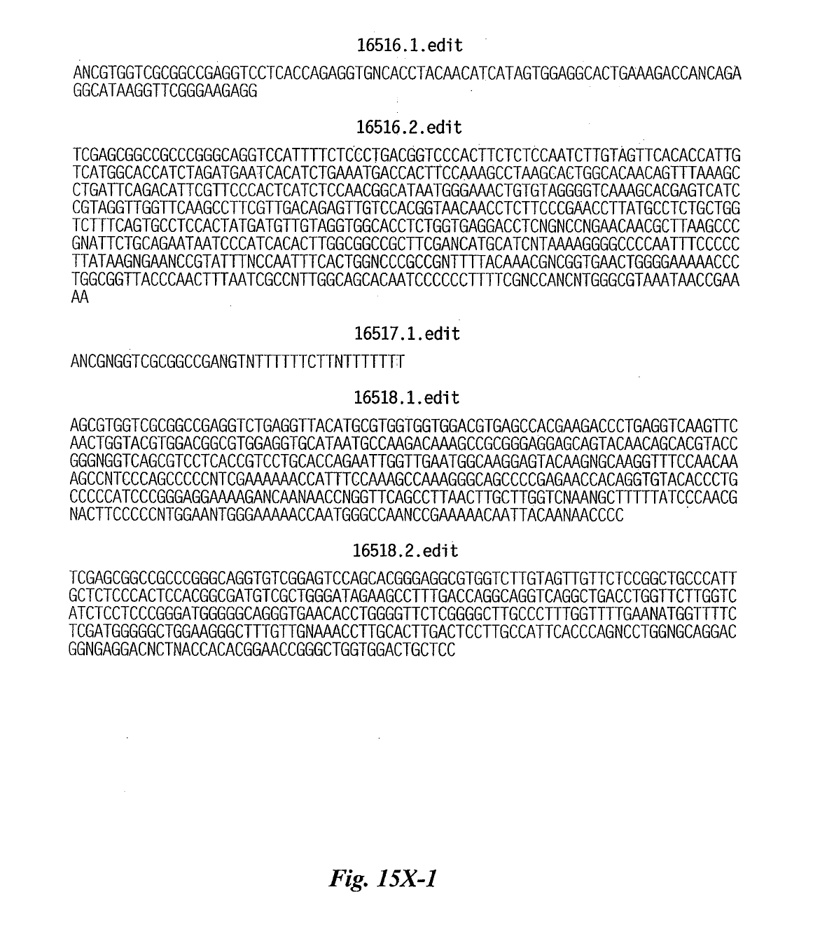

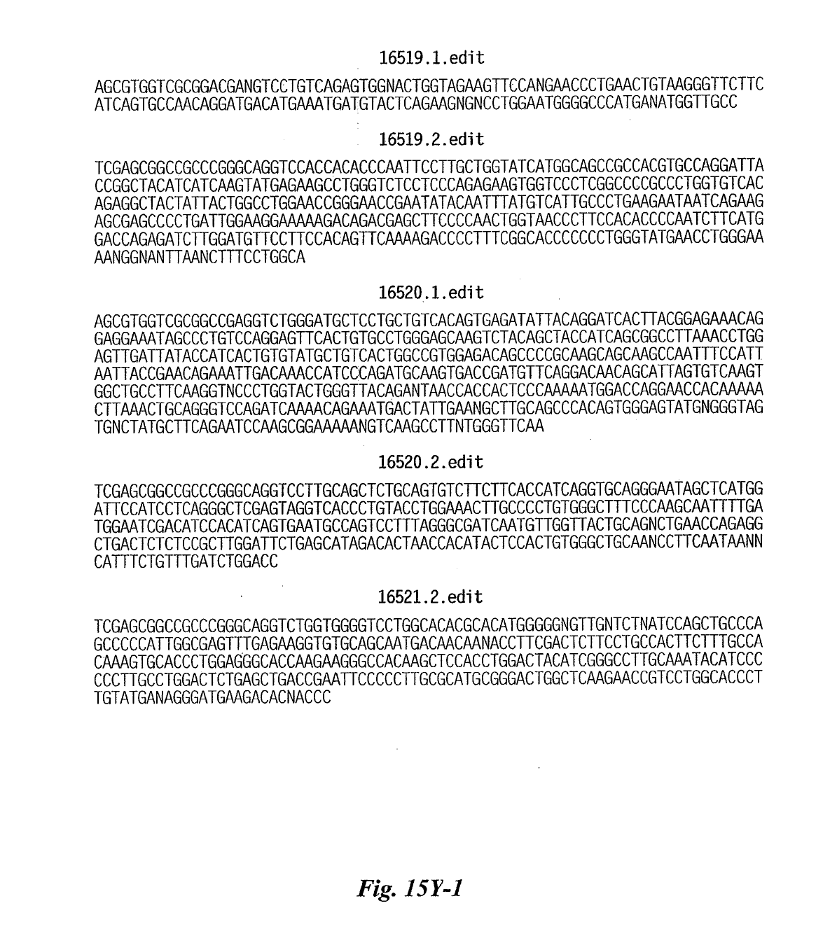

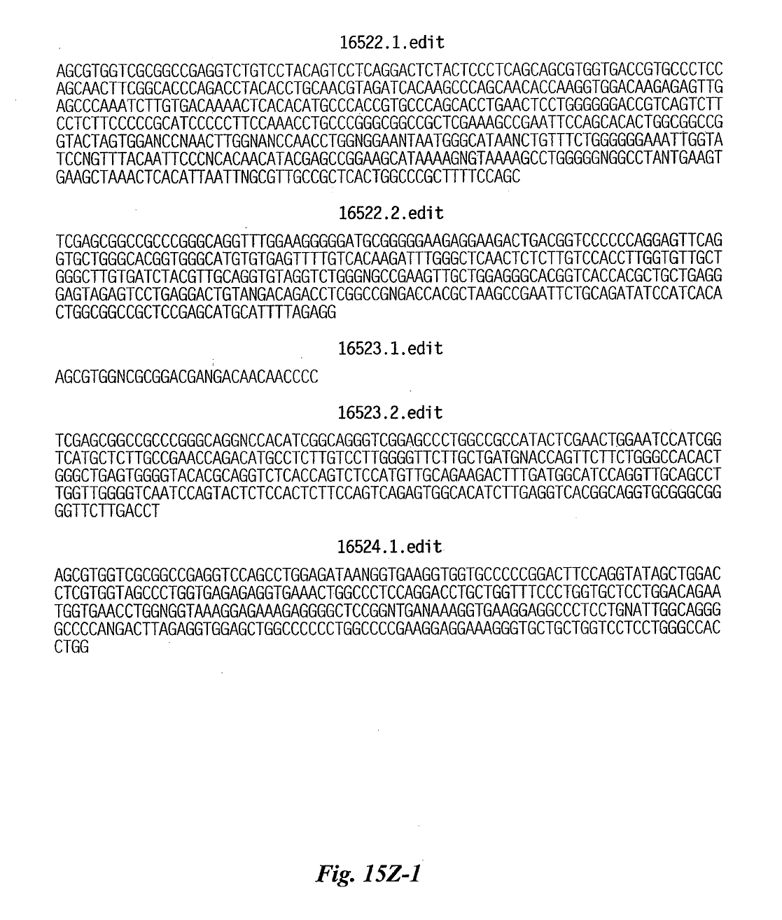

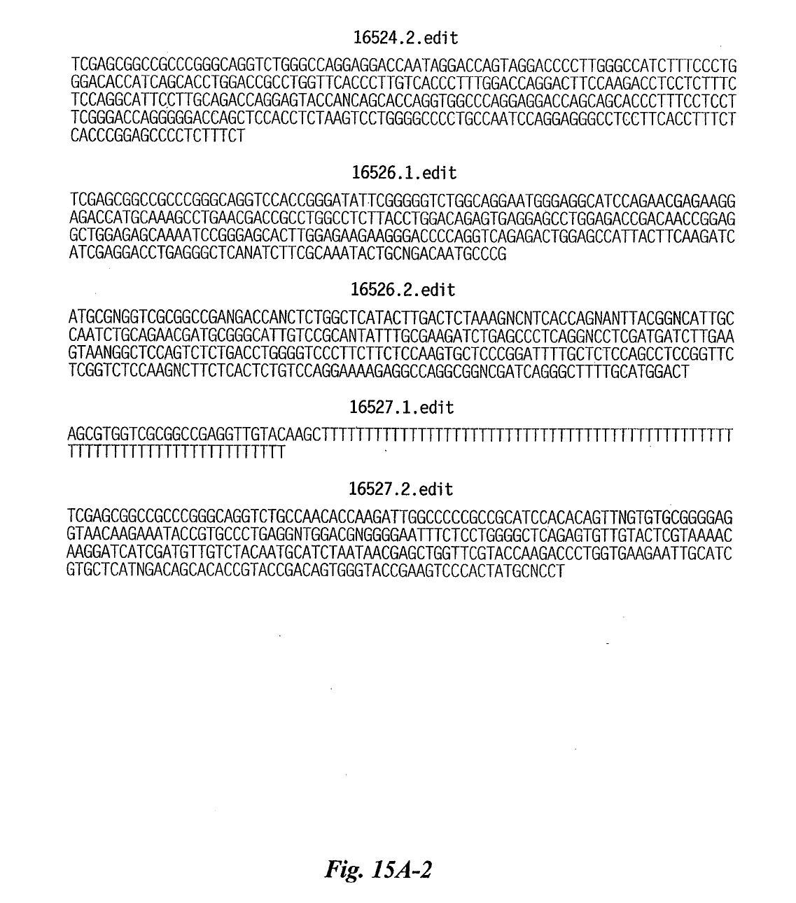

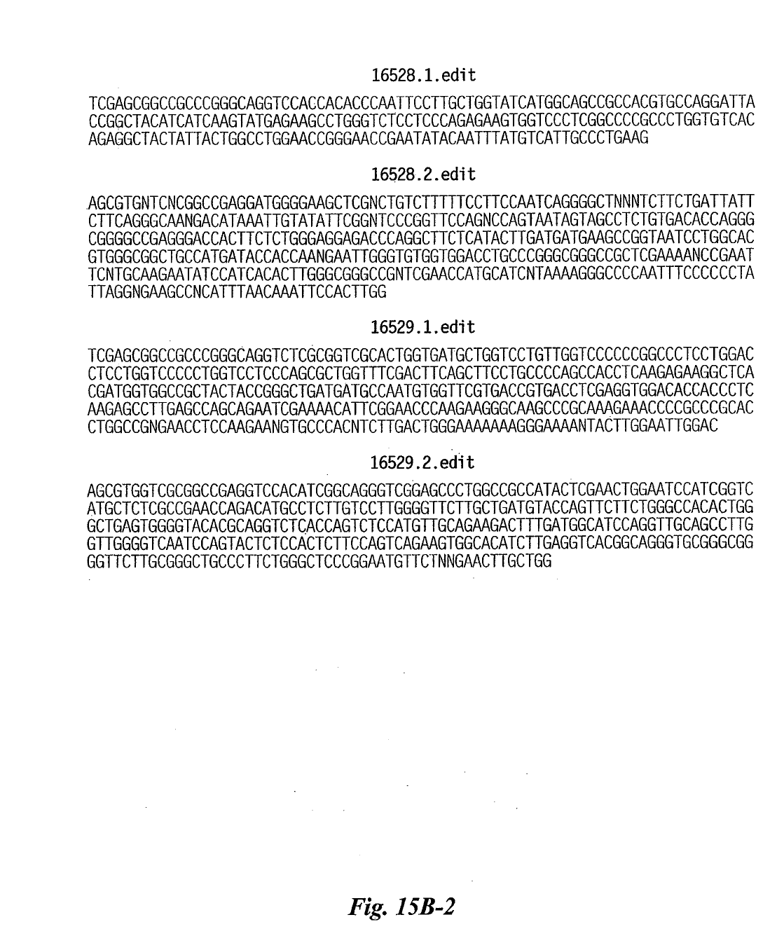

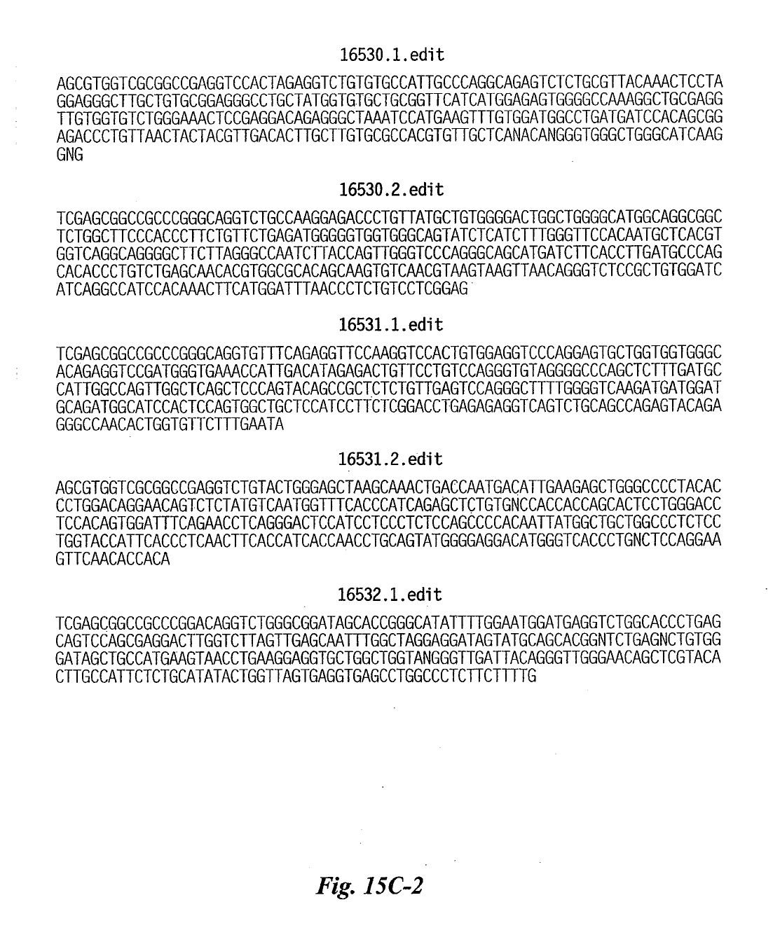

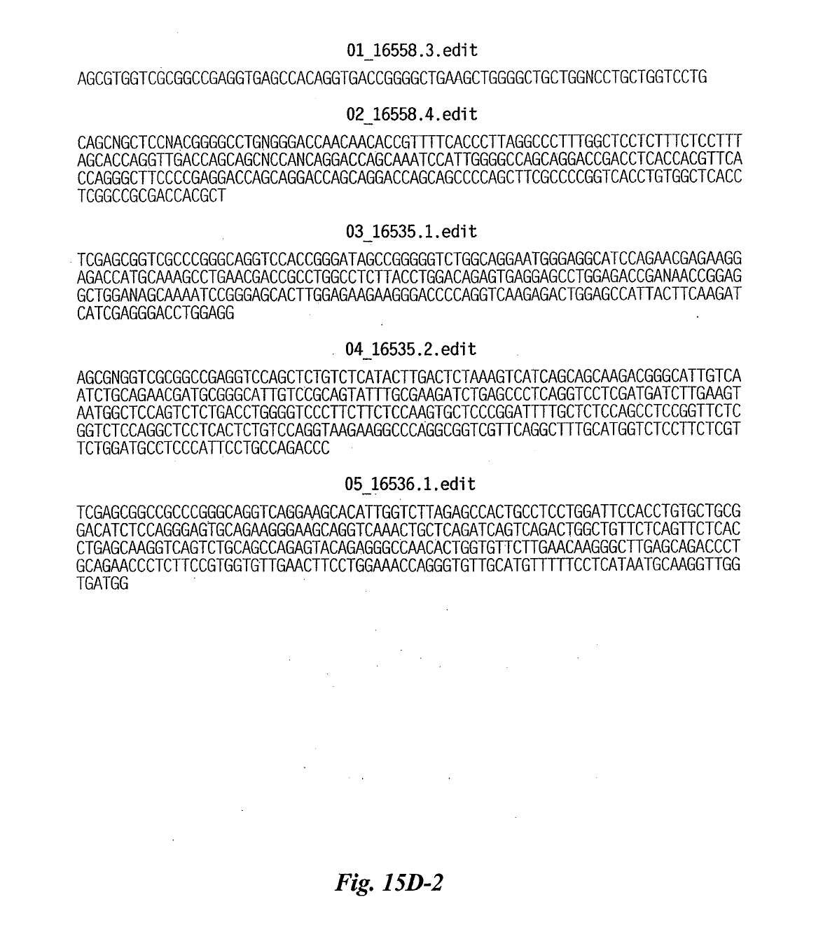

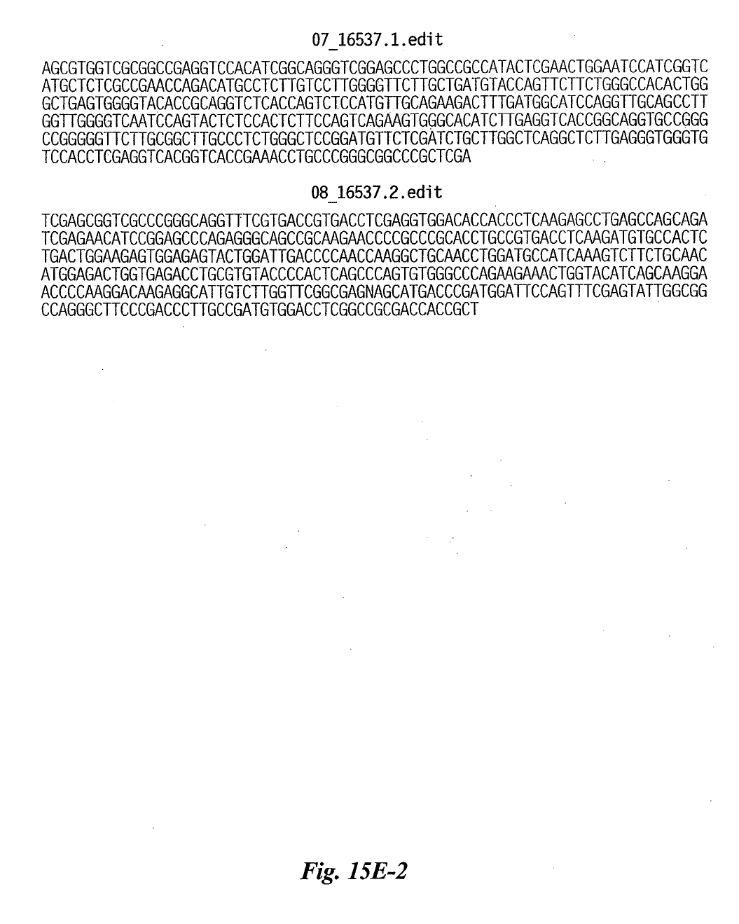

SEQ ID NO:82-310 are ovarian carcinoma antigen polynucleotides shown in FIGS. 15A-15EEE.

SEQ ID NO:311 is a full length sequence of ovarian carcinoma polynucleotide O772P.

SEQ ID NO:312 is the O772P amino acid sequence.

SEQ ID NO:313-384 are ovarian carcinoma antigen polynucleotides.

SEQ ID NO:385 represents the cDNA sequence of a form of the clone O772P, designated 21013.

SEQ ID NO:386 represents the cDNA sequence of a form of the clone O772P, designated 21003.

SEQ ID NO:387 represents the cDNA sequence of a form of the clone O772P, designated 21008.

SEQ ID NOs:388 is the amino acid sequence corresponding to SEQ ID NO:385.

SEQ ID NOs:389 is the amino acid sequence corresponding to SEQ ID NO:386. SEQ ID NOs:390 is the amino acid sequence corresponding to SEQ ID NO:387.

SEQ ID NO:391 is a full length sequence of ovarian carcinoma polynucleotide O8E.

SEQ ID NO:392-393 are protein sequences encoded by O8E.

SEQ ID NO:394-415 are peptide sequences corresponding to the OE8 antibody epitopes.

SEQ ID NO:416-435 are potential HLA-A2 10-mer binding peptides predicted using the full length open-reading frame from OE8.

SEQ ID NO:436-455 are potential HLA-A2 9-mer binding peptides predicted using the full length open-reading frame from OE8.

SEQ ID NO:456 is a truncated nucleotide sequence of the full length Genbank sequence showing homology to O772P

SEQ ID NO:457 is the full length Genbank sequence showing significant homology to O772P

SEQ ID NO:458 is a protein encoding a truncated version of the full length Genbank sequence showing homology to O772P

SEQ ID NO:459 is the full length protein sequence from Genbank showing significant homology to the protein sequence for O772P

SEQ ID NO:460 encodes a unique N-terminal portion of O772P contained in residues 1-70.

SEQ ID NO:461 contains unique sequence and encodes residues 1-313 of SEQ ID NO: 456.

SEQ ID NO:462 is the hypothetical sequence for clone O772P.

SEQ ID NO:463 is the cDNA sequence for clone FLJ14303.

SEQ ID NO:464 is a partial cDNA sequence for clone O772P.

SEQ ID NO:465 is a partial cDNA sequence for clone O772P.

SEQ ID NO:466 is a partial cDNA sequence for clone O772P.

SEQ ID NO:467 is a partial cDNA sequence for clone O772P.

SEQ ID NO:468 is a partial cDNA sequence for clone O772P.

SEQ ID NO:469 is a partial cDNA sequence for clone O772P.

SEQ ID NO:470 is a partial cDNA sequence for clone O772P.

SEQ ID NO:471 is a partial cDNA sequence for clone O772P.

SEQ ID NO:472 is a partial cDNA sequence for clone O772P.

SEQ ID NO:473 is a partial cDNA sequence for clone O772P.

SEQ ID NO:474 is a partial cDNA sequence for clone O772P.

SEQ ID NO:475 is a partial cDNA sequence for clone O772P.

SEQ ID NO:476 is a partial cDNA sequence for clone O772P.

SEQ ID NO:477 represents the novel 5′-end of the ovarian tumor antigen O772P.

SEQ ID NO:478 is the amino acid sequence encoded by SEQ ID NO:462.

SEQ ID NO:479 is the amino acid sequence encoded by SEQ ID NO:463.

SEQ ID NO:480 is a partial amino acid sequence encoded by SEQ ID NO:472.

SEQ ID NO:481 is a partial amino acid sequence encoded by a possible open reading frame of SEQ ID NO:471.

SEQ ID NO:482 is a partial amino acid sequence encoded by a second possible open reading frame of SEQ ID NO:471.

SEQ ID NO:483 is a partial amino acid sequence encoded by SEQ ID NO:467.

SEQ ID NO:484 is a partial amino acid sequence encoded by a possible open reading frame of SEQ ID NO:466.

SEQ ID NO:485 is a partial amino acid sequence encoded by a second possible open reading frame of SEQ ID NO:466.

SEQ ID NO:486 is a partial amino acid sequence encoded by SEQ ID NO:465.

SEQ ID NO:487 is a partial amino acid sequence encoded by SEQ ID NO:464.

SEQ ID NO:488 represents the extracellular, transmembrane and cytoplasmic regions of O772P.

SEQ ID NO:489 represents the predicted extracellular domain of O772P.

SEQ ID NO:490 represents the amino acid sequence of peptide #2 which corresponds to an O772P specific antibody epitope.

SEQ ID NO:491 represents the amino acid sequence of peptide #6 which corresponds to an O772P specific antibody epitope.

SEQ ID NO:492 represents the amino acid sequence of peptide #7 which corresponds to an O772P specific antibody epitope.

SEQ ID NO:493 represents the amino acid sequence of peptide #8 which corresponds to an O772P specific antibody epitope.

SEQ ID NO:494 represents the amino acid sequence of peptide #9 which corresponds to an O772P specific antibody epitope.

SEQ ID NO:495 represents the amino acid sequence of peptide #11 which corresponds to an O772P specific antibody epitope.

SEQ ID NO:496 represents the amino acid sequence of peptide #13 which corresponds to an O772P specific antibody epitope.

SEQ ID NO:497 represents the amino acid sequence of peptide #22 which corresponds to an O772P specific antibody epitope.

SEQ ID NO:498 represents the amino acid sequence of peptide #24 which corresponds to an O772P specific antibody epitope.

SEQ ID NO:499 represents the amino acid sequence of peptide #27 which corresponds to an O772P specific antibody epitope.

SEQ ID NO:500 represents the amino acid sequence of peptide #40 which corresponds to an O772P specific antibody epitope.

SEQ ID NO:501 represents the amino acid sequence of peptide #41 which corresponds to an O772P specific antibody epitope.

SEQ ID NO:502 represents the amino acid sequence of peptide #47 which corresponds to an O772P specific antibody epitope.

SEQ ID NO:503 represents the amino acid sequence of peptide #50 which corresponds to an O772P specific antibody epitope.

SEQ ID NO:504 represents the amino acid sequence of peptide #51 which corresponds to an O772P specific antibody epitope.

SEQ ID NO:505 represents the amino acid sequence of peptide #52 which corresponds to an O772P specific antibody epitope.

SEQ ID NO:506 represents the amino acid sequence of peptide #53 which corresponds to an O772P specific antibody epitope.

SEQ ID NO:507 represents the amino acid sequence of peptide #58 which corresponds to an O772P specific antibody epitope.

SEQ ID NO:508 represents the amino acid sequence of peptide #59 which corresponds to an O772P specific antibody epitope.

SEQ ID NO:509 represents the amino acid sequence of peptide #60 which corresponds to an O772P specific antibody epitope.

SEQ ID NO:510 represents the amino acid sequence of peptide #61 which corresponds to an O772P specific antibody epitope.

SEQ ID NO:511 represents the amino acid sequence of peptide #71 which corresponds to an O772P specific antibody epitope.

SEQ ID NO:512 (O772P repeat1) represents an example of a cDNA sequence corresponding to repeat number 21 from the 5′ variable region of O772P.

SEQ ID NO:513 (O772P repeat2) represents an example of a cDNA sequence corresponding to repeat number 20 from the 5′ variable region of O772P.

SEQ ID NO:514 (O772P repeat3) represents an example of a cDNA sequence corresponding to repeat number 19 from the 5′ variable region of O772P.

SEQ ID NO:515 (O772P repeat4) represents an example of a cDNA sequence corresponding to repeat number 18 from the 5′ variable region of O772P.

SEQ ID NO:516 (O772P repeat5) represents an example of a cDNA sequence corresponding to repeat number 17 from the 5′ variable region of O772P.

SEQ ID NO:517 (HB repeat1) represents an example of a cDNA sequence corresponding to repeat number 21 from the 5′ variable region of O772P.

SEQ ID NO:518 (HB repeat2) represents an example of a cDNA sequence corresponding to repeat number 20 from the 5′ variable region of O772P.

SEQ ID NO:519 (HB repeat3) represents an example of a cDNA sequence corresponding to repeat number 19 from the 5′ variable region of O772P.

SEQ ID NO:520 (HB repeat4) represents an example of a cDNA sequence corresponding to repeat number 18 from the 5′ variable region of O772P.

SEQ ID NO:521 (HB repeat5) represents an example of a cDNA sequence corresponding to repeat number 17 from the 5′ variable region of O772P.

SEQ ID NO:522 (HB repeat6 5′-end) represents an example of a cDNA sequence corresponding to repeat number 16 from the 5′ variable region of O772P.

SEQ ID NO:523 (1043400.1 repeat1) represents an example of a cDNA sequence corresponding to repeat number 9 from the 5′ variable region of O772P.

SEQ ID NO:524 (1043400.1 repeat2) represents an example of a cDNA sequence corresponding to repeat number 10 from the 5′ variable region of O772P.

SEQ ID NO:525 (1043400.1 repeat3) represents an example of a cDNA sequence corresponding to repeat No. 10/11 from the 5′ variable region of O772P.

SEQ ID NO:526 (1043400.1 repeat4) represents an example of a cDNA sequence corresponding to repeat number 11 from the 5′ variable region of O772P.

SEQ ID NO:527 (1043400.1 repeat5) represents an example of a cDNA sequence corresponding to repeat number 14 from the 5′ variable region of O772P.

SEQ ID NO:528 (1043400.1 repeat6) represents an example of a cDNA sequence corresponding to repeat number 17 from the 5′ variable region of O772P.

SEQ ID NO:529 (1043400.3 repeat1) represents an example of a cDNA sequence corresponding to repeat number 20 from the 5′ variable region of O772P.

SEQ ID NO:530 (1043400.3 repeat2) represents an example of a cDNA sequence corresponding to repeat number 21 from the 5′ variable region of O772P.

SEQ ID NO:531 (1043400.5 repeat1) represents an example of a cDNA sequence corresponding to repeat number 8 from the 5′ variable region of O772P.

SEQ ID NO:532 (1043400.5 repeat2) represents an example of a cDNA sequence corresponding to repeat number 9 from the 5′ variable region of O772P, in addition containing intron sequence.

SEQ ID NO:533 (1043400.5 repeat2) represents an example of a cDNA sequence corresponding to repeat number 9 from the 5′ variable region of O772P.

SEQ ID NO:534 (1043400.8 repeat1) represents an example of a cDNA sequence corresponding to repeat number 17 from the 5′ variable region of O772P.

SEQ ID NO:535 (1043400.8 repeat2) represents an example of a cDNA sequence corresponding to repeat number 18 from the 5′ variable region of O772P.

SEQ ID NO:536 (1043400.8 repeat3) represents an example of a cDNA sequence corresponding to repeat number 19 from the 5′ variable region of O772P.

SEQ ID NO:537 (1043400.9 repeat1) represents an example of a cDNA sequence corresponding to repeat number 4 from the 5′ variable region of O772P.

SEQ ID NO:538 (1043400.9 repeat2) represents an example of a cDNA sequence corresponding to repeat number 5 from the 5′ variable region of O772P.

SEQ ID NO:539 (1043400.9 repeat3) represents an example of a cDNA sequence corresponding to repeat number 7 from the 5′ variable region of O772P.

SEQ ID NO:540 (1043400.9 repeat4) represents an example of a cDNA sequence corresponding to repeat number 8 from the 5′ variable region of O772P.

SEQ ID NO:541 (1043400.11 repeat1) represents an example of a cDNA sequence corresponding to repeat number 1 from the 5′ variable region of O772P.

SEQ ID NO:542 (1043400.11 repeat2) represents an example of a cDNA sequence corresponding to repeat number 2 from the 5′ variable region of O772P.

SEQ ID NO:543 (1043400.11 repeat3) represents an example of a cDNA sequence corresponding to repeat number 3 from the 5′ variable region of O772P.

SEQ ID NO:544 (1043400.11 repeat4) represents an example of a cDNA sequence corresponding to repeat number 11 from the 5′ variable region of O772P.

SEQ ID NO:545 (1043400.11 repeat5) represents an example of a cDNA sequence corresponding to repeat number 12 from the 5′ variable region of O772P.

SEQ ID NO:546 (1043400.12 repeat1) represents an example of a cDNA sequence corresponding to repeat number 20 from the 5′ variable region of O772P.

SEQ ID NO:547 (PB repeatA) represents an example of a cDNA sequence corresponding to repeat number 1 from the 5′ variable region of O772P.

SEQ ID NO:548 (PB repeatB) represents an example of a cDNA sequence corresponding to repeat number 2 from the 5′ variable region of O772P.

SEQ ID NO:549 (PB repeatE) represents an example of a cDNA sequence corresponding to repeat number 3 from the 5′ variable region of O772P.

SEQ ID NO:550 (PB repeatG) represents an example of a cDNA sequence corresponding to repeat number 4 from the 5′ variable region of O772P.

SEQ ID NO:551 (PB repeatC) represents an example of a cDNA sequence corresponding to repeat number 4 from the 5′ variable region of O772P.

SEQ ID NO:552 (PB repeatH) represents an example of a cDNA sequence corresponding to repeat number 6 from the 5′ variable region of O772P.

SEQ ID NO:553 (PB repeatJ) represents an example of a cDNA sequence corresponding to repeat number 7 from the 5′ variable region of O772P.

SEQ ID NO:554 (PB repeatK) represents an example of a cDNA sequence corresponding to repeat number 8 from the 5′ variable region of O772P.

SEQ ID NO:555 (PB repeatD) represents an example of a cDNA sequence corresponding to repeat number 9 from the 5′ variable region of O772P.

SEQ ID NO:556 (PB repeatl) represents an example of a cDNA sequence corresponding to repeat number 10 from the 5′ variable region of O772P.

SEQ ID NO:557 (PB repeatM) represents an example of a cDNA sequence corresponding to repeat number 11 from the 5′ variable region of O772P.

SEQ ID NO:558 (PB repeat9) represents an example of a cDNA sequence corresponding to repeat number 12 from the 5′ variable region of O772P.

SEQ ID NO:559 (PB repeat8.5) represents an example of a cDNA sequence corresponding to repeat number 13 from the 5′ variable region of O772P.

SEQ ID NO:560 (PB repeat8) represents an example of a cDNA sequence corresponding to repeat number 14 from the 5′ variable region of O772P.

SEQ ID NO:561 (PB repeat7) represents an example of a cDNA sequence corresponding to repeat number 15 from the 5′ variable region of O772P.

SEQ ID NO:562 (PB repeat6) represents an example of a cDNA sequence corresponding to repeat number 16 from the 5′ variable region of O772P.

SEQ ID NO:563 (PB repeat5) represents an example of a cDNA sequence corresponding to repeat number 17 from the 5′ variable region of O772P.

SEQ ID NO:564 (PB repeat4) represents an example of a cDNA sequence corresponding to repeat number 18 from the 5′ variable region of O772P.

SEQ ID NO:565 (PB repeat3) represents an example of a cDNA sequence corresponding to repeat number 19 from the 5′ variable region of O772P.

SEQ ID NO:566 (PB repeat2) represents an example of a cDNA sequence corresponding to repeat number 20 from the 5′ variable region of O772P.

SEQ ID NO:567 (PB repeat1) represents an example of a cDNA sequence corresponding to repeat number 21 from the 5′ variable region of O772P.

SEQ ID NO:568 represents the cDNA sequence form the 3′ constant region.

SEQ ID NO:569 represents a cDNA sequence containing the consensus sequences of the 21 repeats, the 3′ constant region and the 3′ untranslated region.

SEQ ID NO:570 represents the cDNA sequence of the consensus repeat sequence.

SEQ ID NO:571 represents the consensus amino acid sequence of one potential open reading frame of repeat number 1 from the 5′ variable region of O772P.

SEQ ID NO:572 represents the consensus amino acid sequence of a second potential open reading frame of repeat number 1 from the 5′ variable region of O772P.

SEQ ID NO:573 represents the consensus amino acid sequence of a third potential open reading frame of repeat number 1 from the 5′ variable region of O772P.

SEQ ID NO:574 represents the consensus amino acid sequence of repeat number 2 from the 5′ variable region of O772P.

SEQ ID NO:575 represents the consensus amino acid sequence of repeat number 3 from the 5′ variable region of O772P.

SEQ ID NO:576 represents the consensus amino acid sequence of repeat number 4 from the 5′ variable region of O772P.

SEQ ID NO:577 represents the consensus amino acid sequence of repeat number 5 from the 5′ variable region of O772P.

SEQ ID NO:578 represents the consensus amino acid sequence of repeat number 6 from the 5′ variable region of O772P.

SEQ ID NO:579 represents the consensus amino acid sequence of repeat number 7 from the 5′ variable region of O772P.

SEQ ID NO:580 represents the consensus amino acid sequence of repeat number 8 from the 5′ variable region of O772P.

SEQ ID NO:581 represents the consensus amino acid sequence of repeat number 9 from the 5′ variable region of O772P.

SEQ ID NO:582 represents the consensus amino acid sequence of repeat number 10 from the 5′ variable region of O772P.

SEQ ID NO:583 represents the consensus amino acid sequence of repeat number 11 from the 5′ variable region of O772P.

SEQ ID NO:584 represents the consensus amino acid sequence of repeat number 12 from the 5′ variable region of O772P.

SEQ ID NO:585 represents the consensus amino acid sequence of repeat number 13 from the 5′ variable region of O772P.

SEQ ID NO:586 represents the consensus amino acid sequence of repeat number 14 from the 5′ variable region of O772P.

SEQ ID NO:587 represents the consensus amino acid sequence of repeat number 15 from the 5′ variable region of O772P.

SEQ ID NO:588 represents the consensus amino acid sequence of repeat number 16 from the 5′ variable region of O772P.

SEQ ID NO:589 represents the consensus amino acid sequence of repeat number 17 from the 5′ variable region of O772P.

SEQ ID NO:590 represents the consensus amino acid sequence of repeat number 18 from the 5′ variable region of O772P.

SEQ ID NO:591 represents the consensus amino acid sequence of repeat number 19 from the 5′ variable region of O772P.

SEQ ID NO:592 represents the consensus amino acid sequence of repeat number 20 from the 5′ variable region of O772P.

SEQ ID NO:593 represents the consensus amino acid sequence of repeat number 21 from the 5′ variable region of O772P.

SEQ ID NO:594 represents the amino acid sequence of the 3′ constant region.

SEQ ID NO:595 represents an amino acid sequence containing the consensus sequences of the 21 repeats and the 3′ constant region.

SEQ ID NO:596 represents the amino acid sequence of the consensus repeat sequence.

SEQ ID NO:597 represents the amino acid sequence for Peptide #1, a 30-mer peptide that corresponds to the predicted extracellular domain of O772P.

SEQ ID NO:598 represents the amino acid sequence for Peptide #2, a 30-mer peptide that corresponds to the predicted extracellular domain of O772P.

SEQ ID NO:599 represents the amino acid sequence for Peptide #3, a 30-mer peptide that corresponds to the predicted extracellular domain of O772P.

SEQ ID NO:600 represents the amino acid sequence of Peptide #1 from O8E, which corresponds to amino acids 1-20.

SEQ ID NO:601 represents the amino acid sequence of Peptide #2 from O8E, which corresponds to amino acids 16-35.

SEQ ID NO:602 represents the amino acid sequence of Peptide #3 from O8E, which corresponds to amino acids 31-50.

SEQ ID NO:603 represents the amino acid sequence of Peptide #4 from O8E, which corresponds to amino acids 46-65.

SEQ ID NO:604 represents the amino acid sequence of Peptide #5 from O8E, which corresponds to amino acids 61-80.

SEQ ID NO:605 represents the amino acid sequence of Peptide #6 from O8E, which corresponds to amino acids 76-95.

SEQ ID NO:606 represents the amino acid sequence of Peptide #7 from O8E, which corresponds to amino acids 91-110.

SEQ ID NO:607 represents the amino acid sequence of Peptide #8 from O8E, which corresponds to amino acids 106-125.

SEQ ID NO:608 represents the amino acid sequence of Peptide #9 from O8E, which corresponds to amino acids 120-140.

SEQ ID NO:609 represents the amino acid sequence of Peptide #10 from O8E, which corresponds to amino acids 136-155.

SEQ ID NO:610 represents the amino acid sequence of Peptide #11 from O8E, which corresponds to amino acids 151-170.

SEQ ID NO:611 represents the amino acid sequence of Peptide #12 from O8E, which corresponds to amino acids 166-185.

SEQ ID NO:612 represents the amino acid sequence of Peptide #13 from O8E, which corresponds to amino acids 181-200.

SEQ ID NO:613 represents the amino acid sequence of Peptide #14 from O8E, which corresponds to amino acids 196-215.

SEQ ID NO:614 represents the amino acid sequence of Peptide #15 from O8E, which corresponds to amino acids 211-230.

SEQ ID NO:615 represents the amino acid sequence of Peptide #16 from O8E, which corresponds to amino acids 225-245.

SEQ ID NO:616 represents the amino acid sequence of Peptide #17 from O8E, which corresponds to amino acids 241-260.

SEQ ID NO:617 represents the amino acid sequence of Peptide #18 from O8E, which corresponds to amino acids 256-275.

SEQ ID NO:618 represents the amino acid sequence of Peptide #19 from O8E, which corresponds to amino acids 263-282.

SEQ ID NO:619 is the DNA sequence for the O8E PCR primer, O8E-UP1.

SEQ ID NO:620 is the DNA sequence for the O8E reverse PCR primer designated O8E-DN1.

SEQ ID NO:621 is a DNA sequence corresponding to the O8E Rhesus orthologs.

SEQ ID NO:622 is a DNA sequence corresponding to the O8E mouse ortholog.

SEQ ID NO:623 is an amino acid sequence corresponding to the O8E Rhesus orthologs.

SEQ ID NO:624 is an amino acid sequence corresponding to the O8E mouse ortholog.

### DETAILED DESCRIPTION OF THE INVENTION

U.S. patents, U.S. patent application publications, U.S. patent applications, foreign patents, foreign patent applications and non-patent publications referred to in this specification and/or listed in the Application Data Sheet, are incorporated herein by reference, in their entirety.

The present invention is directed generally to compositions and their use in the therapy and diagnosis of cancer, particularly ovarian cancer. As described further below, illustrative compositions of the present invention include, but are not restricted to, polypeptides, particularly immunogenic polypeptides, polynucleotides encoding such polypeptides, antibodies and other binding agents, antigen presenting cells (APCs) and immune system cells (e.g., T cells).

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

### Polypeptide Compositions

As used herein, the term “polypeptide” is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

Particularly illustrative polypeptides of the present invention comprise those encoded by a polynucleotide sequence set forth in any one of SEQ ID NOs: 1-311, 313-387, 391, 457, 460-477, 512-570 and 619-622, or a sequence that hybridizes under moderately stringent conditions, or, alternatively, under highly stringent conditions, to a polynucleotide sequence set forth in any one of SEQ ID NOs: 1-311, 313-387, 391, 457, 460-477, 512-570 and 619-622. Certain other illustrative polypeptides of the invention comprise amino acid sequences as set forth in any one of SEQ ID NOs: 312, 388-389, 392-455, 458-459, 478-511, and 571-618.

The polypeptides of the present invention are sometimes herein referred to as ovarian tumor proteins or ovarian tumor polypeptides, as an indication that their identification has been based at least in part upon their increased levels of expression in ovarian tumor samples. Thus, an “ovarian tumor polypeptide” or “ovarian tumor protein,” refers generally to a polypeptide sequence of the present invention, or a polynucleotide sequence encoding such a polypeptide, that is expressed in a substantial proportion of ovarian tumor samples, for example preferably greater than about 20%, more preferably greater than about 30%, and most preferably greater than about 50% or more of ovarian tumor samples tested, at a level that is at least two fold, and preferably at least five fold, greater than the level of expression in normal tissues, as determined using a representative assay provided herein. A ovarian tumor polypeptide sequence of the invention, based upon its increased level of expression in tumor cells, has particular utility both as a diagnostic marker as well as a therapeutic target, as further described below.

In certain preferred embodiments, the polypeptides of the invention are immunogenic, i.e., they react detectably within an immunoassay (such as an ELISA or T-cell stimulation assay) with antisera and/or T-cells from a patient with ovarian cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one preferred embodiment, an immunogenic portion of a polypeptide of the present invention is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N- and/or C-terminal deletion (e.g., 1-30 amino acids, preferably 5-15 amino acids), relative to the mature protein.

In another embodiment, a polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous nucleic acid sequences contained in the polynucleotide sequences disclosed herein, or immunogenic fragments or variants thereof, or to one or more nucleic acid sequences which hybridize to one or more of these sequences under conditions of moderate to high stringency.

The present invention, in another aspect, provides polypeptide fragments comprising at least about 5, 10, 15, 20, 25, 50, or 100 contiguous amino acids, or more, including all intermediate lengths, of a polypeptide compositions set forth herein, such as those set forth in SEQ ID NOs: 312, 388-389, 392-455, 458-459, 478-511, and 571-618, or those encoded by a polynucleotide sequence set forth in a sequence of SEQ ID NOs: 1-311, 313-387, 391, 457, 460-477, 512-570 and 619-622.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein.

In one preferred embodiment, the polypeptide fragments and variants provided by the present invention are immunologically reactive with an antibody and/or T-cell that reacts with a full-length polypeptide specifically set forth herein.

In another preferred embodiment, the polypeptide fragments and variants provided by the present invention exhibit a level of immunogenic activity of at least about 50%, preferably at least about 70%, and most preferably at least about 90% or more of that exhibited by a full-length polypeptide sequence specifically set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein and/or using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N- and/or C-terminal of the mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O., (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes, pp. 626-645 Methods in Enzymology vol. 183, Academic Press, Inc., San Diego, Calif.; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Saitou, N. Nei, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, Calif. (1973); Wilbur, W. J. and Lipman, D. J., Proc. Natl. Acad., ScL USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, a polypeptide may be a xenogeneic polypeptide that comprises an polypeptide having substantial sequence identity, as described above, to the human polypeptide (also termed autologous antigen) which served as a reference polypeptide, but which xenogeneic polypeptide is derived from a different, non-human species. One skilled in the art will recognize that “self” antigens are often poor stimulators of CD8+ and CD4+ T-lymphocyte responses, and therefore efficient immunotherapeutic strategies directed against tumor polypeptides require the development of methods to overcome immune tolerance to particular self tumor polypeptides. For example, humans immunized with prostase protein from a xenogeneic (non human) origin are capable of mounting an immune response against the counterpart human protein, e.g., the human prostase tumor protein present on human tumor cells. Accordingly, the present invention provides methods for purifying the xenogeneic form of the tumor proteins set forth herein, such as the polypeptides set forth in SEQ ID NOs: 312, 388-389, 392-455, 458-459, 478-511, and 571-618, or those encoded by polynucleotide sequences set forth in SEQ ID NOs: 1-311, 313-387, 391, 457, 460-477, 512-570 and 619-622.

Therefore, one aspect of the present invention provides xenogeneic variants of the polypeptide compositions described herein. Such xenogeneic variants generally encompassed by the present invention will typically exhibit at least about 70%, 75%, 80%, 85%, 90%, 91%, 92%, 93%, 94%, 95%, 96%, 97%, 98%, or 99% or more identity along their lengths, to a polypeptide sequences set forth herein.

More particularly, the invention is directed to mouse, rat, monkey, porcine and other non-human polypeptides which can be used as xenogeneic forms of human polypeptides set forth herein, to induce immune responses directed against tumor polypeptides of the invention.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application 60/158,585; see also, Skeiky et al., Infection and Immun. 67:3998-4007 (1999), incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

Polypeptides of the invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

### Polynucleotide Compositions

The present invention, in other aspects, provides polynucleotide compositions. The terms “DNA” and “polynucleotide” are used essentially interchangeably herein to refer to a DNA molecule that has been isolated free of total genomic DNA of a particular species. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be also recognized by the skilled artisan, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide/protein of the invention or a portion thereof) or may comprise a sequence that encodes a variant or derivative, preferably and immunogenic variant or derivative, of such a sequence.

Therefore, according to another aspect of the present invention, polynucleotide compositions are provided that comprise some or all of a polynucleotide sequence set forth in any one of SEQ ID NOs: 1-311, 313-387, 391, 457, 460-477, 512-570 and 619-622, complements of a polynucleotide sequence set forth in any one of SEQ ID NOs: 1-311, 313-387, 391, 457, 460-477, 512-570 and 619-622, and degenerate variants of a polynucleotide sequence set forth in any one of SEQ ID NOs: 1-311, 313-387, 391, 457, 460-477, 512-570 and 619-622. In certain preferred embodiments, the polynucleotide sequences set forth herein encode immunogenic polypeptides, as described above.

In other related embodiments, the present invention provides polynucleotide variants having substantial identity to the sequences disclosed herein in SEQ ID NOs: 1-311, 313-387, 391, 457, 460-477, 512-570 and 619-622, for example those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the immunogenicity of the polypeptide encoded by the variant polynucleotide is not substantially diminished relative to a polypeptide encoded by a polynucleotide sequence specifically set forth herein). The term “variants” should also be understood to encompasses homologous genes of xenogenic origin.

In additional embodiments, the present invention provides polynucleotide fragments comprising or consisting of various lengths of contiguous stretches of sequence identical to or complementary to one or more of the sequences disclosed herein. For example, polynucleotides are provided by this invention that comprise or consist of at least about 10, 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of the disclosed sequence or at both ends of the disclosed sequence.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C. or 65-70° C.

In certain preferred embodiments, the polynucleotides described above, e.g., polynucleotide variants, fragments and hybridizing sequences, encode polypeptides that are immunologically cross-reactive with a polypeptide sequence specifically set forth herein. In other preferred embodiments, such polynucleotides encode polypeptides that have a level of immunogenic activity of at least about 50%, preferably at least about 70%, and more preferably at least about 90% of that for a polypeptide sequence specifically set forth herein.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., Unified Approach to Alignment and Phylogenes, pp. 626-645 (1990); Methods in Enzymology vol. 183, Academic Press, Inc., San Diego, Calif.; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, Calif. (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity for the polynucleotides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment of the invention, a mutagenesis approach, such as site-specific mutagenesis, is employed for the preparation of immunogenic variants and/or derivatives of the polypeptides described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the immunogenicity of a polypeptide vaccine. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants of the present invention, recursive sequence recombination, as described in U.S. Pat. No. 5,837,458, may be employed. In this approach, iterative cycles of recombination and screening or selection are performed to “evolve” individual polynucleotide variants of the invention having, for example, enhanced immunogenic activity.

In other embodiments of the present invention, the polynucleotide sequences provided herein can be advantageously used as probes or primers for nucleic acid hybridization. As such, it is contemplated that nucleic acid segments that comprise or consist of a sequence region of at least about a 15 nucleotide long contiguous sequence that has the same sequence as, or is complementary to, a 15 nucleotide long contiguous sequence disclosed herein will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

The ability of such nucleic acid probes to specifically hybridize to a sequence of interest will enable them to be of use in detecting the presence of complementary sequences in a given sample. However, other uses are also envisioned, such as the use of the sequence information for the preparation of mutant species primers, or primers for use in preparing other genetic constructions.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer. The choice of probe and primer sequences may be governed by various factors. For example, one may wish to employ primers from towards the termini of the total sequence.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202 (incorporated herein by reference), by introducing selected sequences into recombinant vectors for recombinant production, and by other recombinant DNA techniques generally known to those of skill in the art of molecular biology.

The nucleotide sequences of the invention may be used for their ability to selectively form duplex molecules with complementary stretches of the entire gene or gene fragments of interest. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Of course, for some applications, for example, where one desires to prepare mutants employing a mutant primer strand hybridized to an underlying template, less stringent (reduced stringency) hybridization conditions will typically be needed in order to allow formation of the heteroduplex. In these circumstances, one may desire to employ salt conditions such as those of from about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

According to another embodiment of the present invention, polynucleotide compositions comprising antisense oligonucleotides are provided. Antisense oligonucleotides have been demonstrated to be effective and targeted inhibitors of protein synthesis, and, consequently, provide a therapeutic approach by which a disease can be treated by inhibiting the synthesis of proteins that contribute to the disease. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829). Further, examples of antisense inhibition have been demonstrated with the nuclear protein cyclin, the multiple drug resistance gene (MDG1), ICAM-1, E-selectin, STK-1, striatal GABA_{A} receptor and human EGF (Jaskulski et al., Science 1988 Jun. 10; 240(4858):1544-6; Vasanthakumar and Ahmed, Cancer Commun. 1989; 1(4):225-32; Peris et al., Brain Res Mol Brain Res. 1998 Jun. 15; 57(2):310-20; U.S. Pat. No. 5,801,154; U.S. Pat. No. 5,789,573; U.S. Pat. No. 5,718,709 and U.S. Pat. No. 5,610,288). Antisense constructs have also been described that inhibit and can be used to treat a variety of abnormal cellular proliferations, e.g., cancer (U.S. Pat. No. 5,747,470; U.S. Pat. No. 5,591,317 and U.S. Pat. No. 5,783,683).

Therefore, in certain embodiments, the present invention provides oligonucleotide sequences that comprise all, or a portion of, any sequence that is capable of specifically binding to polynucleotide sequence described herein, or a complement thereof. In one embodiment, the antisense oligonucleotides comprise DNA or derivatives thereof. In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of polynucleotides disclosed herein. Selection of antisense compositions specific for a given gene sequence is based upon analysis of the chosen target sequence and determination of secondary structure, Tₘ, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA, are those which are at or near the AUG translation initiation codon, and those sequences which are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. 1997 Jul. 15; 25(14):2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

According to another embodiment of the invention, the polynucleotide compositions described herein are used in the design and preparation of ribozyme molecules for inhibiting expression of the tumor polypeptides and proteins of the present invention in tumor cells. Ribozymes are RNA-protein complexes that cleave nucleic acids in a site-specific fashion. Ribozymes have specific catalytic domains that possess endonuclease activity (Kim and Cech, Proc. Natl. Acad. Sci. USA. 1987 December; 84(24):8788-92; Forster and Symons, Cell. 1987 Apr. 24; 49(2):211-20). For example, a large number of ribozymes accelerate phosphoester transfer reactions with a high degree of specificity, often cleaving only one of several phosphoesters in an oligonucleotide substrate (Cech et al., Cell. 1981 December; 27(3 Pt 2):487-96; Michel and Westhof, J Mol. Biol. 1990 Dec. 5; 216(3):585-610; Reinhold-Hurek and Shub, Nature. 1992 May 14; 357(6374):173-6). This specificity has been attributed to the requirement that the substrate bind via specific base-pairing interactions to the internal guide sequence (“IGS”) of the ribozyme prior to chemical reaction.

Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc. Natl. Acad. Sci. USA. 1992 Aug. 15; 89(16):7305-9). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al. Nucleic Acids Res. 1992 Sep. 11; 20(17):4559-65. Examples of hairpin motifs are described by Hampel et al. (Eur. Pat. Appl. Publ. No. EP 0360257), Hampel and Tritz, Biochemistry 1989 Jun. 13; 28(12):4929-33; Hampel et al., Nucleic Acids Res. 1990 Jan. 25; 18(2):299-304 and U.S. Pat. No. 5,631,359. An example of the hepatitis δ virus motif is described by Perrotta and Been, Biochemistry. 1992 Dec. 1; 31(47):11843-52; an example of the RNaseP motif is described by Guerrier-Takada et al., Cell. 1983 December; 35(3 Pt 2):849-57; Neurospora VS RNA ribozyme motif is described by Collins (Saville and Collins, Cell. 1990 May 18; 61(4):685-96; Saville and Collins, Proc. Natl. Acad. Sci. USA, 88(19):8826-30 (Oct. 1 1991); Collins and Olive, Biochemistry 32(11):2795-9 (Mar. 23 1993); and an example of the Group I intron is described in (U.S. Pat. No. 4,987,071). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Sullivan et al. (Int. Pat. Appl. Publ. No. WO 94/02595) describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Another means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells Ribozymes expressed from such promoters have been shown to function in mammalian cells. Such transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, semliki forest virus, sindbis virus vectors).

In another embodiment of the invention, peptide nucleic acids (PNAs) compositions are provided. PNA is a DNA mimic in which the nucleobases are attached to a pseudopeptide backbone (Good and Nielsen, Antisense Nucleic Acid Drug Dev. 1997 7(4) 431-37). PNA is able to be utilized in a number methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 15(6):224-9 (June 1997)). As such, in certain embodiments, one may prepare PNA sequences that are complementary to one or more portions of the ACE mRNA sequence, and such PNA compositions may be used to regulate, alter, decrease, or reduce the translation of ACE-specific mRNA, and thereby alter the level of ACE activity in a host cell to which such PNA compositions have been administered.

PNAs have 2-aminoethyl-glycine linkages replacing the normal phosphodiester backbone of DNA (Nielsen et al., Science 254(5037):1497-500 (Dec. 6 1991); Hanvey et al., Science 258(5087):1481-5 (Nov. 27 1992); Hyrup and Nielsen, Bioorg. Med. Chem. 4(1):5-23 (January 1996). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg. Med. Chem. 3(4):437-45 (April 1995)). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem 3(4):437-45 (April 1995); Petersen et al., J Pept Sci 1(3):175-83 (May-June 1995); Orum et al., Biotechniques 19(3):472-80 (September 1995); Footer et al., Biochemistry. 1996 Aug. 20; 35(33):10673-9; Griffith et al., Nucleic Acids Res 23(15):3003-8 (Aug. 11 1995); Pardridge et al., Proc. Natl. Acad. Sci. USA. 92(12):5592-6 (Jun. 6 1995); Boffa et al., Proc. Natl. Acad. Sci. USA. 92(6):1901-5 (Mar. 14 1995); Gambacorti-Passerini et al., Blood 88(4):1411-7 (Aug. 15 1996); Armitage et al., Proc. Natl. Acad. Sci. USA. 94(23):12320-5 (Nov. 11 1997); Seeger et al., Biotechniques 23(3):512-7 (September 1997)). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem 65(24):3545-9 (Dec. 15 1993) and Jensen et al. (Biochemistry. 1997 Apr. 22; 36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

### Polynucleotide Identification, Characterization and Expression

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references). For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using the microarray technology of Affymetrix, Inc. (Santa Clara, Calif.) according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997). Alternatively, polynucleotides may be amplified from cDNA prepared from cells expressing the proteins described herein, such as tumor cells.

Many template dependent processes are available to amplify a target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR™ amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Publ. No. PCT/US87/00880; Strand Displacement Amplification (SDA) and Repair Chain Reaction (RCR). Still other amplification methods are described in Great Britain Pat. Appl. No. 2 202 328, and in PCT Intl. Pat. Appl. Publ. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE” (Frohman, 1990), and “one-sided PCR” (Ohara, 1989) are also well-known to those of skill in the art.

An amplified portion of a polynucleotide of the present invention may be used to isolate a full length gene from a suitable library (e.g., a tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, amplification techniques, such as those described above, can be useful for obtaining a full length coding sequence from a partial cDNA sequence. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of a polypeptide, or a portion thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, T. (1983) Proteins, Structures and Molecular Principles, WH Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as pBLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke, G. and S. M. Schuster (1989) J. Biol. Chem. 264:5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-311. Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91:3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation. glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3:263-281) while the enterokinase cleavage site provides a means for purifying the desired polypeptide from the fusion protein. A discussion of vectors which contain fusion proteins is provided in Kroll, D. J. et al. (1993; DNA Cell Biol. 12:441-453).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

### Antibody Compositions, Fragments Thereof and Other Binding Agents

According to another aspect, the present invention further provides binding agents, such as antibodies and antigen-binding fragments thereof, that exhibit immunological binding to a tumor polypeptide disclosed herein, or to a portion, variant or derivative thereof. An antibody, or antigen-binding fragment thereof, is said to “specifically bind,” “immunogically bind,” and/or is “immunologically reactive” to a polypeptide of the invention if it reacts at a detectable level (within, for example, an ELISA assay) with the polypeptide, and does not react detectably with unrelated polypeptides under similar conditions.

Immunological binding, as used in this context, generally refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

An “antigen-binding site,” or “binding portion” of an antibody refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Binding agents may be further capable of differentiating between patients with and without a cancer, such as ovarian cancer, using the representative assays provided herein. For example, antibodies or other binding agents that bind to a tumor protein will preferably generate a signal indicating the presence of a cancer in at least about 20% of patients with the disease, more preferably at least about 30% of patients. Alternatively, or in addition, the antibody will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. Preferably, a statistically significant number of samples with and without the disease will be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of therapeutically useful molecules are known in the art which comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both antigen-binding sites. An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J. Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

In another embodiment of the invention, monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

### T Cell Compositions

The present invention, in another aspect, provides T cells specific for a tumor polypeptide disclosed herein, or for a variant or derivative thereof. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures.

T cells may be stimulated with a polypeptide, polynucleotide encoding a polypeptide and/or an antigen presenting cell (APC) that expresses such a polypeptide. Such stimulation is performed under conditions and for a time sufficient to permit the generation of T cells that are specific for the polypeptide of interest. Preferably, a tumor polypeptide or polynucleotide of the invention is present within a delivery vehicle, such as a microsphere, to facilitate the generation of specific T cells.

T cells are considered to be specific for a polypeptide of the present invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor polypeptide (100 ng/ml-100 μg/ml, preferably 200 ng/ml-25 μg/ml) for 3-7 days will typically result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a tumor polypeptide, polynucleotide or polypeptide-expressing APC may be CD4⁺ and/or CD8⁺. Tumor polypeptide-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a tumor polypeptide, polynucleotide or APC can be expanded in number either in vitro or in vivo. Proliferation of such T cells in vitro may be accomplished in a variety of ways. For example, the T cells can be re-exposed to a tumor polypeptide, or a short peptide corresponding to an immunogenic portion of such a polypeptide, with or without the addition of T cell growth factors, such as interleukin-2, and/or stimulator cells that synthesize a tumor polypeptide. Alternatively, one or more T cells that proliferate in the presence of the tumor polypeptide can be expanded in number by cloning. Methods for cloning cells are well known in the art, and include limiting dilution.

### T Cell Receptor Compositions

The T cell receptor (TCR) consists of 2 different, highly variable polypeptide chains, termed the T-cell receptor α and β chains, that are linked by a disulfide bond (Janeway, Travers, Walport. Immunobiology. Fourth Ed., 148-159. Elsevier Science Ltd/Garland Publishing. 1999). The α/β heterodimer complexes with the invariant CD3 chains at the cell membrane. This complex recognizes specific antigenic peptides bound to MHC molecules. The enormous diversity of TCR specificities is generated much like immunoglobulin diversity, through somatic gene rearrangement. The β chain genes contain over 50 variable (V), 2 diversity (D), over 10 joining (J) segments, and 2 constant region segments (C). The α chain genes contain over 70 V segments, and over 60 J segments but no D segments, as well as one C segment. During T cell development in the thymus, the D to J gene rearrangement of the β chain occurs, followed by the V gene segment rearrangement to the DJ. This functional VDJβ exon is transcribed and spliced to join to a Cβ. For the α chain, a Vα gene segment rearranges to a Jα gene segment to create the functional exon that is then transcribed and spliced to the Cα. Diversity is further increased during the recombination process by the random addition of P and N-nucleotides between the V, D, and J segments of the b chain and between the V and J segments in the α chain (Janeway, Travers, Walport. Immunobiology. Fourth Ed., 98 and 150. Elsevier Science Ltd/Garland Publishing. 1999).

The present invention, in another aspect, provides TCRs specific for a polypeptide disclosed herein, or for a variant or derivative thereof. In accordance with the present invention, polynucleotide and amino acid sequences are provided for the V-J or V-D-J junctional regions or parts thereof for the alpha and beta chains of the T-cell receptor which recognize tumor polypeptides described herein. In general, this aspect of the invention relates to T-cell receptors which recognize or bind tumor polypeptides presented in the context of MHC. In a preferred embodiment the tumor antigens recognized by the T-cell receptors comprise a polypeptide of the present invention. For example, cDNA encoding a TCR specific for a ovarian tumor peptide can be isolated from T cells specific for a tumor polypeptide using standard molecular biological and recombinant DNA techniques.

This invention further includes the T-cell receptors or analogs thereof having substantially the same function or activity as the T-cell receptors of this invention which recognize or bind tumor polypeptides. Such receptors include, but are not limited to, a fragment of the receptor, or a substitution, addition or deletion mutant of a T-cell receptor provided herein. This invention also encompasses polypeptides or peptides that are substantially homologous to the T-cell receptors provided herein or that retain substantially the same activity. The term “analog” includes any protein or polypeptide having an amino acid residue sequence substantially identical to the T-cell receptors provided herein in which one or more residues, preferably no more than 5 residues, more preferably no more than 25 residues have been conservatively substituted with a functionally similar residue and which displays the functional aspects of the T-cell receptor as described herein.

The present invention further provides for suitable mammalian host cells, for example, non-specific T-cells, that are transfected with a polynucleotide encoding TCRs specific for a polypeptide described herein, thereby rendering the host cell specific for the polypeptide. The α and β chains of the TCR may be contained on separate expression vectors or alternatively, on a single expression vector that also contains an internal ribosome entry site (IRES) for cap-independent translation of the gene downstream of the IRES. Said host cells expressing TCRs specific for the polypeptide may be used, for example, for adoptive immunotherapy of ovarian cancer as discussed further below.

In further aspects of the present invention, cloned TCRs specific for a polypeptide recited herein may be used in a kit for the diagnosis of ovarian cancer. For example, the nucleic acid sequence or portions thereof, of tumor-specific TCRs can be used as probes or primers for the detection of expression of the rearranged genes encoding the specific TCR in a biological sample. Therefore, the present invention further provides for an assay for detecting messenger RNA or DNA encoding the TCR specific for a polypeptide.

### Pharmaceutical Compositions

In additional embodiments, the present invention concerns formulation of one or more of the polynucleotide, polypeptide, T-cell, TCR, and/or antibody compositions disclosed herein in pharmaceutically-acceptable carriers for administration to a cell or an animal, either alone, or in combination with one or more other modalities of therapy.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Therefore, in another aspect of the present invention, pharmaceutical compositions are provided comprising one or more of the polynucleotide, polypeptide, antibody, TCR, and/or T-cell compositions described herein in combination with a physiologically acceptable carrier. In certain preferred embodiments, the pharmaceutical compositions of the invention comprise immunogenic polynucleotide and/or polypeptide compositions of the invention for use in prophylactic and therapeutic vaccine applications. Vaccine preparation is generally described in, for example, M. F. Powell and M. J. Newman, eds., “Vaccine Design (the subunit and adjuvant approach),” Plenum Press (NY, 1995). Generally, such compositions will comprise one or more polynucleotide and/or polypeptide compositions of the present invention in combination with one or more immunostimulants.

It will be apparent that any of the pharmaceutical compositions described herein can contain pharmaceutically acceptable salts of the polynucleotides and polypeptides of the invention. Such salts can be prepared, for example, from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

In another embodiment, illustrative immunogenic compositions, e.g., vaccine compositions, of the present invention comprise DNA encoding one or more of the polypeptides as described above, such that the polypeptide is generated in situ. As noted above, the polynucleotide may be administered within any of a variety of delivery systems known to those of ordinary skill in the art. Indeed, numerous gene delivery techniques are well known in the art, such as those described by Rolland, Crit. Rev. Therap. Drug Carrier Systems 15:143-198, 1998, and references cited therein. Appropriate polynucleotide expression systems will, of course, contain the necessary regulatory DNA regulatory sequences for expression in a patient (such as a suitable promoter and terminating signal). Alternatively, bacterial delivery systems may involve the administration of a bacterium (such as Bacillus-Calmette-Guerrin) that expresses an immunogenic portion of the polypeptide on its cell surface or secretes such an epitope.

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the polynucleotides encoding polypeptides of the present invention by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA 87:6743-6747 (1990); Fuerst et al., Proc. Natl. Acad. Sci. USA 83:8122-8126 (1986).

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al. J. Biol. Chem. 268:6866-6869 (1993) and Wagner et al., Proc. Natl. Acad. Sci. USA 89:6099-6103 (1992), can also be used for gene delivery under the invention.

Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

In another embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In still another embodiment, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

According to another embodiment, the pharmaceutical compositions described herein will comprise one or more immunostimulants in addition to the immunogenic polynucleotide, polypeptide, antibody, T-cell, TCR, and/or APC compositions of this invention. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Within certain embodiments of the invention, the adjuvant composition is preferably one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Certain preferred adjuvants for eliciting a predominantly Th1-type response include, for example, a combination of monophosphoryl lipid A, preferably 3-de-O-acylated monophosphoryl lipid A, together with an aluminum salt. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol® to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 is disclosed in WO 00/09159. Preferably the formulation additionally comprises an oil in water emulsion and tocopherol.

Additional illustrative adjuvants for use in the pharmaceutical compositions of the invention include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula

HO(CH₂CH₂O)ₙ-A-R, (I)

wherein, n is 1-50, A is a bond or —C(O)—, R is C₁₋₅₀ alkyl or Phenyl O₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably O₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

According to another embodiment of this invention, an immunogenic composition described herein is delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1 BB).

APCs may generally be transfected with a polynucleotide of the invention (or portion or other variant thereof) such that the encoded polypeptide, or an immunogenic portion thereof, is expressed on the cell surface. Such transfection may take place ex vivo, and a pharmaceutical composition comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems. such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

In another illustrative embodiment, calcium phosphate core particles are employed as carriers, vaccine adjuvants, or as controlled release matrices for the compositions of this invention. Exemplary calcium phosphate particles are disclosed, for example, in published patent application No. WO/0046147.

The pharmaceutical compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., Nature 1997 Mar. 27; 386(6623):410-4; Hwang et al., Crit. Rev Ther Drug Carrier Syst 1998; 15(3):243-84; U.S. Pat. No. 5,641,515; U.S. Pat. No. 5,580,579 and U.S. Pat. No. 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al., J Controlled Release 1998 Mar. 2; 52(1-2):81-7) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol 1998 July; 16(7):307-21; Takakura, Nippon Rinsho 1998 March; 56(3):691-5; Chandran et al., Indian J Exp Biol. 1997 August; 35(8):801-9; Margalit, Crit. Rev Ther Drug Carrier Syst. 1995; 12(2-3):233-61; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J Biol. Chem. 1990 Sep. 25; 265(27):16337-42; Muller et al., DNA Cell Biol. 1990 April; 9(3):221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, the use of liposomes does not appear to be associated with autoimmune responses or unacceptable toxicity after systemic delivery.

In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev Ind Pharm. 1998 December; 24(12):1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit. Rev Ther Drug Carrier Syst. 1988; 5(1):1-20; zur Muhlen et al., Eur J Pharm Biopharm. 1998 March; 45(2):149-55; Zambaux et al. J Controlled Release. 1998 Jan. 2; 50(1-3):31-40; and U.S. Pat. No. 5,145,684.

### Cancer Therapeutic Methods

Immunologic approaches to cancer therapy are based on the recognition that cancer cells can often evade the body's defenses against aberrant or foreign cells and molecules, and that these defenses might be therapeutically stimulated to regain the lost ground, e.g., pgs. 623-648 in Klein, Immunology (Wiley-Interscience, New York, 1982). Numerous recent observations that various immune effectors can directly or indirectly inhibit growth of tumors has led to renewed interest in this approach to cancer therapy, e.g., Jager, et al., Oncology 2001; 60(1):1-7; Renner, et al., Ann Hematol 2000 December; 79(12):651-9.

Four-basic cell types whose function has been associated with antitumor cell immunity and the elimination of tumor cells from the body are: i) B-lymphocytes which secrete immunoglobulins into the blood plasma for identifying and labeling the nonself invader cells; ii) monocytes which secrete the complement proteins that are responsible for lysing and processing the immunoglobulin-coated target invader cells; iii) natural killer lymphocytes having two mechanisms for the destruction of tumor cells, antibody-dependent cellular cytotoxicity and natural killing; and iv) T-lymphocytes possessing antigen-specific receptors and having the capacity to recognize a tumor cell carrying complementary marker molecules (Schreiber, H., 1989, in Fundamental Immunology (ed). W. E. Paul, pp. 923-955).

Cancer immunotherapy generally focuses on inducing humoral immune responses, cellular immune responses, or both. Moreover, it is well established that induction of CD4⁺ T helper cells is necessary in order to secondarily induce either antibodies or cytotoxic CD8⁺ T cells. Polypeptide antigens that are selective or ideally specific for cancer cells, particularly ovarian cancer cells, offer a powerful approach for inducing immune responses against ovarian cancer, and are an important aspect of the present invention.

Therefore, in further aspects of the present invention, the pharmaceutical compositions described herein may be used to stimulate an immune response against cancer, particularly for the immunotherapy of ovarian cancer. Within such methods, the pharmaceutical compositions described herein are administered to a patient, typically a warm-blooded animal, preferably a human. A patient may or may not be afflicted with cancer. Pharmaceutical compositions and vaccines may be administered either prior to or following surgical removal of primary tumors and/or treatment such as administration of radiotherapy or conventional chemotherapeutic drugs. As discussed above, administration of the pharmaceutical compositions may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Monoclonal antibodies may be labeled with any of a variety of labels for desired selective usages in detection, diagnostic assays or therapeutic applications (as described in U.S. Pat. Nos. 6,090,365; 6,015,542; 5,843,398; 5,595,721; and 4,708,930, hereby incorporated by reference in their entirety as if each was incorporated individually). In each case, the binding of the labelled monoclonal antibody to the determinant site of the antigen will signal detection or delivery of a particular therapeutic agent to the antigenic determinant on the non-normal cell. A further object of this invention is to provide the specific monoclonal antibody suitably labelled for achieving such desired selective usages thereof.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment.

### Cancer Detection and Diagnostic Compositions, Methods and Kits

In general, a cancer may be detected in a patient based on the presence of one or more ovarian tumor proteins and/or polynucleotides encoding such proteins in a biological sample (for example, blood, sera, sputum urine and/or tumor biopsies) obtained from the patient. In other words, such proteins may be used as markers to indicate the presence or absence of a cancer such as ovarian cancer. In addition, such proteins may be useful for the detection of other cancers. The binding agents provided herein generally permit detection of the level of antigen that binds to the agent in the biological sample.

Polynucleotide primers and probes may be used to detect the level of mRNA encoding a tumor protein, which is also indicative of the presence or absence of a cancer. In general, a tumor sequence should be present at a level that is at least two-fold, preferably three-fold, and more preferably five-fold or higher in tumor tissue than in normal tissue of the same type from which the tumor arose. Expression levels of a particular tumor sequence in tissue types different from that in which the tumor arose are irrelevant in certain diagnostic embodiments since the presence of tumor cells can be confirmed by observation of predetermined differential expression levels, e.g., 2-fold, 5-fold, etc, in tumor tissue to expression levels in normal tissue of the same type.

Other differential expression patterns can be utilized advantageously for diagnostic purposes. For example, in one aspect of the invention, overexpression of a tumor sequence in tumor tissue and normal tissue of the same type, but not in other normal tissue types, e.g., PBMCs, can be exploited diagnostically. In this case, the presence of metastatic tumor cells, for example in a sample taken from the circulation or some other tissue site different from that in which the tumor arose, can be identified and/or confirmed by detecting expression of the tumor sequence in the sample, for example using RT-PCR analysis. In many instances, it will be desired to enrich for tumor cells in the sample of interest, e.g., PBMCs, using cell capture or other like techniques.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length ovarian tumor proteins and polypeptide portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with ovarian least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as ovarian cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use tumor polypeptides to detect antibodies that bind to such polypeptides in a biological sample. The detection of such tumor protein specific antibodies may correlate with the presence of a cancer.

A cancer may also, or alternatively, be detected based on the presence of T cells that specifically react with a tumor protein in a biological sample. Within certain methods, a biological sample comprising CD4⁺ and/or CD8⁺ T cells isolated from a patient is incubated with a tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of tumor polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding a tumor protein in a biological sample. For example, at least two oligonucleotide primers may be employed in a polymerase chain reaction (PCR) based assay to amplify a portion of a tumor cDNA derived from a biological sample, wherein at least one of the oligonucleotide primers is specific for (i.e., hybridizes to) a polynucleotide encoding the tumor protein. The amplified cDNA is then separated and detected using techniques well known in the art, such as gel electrophoresis.

Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a tumor protein of the invention that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another aspect of the present invention, cell capture technologies may be used in conjunction, with, for example, real-time PCR to provide a more sensitive tool for detection of metastatic cells expressing ovarian tumor antigens. Detection of ovarian cancer cells in biological samples, e.g., bone marrow samples, peripheral blood, and small needle aspiration samples is desirable for diagnosis and prognosis in ovarian cancer patients.

Immunomagnetic beads coated with specific monoclonal antibodies to surface cell markers, or tetrameric antibody complexes, may be used to first enrich or positively select cancer cells in a sample. Various commercially available kits may be used, including Dynabeads® Epithelial Enrich (Dynal Biotech, Oslo, Norway), StemSep™ (StemCell Technologies, Inc., Vancouver, BC), and RosetteSep (StemCell Technologies). A skilled artisan will recognize that other methodologies and kits may also be used to enrich or positively select desired cell populations. Dynabeads® Epithelial Enrich contains magnetic beads coated with mAbs specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses.

RosetteSep can be used to enrich cells directly from a blood sample and consists of a cocktail of tetrameric antibodies that targets a variety of unwanted cells and crosslinks them to glycophorin A on red blood cells (RBC) present in the sample, forming rosettes. When centrifuged over Ficoll, targeted cells pellet along with the free RBC. The combination of antibodies in the depletion cocktail determines which cells will be removed and consequently which cells will be recovered. Antibodies that are available include, but are not limited to: CD2, CD3, CD4, CD5, CD8, CD10, CD11b, CD14, CD15, CD16, CD19, CD20, CD24, CD25, CD29, CD33, CD34, CD36, CD38, CD41, CD45, CD45RA, CD45RO, CD56, CD66B, CD66e, HLA-DR, IgE, and TCRαβ.

Additionally, it is contemplated in the present invention that mAbs specific for ovarian tumor antigens can be generated and used in a similar manner. For example, mAbs that bind to tumor-specific cell surface antigens may be conjugated to magnetic beads, or formulated in a tetrameric antibody complex, and used to enrich or positively select metastatic ovarian tumor cells from a sample. Once a sample is enriched or positively selected, cells may be lysed and RNA isolated. RNA may then be subjected to RT-PCR analysis using ovarian tumor-specific primers in a real-time PCR assay as described herein. One skilled in the art will recognize that enriched or selected populations of cells may be analyzed by other methods (e.g., in situ hybridization or flow cytometry).

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a tumor protein.

The following Examples are offered by way of illustration and not by way of limitation.

### EXAMPLES

### Example 1

### Identification of Representative Ovarian Carcinoma Protein cDNAs

This Example illustrates the identification of cDNA molecules encoding ovarian carcinoma proteins.

Anti-SCID mouse sera (generated against sera from SCID mice carrying late passage ovarian carcinoma) was pre-cleared of E. coli and phage antigens and used at a 1:200 dilution in a serological expression screen. The library screened was made from a SCID-derived human ovarian tumor (OV9334) using a directional RH oligo(dT) priming cDNA library construction kit and the λScreen vector (Novagen). A bacteriophage lambda screen was employed. Approximately 400,000 pfu of the amplified OV9334 library were screened.

196 positive clones were isolated. Certain sequences that appear to be novel are provided in FIGS. 1A-1S and SEQ ID NO:1 to 71. Three complete insert sequences are shown in FIGS. 2A-2C (SEQ ID NO:72 to 74). Other clones having known sequences are presented in FIGS. 15A-15EEE (SEQ ID NO:82 to 310). Database searches identified the following sequences that were substantially identical to the sequences presented in FIGS. 15A-15EEE.

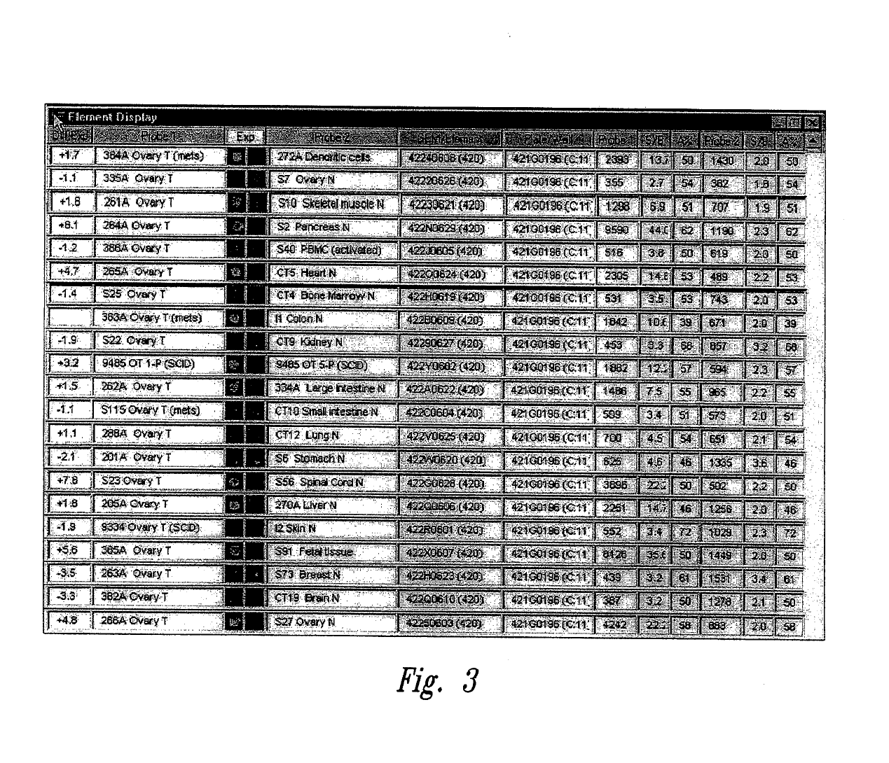

These clones were further characterized using microarray technology to determine mRNA expression levels in a variety of tumor and normal tissues. Such analyses were performed using a Synteni (Palo Alto, Calif.) microarray, according to the manufacturer's instructions. PCR amplification products were arrayed on slides, with each product occupying a unique location in the array. mRNA was extracted from the tissue sample to be tested, reverse transcribed and fluorescent-labeled cDNA probes were generated. The microarrays were probed with the labeled cDNA probes and the slides were scanned to measure fluorescence intensity. Data was analyzed using Synteni's provided GEMtools software. The results for one clone (13695, also referred to as O8E) are shown in FIG. 3.

### Example 2

### Identification of Ovarian Carcinoma cDNAs Using Microarray Technology

This Example illustrates the identification of ovarian carcinoma polynucleotides by PCR subtraction and microarray analysis. Microarrays of cDNAs were analyzed for ovarian tumor-specific expression using a Synteni (Palo Alto, Calif.) microarray, according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997).

A PCR subtraction was performed using a tester comprising cDNA of four ovarian tumors (three of which were metastatic tumors) and a driver of cDNA form five normal tissues (adrenal gland, lung, pancreas, spleen and brain). cDNA fragments recovered from this subtraction were subjected to DNA microarray analysis where the fragments were PCR amplified, adhered to chips and hybridized with fluorescently labeled probes derived from mRNAs of human ovarian tumors and a variety of normal human tissues. In this analysis, the slides were scanned and the fluorescence intensity was measured, and the data were analyzed using Synteni's GEMtools software. In general, sequences showing at least a 5-fold increase in expression in tumor cells (relative to normal cells) were considered ovarian tumor antigens. The fluorescent results were analyzed and clones that displayed increased expression in ovarian tumors were further characterized by DNA sequencing and database searches to determine the novelty of the sequences.

Using such assays, an ovarian tumor antigen was identified that is a splice fusion between the human T-cell leukemia virus type I oncoprotein TAX (see Jin et al., Cell 93:81-91, 1998) and an extracellular matrix protein called osteonectin. A splice junction sequence exists at the fusion point. The sequence of this clone is presented in FIG. 4 and SEQ ID NO:75. Osteonectin, unspliced and unaltered, was also identified from such assays independently.

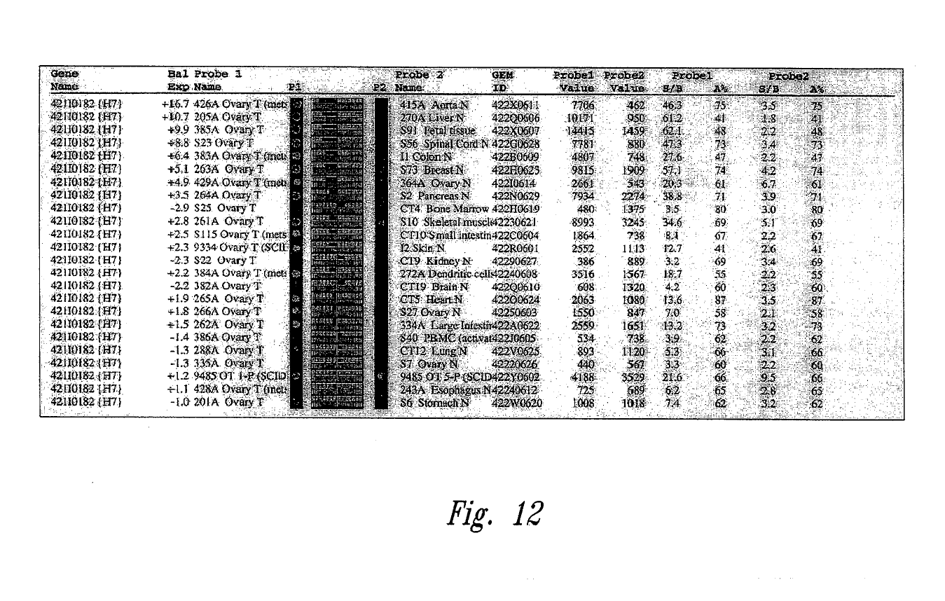

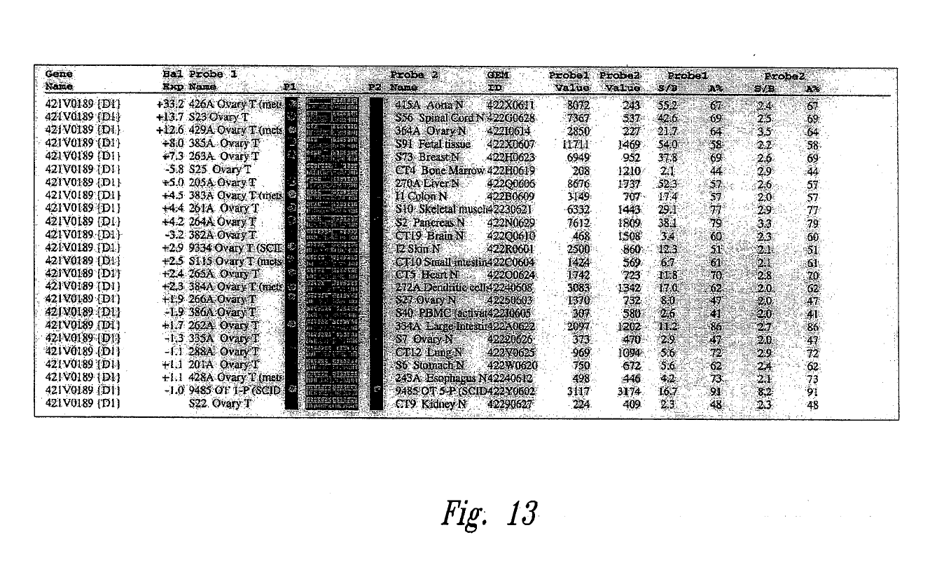

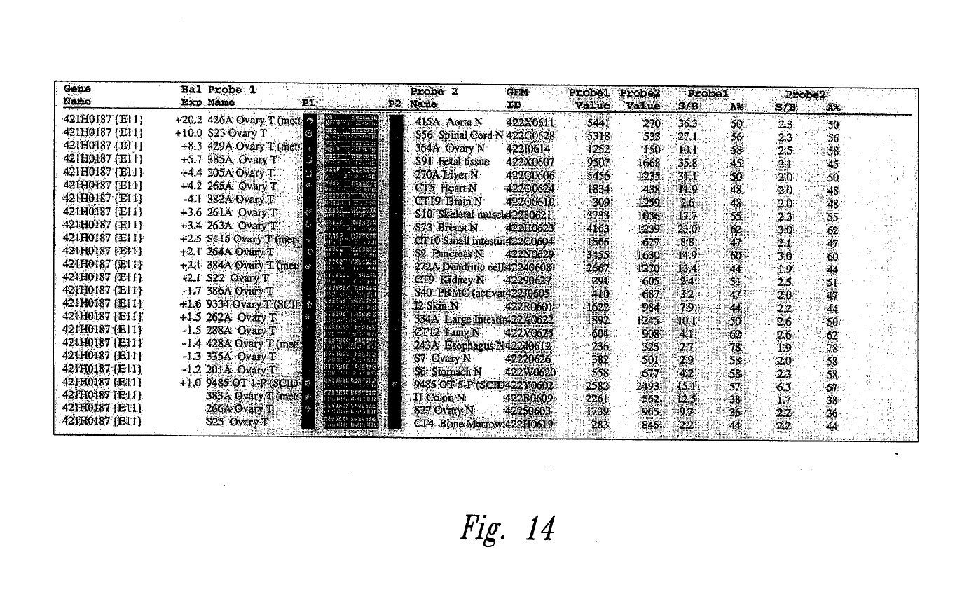

Further clones identified by this method are referred to herein as 3f, 6b, 8e, 8h, 12c and 12h. Sequences of these clones are shown in FIGS. 5 to 9 and SEQ ID NO:76 to 81. Microarray analyses were performed as described above, and are presented in FIGS. 10 to 14. A full length sequence encompassing clones 3f, 6b, 8e and 12h was obtained by screening an ovarian tumor (SCID-derived) cDNA library. This 2996 base pair sequence (designated O772P) is presented in SEQ ID NO:311, and the encoded 914 amino acid protein sequence is shown in SEQ ID NO:312. PSORT analysis indicates a Type 1a transmembrane protein localized to the plasma membrane.

In addition to certain of the sequences described above, this screen identified the following sequences which are described in detail in Table 2:

TABLE 2

| Sequence | | Comments |
|---|---|---|
| OV4vG11 | (SEQ ID NO: 313) | human clone 1119D9 on chromosome 20p12 |
| OV4vB11 | (SEQ ID NO: 314) | human UWGC: y14c094 from chromosome |
| | | 6p21 |
| OV4vD9 | (SEQ ID NO: 315) | human clone 1049G16 chromosome 20g12- |
| | | 13.2 |
| OV4vD5 | (SEQ ID NO: 316) | human KIAA0014 gene |
| OV4vC2 | (SEQ ID NO: 317) | human KIAA0084 gene |
| OV4vF3 | (SEQ ID NO: 318) | human chromosome 19 cosmid R31167 |
| OV4VC1 | (SEQ ID NO: 319) | Novel |
| OV4vH3 | (SEQ ID NO: 320) | Novel |
| OV4vD2 | (SEQ ID NO: 321) | novel |
| O815P | (SEQ ID NO: 322) | novel |
| OV4vC12 | (SEQ ID NO: 323) | novel |
| OV4vA4 | (SEQ ID NO: 324) | novel |
| OV4vA3 | (SEQ ID NO: 325) | novel |
| OV4v2A5 | (SEQ ID NO: 326) | novel |
| O819P | (SEQ ID NO: 327) | novel |
| O818P | (SEQ ID NO: 328) | novel |
| O817P | (SEQ ID NO: 329) | novel |
| O816P | (SEQ ID NO: 330) | novel |
| Ov4vC5 | (SEQ ID NO: 331) | novel |
| 21721 | (SEQ ID NO: 332) | human lumican |
| 21719 | (SEQ ID NO: 333) | human retinoic acid-binding protein II |
| 21717 | (SEQ ID NO: 334) | human26S proteasome ATPase subunit |
| 21654 | (SEQ ID NO: 335) | human copine I |
| 21627 | (SEQ ID NO: 336) | human neuron specific gamma-2 enolase |
| 21623 | (SEQ ID NO: 337) | human geranylgeranyl transferase II |
| 21621 | (SEQ ID NO: 338) | human cyclin-dependent protein kinase |
| 21616 | (SEQ ID NO: 339) | human prepro-megakaryocyte potentiating |
| | | factor |
| 21612 | (SEQ ID NO: 340) | human UPH1 |
| 21558 | (SEQ ID NO: 341) | human RaIGDS-like 2 (RGL2) |
| 21555 | (SEQ ID NO: 342) | human autoantigen P542 |
| 21548 | (SEQ ID NO: 343) | human actin-related protein (ARP2) |
| 21462 | (SEQ ID NO: 344) | human huntingtin interacting protein |
| 21441 | (SEQ ID NO: 345) | human 90 K product (tumor associated antigen) |
| 21439 | (SEQ ID NO: 346) | human guanine nucleotide regulator protein |
| | | (tim1) |
| 21438 | (SEQ ID NO: 347) | human Ku autoimmune (p70/p80) antigen |
| 21237 | (SEQ ID NO: 348) | human S-laminin |
| 21436 | (SEQ ID NO: 349) | human ribophorin I |
| 21435 | (SEQ ID NO: 350) | human cytoplasmic chaperonin hTRiC5 |
| 21425 | (SEQ ID NO: 351) | humanEMX2 |
| 21423 | (SEQ ID NO: 352) | human p87/p89 gene |
| 21419 | (SEQ ID NO: 353) | human HPBRII-7 |
| 21252 | (SEQ ID NO: 354) | human T1-227H |
| 21251 | (SEQ ID NO: 355) | human cullin I |
| 21247 | (SEQ ID NO: 356) | kunitz type protease inhibitor (KOP) |
| 21244-1 | (SEQ ID NO: 357) | human protein tyrosine phosphatase receptor |
| | | F (PTPRF) |
| 21718 | (SEQ ID NO: 358) | human LTR repeat |
| OV2-90 | (SEQ ID NO: 359) | novel |
| Human zinc finger | (SEQ ID NO: 360) | |
| Human polyA binding protein | (SEQ ID NO: 361) | |
| Human pleitrophin | (SEQ ID NO: 362) | |
| Human PAC clone 278C19 | (SEQ ID NO: 363) | |
| Human LLRep3 | (SEQ ID NO: 364) | |
| Human Kunitz type protease inhib | (SEQ ID NO: 365) | |
| Human KIAA0106 gene | (SEQ ID NO: 366) | |
| Human keratin | (SEQ ID NO: 367) | |
| Human HIV-1TAR | (SEQ ID NO: 368) | |
| Human glia derived nexin | (SEQ ID NO: 369) | |
| Human fibronectin | (SEQ ID NO: 370) | |
| Human ECMproBM40 | (SEQ ID NO: 371) | |
| Human collagen | (SEQ ID NO: 372) | |
| Human alpha enolase | (SEQ ID NO: 373) | |
| Human aldolase | (SEQ ID NO: 374) | |
| Human transf growth factor BIG H3 | (SEQ ID NO: 375) | |
| Human SPARC osteonectin | (SEQ ID NO: 376) | |
| Human SLP1 leucocyte protease | (SEQ ID NO: 377) | |
| Human mitochondrial ATP synth | (SEQ ID NO: 378) | |
| Human DNA seq clone 461P17 | (SEQ ID NO: 379) | |
| Human dbpB pro Y box | (SEQ ID NO: 380) | |
| Human 40 kDa keratin | (SEQ ID NO: 381) | |
| Human arginosuccinate synth | (SEQ ID NO: 382) | |
| Human acidic ribosomal phosphoprotein | (SEQ ID NO: 383) | |
| Human colon carcinoma laminin binding pro | (SEQ ID NO: 384) | |

This screen further identified multiple forms of the clone O772P, referred to herein as 21013, 21003 and 21008. PSORT analysis indicates that 21003 (SEQ ID NO:386; translated as SEQ ID NO:389) and 21008 (SEQ ID NO:387; translated as SEQ ID NO:390) represent Type 1a transmembrane protein forms of O772P. 21013 (SEQ ID NO:385; translated as SEQ ID NO:388) appears to be a truncated form of the protein and is predicted by PSORT analysis to be a secreted protein.

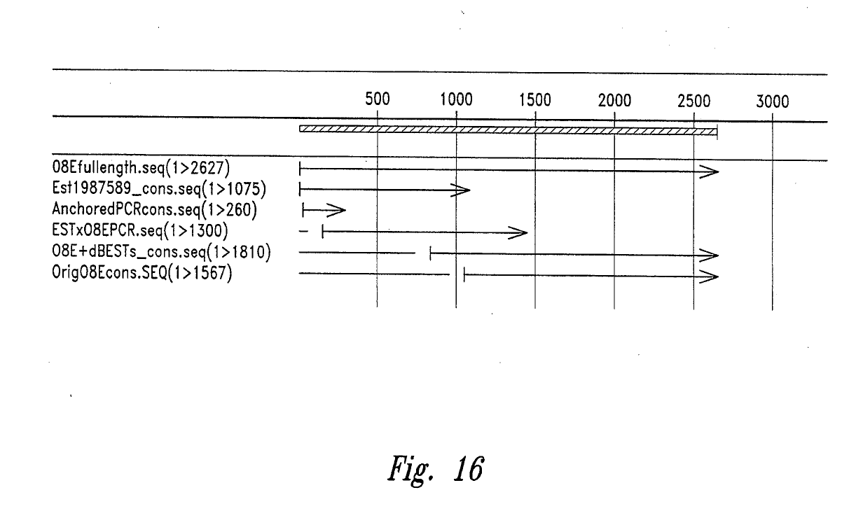

Additional sequence analysis resulted in a full length clone for O8E (2627 bp, which agrees with the message size observed by Northern analysis; SEQ ID NO:391). This nucleotide sequence was obtained as follows: the original O8E sequence (OrigO8Econs) was found to overlap by 33 nucleotides with a sequence from an EST clone (IMAGE#1987589). This clone provided 1042 additional nucleotides upstream of the original O8E sequence. The link between the EST and O8E was confirmed by sequencing multiple PCR fragments generated from an ovary primary tumor library using primers to the unique EST and the O8E sequence (ESTxO8EPCR). Full length status was further indicated when anchored PCR from the ovary tumor library gave several clones (AnchoredPCR cons) that all terminated upstream of the putative start methionine, but failed to yield any additional sequence information. FIG. 16 presents a diagram that illustrates the location of each partial sequence within the full length O8E sequence.

Two protein sequences may be translated from the full length O8E. For “a” (SEQ ID NO:393) begins with a putative start methionine. A second form “b” (SEQ ID NO:392) includes 27 additional upstream residues to the 5′ end of the nucleotide sequence.

### Example 3

This example discloses the identification and characterization of antibody epitopes recognized by the O8E polyclonal anti-sera.

Rabbit anti-sera was raised against E. coli derived O8E recombinant protein and tested for antibody epitope recognition against 20 or 21 mer peptides that correspond to the O8E amino acid sequence. Peptides spanning amino acid regions 31 to 65, 76 to 110, 136 to 200 and 226 to 245 of the full length O8E protein were recognized by an acid eluted peak and/or a salt eluted peak from affinity purified anti-O8E sera. Thus, the corresponding amino acid sequences of the above peptides constitute the antibody epitopes recognized by affinity purified anti-O8E antibodies.

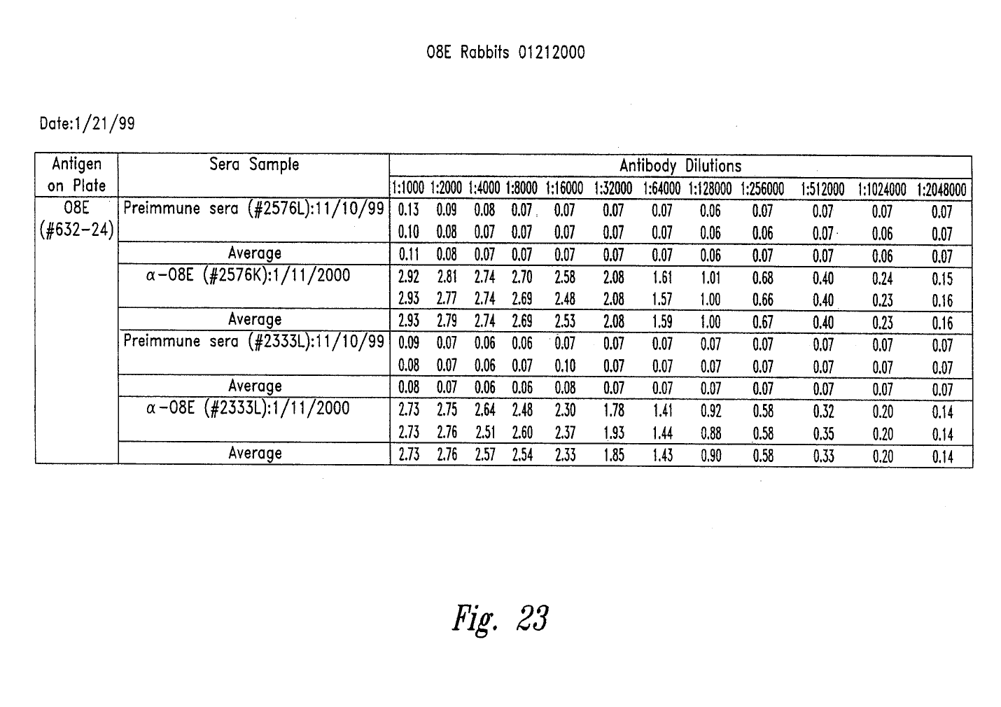

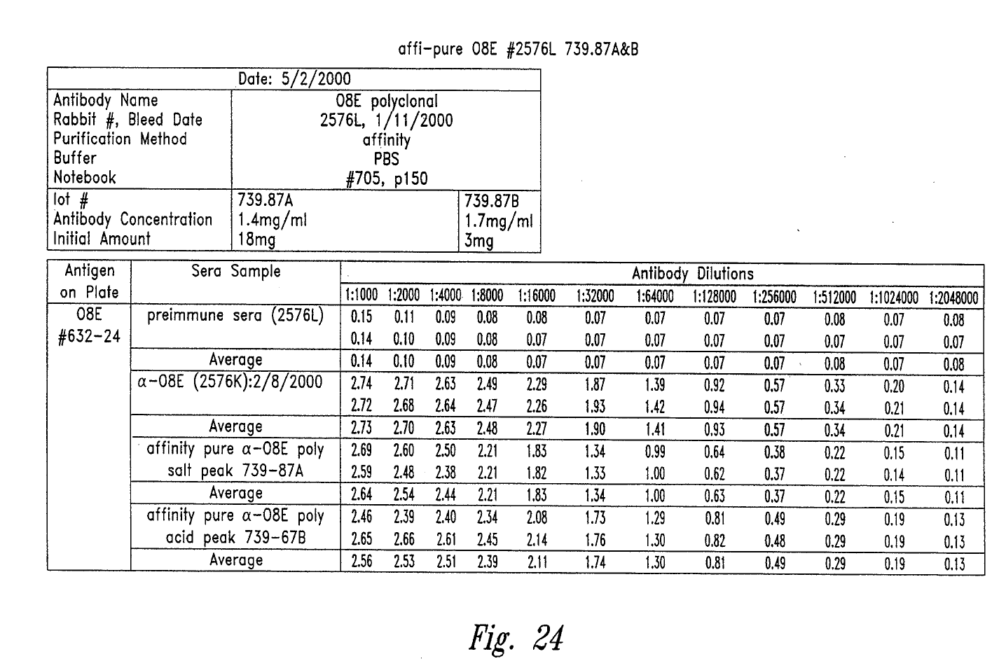

ELISA analysis of anti-O8E rabbit sera is shown in FIG. 23, and ELISA analysis of affinity purified rabbit anti-O8E polyclonal antibody is shown in FIG. 24.

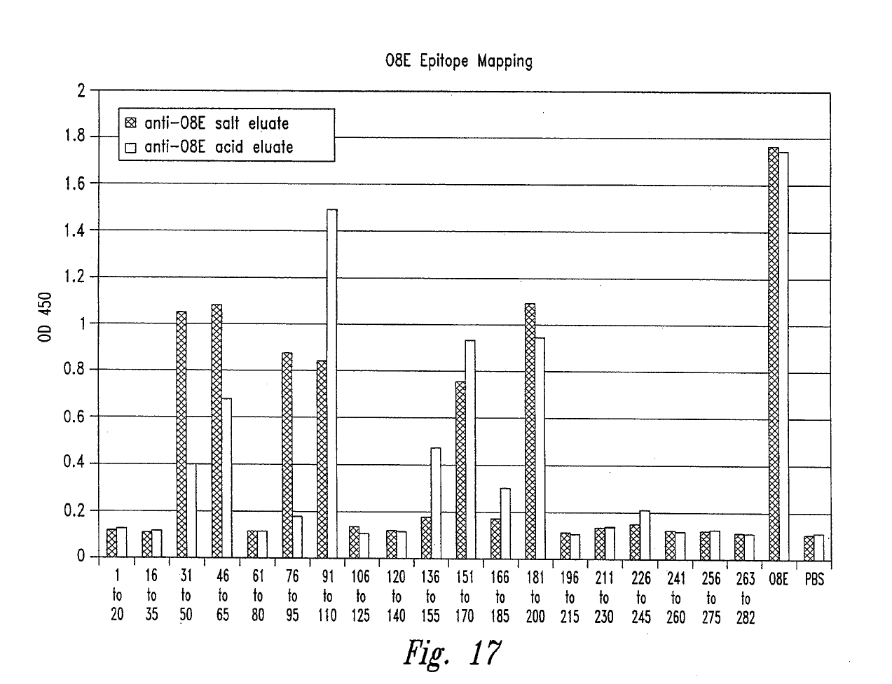

For epitope mapping, 20 or 21 mer peptides corresponding to the O8E protein were synthesized. For antibody affinity purification, rabbit anti-O8E sera was run over an O8E-sepharose column, then antibody was eluted with a salt buffer containing 0.5 M NaCl and 20 mM PO₄, followed by an acid elution step using 0.2 M Glycine, pH 2.3. Purified antibody was neutralized by the addition of 1M Tris, pH 8 and buffer exchanged into phosphate buffered saline (PBS). For enzyme linked immunosorbant assay (ELISA) analysis, O8E peptides and O8E recombinant protein were coated onto 96 well flat bottom plates at 2 μg/ml for 2 hours at room temperature (RT). Plates were then washed 5 times with PBS+0.1% Tween 20 and blocked with PBS+1% bovine serum albumin (BSA) for 1 hour. Affinity purified anti-O8E antibody, either an acid or salt eluted fraction, was then added to the wells at 1 μg/ml and incubated at RT for 1 hr. Plates were again washed, followed by the addition of donkey anti-rabbit-Ig-horseradish peroxidase (HRP) antibody for 1 hour at RT. Plates were washed, then developed by the addition of the chromagenic substrate 3,3′,5,5′-tetramethylbenzidine (TMB) (described by Bos et al., J. of Immunoassay 2:187-204 (1981); available from Sigma (St. Louis, Mo.)). The reaction was incubated 15 minutes at RT and then stopped by the addition of 1 N H₂SO₄. Plates were read at an optical density of 450 (OD450) in an automated plate reader. The sequences of peptides corresponding to the OE8 antibody epitopes are disclosed herein as SEQ ID NO: 394-415. Antibody epitopes recognized by the O8E polyclonal anti-sera are disclosed herein in FIG. 17.

### Example 4

This example discloses IHC analysis of O8E expression in ovarian cancer tissue samples.

For immunohistochemistry studies, paraffin-embedded formalin fixed ovarian cancer tissue was sliced into 8 micron sections. Steam heat induced epitope retrieval (SHIER) in 0.1 M sodium citrate buffer (pH 6.0) was used for optimal staining conditions. Sections were incubated with 10% serum/PBS for 5 minutes. Primary antibody (anti-O8E rabbit affinity purified polyclonal antibody) was added to each section for 25 min followed by a 25 min incubation with an anti-rabbit biotinylated antibody. Endogenous peroxidase activity was blocked by three 1.5 min incubations with hydrogen peroxidase. The avidin biotin complex/horse radish peroxidase system was used along with DAB chromogen to visualize antigen expression. Slides were counterstained with hematoxylin. One (papillary serous carcinoma) of six ovarian cancer tissue sections displayed O8E immunoreactivity. Upon optimization of the staining conditions, ⅘ ovarian cancer samples stained positive using the O8E polyclonal antibody. O8E expression was localized to the plasma membrane.

Six ovarian cancer tissues were analyzed with the anti-O8E rabbit polyclonal antibody. One (papillary serous carcinoma) of six ovarian cancer tissue samples stained positive for O8E expression. O8E expression was localized to the surface membrane.

### Example 5

This example discloses O8E peptides that are predicted to bind HLA-A2 and to be immunogenic for CD8 T cell responses in humans.

Potential HLA-A2 binding peptides of O8E were predicted by using the full-length open-reading frame (ORF) from O8E and running it through “Episeek,” a program used to predict MHC binding peptides. The program used is based on the algorithm published by Parker, K. C. et al., J. Immunol. 152(1):163-175 (1994) (incorporated by reference herein in its entirety). 10-mer and 9-mer peptides predicted to bind HLA-0201 are disclosed herein as SEQ ID NO: 416-435 and SEQ ID NO: 436-455, respectively.

### Example 6

This example discloses O8E cell surface expression measured by fluorescence activated cell sorting.

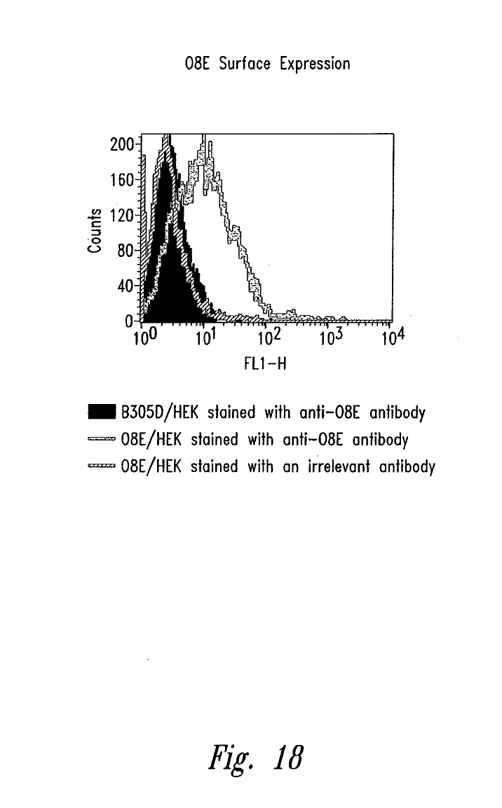

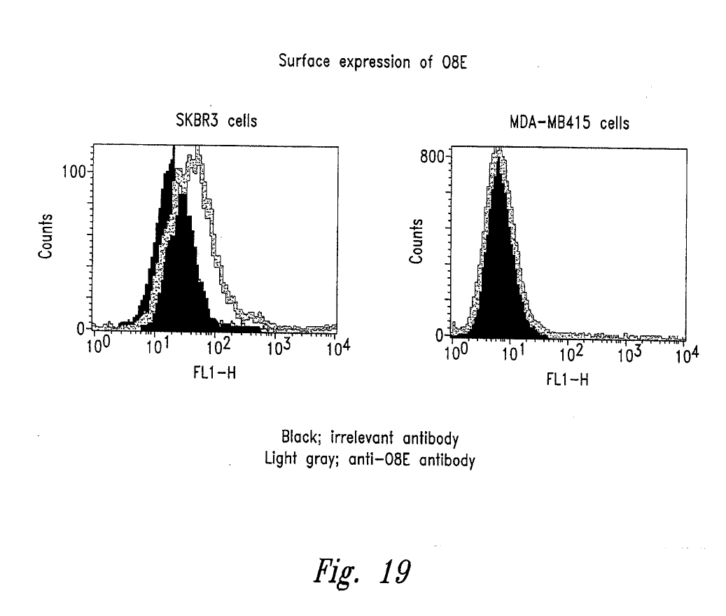

For FACS analysis, cells were washed with ice cold staining buffer (PBS/1% BSA/azide). Next, the cells were incubated for 30 minutes on ice with 10 micrograms/ml of affinity purified rabbit anti-B305D polyclonal antibody. The cells were washed 3 times with staining buffer and then incubated with a 1:100 dilution of a goat anti-rabbit Ig (H+L)-FITC reagent (Southern Biotechnology) for 30 minutes on ice. Following 3 washes, the cells were resuspended in staining buffer containing prodium iodide, a vital stain that allows for identification of permeable cells, and analyzed by FACS. O8E surface expression was confirmed on SKBR3 breast cancer cells and HEK293 cells that stably overexpress the cDNA for O8E. Neither MB415 cells nor HEK293 cells stably transfected with a control irrelevant plasmid DNA showed surface expression of O8E (FIGS. 18 and 19).

### Example 7

This example further evaluates the expression and surface localization of O8E.

For expression and purification of antigen used for immunization, O8E expressed in an E. coli recombinant expression system was grown overnight in LB Broth with the appropriate antibiotics at 37° C. in a shaking incubator. The next morning, 10 ml of the overnight culture was added to 500 ml of 2×YT plus appropriate antibiotics in a 2 L-baffled Erlenmeyer flask. When the Optical Density (at 560 nanometers) of the culture reached 0.4-0.6 the cells were induced with IPTG (1 mM). 4 hours after induction with IPTG the cells were harvested by centrifugation. The cells were then washed with phosphate buffered saline and centrifuged again. The supernatant was discarded and the cells were either frozen for future use or immediately processed. Twenty milliliters of lysis buffer was added to the cell pellets and vortexed. To break open the E. coli cells, this mixture was then run through the French Press at a pressure of 16,000 psi. The cells were then centrifuged again and the supernatant and pellet were checked by SDS-PAGE for the partitioning of the recombinant protein. For protein that localized to the cell pellet, the pellet was resuspended in 10 mM Tris pH 8.0, 1% CHAPS and the inclusion body pellet was washed and centrifuged again. This procedure was repeated twice more. The washed inclusion body pellet was solubilized with either 8 M urea or 6 M guanidine HCl containing 10 mM Tris pH 8.0 plus 10 mM imidazole. The solubilized protein was added to 5 ml of nickel-chelate resin (Qiagen) and incubated for 45 min to 1 hour at room temperature with continuous agitation. After incubation, the resin and protein mixture were poured through a disposable column and the flow through was collected. The column was then washed with 10-20 column volumes of the solubilization buffer. The antigen was then eluted from the column using 8M urea, 10 mM tris pH 8.0 and 300 mM imidazole and collected in 3 ml fractions. A SDS-PAGE gel was run to determine which fractions to pool for further purification. As a final purification step, a strong anion exchange resin such as Hi-Prep Q (Biorad) was equilibrated with the appropriate buffer and the pooled fractions from above were loaded onto the column. Each antigen was eluted off of the column with an increasing salt gradient. Fractions were collected as the column was run and another SDS-PAGE gel was run to determine which fractions from the column to pool. The pooled fractions were dialyzed against 10 mM Tris pH 8.0. This material was then evaluated for acceptable purity as determined by SDS-PAGE or HPLC, concentration as determined by Lowry assay or Amino Acid Analysis, identity as determined by amino terminal protein sequence, and endotoxin level as determined by the Limulus (LAL) assay. The proteins were then vialed after filtration through a 0.22 micron filter and the antigens were frozen until needed for immunization.

For generation of polyclonal anti-sera, 400 micrograms of each prostate antigen was combined with 100 micrograms of muramyldipeptide (MDP). Equal volume of Incomplete Freund's Adjuvant (IFA) was added and then mixed. Every four weeks animals were boosted with 100 micrograms of antigen mixed with an equal volume of IFA. Seven days following each boost the animal was bled. Sera was generated by incubating the blood at 4° C. for 12-24 hours followed by centrifugation.

For characterization of polyclonal antisera, 96 well plates were coated with antigen by incubating with 50 microliters (typically 1 microgram) at 4° C. for 20 hrs. 250 microliters of BSA blocking buffer was added to the wells and incubated at RT for 2 hrs. Plates were washed 6 times with PBS/0.01% tween. Anti-O8E rabbit sera or affinity purified anti-O8e antibody was diluted in PBS. Fifty microliters of diluted antibody was added to each well and incubated at RT for 30 min. Plates were washed as described above before 50 microliters of goat anti-rabbit horse radish peroxidase (HRP) at a 1:10000 dilution was added and incubated at RT for 30 min. Plates were washed as described above and 100 microliters of TMB microwell Peroxidase Substrate was added to each well. Following a 15 minute incubation in the dark at room temperature the colorimetric reaction was stopped with 100 microliters of 1N H2SO4 and read immediately at 450 nm. All polyclonal antibodies showed immunoreactivity to the O8E antigen.

For recombinant expression in mammalian HEK293 cells, full length O8E cDNA was subcloned into the mammalian expression vectors pcDNA3.1+ and pCEP4 (Invitrogen) which were modified to contain His and FLAG epitope tags, respectively. These constructs were transfected into HEK293 cells (ATCC) using Fugene 6 reagent (Roche). Briefly, HEK293 cells were plated at a density of 100,000 cells/ml in DMEM (Gibco) containing 10% FBS (Hyclone) and grown overnight. The following day, 2 ul of Fugene6 was added to 100 ul of DMEM containing no FBS and incubated for 15 minutes at room temperature. The Fugene6/DMEM mixture was then added to 1 ug of O8E/pCEP4 or O8E/pcDNA3.1 plasmid DNA and incubated for 15 minutes at room temperature. The Fugene/DNA mix was then added to the HEK293 cells and incubated for 48-72 hrs at 37° C. with 7% CO2. Cells were rinsed with PBS then collected and pelleted by centrifugation. For Western blot analysis, whole cell lysates were generated by incubating the cells in Triton-X100 containing lysis buffer for 30 minutes on ice. Lysates were then cleared by centrifugation at 10,000 rpm for 5 minutes at 4 C. Samples were diluted with SDS-PAGE loading buffer containing beta-mercaptoethanol, then boiled for 10 minutes prior to loading the SDS-PAGE gel. Protein was transferred to nitrocellulose and probed using anti-O8E rabbit polyclonal sera #2333L at a dilution of 1:750. The blot was revealed with a goat anti-rabbit Ig coupled to HRP followed by incubation in ECL substrate.

For FACS analysis, cells were washed further with ice cold staining buffer (PBS+1% BSA+Azide). Next, the cells were incubated for 30 minutes on ice with 10 ug/ml of Protein A purified anti-O8E polyclonal sera. The cells were washed 3 times with staining buffer and then incubated with a 1:100 dilution of a goat anti-rabbit Ig(H+L)-FITC reagent (Southern Biotechnology) for 30 minutes on ice. Following 3 washes, the cells were resuspended in staining buffer containing Propidium Iodide (PI), a vital stain that allows for the identification of permeable cells, and analyzed by FACS.

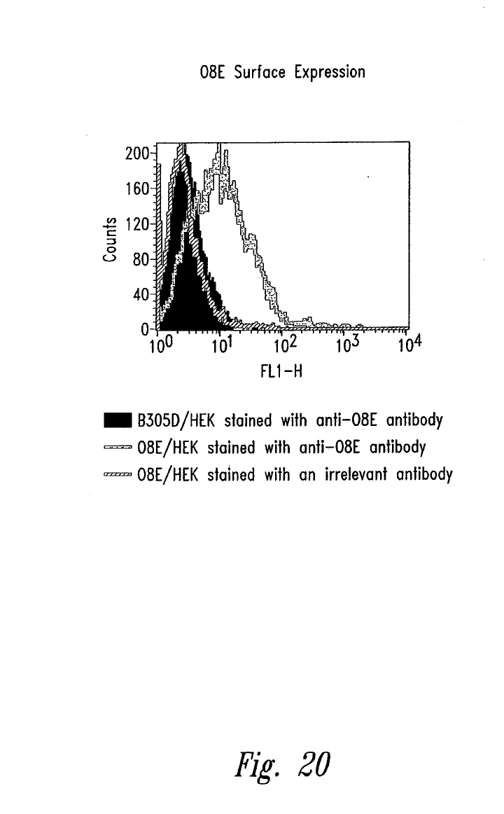

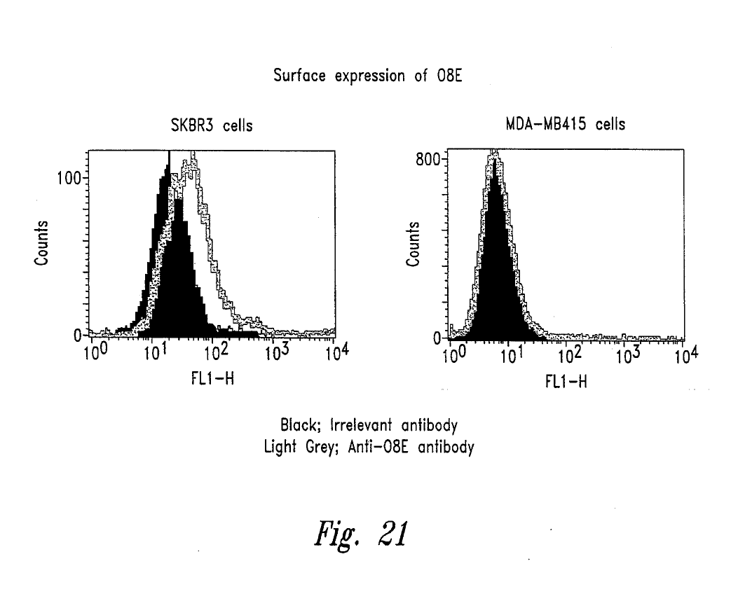

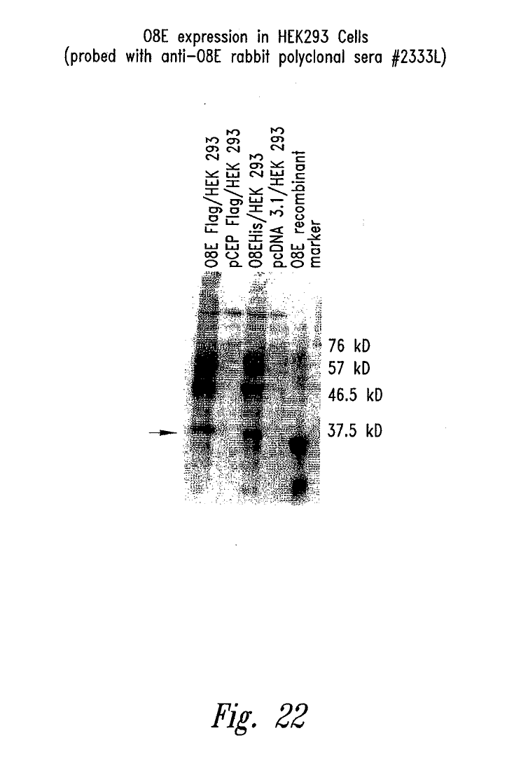

From these experiments, the results of which are illustrated in FIGS. 20-21, O8E expression was detected on the surface of transfected HEK293 cells and SKBR3 cells by FACS analysis using rabbit anti-O8E sera. Expression was also detected in transfected HEK293 cell lysates by Western blot analysis (FIG. 22).

### Example 8

### Generation and Characterization of Anti-O8E mAbs

Mouse monoclonal antibodies were raised against E. coli derived O8E proteins as follows. NJ mice were immunized intraperitoneally (IP) with Complete Freund's Adjuvant (CFA) containing 50 μg recombinant O8E, followed by a subsequent IP boost with Incomplete Freund's Adjuvant (IFA) containing 10 μg recombinant O8E protein. Three days prior to removal of the spleens, the mice were immunized intravenously with approximately 50 μg of soluble O8E recombinant protein. The spleen of a mouse with a positive titer to O8E was removed, and a single-cell suspension made and used for fusion to SP2/0 myeloma cells to generate B cell hybridomas. The supernatants from the hybrid clones were tested by ELISA for specificity to recombinant O8E, and epitope mapped using peptides that spanned the entire O8E sequence. The mAbs were also tested by flow cytometry for their ability to detect O8E on the surface of cells stably transfected with O8E and on the surface of a breast tumor cell line.

For ELISA analysis, 96 well plates were coated with either recombinant O8E protein or overlapping 20-mer peptides spanning the entire O8E molecule at a concentration of either 1-2 μg/ml or 10 μg/ml, respectively. After coating, the plates were washed 5 times with washing buffer (PBS+0.1% Tween-20) and blocked with PBS containing 0.5% BSA, 0.4% Tween-20. Hybrid supernatants or purified mAbs were then added and the plates incubated for 60 minutes at room temperature. The plates were washed 5 times with washing buffer and the secondary antibody, donkey-anti mouse Ig linked to horseradish peroxidase (HRP)(Jackson ImmunoResearch), was added for 60 minutes. The plates were again washed 5 times in washing buffer, followed by the addition of the peroxidase substrate. Of the hybridoma clones generated, 15 secreted mAbs that recognized the entire O8E protein. Epitope mapping revealed that of these 15 clones, 14 secreted mAbs that recognized the O8E amino acid residues 61-80 and one clone secreted a mAb that recognized amino acid residues 151-170.

For flow cytometric analysis, HEK293 cells which had been stably transfected with O8E and SKBR3 cells which express O8E mRNA, were harvested and washed in flow staining buffer (PBS+1% BSA+Azide). The cells were incubated with the supernatant from the mAb hybrids for 30 minutes on ice followed by 3 washes with staining buffer. The cells were incubated with goat-anti mouse Ig-FITC for 30 minutes on ice, followed by three washes with staining buffer before being resuspended in wash buffer containing propidium iodide. Flow cytometric analysis revealed that 15/15 mAbs were able to detect O8E protein expressed on the surface of O8E-transfected HEK293 cells. 6/6 mAbs tested on SKBR3 cells were able to recognize surface expressed O8E.

### Example 9

### Extended DNA and Protein Sequence Analysis of Sequence O772P

A full-length sequence encompassing clones 3f, 6b, 8e, and 12 was obtained by screening an ovarian tumor (SCID-derived) cDNA library described in detail in Example 2. This 2996 base pair sequence, designated O772P, is presented in SEQ ID NO: 311, and the encoded 914 amino acid protein sequence is shown in SEQ ID NO: 312. The DNA sequence O772P was searched against public databases including Genbank and showed a significant hit to Genbank Accession number AK024365 (SEQ ID NO: 457). This Genbank sequence was found to be 3557 base pairs in length and encodes a protein 1156 amino acids in length (SEQ ID NO: 459). A truncated version of this sequence, residues 25-3471, in which residue 25 corresponds to the first ATG initiation codon in the Genbank sequence, (SEQ ID NO: 456), encodes a protein that is 1148 amino acids in length (SEQ ID NO: 458). The published DNA sequence (SEQ ID NO: 457) differs from O772P in that it has a 5 base pair insertion corresponding to bases 958-962 of SEQ ID NO: 457. This insertion results in a frame shift such that SEQ ID NO: 457 encodes an additional N-terminal protein sequence relative to O772P (SEQ ID NO: 312). In addition, O772P encodes a unique N-terminal portion contained in residues 1-79 (SEQ ID NO: 460). The N-terminal portion of SEQ ID NO: 456, residues 1-313, also contains unique sequence and is listed as SEQ ID NO: 461.

### Example 10

### The Generation of Polyclonal Antibodies for Immunohistochemistry and Flow Cytometric Analysis of the Cell Associated Expression Pattern of Molecule O772P

The O772P molecule was identified in Examples 2 and 9 of this application. To evaluate the subcellular localization and specificity of antigen expression in various tissues, polyclonal antibodies were generated against O772P. To produce these antibodies, O772P-1 (amino acids 44-772 of SEQ ID NO:312) and O772P-2 (477-914 of SEQ ID NO:312) were expressed in an E. coli recombinant expression system and grown overnight at 37° C. in LB Broth. The following day, 10 ml of the overnight culture was added to 500 ml of 2xYT containing the appropriate antibiotics. When the optical density of the cultures (560 nanometers) reached 0.4-0.6 the cells were induced with IPTG. Following induction, the cells were harvested, washed, lysed and run through a French Press at a pressure of 16000 psi. The cells were then centrifuged and the pellet checked by SDS-PAGE for the partitioning of the recombinant protein. For proteins that localize to the cell pellet, the pellet was resuspended in 10 mM Tris, pH 8.0, 1% CHAPS and the inclusion body pellet washed and centrifuged. The washed inclusion body was solubilized with either 8M urea or 6M guanidine HCL containing 10 mM Tris, pH 8.0, plus 10 mM imidazole. The solubilized protein was then added to 5 ml of nickel-chelate resin (Qiagen) and incubated for 45 minutes at room temperature.

Following the incubation, the resin and protein mixture was poured through a column and the flow through collected. The column was washed with 10-20 column volumes of buffer and the antigen eluted using 8M urea, 10 mM Tris, pH 8.0, and 300 mM imidazole and collected in 3 ml fractions. SDS-PAGE was run to determine which fractions to pool for further purification. As a final purification step, a strong anion exchange resin was equilibrated with the appropriate buffer and the pooled fractions were loaded onto the column. Each antigen was eluted from the column with an increasing salt gradient. Fractions were collected and analyzed by a SDS-PAGE to determine which fractions from the column to pool. The pooled fractions were dialyzed against 10 mM Tris, pH 8.0, and the resulting protein was submitted for quality control for final release. The release criteria were: (a) purity as determined by SDS-PAGE or HPLC, (b) concentration as determined by Lowry assay or Amino Acid Analysis, (c) identity as determined by amino terminal protein, and (d) endotoxin levels as determined by the Limulus (LAL) assay. The proteins were then filtered through a 0.22 μM filter and frozen until needed for immunizations.

To generate polyclonal antisera, 400 μg of O772P-1 or O772P-2 was combined with 100 μg of muramyldipeptide (MDP). The rabbits were immunized every 4 weeks with 100 μg of antigen mixed with an equal volume of Incomplete Freund's Adjuvant (IFA). Seven days following each boost, the animals were bled and sera was generated by incubating the blood at 4° C. for 12-24 hours followed by centrifugation.

To characterize the antisera, 96 well plates were coated with antigen followed by blocking with BSA. Rabbit sera was diluted in PBS and added to each well. The plates were then washed, and goat anti-rabbit horseradish peroxidase (HRP). The plates were again washed and TMB microwell Peroxidase Substrate was added. Following this incubation, the colormetric reaction was stopped and the plates read immediately at 450 nm. All polyclonal antibodies showed immunoreactivity to the appropriate antigen.

Immunohistochemistry analysis of O772P expression was performed on paraffin-embedded formalin fixed tissue. O772P was found to be expressed in normal ovary and ovarian tumor, but not in normal heart, kidney, colon, lung or liver. Additionally, immunohistochemistry and flow cytometric analysis indicates that O772P is a plasma membrane-associated molecule. O772P contains 1 plasma transmembrane domain predicted to be encoded by amino acids 859-880. The N-terminus of O772P is extracellular and is encoded by amino acids 1-859, while the C-terminus is intracellular. Sequence analysis shows that there are 17 potential N-linked glycosylation sites.

### Example 11

### O772P is Expressed on the Surface of Primary Ovarian Tumor Cells

For recombinant expression in mammalian cells, the O772P-21008 (SEQ ID NO:387) and O772P full length cDNA (SEQ ID NO:311 encoding the protein of SEQ ID NO:312) were subcloned into mammalian expression vectors pBIB or pCEP4 respectively. These constructs were transfected into HEK293 cells using Fugene 6 (Roche). The HEK cells were then plated at a density of 100,000 cells/ml in DMEM containing fetal bovine serum (FBS) and grown overnight. The following day, 2 μl of Fugene 6 was added to 100 μl of DMEM, which contained no FBS, and incubated for 15 minutes at room temperature. The Fugene 6/DMEM mixture was then added to 1 μg of O772P/pBIB or O772P/pCEP4 plasmid DNA and incubated for an additional 15 minutes at room temperature. The Fugene 6/DNA mix was then added to the HEK293 cells and incubated for 48-72 hours at 37° C. with 7% CO₂. The cells were rinsed and pelleted by centrifugation.

For Western Blot analysis, whole cell lysates were generated by incubating the cells in lysis buffer followed by clarification by centrifugation. The samples were diluted and run on SDS-PAGE. The gel was then transferred to nitrocellulose and probed using purified anti-O772P-2 rabbit polyclonal antibody. The blot was revealed with a goat anti-rabbit Ig coupled to HRP followed by incubation in ECL substrate. Western Blot analysis revealed that O772P-21008 could be detected in HEK293 cells that had been transfected with O772P.

To determine the cell expression profile of O772P in cells, primary ovarian tumor cells were grown in SCID mice. The cells were retrieved from the mice and analyzed by flow cytometry. Briefly, cells washed in cold staining buffer containing PBS, 1% BSA, and Na Azide. The cells were incubated for 30 minutes with 10 μg/ml of purified anti-O772P-1 and O772P-2 polyclonal sera. Following this incubation, the cells were washed three times in staining buffer and incubated with goat anti-rabbit Ig (H+L) conjugated to FITC (Southern Biotechnology). The cells were washed and resuspended in staining buffer containing Propidium Iodide (PI), a vital stain that identifies non-viable cells. The cells were then analyzed using Fluorescence Activated Cell Sorting (FACS). FACS analysis revealed that O772P was present on the cells surface. Surface expression of O772P on tumor cells allows for immune targeting by therapeutic antibodies.

### Example 12

### Functional Characterization of Anti-O8E Monoclonal Antibodies

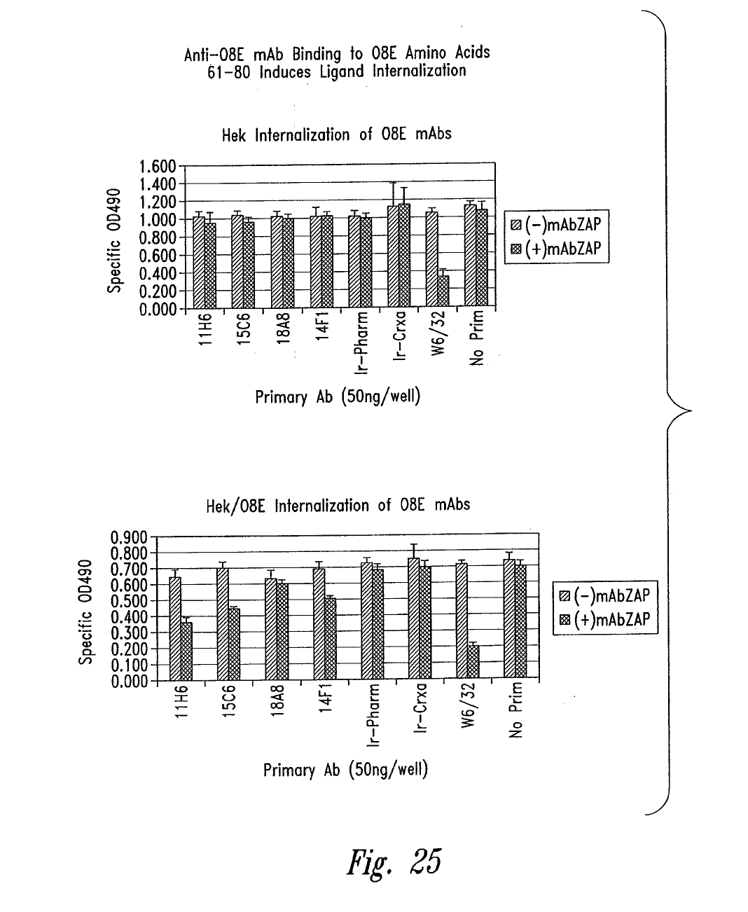

Mouse monoclonal antibodies (mAb) raised against E. coli derived O8E, as described in Example 8, were tested for their ability to promote O8E antigen internalization. Internalization of the antibody was determined using an in vitro cytotoxicity assay. Briefly, HEK293 and O8E/HEK transfected cells were plated into 96 well plates containing DME plus 10% heat-inactivated FBS in the presence of 50 ng/well of purified anti-O8E or control antibodies. The isotype of the anti-O8E mAbs are as follows: 11A6-IgG1/kappa, 15C6-IgG2b/kappa, 18A8-IgG2b/kappa, and 14F1-IgG2a/kappa. W6/32 is a pan anti-human MHC class I mouse monoclonal antibody that serves as a positive control, and two irrelevant mAbs, Ir-Pharm and Ir-Crxa were included as negative controls. Following incubation with the O8E specific antibodies or the relevant controls antibodies, the mAb-zap, a goat anti-mouse Ig-saporin conjugated secondary antibody (Advanced Targeting Systems) was added at a concentration of 100 ng/ml to half of the wells, and the plates were incubated for 48 to 72 hours at 37° C. in a 7% CO₂ incubator. This assay takes advantage of the toxic nature of saporin, a ribozyme inactivating protein, which when internalized has a cytotoxic effect. Following incubation with the mAb-zap, internalization was quantitated by the addition of MTS reagent, followed by reading the OD490 of the plate on a microplate ELISA reader. FIG. 25 depicts the results from these assays. The top panel represents HEK cells that have not been transfected with O8E and therefore O8E antibody should not bind and be internalized. Levels of proliferation were the same in all samples whether they were incubated with or without the mAb-zap, with the exception of the positive control Ab, W6/32. The lower panel represents cells that have been transfected with O8E and therefore should bind O8E specific antibodies. Antibodies from the hybridomas 11H6, 14F1, and 15C6, which recognize the amino acids 61-80 of O8E were able to promote internalization of the O8E surface protein as measured by decreased levels of proliferation due to the toxic nature of the mAb-zap (See FIG. 25). The antibody generated by the hybridoma 18A8, which recognizes amino acids 151-170 of O8E, was unable to promote internalization as determined by normal levels of proliferation either in the absence or presence of the mAb-zap.

### Example 13

### Characterization of the Ovarian Tumor Antigen, O772P

The cDNA and protein sequences for multiple forms of the ovarian tumor antigen O772P have been described in the above (e.g., Examples 2 and 9). A Genbank search indicated that O772P has a high degree of similarity with FLJ14303 (Accession # AK024365; SEQ ID NO:457 and 463). Protein sequences corresponding to O772P and FLJ14303 are disclosed in SEQ ID NO:478 and 479, respectively. FLJ14303 was identical to the majority of O772P, with much of the 3′-end showing 100% homology. However, the 5′-end of FLJ14303 was found to extend further 5′ than O772P. In addition, FLJ14303 contained a 5 bp insert (SEQ ID NO:457) resulting in a frame shift of the amino-terminus protein sequence such that FLJ14303 utilizes a different starting methionine than O772P and therefore encodes a different protein. This insertion was present in the genomic sequence and seen in all EST clones that showed identity to this region, suggesting that FLJ14303 (SEQ ID NO:457) represents a splice variant of O772P, with an ORF that contains an extended and different amino-terminus. The additional 5′-nucleotide sequence included repeat sequences that were identified during the genomic mapping of O772P. The 5′-end of O772P and the corresponding region of FLJ14303 showed between 90-100% homology. Taken together, this suggests that O772P and FLJ14303 are different splice variants of the same gene, with different unique repeat sequences being spliced into the 5′-end of the gene.

The identification of an additional ten or more repeat sequences within the same region of chromosome 19, indicates that there may be many forms of O772P, each with a different 5′-end, due to differential splicing of different repeat sequences. Northern blot analysis of O772P demonstrated multiple O772P-hybridizing transcripts of different sizes, some in excess 10 kb.

Upon further analysis, 13 additional O772P-related sequences were identified, the cDNA and amino acid sequences of which are described in Table 3.

TABLE 3

| SEQ ID NO: | Description | Transmembrane Domains |
|---|---|---|
| 464 | LS #1043400.1 (cDNA) | nd |
| 465 | LS #1043400.10 (cDNA) | 0 |
| 466 | LS #1043400.11 (cDNA) | 2 |
| 467 | LS #1043400.12 (cDNA) | 2 |
| 468 | LS #1043400.2 (cDNA) | nd |
| 469 | LS #1043400.3 (cDNA) | |
| 470 | LS #1043400.5 (cDNA) | nd |
| 471 | LS #1043400.8 (cDNA) | 1 |
| 472 | LS #1043400.9 (cDNA) | 0 |
| 473 | LS #1043400.6 (cDNA) | nd |
| 474 | LS #1043400.7 (cDNA) | nd |
| 475 | LS #1043400.4 (cDNA) | nd |
| 476 | LS #1397610.1 (cDNA) | 0 |
| 477 | 1043400.10 Novel 5′ (cDNA) | — |
| 480 | LS #1043400.9 (amino acid) | — |
| 481 | LS #1043400.8B (amino acid) | — |
| | Contains a transmembrane | |
| | domain | |
| 482 | LS #1043400.8A (amino acid) | — |
| 483 | LS #1043400.12 (amino acid) | — |
| | Contains a transmembrane | |
| | domain | |
| 484 | LS #1043400.11B (amino acid) | — |
| | Contains a transmembrane | |
| | domain | |
| 485 | LS #1043400.11A (amino acid) | — |
| 486 | LS #1043400.10 (amino acid) | — |
| 487 | LS #1043400.1 (amino acid) | — |

nd = not determined

Initially it appeared that these sequences represented overlapping and/or discrete sequences of O772P splice forms that were capable of encoding polypeptides unique to the specific splice forms of O772P. However, nucleotide alignment of these sequences failed to identify any identical regions within the repeat elements. This indicates that the sequences may represent different specific regions of a single O772P gene, one that contains 16 or more repeat domains, all of which form a single linear transcript. The 5′-end of sequence LS #1043400.10 (Table 2; SEQ ID NO:465) is unique to both O772P and F1114303 and contains no repeat elements, indicating that this sequence may represent the 5′-end of O772P.

Previously, transmembrane prediction analysis had indicated that O772P contained between 1 and 3 transmembrane spanning domains. This was verified by the use of immunohistochemistry and flow cytometry, which demonstrated the existence of a plasma membrane-associated molecule representing O772P. However, immunohistochemistry also indicated the presence of secreted form(s) of O772P, possibly resulting from an alternative splice form of O772P or from a post-translational cleavage event. Analysis of several of the sequences presented in Table 2 showed that sequences 1043400B.12, 1043400.8B, and 1043400.11B all contained transmembrane regions, while 1043400.8A, 1043400.10, 1043400.1, 1043400.11A, and 1043400.9 were all lacking transmembrane sequences, suggesting that these proteins may be secreted.

Analysis indicates a part of O772P is expressed and/or retained on the plasma membrane, making O772P an attractive target for directing specific immunotherapies, e.g., therapeutic antibodies, against this protein. The predicted extracellular domain of O772P is disclosed in SEQ ID NO:489 and secretion of O772P is likely to occur as a result of a cleavage event within the sequence:

SLVEQVFLDKTLNASFHWLGSTYQLVDIHVTEMESSVYQP.

Proteolytic cleavage is most likely to occur at the Lysine (K) at position 10 of SEQ ID NO:489. The extracellular, transmembrane, and cytoplasmic regions of O772P are all disclosed in SEQ ID NO:488:

Extracellular:
SLVEQVFLDKTLNASFHWLGSTYQLVDIHVTEMESSVYQPTSSSSTQHFY
LNFTITNLPYSQDKAQPGTTNYQRNKRNIEDALNQLFRNSSIKSYFSDCQ
VSTFRSVPNRHHTGVDSLCNFSPLARRVDRVAIYEEFLRMTRNGTQLQNF
TLDRSSVLVDGYFPNRNEPLTGNSDLPF
Transmembrane:
WAVILIGLAGLLGLITCLICGVLVTT
Cytoplasmic:
RRRKKEGEYNVQQQCPGYYQSHLDLEDLQ

### Example 14

### Immunohistochemistry (IHC) Analysis of O8E Expression in Ovarian Cancer and Normal Tissues

In order to determine which tissues express the ovarian cancer antigen O8E, IHC analysis was performed on a diverse range of tissue sections using both polyclonal and monoclonal antibodies specific for O8E. The generation of O8E specific polyclonal antibodies is described in detail in Example 8. The monoclonal antibodies used for staining were 11A6 and 14F1, both of which are specific for amino acids 61-80 of O8E and 18A8, which recognizes amino acids 151-170 of O8E (see Example 12 for details on generation).

To perform staining, tissue samples were fixed in formalin solution for 12-24 hours and embedded in paraffin before being sliced into 8 micron sections. Steam heat induced epitope retrieval (SHEIR) in 0.1M sodium citrate buffer (pH 6.0) was used for optimal staining conditions. Sections were incubated with 10% serum/PBS for 5 minutes. Primary antibody was then added to each section for 25 minutes followed by 25 minutes of incubation with either anti-rabbit or anti-mouse biotinylated antibody. Endogenous peroxidase activity was blocked by three 1.5 minute incubations with hydrogen peroxidase. The avidin biotin complex/horse radish peroxidase (ABC/HRP) system was used along with DAB chromogen to visualize the antigen expression. Slides were counterstained with hematoxylin to visualize the cell nuclei.

Results using rabbit affinity purified polyclonal antibody to O8E (a.a. 29-283; for details on the generation of this Ab, see Example 3) are presented in Table 3. Results using the three monoclonal antibodies are presented in Table 4.

TABLE 4
Immunohistochemistry analysis of O8E using polyclonal antibodies

| Tissue | O8E Expression |
|---|---|
| Ovarian Cancer | Positive |
| Breast Cancer | Positive |
| Normal Ovary | Positive |
| Normal Breast | Positive |
| Blood Vessel | Positive |
| Kidney | Negative |
| Lung | Negative |
| Colon | Negative |
| Liver | Negative |
| Heart | Negative |

TABLE 5
Immunohistochemistry analysis of O8E using monoclonal antibodies

| Normal | 11A6 | | 18A8 | | 14F1 | |
|---|---|---|---|---|---|---|
| Tissue | Endothelial | Epithelial | Endothelial | Epithelial | Endothelial | Epithelial |
| Skin | 2 | 2 | 0 | 0 | 1 | 1 |
| Skin | 1 | 1 | 0 | 0 | 1 | 1 |
| Breast | 0 | 1 | n/a | n/a | 1 | 1 |
| Colon | 0 | 0 | 0 | 0 | 0 | 0 |
| Jejunum | 0 | 0 | 0 | 0 | 0 | 0 |
| Colon | 0 | 0 | 0 | 0 | 0 | 0 |
| Colon | 0 | 0 | 0 | 0 | 0 | 0 |
| Ovary | 0 | 0 | 0 | 0 | 1 | 0 |
| Colon | 0 | 0 | 0 | 0 | 0 | 1 |
| Liver | 0 | 0 | 0 | 0 | 1 | 2 |
| Skin | 0 | 0 | 0 | 0 | 1 | 0 |
| Duodenum | 0 | 0 | 0 | 0 | 0 | 0 |
| and | | | | | | |
| Pancreas | | | | | | |
| Appendix | 0 | 0 | 0 | 0 | 0 | 0 |
| Ileum | 0 | 0 | 0 | 0 | 0 | 0 |

0 = no staining, 1 = light staining, 2 = moderate staining, n/a = not available

### Example 15

### Epitope Mapping of O772P Polyclonal Antibodies

To perform epitope mapping of O772P, peptides were generated, the sequences of which were derived from the sequence of O772P. These peptides were 15 mers that overlapped by 5 amino acids and were generated via chemical synthesis on membrane supports. The peptides were covalently bound to Whatman 50 cellulose support by their C-terminus with the N-terminus unbound. In order to determine epitope specificity, the membranes were wet with 100% ethanol for 1 minute, and then blocked for 16 hours in TBS/Tween/Triton buffer (50 mM Tris, 137 mM NaCl, 2.7 mM KCl, 0.5% BSA, 0.05% Tween 20, 0.05% Triton X-100, pH 7.5). The peptides were then probed with 2 O772P specific antibodies, O772P-1 (amino acids 44-772 of SEQ ID NO:312) and O772P-2 (477-914 of SEQ ID NO:312; see Example 10 for details of antibody generation), as well as irrelevant rabbit antibodies for controls. The antibodies were diluted to 1 μg/ml and incubated with the membranes for 2 hours at room temperature. The membranes were then washed for 30 minutes in TBS/Tween/Triton buffer, prior to being incubated with a 1:10,000 dilution of HRP-conjugated anti-rabbit secondary antibody for 2 hours. The membranes were again washed for 30 minutes in TBS/Tween/Triton and anti-peptide reactivity was visualized using ECL. Specific epitope binding specificity for each of the O772P-polyclonal antibodies is described in Table 6.

TABLE 6

| SEQ | | | | |
|---|---|---|---|---|
| ID | | | | |
| NO: | Peptide # | Anti-O772P1 | Anti-O772P2 | Peptide Sequence |
| 490 | 2 | *** | — | TCGMRRTCSTLAPGS |
| 491 | 6 | * | */— | CRLTLLRPEKDGTAT |
| 492 | 7 | * | — | DGTATGVDAICTHHP |
| 493 | 8 | — | — | CTHHPDPKSPRLDRE |
| 494 | 9 | *** | *** | RLDREQLYWELSQLT |
| 495 | 11 | */— | — | LGPYALDNDSLFVNG |
| 496 | 13 | **** | — | SVSTTSTPGTPTYVL |
| 497 | 22 | — | — | LRPEKDGEATGVDAI |
| 498 | 24 | ** | */— | DPTGPGLDREQLYLE |
| 499 | 27 | */— | — | LDRDSLYVNGFTHRS |
| 500 | 40 | */— | — | GPYSLDKDSLYLNGY |
| 501 | 41 | — | — | YLNGYNEPGPDEPPT |
| 502 | 47 | *** | *** | ATFNSTEGVLQHLLR |
| 503 | 50 | — | *** | QLISLRPEKDGAATG |
| 504 | 51 | — | ** | GAATGVDTTCTYHPD |
| 505 | 52 | — | */— | TYHPDPVGPGLDIQQ |
| 506 | 53 | — | * | LDIQQLYWELSQLTH |
| 507 | 58 | — | * | HIVNWNLSNPDPTSS |
| 508 | 59 | — | * | DPTSSEYITLLRDIQ |
| 509 | 60 | — | * | LRDIQDKVTTLYKGS |
| 510 | 61 | — | *** | LYKGSQLHDTFRFCL |
| 511 | 71 | — | ** | DKAQPGTTNYQRNKR |

* = relative reactive level, —; no binding, ****; maximal binding

### Example 16

### Identification of a Novel N-Terminal Repeat Structure Associated with O772P

Various O772P cDNA and protein forms have been identified and characterized as detailed above (e.g., Examples 1, 2, 9, and 14). Importantly, O772P RNA and protein have been demonstrated to be over-expressed in ovarian cancer tissue relative to normal tissues and thus represents an attractive target for ovarian cancer diagnostic and therapeutic applications.

Using bioinformatic analysis of open reading frames (ORFs) from genomic nucleotide sequence identified previously as having homology with O772P, multiple nucleotide repeat sequences were identified in the 5′ region of the gene encoding the O772P protein. A number of these repeat sequences were confirmed by RT-PCR using primers specific for the individual repeats. Fragments which contained multiple repeats were amplified from cDNA, thus confirming the presence of specific repeats and allowing an order of these repeats to be established.

Unexpectedly, when various sets of O772P sequences derived from different database and laboratory sources were analyzed, at least 20 different repeat structures, each having substantial levels of identity with each other (see Table 6), were identified in the 5′ region of the O772P gene and the corresponding N-terminal region of the O772P protein. Each repeat comprises a contiguous open reading frame encoding a polypeptide unit that is capable of being spliced to one or more other repeats such that concatomers of the repeats are formed in differing numbers and orders. Interestingly, other molecules have been described in the scientific literature that have repeating structural domains analogous to those described herein for O772P. For example, the mucin family of proteins, which are the major glycoprotein component of the mucous which coats the surfaces of cells lining the respiratory, digestive and urogenital tracts, have been shown to be composed of tandemly repeated sequences that vary in number, length and amino acid sequence from one mucin to another (Perez-Vilar and Hill, J. Biol. Chem. 274(45):31751-31754, 1999).

The various identified repeat structures set forth herein are expected to give rise to multiple forms of O772P, most likely by alternative splicing. The cDNA sequences of the identified repeats are set forth in SEQ ID NOs:513-540, 542-546, and 548-567. The encoded amino acid sequences of the repeats are set forth in SEQ ID NOs:574-593. In many instances these amino acid sequences represent consensus sequences that were derived from the alignment of more than one experimentally derived sequence.

Each of these splice forms is capable of encoding a unique O772P protein with multiple repeat domains attached to a constant carboxy terminal protein portion of O772P that contains a trans membrane region. The cDNA sequence of the O772P constant region is set forth in SEQ ID NO:568 and the encoded amino acid sequence is set forth in SEQ ID NO:594.

All of the available O772P sequences that were obtained were broken down into their identifiable repeats and these sequences were compared using the Clustal method with weighted residue weight table (MegAlign software within DNASTAR sequence analysis package) to identify the relationship between the repeat sequences. Using this information, the ordering data provided by the RT-PCR, and sequence alignments (automatic and manual) using SeqMan (DNASTAR), one illustrative consensus full length O772P contig was identified comprising 20 distinct repeat units. The cDNA for this O772P cDNA contig is set forth in SEQ ID NO:569 and the encoded amino acid sequence is set forth in SEQ ID NO:595. This form of the O772P protein includes the following consensus repeat structures in the following order:

SEQ ID NO:572-SEQ ID NO:574-SEQ ID NO:575-SEQ ID NO:576-SEQ ID NO:577-SEQ ID NO:578-SEQ ID NO:579-SEQ ID NO:580-SEQ ID NO:581-SEQ ID NO:582-SEQ ID NO:583-SEQ ID NO:584-SEQ ID NO:585-SEQ ID NO:586-SEQ ID NO:587-SEQ ID NO:588-SEQ ID NO:589-SEQ ID NO:590-SEQ ID NO:591-SEQ ID NO:592-SEQ ID NO:593.

SEQ ID NO:595, therefore, represents one illustrative full-length consensus sequence for the O772P protein. As discussed above, however, based on current knowledge of this protein and based upon scientific literature describing proteins containing analogous repeating structures, many other forms of O772P are expected to exist with either more or less repeats. In addition, many forms of O772P are expected to have differing arrangements, e.g., different orders, of these N-terminal repeat structures. The existence of multiple forms of O772P having differing numbers of repeats is supported by Northern analysis of O772P. In this study, Northern hybridization of a O772P-specific probe resulted in a smear of multiple O772P-hybridizing transcripts, some in excess 10 kb.

Thus, the variable repeat region of the 0772 protein can be illustratively represented by the structure Xn-Y, wherein X comprises a repeat structure having at least 50% identity with the consensus repeat sequence set forth in SEQ ID NO:596; n is the number of repeats present in the protein and is expected to typically be a integer from 1 to about 35; Y comprise the O772P constant region sequence set forth in SEQ ID NO:594 or sequences having at least 80% identity with SEQ ID NO:594. Each X present in the Xn repeat region of the O772 molecule is different.

To determine the consensus sequences of each of the 20 repeat regions, sequences that were experimentally determined for a discrete repeat region were aligned and a consensus sequence determined. In addition to determining the consensus sequences for individual repeat regions, a consensus repeat sequence was also determined. This sequence was obtained by aligning the 20 individual consensus sequences. Variability of the repeats was determined by aligning the consensus amino acid sequences from each of the individual repeat regions with the over all repeat consensus sequence. Identity data is presented in Table 6.

TABLE 7
Percent identities of Repeat Sequences with
Reference to the Consensus Repeat Sequence

| Repeat Number | | Percent Identity to Consensus |
|---|---|---|
| (amino acid) | SEQ ID NO: | Repeat Sequence |
| 2 | 574 | 88 |
| 3 | 575 | 84 |
| 4 | 576 | 88 |
| 5 | 577 | 89 |
| 6 | 578 | 93 |
| 7 | 579 | 90 |
| 8 | 580 | 91 |
| 9 | 581 | 88 |
| 10 | 582 | 85 |
| 11 | 583 | 86 |
| 12 | 584 | 87 |
| 13 | 585 | 87 |
| 14 | 586 | 89 |
| 15 | 587 | 89 |
| 16 | 588 | 89 |
| 17 | 589 | 83 |
| 18 | 590 | 84 |
| 19 | 591 | 83 |
| 20 | 592 | 57 |
| 21 | 593 | 68 |

### Example 17

### Generation and Characterization of Anti-O772P Monoclonal Antibodies

Monoclonal antibodies were generated against the O772P-2 protein, specifically amino acid residues 447-914 of SEQ ID NO:312. To produce these antibodies, NJ mice were immunized i.p. with 50 μg of recombinant O772P-2 (rO772P-2) mixed with Complete Freud's Adjuvant, followed by a second immunization with 10 μg of rO772P-2 in Incomplete Freud's Adjuvant. Three days prior to the animals being sacrificed, the mice were immunized i.v. with 50 μg of soluble rO772P-2 protein. The spleens from mice with positive titers of O772P-2 were harvested, and a single cell suspension was made and used for fusion to SP2/0 myeloma cells to generate B cell hybridomas.

The supernatants from the resulting hybrid clones were tested by ELISA for specificity to the rO772P-2 protein. Briefly, 96 well plates were coated with rO772P-2 or with one of three 30 mer peptides, which corresponded to the extracellular domain of the O772P protein. Sequences corresponding to these peptides are disclosed in SEQ ID NO:597-599. The rO772P protein or peptides were coated at a concentration of 1-2 μg/ml and 10 μg/ml, respectively, and allowed to incubate for 60 minutes at room temperature. Following coating, the plates were washed five times with PBS containing 0.1% Tween-20, and then blocked with PBS containing 0.5% BSA and 0.4% Tween-20 for 1-2 hours at room temperature. Following the addition of the hybridoma supernatants the plates were again incubated for 60 minutes at room temperature. The plates were then washed as above and donkey-anti-mouse Ig-HRP linked secondary antibody (Jackson ImmunoResearch) was added and incubated for 60 minutes at room temperature, followed by a final wash. TMB peroxidase substrate was then added and incubated for 5-15 minutes at room temperature in the dark. The reaction was stopped by the addition of 1N H₂SO₄ and the optical density was read at 450 nm. Epitope mapping using ELISA revealed that there was 1 hybridoma that recognized peptide #1 (SEQ ID NO:597).

The hybrid supernatants were also tested using flow cytometry to determine if they were capable of recognizing surface expressed epitopes of the O772P protein. Briefly, transiently transfected O772P-08/HEK293, O772P-3rpt/HEK293, O772P-7rpt/HEK293 cells were harvested and washed, followed by incubation with the hybridoma supernatants on ice for 30 minutes. The cells were then washed in staining buffer (1×PBS, 0.5% BSA and 0.01% sodium azide), followed by incubation with goat-anti-mouse Ig-FITC for 30 minutes on ice. The cells were again washed, and resuspended in staining buffer containing 1% propidium iodide (to determine cell viability). The cells were then analyzed for surface expression of the mAbs. Flow cytometry analysis revealed that the 3 of the hybridomas tested were able to detect O772P protein expressed in the surface of O772P-transfected HEK293 cells.

### Example 18

### Characterization of Human Anti-O8E Monoclonal Antibodies

Monoclonal antibodies (mAb) were generated against the O8E protein, the amino acid sequence of which is disclosed in SEQ ID NO:392. To produce these mAbs, Medarex mice were i.p. immunized repeatedly with multiple forms if recombinant O8E antigens, including O8E protein produced using E. coli, O8E plasmid DNA and O8E expressing CHO-1 cells. Spleens from mice with positive titers to O8E were collected and used for fusion to myeloma cells in order to generate B cell hybrids. The supernatants from the resulting hybrids were tested by ELISA for specificity to O8E recombinant protein as well as being tested by flow cytometry for recognition of surface expressed epitopes of the O8E protein and in internalization assays.

For ELISA analysis, 96 well plates were coated with recombinant O8E (rO8E) protein or with pools of 5 over-lapping 20-mer peptides that span the entire O8E molecule (peptides 1-19, the sequences of which are disclosed in SEQ ID NOs:600-618). Recombinant proteins and peptides were coated at a concentration of 1-2 μg/ml and 10 μg/ml, respectively, and then allowed to incubate for 60 minutes at room temperature. After coating, the plates were washed five times with PBS containing 0.1% Tween-20 and then blocked with PBS containing 0.5% BSA and 0.4% Tween-20 for 1-2 hours at room temperature. Following the addition of the hybridoma supernatants, the plates were incubated for 60 minutes at room temperature. The plates were washed as above and mouse anti-human IgG-HRP linked secondary antibody was added and incubated for 60 minutes at room temperature, followed by a final washing as above. TMB peroxidase substrate was added and incubated for 5-15 minutes at room temperature in the dark. The reaction was stopped by the addition of 1N H₂SO₄ and the OD was read at 450 nm. Hybridoma 7F5 was shown to react with peptides 11-15, corresponding to amino acids 151-230. No other hybridoma demonstrated reactivity against the O8E specific peptides. Results from this study are presented in Table 7, column 2.

For flow cytometric analysis, HEK293 cells stably transfected with O8E or SKBR3 cells, which naturally express O8E, were harvested and washed, then incubated with the hybridoma supernatants for 30 minutes on ice. Following this incubation, the cells were washed with staining buffer (1×PBS, 0.5% BSA, and 0.01% sodium azide). Next, mouse anti-human kappa-FITC was added to the cells and allowed to incubate for 30 minutes on ice. Again, the cells were washed followed by resuspension in wash buffer containing 1% propidium iodide. The cells were then subjected to flow cytometric analysis. Results from these experiments are presented in Table 8, column 3.

For internalization assays, 1×10³ SKBR3 cells/well were plated into 96 well plates containing DME plus 10% heat inactivated fetal bovine serum in the presence of 50 ng/well of human anti-O8E hybridoma supernatants or a control antibody. A mouse anti-human Ig-saporin conjugated secondary antibody was then added at various concentrations to the wells, and the plates were incubated for 4 days at 37° C. in a 7% CO₂ incubator. To measure any decreases in the amount of proliferation MTS (20 μl/ml: Promega) was added to the cells for 1 to 2 hours. Proliferation was then measured by reading the OD490 of the plate. The majority of the hybridomas tested were internalized resulting in a decrease in the amount of proliferation detected. The results from these studies are summarized in Table 8, column 6. These findings indicate that the hybridomas can be used to deliver toxins, either directly or indirectly conjugated to the anti-O8E antibodies, to cells that express O8E on their cell surface. This allows for the specific targeting and death of cells that express the ovarian tumor antigen, O8E.

TABLE 8
Summary of anti-O8E hybridoma data

| | ELISA | | HEK | O8E/HEK | |
|---|---|---|---|---|---|
| Clone | (O8E Ag) | Facs* | (MFI) | (MFI) | SKBR3 % death |
| 7H4 | − | + | 2.29 | 96.08 | 50 |
| 9C10 | − | + | 2.85 | 105.98 | 40 |
| 12B9 | − | + | 2.22 | 113.63 | 12 |
| 6F9 | − | + | 4.78 | 100.36 | 25 |
| 11C11 | − | nt | 2.44 | 26.64 | 20 |
| 2A7 | + | + | 2.52 | 153.61 | 25 |
| 5A7 | + | + | 2.45 | 22.76 | 25 |
| 14D2 | − | + | 3.92 | 55.45 | 15 |
| 12B10 | − | + | 2.21 | 23.74 | 40 |
| 12E.1 | + | + | 2.27 | 10.87 | 50 |
| 6E.2 | − | + | 2.45 | 31.89 | 12 |
| 2G6 | − | + | 2.43 | 86.7 | 30.7 |
| 3C7 | − | + | 2.61 | 112.2 | 5.6 |
| 3D8 | − | + | 2.47 | 105.02 | 24.2 |
| 4B8 | − | + | 2.74 | 128 | 29.6 |
| 4G5 | − | + | 2.64 | 68.56 | 14.1 |
| 8D5 | − | + | 2.55 | 131.99 | 8.2 |
| 8G9 | − | + | 2.64 | 123.4 | 18 |
| 9A5 | − | + | 2.43 | 89.43 | 12.3 |
| 9B6 | − | + | 2.51 | 175.81 | 16.3 |
| 14A4 | − | + | 2.52 | 197.33 | 4.5 |
| 18H6 | − | + | 2.58 | 90.29 | −16.2 |
| 19C9 | − | + | 2.44 | 47.11 | −14.1 |
| 20E8 | − | + | 2.51 | 111.2 | −14.9 |
| 21B9 | − | + | 2.46 | 137.36 | −2.2 |
| 22G5 | − | + | 2.88 | 54.66 | 16.3 |
| 23F9 | − | + | 2.53 | 30.02 | 13.5 |
| 24C2 | − | + | 2.59 | 57.9 | 24 |
| 2A7.G4 | + | + | 2.45 | 42.87 | 40.5 |
| 2A7.G7 | + | + | 2.48 | 47.28 | 46.4 |
| 2A7.H5 | + | + | 2.47 | 43.57 | 37 |
| 2A7.H8 | + | + | 2.42 | 45.76 | 40 |
| 2A7.D12 | + | + | 2.67 | 51.83 | 44.3 |
| 1E9 | − | + | 2.67 | 26.14 | 68.5 |
| 2A2 | − | + | 2.59 | 156.82 | 49.4 |
| 2D4 | − | + | 2.56 | 115.79 | 50 |
| 2E4 | − | + | 2.54 | 32.5 | 58.1 |
| 2F9 | − | + | 2.46 | 92.78 | 59.5 |
| 4C7 | − | + | nt | nt | nt |
| 5C6 | − | + | 2.54 | 88.19 | 64.2 |
| 5H5 | − | + | 2.66 | 100.82 | 52 |
| 6F1 | − | + | 2.45 | 57.81 | 49.8 |
| 7H12 | − | + | 2.56 | 172 | 45 |
| 8A10 | − | + | 2.41 | 16.47 | 58.3 |
| 8B7 | − | + | nt | nt | nt |
| 9E11 | − | + | nt | nt | nt |
| 10D4 | − | + | nt | nt | nt |
| 10G7 | − | + | 2.5 | 3.04 | 59.1 |
| 11H8 | − | + | 2.43 | 5.22 | 9 |
| 13A4 | − | + | 2.45 | 53.58 | 37.5 |

MFI = mean fluorescence intensity;
Facs = fluorescence activated cells sorter
Internalization assay - 1% death = average OD (+) toxin/average OD (−) toxins × 100

### Example 19

### Immunohistochemical Analysis Ovarian Cancer Using Human Anti-O8E Monoclonal Antibodies

Anti-O8E immunoreactivity was tested in breast cancer, ovarian cancer and normal tissues. In order to perform this analysis, paraffin embedded formalin fixed tissues were sliced into 8 micron sections. Steam heat induced epitope retrieval (SHIER) in 0.1 M sodium, citrate buffer (pH 6.0) was used for optimal staining conditions. Sections were incubated in PBS containing 10% serum for 5 minutes. Primary O8E mAb (described in detail in Example 18) was then added to each section for 25 minutes followed by a 25 minute incubation with an anti-mouse biotinylated antibody. Endogenous peroxidase was blocked by three 1.5 minute incubations with hydrogen peroxidase. The avidin biotin complex/horse radish peroxidase (ABC/HRP) system was used along with DAB chromogen to visualize antigen expression. Slides were counterstained with hematoxylin.

O8E expression was detected in the majority of ovarian tumor samples but not in normal ovary. O8E expression was also observed in breast cancer samples and at very low levels in normal breast. Of the normal tissues tested (blood vessel, heart, liver, kidney, colon, stomach, skin and lung), only stomach and skin tested positive.

### Example 20

### Expression of O8E Orthologs

The identification and characterization of related forms of the ovarian specific antigen O8E has been described above, one representative DNA sequence for which is set forth in SEQ ID NO:391, encoding amino acid sequences set forth in SEQ ID NOs:392-393. O8E is a plasma membrane associated protein that is over-expressed in ovarian and other cancers. In this example, O8E orthologs from monkey and mouse are identified.

For the cloning of the Rhesus monkey O8E, a PCR primer set was designed using the human O8E open reading frame sequence. The O8E forward primer, designated O8E-UP1, 5′CAGAAGCTTATGGCTTCCCTGGGGCAGACT-3′(SEQ ID NO:619) corresponds to the first 21 nucleotides of the human O8E ORF including the start codon, with a HindIII restriction site added at the 5′-end. The O8E reverse primer, designated O8E-DN1, 5′CAGCGGCCGCTTATTTTAGCATCAGGTAAGG-3′ (SEQ ID NO:620) corresponds to the last 21 nucleotides of the human O8E ORF including the stop codon, with a NotI restriction site added to the 5′-end. The monkey O8E was then amplified from Rhesus monkey placenta cDNA sample using the above-described primer set and cloned into pCEP4 mammalian expression vector. Sequence analysis showed that the O8E sequence derived from the Rhesus (rhO8E) cDNA demonstrated 97.9% identity at the cDNA level and 98.2% identity at the protein level when compared to human O8E. Representative protein and cDNA sequences are disclosed in SEQ ID NOs: 623 and 621, respectively.

For the cloning of the mouse O8E cDNA, the moue EST database was searched using the human O8E sequence. This search resulted in the identification of several mouse ESTs that shared greater than 80% identity to the human O8E cDNA sequence. Three of the mouse EST clones were obtained and sequenced. One clone, 557246, (Genebank Accession number AA117088) contained the full-length cDNA insert for the mouse O8E in a mammalian expression vector, pCMV-SPORT2. Sequence analysis showed that the mouse O8E (rmO8E) shared 82.7% identity at the cDNA level and 86.6% identity at the protein level with human O8E. Representative protein and cDNA sequences are disclosed in SEQ ID NOs:624 and 622, respectively.

### Example 21

### Characterization of the Expression Profile of O8E Orthologs

This example demonstrates that mAbs generated against human O8E were capable of recognizing cells transfected with either Rhesus O8E or mouse O8E.

For expression of the O8E orthologs, HEK293 cells were plated at a density of 250,000 cells/well in DMEM containing 10% FBS and incubated for 4 hours. At the end of the incubation period, 2 μl of Lipofectamine 2000 (Invitrogen) was added to 50 μl of Optimen 1 (Invitrogen) containing no FBS and incubated for 5 minutes at room temperature. In a different tube 50 μl of Optimen 1 was mixed with ˜1.0 μg of pCEP4 vector, pCEP/rhesus monkey O8E, or pCMV-Spert2/mouse O8E plasmid DNA and the mixture was transferred to the Lipofectamine 2000/Optimen mix. The combined mixture was incubated for 20 minutes at room temperature and then transferred to the HEK293 cells containing 2 ml of fresh DMEM containing 10% FBS. The transformed HEK293 cells were incubated for approximately 72 hours at 37° C. with 7% CO₂.

For Fluorescence Activated Cell Sorting (FACS) analysis, cells were collected and washed with ice cold staining buffer (PBS containing 1% BSA and Azide). HEK293/human O8E stable transfectants were used as a positive control for the FACS analysis. The cells were incubated for 45 minutes at room temperature with human mAbs generated against the human O8E protein (identified in Example 18) then washed 2 times with staining buffer followed by incubation with 20 μl of anti-human IgG-FITC reagent (Pharminigen) for 30 minutes ay room temperature. Following 2 washes, the cells were resuspended in staining buffer containing Propidium Iodide (PI), a vital stain that allows for identification of dead cells, and analyzed by FACS.

Cells transfected with human O8E, monkey O8E and mouse O8E demonstrated O8E specific staining with all human O8E mAbs tested. The results of this study are summarized in Table 9.

TABLE 9
Summary of O8E Staining Using Cells Expressing
Human O8E, Mouse O8E, and Rhesus O8E Orthologs

| | Geometric Mean Fluorescent Intensity (GeoMFI) | | | |
|---|---|---|---|---|
| | HEK293- | HEK293- | HEK293- | |
| Human mAb | pCEP4 | huO8E | rmO8E | HEK293-mO8E |
| Human IgG | 24.45 | 24.88 | 38.96 | 49.65 |
| (control) | | | | |
| 1G11 | 26.40 | 275.60 | 301.06 | 300.97 |
| 2A7 | 26.35 | 419.46 | 474.83 | 448.04 |
| 2D4 | 28.57 | 352.71 | 416.72 | 436.11 |
| 2F9 | 28.51 | 381.29 | 446.81 | 426.47 |
| 5A4 | 27.08 | 77.78 | 72.64 | 141.51 |
| 5C6 | 31.47 | 395.61 | 464.40 | 377.79 |
| 5H5 | 32.58 | 220.14 | 302.87 | 323.77 |
| 6F1 | 27.01 | 214.72 | 211.47 | 157.42 |
| 6F9 | 29.22 | 241.65 | 272.48 | 212.39 |
| 7H12 | 27.48 | 365.52 | 416.14 | 403.88 |
| 7H4 | 27.58 | 279.54 | 333.76 | 305.97 |
| 8A10 | 29.59 | 371.75 | 438.83 | 402.82 |
| 9B6 | 26.04 | 267.77 | 442.83 | 415.75 |
| 9C10 | 28.34 | 260.90 | 273.98 | 113.19 |
| 12B10 | 25.38 | 199.61 | 258.99 | 99.53 |
| 12B9 | 23.26 | 272.40 | 284.54 | 116.58 |
| 12E1 | 25.74 | 302.00 | 68.24 | 47.49 |
| 13A12 | 27.03 | 172.35 | 196.80 | 99.19 |
| 13D8 | 25.03 | 386.35 | 462.70 | 430.94 |
| 14A4 | 27.45 | 408.81 | 471.49 | 440.18 |
| 14D2 | 27.58 | 243.74 | 295.29 | 212.84 |
| 15C8 | 30.12 | 392.47 | 448.20 | 440.61 |
| 18H6 | 26.27 | 160.84 | 222.63 | 213.72 |

### Example 22

### Analysis of cDNA Expression Using Real-Time PCR

Real-time PCR (see Gibson et al., Genome Research 6:995-1001, 1996; Heid et al., Genome Research 6:986-994, 1996) is a technique that evaluates the level of PCR product accumulation during amplification. This technique permits quantitative evaluation of mRNA levels in multiple samples. Briefly, mRNA is extracted from tumor and normal tissue and cDNA is prepared using standard techniques. Real-time PCR is performed, for example, using a Perkin Elmer/Applied Biosystems (Foster City, Calif.) 7700 Prism instrument. Matching primers and fluorescent probes are designed for genes of interest using, for example, the primer express program provided by Perkin Elmer/Applied Biosystems (Foster City, Calif.). Optimal concentrations of primers and probes are initially determined by those of ordinary skill in the art, and control (e.g., β-actin) primers and probes are obtained commercially from, for example, Perkin Elmer/Applied Biosystems (Foster City, Calif.). To quantitate the amount of specific RNA in a sample, a standard curve is generated using a plasmid containing the gene of interest. Standard curves are generated using the Ct values determined in the real-time PCR, which are related to the initial cDNA concentration used in the assay. Standard dilutions ranging from 10-10⁶ copies of the gene of interest are generally sufficient. In addition, a standard curve is generated for the control sequence. This permits standardization of initial RNA content of a tissue sample to the amount of control for comparison purposes.

An alternative real-time PCR procedure can be carried out as follows: The first-strand cDNA to be used in the quantitative real-time PCR is synthesized from 20 μg of total RNA that is first treated with DNase I (e.g., Amplification Grade, Gibco BRL Life Technology, Gaitherburg, Md.), using Superscript Reverse Transcriptase (RT) (e.g., Gibco BRL Life Technology, Gaitherburg, Md.). Real-time PCR is performed, for example, with a GeneAmp™ 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence is monitored during the whole amplification process. The optimal concentration of primers is determined using a checkerboard approach and a pool of cDNAs from ovarian tumors is used in this process. The PCR reaction is performed in 25 μl volumes that include 2.5 μl of SYBR green buffer, 2 μI of cDNA template and 2.50 each of the forward and reverse primers for the gene of interest. The cDNAs used for RT reactions are diluted approximately 1:10 for each gene of interest and 1:100 for the β-actin control. In order to quantitate the amount of specific cDNA (and hence initial mRNA) in the sample, a standard curve is generated for each run using the plasmid DNA containing the gene of interest. Standard curves are generated using the Ct values determined in the real-time PCR which are related to the initial cDNA concentration used in the assay. Standard dilution ranging from 20-2×10⁶ copies of the gene of interest are used for this purpose. In addition, a standard curve is generated for β-actin ranging from 200fg-2000fg. This enables standardization of the initial RNA content of a tissue sample to the amount of β-actin for comparison purposes. The mean copy number for each group of tissues tested is normalized to a constant amount of β-actin, allowing the evaluation of the over-expression levels seen with each of the genes.

### Example 23

### Peptide Priming of T-Helper Lines

Generation of CD4⁺ T helper lines and identification of peptide epitopes derived from tumor-specific antigens that are capable of being recognized by CD4⁺ T cells in the context of HLA class II molecules, is carried out as follows:

Fifteen-mer peptides overlapping by 10 amino acids, derived from a tumor-specific antigen, are generated using standard procedures. Dendritic cells (DC) are derived from PBMC of a normal donor using GM-CSF and IL-4 by standard protocols. CD4⁺ T cells are generated from the same donor as the DC using MACS beads (Miltenyi Biotec, Auburn, Calif.) and negative selection. DC are pulsed overnight with pools of the 15-mer peptides, with each peptide at a final concentration of 0.25 μg/ml. Pulsed DC are washed and plated at 1×10⁴ cells/well of 96-well V-bottom plates and purified CD4⁺ T cells are added at 1×10⁵/well. Cultures are supplemented with 60 ng/ml IL-6 and 10 ng/ml IL-12 and incubated at 37° C. Cultures are restimulated as above on a weekly basis using DC generated and pulsed as above as antigen presenting cells, supplemented with 5 ng/ml IL-7 and 10 U/ml IL-2. Following 4 in vitro stimulation cycles, resulting CD4⁺ T cell lines (each line corresponding to one well) are tested for specific proliferation and cytokine production in response to the stimulating pools of peptide with an irrelevant pool of peptides used as a control.

### Example 24

### Generation of Tumor-Specific CTL Lines Using In Vitro Whole-Gene Priming

Using in vitro whole-gene priming with tumor antigen-vaccinia infected DC (see, for example, Yee et al, The Journal of Immunology, 157(9):4079-86, 1996), human CTL lines are derived that specifically recognize autologous fibroblasts transduced with a specific tumor antigen, as determined by interferon-γ ELISPOT analysis. Specifically, dendritic cells (DC) are differentiated from monocyte cultures derived from PBMC of normal human donors by growing for five days in RPMI medium containing 10% human serum, 50 ng/ml human GM-CSF and 30 ng/ml human IL-4. Following culture, DC are infected overnight with tumor antigen-recombinant vaccinia virus at a multiplicity of infection (M.O.I) of five, and matured overnight by the addition of 3 μg/ml CD40 ligand. Virus is then inactivated by UV irradiation. CD8+ T cells are isolated using a magnetic bead system, and priming cultures are initiated using standard culture techniques. Cultures are restimulated every 7-10 days using autologous primary fibroblasts retrovirally transduced with previously identified tumor antigens. Following four stimulation cycles, CD8+ T cell lines are identified that specifically produce interferon-γ when stimulated with tumor antigen-transduced autologous fibroblasts. Using a panel of HLA-mismatched B-LCL lines transduced with a vector expressing a tumor antigen, and measuring interferon-γ production by the CTL lines in an ELISPOT assay, the HLA restriction of the CTL lines is determined.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1.-17. (canceled)

18. An isolated antibody or antigen binding fragment thereof, which specifically binds to the O8E polypeptide of SEQ ID NO: 392 or SEQ ID NO: 393.

19. An isolated antibody or antigen binding fragment thereof of claim 18, which specifically binds to an amino acid sequence selected from the group consisting of SEQ ID NOs: 396-400, 403-406, 409 and 413-415.

20. An isolated antibody or antigen binding fragment thereof of claim 18, which specifically binds to an amino acid sequence selected from the group consisting of SEQ ID NOs: 398, 414 and 415.

21. An isolated antibody or antigen binding fragment of claim 18, wherein the antibody or antigen binding fragment is conjugated to a toxin.

22. An isolated antibody or antigen binding fragment of claim 21, wherein the toxin is selected from the group consisting of a ricin toxin, abrin toxin, diptheria toxin, cholera toxin, gelonin toxin, Pseudomonas exotoxin, Shigella toxin, and pokeweed antiviral protein.

23. An antibody or antigen binding fragment of claim 18, wherein the antibody or antigen binding fragment is a human antibody or antigen binding fragment.

24. An isolated polypeptide comprising an amino acid sequence selected from the group consisting of: SEQ ID NOs: 392, 393, 396-400, 403-406, 409, and 413-415.

25. An isolated polynucleotide sequence encoding a polypeptide of claim 24.

26. A composition comprising a first component selected from (a) an isolated antibody or antigen binding fragment of claim 18; (b) a polypeptide of claim 24, or (c) a polynucleotide of claim 25; and a second component selected from a physiologically acceptable carrier and an immunostimulant.

27. A method for inhibiting the growth of ovarian tumor cells comprising contacting the cells with a composition of claim 26.

28. A method for the treatment of an ovarian cancer in a patient, comprising administering to the patient a composition of claim 26.
